(19) United States
(12) Patent Application Publication
(10) Pub. No.: US 2020/0377510 A1
(43) Pub. Date: Dec. 3, 2020

(54) DERIVATIVES OF PIPERLONGUMINE AND USES THEREOF

(71) Applicant: Auransa Inc., Palo Alto, CA (US)

(72) Inventors: Andrew Asher PROTTER, Palo Alto, CA (US); Pek Yee LUM, Palo Alto, CA (US); Gregory R. LUEDTKE, Palo Alto, CA (US)

(21) Appl. No.: 16/887,785

(22) Filed: May 29, 2020

Related U.S. Application Data
(60) Provisional application No. 62/854,914, filed on May 30, 2019.

(51) Int. Cl. C07D 491/056; C07D 405/10; C07D 211/94; C07D 401/06; C07D 403/06; C07D 401/12; C07D 223/10; C07D 405/06; C07D 405/14; C07D 403/14; C07D 401/14; C07D 405/12; A61P 35/00; A61P 37/02

(52) U.S. Cl. CPC C07D 491/056; C07D 405/10; C07D 211/94; C07D 401/06; C07D 403/06; C07D 401/12; A61P 37/02; C07D 405/06; C07D 405/14; C07D 403/14; C07D 401/14; C07D 405/12; A61P 35/00; C07D 223/10

(57) ABSTRACT

The present invention relates to a group of 1-[(E)-3-(3,4,5-trimethoxyphenyl)prop-2-enoyl]-2,3-dihydropyridin-6-one (piperlongumine) derivatives, analogs and pharmaceutically acceptable salts thereof. The present invention also relates to a pharmaceutical composition and formulation containing a derivative of piperlongumine; and use of the derivatives and analogs for treating cancer, reducing inflammation and/or treating an autoimmune or inflammatory disease.

### CROSS REFERENCE TO RELATED APPLICATIONS

This application claims the benefit of priority to U.S. Provisional Patent Application No. 62/854,914 filed May 30, 2019, the disclosures of which is incorporated herein by reference in its entirety.

### FIELD OF THE INVENTION

The present invention relates to organic compounds useful for therapy or prophylaxis in a subject, and in particular to the treatment of various types of cancer as well as autoimmune or inflammatory diseases.

### BACKGROUND OF THE INVENTION

Piperlongumine is a small molecule alkaloid isolated from a number of Piper plant species, including the long pepper plant Piper longum L. This molecule has gained increasing attention for its antitumor properties and has been reported to be selectively toxic by increasing the level of reactive oxygen species (ROS) in tumor cells and inducing downstream apoptotic cell death. However, the precise mechanism of piperlongumine's effects remains largely unknown.

Piperlongumine is a chalcone-type compound, consisting of two ring systems linked by an α-β-unsaturated carbonyl chain. One of the two rings of piperlongumine is an aromatic phenyl substituted with three methoxy groups, while the second is a 2-piperidinone-type ring containing a conjugated alkene.

Various analogues of piperlongumine having substitutions around the two ring structures as well as in the linker have been described (U.S. Pat. Nos. 9,108,923; 8,318,737; US2009/0312373; US 2012/0059004; WO2016014625; Lad et al., European J. Med. Chem. (2017) 126:870-878; Meegan et al., European J. Med. Chem. (2017) 125:453-463; Wu et al., European J. Med. Chem. (2014) 82:545-551; and Han et al., Org. Biomol. Chem. (2016) 14:7585-7593). Additional analogues of piperlongumine have been described in Zou et al., European J. Med. Chem. (2017) 138:313-319 and Sun et al., Biochem Pharmacol. (2015): 156-169. Although analogues of piperlongumine were reported to exhibit increased activity in vitro, none of these compounds has demonstrated any improvement in vivo over the naturally occurring piperlongumine, particularly their efficacy and other pharmacological properties that are desirable.

Therefore, there exists a need for derivatives and analogues of piperlongumine that exhibit pharmacological properties superior to piperlongumine.

### BRIEF DESCRIPTION OF THE DRAWINGS

FIG. 1 illustrates plasma pharmacokinetics of Compound 1 ((E)-1-(3-(8-methoxy-2,3-dihydrobenzo[b][1,4]dioxin-6-yl)acryloyl)-5,6-dihydropyridin-2(1H)-one) in mice dosed intravenously (5 mg/kg), intraperitoneally (10 mg/kg) and orally (10 mg/kg) (n=5 per each dosing category).

FIGS. 2A-2B show ankle diameters measured over time for each treatment condition (FIG. 2A) and resulting area under the curve (AUC) calculations for days 9-17 (FIG. 2B) for a rat model of Rheumatoid Arthritis treated with an exemplary compound.

FIG. 3 shows measurements of spleen weight relative to body weight for a rat model of Rheumatoid Arthritis (RA) treated with an exemplary compound.

### BRIEF SUMMARY

The present disclosure provides compositions of piperlongumine derivatives and methods of use thereof. The present invention is based on a discovery that derivatives of piperlongumine are useful for treating cancer, reducing inflammation and/or treating autoimmune or inflammatory disease and have other beneficial pharmacological effect, inter alias, bioavailability and metabolic stability to overcome the shortcomings of piperlongumine as a therapeutic compound.

In some embodiments, the compounds and compositions described herein are to be administered to a subject in need of treatment for a cell proliferation disorder such as cancer. Non-limiting examples of cancer include leukemia, lymphoma, lung cancer, liver cancer, gastric cancer, colon cancer, CNS cancer, melanoma, ovarian cancer, renal cancer, prostate cancer, breast cancer, head and neck cancers, brain cancer and pancreatic cancer.

The subject is typically a mammal diagnosed as being in need of treatment for one or more of such proliferative disorders, and frequently the subject is a human. The methods comprise administering an effective amount of at least one compound of the invention; optionally the compound may be administered in combination with one or more additional therapeutic agents, particularly therapeutic agents known to be useful for treating the cancer or proliferative disorder afflicting the particular subject.

In some embodiments, the compounds and compositions described herein are to be administered to a subject in need of reducing inflammation and/or treatment for an autoimmune or inflammatory disease. Non-limiting examples of autoimmune or inflammatory diseases include acute coronary syndrome, acute inflammation, acute respiratory distress syndrome (ARDS), allergic rhinitis, allergy, alopecia areata, Alzheimer's disease, ankylosing spondylitis (AS), antiphospholipid syndrome, asthma, atherosclerosis, atopic allergy, atopic dermatitis, autoimmune Addison's disease, autoimmune chronic active hepatitis, autoimmune hemolytic anemia, autoimmune hepatitis, autoimmune inner ear disease (AIED), autoimmune lymphoproliferative syndrome (ALPS), autoimmune thrombocytopenia, Behcet's disease, bronchial asthma, cardiomyopathy, celiac spruedermatitis hepetiformis, chronic fatigue immune dysfunction syndrome (CFIDS), chronic inflammation, chronic inflammatory demyelinating polyneuropathy (CIPD), chronic obstructive pulmonary disease, cicatricial pemphigold, cold agglutinin disease, contact dermatitis, crest syndrome, Crohn's Disease, Degos' disease, dermatomyositis, type-1 diabetes mellitus, diabetic retinopathy, discoid lupus, eczema, endometriosis, eosinophilic pneumonia, erythema multiforme, essential mixed cryoglobulinemia, fibromyalgia-fibromyositis, food hypersensitivity, glomerulonephritis, gluten-sensitive enteropathy, graft rejection, graft versus host disease, granulomatous hepatitis, Graves' disease, Guillain-Barre syndrome, Hashimoto's thyroiditis, hemolytic anemias, hepatobiliary diseases, hidradenitis suppurativa, hypersensitivity pneumonitis, idiopathic demyelinating polyneuropathy or Guillain-Barre syndrome, idiopathic inflammatory myopathies, idiopathic pulmonary fibrosis, idiopathic thrombocytopenia purpura (ITP), IgA nephropathy, immune mediated renal disease, inflammatory bowel disease (IBD), insulin-dependent diabetes mellitus, ischemic reperfusion, juvenile chronic arthritis, juvenile idiopathic arthritis, juvenile rheumatoid arthritis, Meniere's disease, mixed connective tissue disease, multiple sclerosis, myasthenia gravis, inflammatory autoimmune myositis, osteoarthritis, pemacious anemia, plaque psoriasis (PP), polyarteritis nodosa, polychondritis, polyglandular syndromes, polymyalgia rheumatica, polymyositis, primary agammaglobulinemia, primary biliary cirrhosis, progressive systemic sclerosis, psoriasis, psoriatic arthritis (PsA), Raynaud's phenomena, Reiter's syndrome, rheumatic fever, rheumatoid arthritis (RA), sarcoidosis, scleroderma, progressive systemic sclerosis (PSS), sclerosing cholangitis, sepsis, Sjogren's syndrome, spondyloarthropathy, stiffman syndrome, stroke, systemic lupus erythematosus, systemic sclerosis, systemic vasculitis, Takayasu arteritis, temporal arteritis/giant cell arteritis, thyroiditis, transplant rejection, ulcerative colitis (UC), urticaria, uveitis, vasculitis, ventilator induced lung injury, vitiligo granulomatosis, Wegener's granulomatosis, or Whipple's disease. The methods comprise administering an effective amount of at least one compound of the invention; optionally the compound may be administered in combination with one or more additional therapeutic agents, particularly therapeutic agents known to be useful for reducing inflammation or treating the autoimmune or inflammatory disease afflicting the subject.

The present disclosure provides compounds of Formula (I), and pharmaceutically acceptable salts thereof.

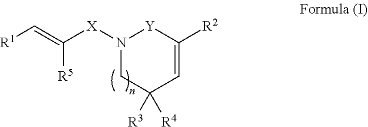

When n is an integer of 1 according to Formula (I), then
X or Y is independently C(O) or S(O)₂;
R¹ is selected from the group consisting of:
   i) a 3-9 member saturated or partially unsaturated cycloalkyl group having 0-5 heteroatom(s);
   ii) a 5 member monocyclic heteroaryl group having 2-3 heteroatoms;
   iii) a 6 member monocyclic heteroaryl group having 1-3 heteroatom(s), wherein said 6 member monocyclic heteroaryl group has:
   a) 1-3 heteroatom(s) when X or Y is S(O)₂ or when at least one of R², R³ and R⁴ is not hydrogen;
   b) two heteroatoms when said 6 member monocyclic heteroaryl group is pyridazinyl or pyrimidyl; or
   c) three heteroatoms when at least one of X and Y is independently C(O);
   iv) a 7-9 member monocyclic unsaturated cycloheteroalkyl group having 1-3 heteroatom(s);
   v) a 7-13 member polycyclic heteroaryl group having 1-5 heteroatom(s), wherein said 7-13 member heteroaryl group has:
   a) 1-5 heteroatom(s) when at least one of X or Y is S(O)₂ and at least one of R², R³ and R⁴ is not hydrogen; or
   b) 2-5 heteroatoms when X and Y are C(O);
   vi) a C₁-C₄ alkyl group;
   vii) a phenyl group when at least one of R², R³ and R⁴ is —CN,
   wherein said heteroatom(s) contained in each cyclic group is independently selected from the group consisting of N, O, and S; and
   wherein each cyclic group is optionally substituted with halo, alkyl, alkenyl, alkynyl, alkoxy, acyl, acyloxy, carboxylic acid, ester, amine, amide, carbonate, carbamate, carbonyl, sulfonyl, cyano, nitro, thioether, thioester, cycloalkyl, cycloheteroalkyl, heterocyclyl, aryl, or heteroaryl, any of which is unsubstituted or substituted with halo, hydroxyl, sulfhydryl, nitro, nitroso, cyano, azido, alkyl, alkenyl, cycloalkyl, cycloheteroalkyl, heterocyclyl, aryl or heteroaryl;
R² is selected from the group consisting of hydrogen, halo, hydroxyl, alkyl, alkenyl, alkynyl, alkoxy, acyl, acyloxy, carboxylic acid, ester, amine, amide, carbonate, carbamate, cyano, nitro, thioether, thioester, cycloalkyl, cycloheteroalkyl, heterocyclyl, aryl, and heteroaryl, each of which is optionally substituted with halo, hydroxyl, sulfhydryl, nitro, nitroso, cyano, azido, alkyl, alkenyl, cycloalkyl, cycloheteroalkyl, heterocyclyl, aryl or heteroaryl;
each of R³ and R⁴ is independently selected from the group consisting of hydrogen, halo, hydroxyl, alkyl, alkenyl, and alkynyl, any of which is optionally substituted with halo, hydroxyl, alkyl, or cycloalkyl, or R³ taken together with one or more atoms of R⁴ forms a cycloalkyl or cycloheteroalkyl, each of which is optionally substituted with halo, hydroxyl, alkyl, alkenyl, alkynyl, or alkoxy; and
R⁵ is selected from the group consisting of hydrogen, halo, hydroxyl, alkyl, alkenyl, alkynyl, alkoxy, acyl, acyloxy, carboxylic acid, ester, amine, amide, carbonate, carbamate, cyano, nitro, thioether, thioester, cycloalkyl, cycloheteroalkyl, heterocyclyl, aryl, and heteroaryl, each of which is optionally substituted with halo, hydroxyl, sulfhydryl, nitro, nitroso, cyano, azido, alkyl, alkenyl, cycloalkyl, cycloheteroalkyl, heterocyclyl, aryl or heteroaryl; or when n is an integer of 0 or 2 according to Formula (I), then
X or Y is independently C(O) or S(O)₂;
R¹ is selected from the group consisting of:
   a) branched C₁-C₄ alkyl (e.g., branched C₂-C₃ alkyl, or branched C₂-C₃ alkyl)
   b) saturated or partially unsaturated C₃-C₉ cycloalkyl having no heteroatom,
   c) saturated, partially unsaturated or unsaturated 3-13 membered heterocyclyl having 1-5 heteroatom(s) independently selected from the group consisting of N, O, and S, and
   d) a phenyl group when at least one of R², R³ and R⁴ is —CN,
   wherein each group is optionally substituted with halo, alkyl, alkenyl, alkynyl, alkoxy, acyl, acyloxy, carboxylic acid, ester, amine, amide, carbonate, carbamate, carbonyl, sulfonyl, cyano, nitro, thioether, thioester, cycloalkyl, cycloheteroalkyl, aryl, heteroaryl, or heterocyclyl, any of which is unsubstituted or substituted with halo, hydroxyl, sulfhydryl, nitro, nitroso, cyano, azido, alkyl, alkenyl, cycloalkyl, cycloheteroalkyl, aryl, heteroaryl, or heterocyclyl;
R² is selected from the group consisting of hydrogen, halo, hydroxyl, alkyl, alkenyl, alkynyl, alkoxy, acyl, acyloxy, carboxylic acid, ester, amine, amide, carbonate, carbamate, cyano, nitro, thioether, thioester, cycloalkyl, cycloheteroalkyl, heterocyclyl, aryl, and heteroaryl, each of which is optionally substituted with halo, hydroxyl, sulfhydryl, nitro, nitroso, cyano, azido, alkyl, alkenyl, cycloalkyl, cycloheteroalkyl, heterocyclyl, aryl or heteroaryl;
each of R³ and R⁴ is independently selected from the group consisting of hydrogen, halo, hydroxyl, alkyl, alkenyl, alkynyl, alkoxy, cycloalkyl, and cycloheteroalkyl, any of which is optionally substituted with halo, hydroxyl, alkyl, or cycloalkyl; or R³ taken together with one or more atoms of R⁴ forms a cycloalkyl or cycloheteroalkyl, each of which is optionally substituted with halo, hydroxyl, alkyl, alkenyl, alkynyl, or alkoxy; and
R⁵ is selected from the group consisting of hydrogen, halo, hydroxyl, alkyl, alkenyl, alkynyl, alkoxy, acyl, acyloxy, carboxylic acid, ester, amine, amide, carbonate, carbamate, cyano, nitro, thioether, thioester, cycloalkyl, cycloheteroalkyl, heterocyclyl, aryl, and heteroaryl, each of which is optionally substituted with halo, hydroxyl, sulfhydryl, nitro, nitroso, cyano, azido, alkyl, alkenyl, cycloalkyl, cycloheteroalkyl, heterocyclyl, aryl or heteroaryl.

In some aspects, each of X and Y is independently C(O) or S(O)₂ according to the provisos that govern the integer n.

In one aspect, R¹ can be a 3-9 member saturated cycloalkyl group having no heteroatom.

In another aspect, R¹ can be a 3-9 member saturated cycloheteroalkyl group having 1-5 heteroatom(s) is selected from the group consisting of aziridinyl, diaziridinyl, oxiranyl, dioxiranyl, oxaziridinyl, thiiranyl, azetidinyl, diazetidinyl, oxetanyl, dioxetanyl, thietanyl, dithietanyl, pyrrolidinyl, imidazolidinyl, pyrazolidinyl, tetrahydropyranyl, tetrahydrofuranyl, oxolanyl, oxazolidinyl, thiolanyl, thiazolidinyl, piperidinyl, piperazinyl, morpholinyl, thiomorpholinyl, oxanyl, dioxanyl, thianyl, dithianyl, hexahydro-1,3,5-triazinyl, trioxanyl, trithianyl, azepanyl, diazepanyl, oxepanyl, thiepanyl, azocanyl, oxocanyl, thiocanyl, azonanyl, oxonanyl, quinolizinyl, and thionanyl.

In one embodiment, R¹ can be piperidinyl, piperazinyl, morpholinyl, thiomorpholinyl, dioxanyl, tetrahydropyranyl, tetrahydrofuranyl, oxanyl, oxolanyl, or oxazolidinyl.

In one aspect, R¹ can be a 3-9 member partially unsaturated cycloalkyl group selected from the group consisting of pyranyl, pyrrolinyl, pyrazolinyl, imidazolinyl, pyridonyl, dihydropyranyl, dihydropyrrolyl, dihydroimidazolyl, dihydropyrimidinyl, dihydropyrazinyl, dihydrotriazinyl, dihydropyridinyl, dihydrooxadiazolyl, thiazolinyl, dioxinyl, oxazinyl, and oxazolyl. In a preferred embodiment, R¹ is pyridonyl, optionally substituted with an alkyl.

In one aspect, R¹ can be a 5 member unsaturated monocyclic heteroaryl group selected from the group consisting of pyrazolyl, imidazolyl, triazolyl, tetrazolyl, thiazolyl, dithiazolyl, isothiazolyl, thiadiazolyl, furazanyl, oxazolyl, isoxazolyl, and oxadiazolyl, with the proviso that R¹ is not furanyl or thiophenyl particularly when Y is S(O)₂. In one embodiment, the 5 member unsaturated monocyclic heteroaryl group is furazanyl. In another embodiment, the 5 member unsaturated monocyclic heteroaryl group is pyrazolyl. In another embodiment, the 5 member unsaturated monocyclic heteroaryl group is triazolyl. In another embodiment, the 5 member unsaturated monocyclic heteroaryl group is imidazolyl. In a preferred embodiment, the 5 member unsaturated monocyclic heteroaryl group is oxadiazolyl, for example, 1,2,3-oxadiazolyl, 1,2,4-oxadiazolyl, and 1,3,4-oxadiazolyl.

In one aspect, R¹ can be a 6 member monocyclic heteroaryl group selected from the group consisting of pyridinyl, diazinyl (e.g., pyrazinyl, pyrimidinyl, or pyridazinyl), triazinyl (e.g., 1,2,3-triazinyl, 1,2,4-triazinyl, or 1,3,5-triazinyl), pyranyl, oxazinyl (e.g., 1,2-oxazinyl; 1,3-oxazinyl, or 1,4-oxazinyl), thiazinyl, dioxinyl, dithiinyl, and triazinyl, with the proviso that R¹ is not pyridinyl or parazinyl when both X and Y are independently C(O).

In one aspect, R¹ can be a 7-9 member unsaturated monocyclic cycloheteroalkyl group having 1-3 heteroatom(s) selected from azepinyl, diazepinyl, oxepinyl, thiepinyl, thiazepinyl, azocinyl, oxocinyl, thiocinyl, azoninyl, oxoninyl, or thioninyl.

In one aspect, R¹ can be a polycyclic heteroaryl group is selected from the group consisting of dihydrobenzodioxinyl, dihydrodioxinopyridinyl, dihydrodioxinopyridazinyl, dihydrodioxinopyrimidinyl, dihydrodioxinopyrazinyl, dihydropyrrolopyridinyl, tetrahydronaphthyridinyl, tetrahydropyridopyridazinyl, tetrahydropyridopyrazinyl, tetrahydropyridopyrimidinyl, benzodioxinyl, purinyl, isoindolyl, isoquinolinyl, quinoxalinyl, quinazolinyl, 1,8-naphthyridinyl, pyrido[3,2-d]pyrimidinyl, pyrido[4,3-d]pyrimidinyl, pyrido[3,4-b]pyrazinyl, pyrido[2,3-b]pyrazinyl, pteridinyl, cinnolinyl, phthalazinyl, benzimidazolyl, phenazinyl, phenoxazinyl, phenothiazinyl, phenoxathiinyl, benzazepinyl, benzodiazepinyl, benzofuranyl, dibenzofuranyl, isobenzofuranyl, benzothiophenyl, benzooxazinyl, quinolin-2(1H)-onyl, isoquinolin-1(2H)-onyl, indazolyl, benzoxazolyl, benzisoxazolyl, benzothiazolyl, dibenzazepinyl, dibenzoxepinyl, dibenzothiazepinyl, dibenzothiepinyl, carbazolyl, and fluorenyl, with the proviso that R¹ is not indolyl or imidazopyridinyl when Y is S(O)₂ or that R¹ is not quinolinyl when both X and Y are C(O). In one aspect, the polycyclic heteroaryl is a 7-13 member polycyclic heteroaryl group formed by covalently bonding a heteroaryl group to a cycloheteroalkyl group, for example, fusing pyridinyl, pyridazinyl, pyrimidinyl, or pyrazinyl with dioxanyl. In one embodiment, R¹ is dihydrobenzodioxinyl. In one embodiment, R¹ is dihydrodioxinopyridinyl. In one embodiment, R¹ is dihydrodioxinopyridazinyl. In one embodiment, R¹ is dihydrodioxinopyrimidinyl. In one embodiment, R¹ is dihydrodioxinopyrazinyl. In one embodiment, R¹ is dihydropyrrolopyridinyl. In one embodiment, R¹ is tetrahydronaphthyridinyl. In one embodiment, R¹ is tetrahydropyridopyridazinyl. In one embodiment, R¹ is tetrahydropyridopyrazinyl. In one embodiment, R¹ is tetrahydropyridopyrimidinyl. In one embodiment, R¹ is benzodioxinyl.

In one aspect, R¹ can be C₁-C₄ alkyl. In some embodiments, R¹ is a C₁-C₃ alkyl group. In one embodiment, R¹ is a branched C₁-C₄ alkyl group, optionally substituted with halo, hydroxyl, alkyl, alkenyl, alkynyl, alkoxy, cycloalkyl, cycloheteroalkyl, aryl, heteroaryl, or heterocyclyl.

In one aspect, R² can be hydrogen, halo, cyano, or alkynyl.

In one aspect, R³ can be hydrogen, halo, hydroxyl, alkyl, alkenyl, or alkynyl.

In one aspect, R⁴ can be hydrogen, halo, hydroxyl, alkyl, alkenyl, or alkynyl.

In another aspect, R³ taken together with one or more atoms of R⁴ forms a cycloalkyl or cycloheteroalkyl.

In yet another aspect, R², R³, or R⁴ is optionally and independently substituted with halo, cyano, hydroxyl, alkyl, alkenyl, alkynyl, alkoxy, cycloalkyl, cycloheteroalkyl, aryl, heteroaryl, or heterocyclyl.

In one aspect, R⁵ is selected from the group consisting of hydrogen, halo, hydroxyl, alkyl, alkenyl, alkynyl, alkoxy, acyl, acyloxy, carboxylic acid, ester, amine, amide, carbonate, carbamate, cyano, nitro, thioether, thioester, cycloalkyl, cycloheteroalkyl, heterocyclyl, aryl, and heteroaryl, each of which is optionally substituted with halo, hydroxyl, sulfhydryl, nitro, nitroso, cyano, azido, alkyl, alkenyl, cycloalkyl, cycloheteroalkyl, heterocyclyl, aryl or heteroaryl.

In one embodiment, R⁵ is hydrogen.

In one embodiment, R⁵ is cyano.

In another embodiment, R⁵ is alkyl optionally substituted with halo, hydroxyl, sulfhydryl, nitro, nitroso, cyano, azido, alkyl, alkenyl, cycloalkyl, cycloheteroalkyl, heterocyclyl, aryl or heteroaryl. In one particular embodiment, R⁵ is methyl. In another particular embodiment, R⁵ is methyl substituted with phenyl. In another embodiment, R⁵ is ethyl.

In one embodiment, R⁵ can be in geometric isomerism. For example, R⁵ can be attached in a cis or trans conformation with respect to R¹.

In some other aspects, n can be any integer of 1, 2, or 3 with the proviso that at least one of X or Y is S(O)₂ and at least one of R², R³ and R⁴ is not hydrogen. In one aspect, R¹ can be a 3-13 member substituted or unsubstituted heterocyclyl group having 1-5 heteroatom(s) independently selected from the group consisting of N, O, and S.

In one aspect, R¹ can be a 3-9 member saturated cycloheteroalkyl group having 1-5 heteroatom(s) selected from the group consisting of aziridinyl, diaziridinyl, oxiranyl, dioxiranyl, oxaziridinyl, thiiranyl, azetidinyl, diazetidinyl, oxetanyl, dioxetanyl, thietanyl, dithietanyl, pyrrolidinyl, imidazolidinyl, pyrazolidinyl, tetrahydropyranyl, tetrahydrofuranyl, oxolanyl, oxazolidinyl, thiolanyl, thiazolidinyl, piperidinyl, piperazinyl, morpholinyl, thiomorpholinyl, oxanyl, dioxanyl, thianyl, dithianyl, hexahydro-1,3,5-triazinyl, trioxanyl, trithianyl, azepanyl, diazepanyl, oxepanyl, thiepanyl, azocanyl, oxocanyl, thiocanyl, azonanyl, oxonanyl, quinolizinyl, and thionanyl. In one embodiment, R¹ can be piperidinyl, piperazinyl, morpholinyl, thiomorpholinyl, dioxanyl, tetrahydropyranyl, tetrahydrofuranyl, oxanyl, oxolanyl, or oxazolidinyl.

In another aspect, R¹ can be a 3-9 member partially unsaturated cycloheteroalkyl group selected from the group consisting of pyranyl, pyrrolinyl, pyrazolinyl, imidazolinyl, pyridonyl, dihydropyranyl, dihydropyrrolyl, dihydroimidazolyl, dihydropyrimidinyl, dihydropyrazinyl, dihydrotriazinyl, dihydropyridinyl, dihydrooxadiazolyl, thiazolinyl, dioxinyl, oxazinyl, and oxazolyl. In a preferred embodiment, R¹ is pyridonyl, optionally substituted with an alkyl.

In another aspect, R¹ can be an unsaturated 3-4 member monocyclic heterocyclyl group having 1-3 heteroatom(s), including, for example, azirinyl, oxirenyl, thiirenyl, diazirinyl, azetyl, diazetyl, oxetyl, dioxetyl, thietyl, and dithietyl.

In another aspect, R¹ can be a 5 member unsaturated monocyclic heteroaryl group having 2-3 heteroatoms. In one embodiment, the 5 member monocyclic heteroaryl group is selected from the group consisting of pyrrolyl, pyrrolinyl, pyrazolyl, pyrazolinyl, imidazolyl, imidazolinyl, triazolyl (e.g., 1,2,3-triazolyl and 1,2,4-triazolyl), tetrazolyl, thiophenyl, thiazolyl, dithiazolyl, thiazolinyl, isothiazolyl, thiadiazolyl, furanyl, furazanyl, oxazolyl, isoxazolyl, and oxadiazolyl (e.g., 1,2,5-oxidiazolyl, 1,2,3-oxidiazolyl, and 1,2,4-oxidiazolyl), optionally substituted. In one embodiment, the 5 member unsaturated monocyclic heteroaryl group is furazanyl. In one embodiment, the 5 member unsaturated monocyclic heteroaryl group is oxadiazolyl, for example, 1,2,3-oxadiazolyl, 1,2,4-oxadiazolyl, and 1,3,4-oxadiazolyl. In one preferred embodiment, the 5 member unsaturated monocyclic heteroaryl group is 1,2,4-oxadiazolyl which is optionally substituted. In another embodiment, the 5 member unsaturated monocyclic heteroaryl group is triazolyl. In one preferred embodiment, the triazolyl is 1,2,3-triazolyl or 1,2,4-triazolyl, each of which is optionally substituted. In yet another embodiment, R¹ is tetrazolyl. In yet another embodiment, R¹ is imidazolyl optionally substituted. In yet another preferred embodiment, R¹ is pyrazonyl optionally substituted. The 5 member monocyclic heteroaryl group may be optionally and independently substituted with halo, alkyl, alkenyl, alkynyl, alkoxy, acyl, acyloxy, carboxylic acid, ester, amine, amide, carbonate, carbamate, carbonyl, cyano, nitro, cycloalkyl, cycloheteroalkyl, heterocyclyl, aryl, or heteroaryl, any of which is optionally substituted with halo, hydroxyl, sulfhydryl, nitro, nitroso, cyano, azido, alkyl, alkenyl, cycloalkyl, cycloheteroalkyl, heterocyclyl, aryl or heteroaryl.

In another aspect, the 3-13 member heterocyclyl group of R¹ can be a 6 member unsaturated monocyclic heteroaryl group selected from the group consisting of pyridyl, pyridinyl, diazinyl (e.g., pyrazinyl, pyrimidinyl, or pyridazinyl), and triazinyl (e.g., 1,2,3-triazinyl, 1,2,4-triazinyl, or 1,3,5-triazinyl).

In another aspect, the 3-13 member heterocyclyl group of R¹ can be a 7-9 member unsaturated monocyclic cycloheteroalkyl having 1-3 heteroatom(s) selected from azepinyl, diazepinyl, oxepinyl, thiepinyl, thiazepinyl, azocinyl, oxocinyl, thiocinyl, azoninyl, oxoninyl, or thioninyl.

In another aspect, the 3-13 member heterocyclyl group of R¹ is a 7-13 member polycyclic heterocycle group having 1-5 heteroatom(s). In one embodiment, the 7-13 member polycyclic heterocycle group can be a polycyclic heteroaryl is selected from the group consisting of dihydrobenzodioxinyl, dihydrodioxinopyridinyl, dihydrodioxinopyridazinyl, dihydrodioxinopyrimidinyl, dihydrodioxinopyrazinyl, dihydropyrrolopyridinyl, tetrahydronaphthyridinyl, tetrahydropyridopyridazinyl, tetrahydropyridopyrazinyl, tetrahydropyridopyrimidinyl, benzodioxinyl, purinyl, isoindolyl, isoquinolinyl, quinoxalinyl, quinazolinyl, 1,8-naphthyridinyl, pyrido[3,2-d]pyrimidinyl, pyrido[4,3-d]pyrimidinyl, pyrido[3,4-b]pyrazinyl, pyrido[2,3-b]pyrazinyl, pteridinyl, cinnolinyl, phthalazinyl, benzimidazolyl, phenazinyl, phenoxazinyl, phenothiazinyl, phenoxathiinyl, benzazepinyl, benzodiazepinyl, benzofuranyl, dibenzofuranyl, isobenzofuranyl, benzothiophenyl, benzooxazinyl, quinolin-2(1H)-onyl, isoquinolin-1(2H)-onyl, indazolyl, benzoxazolyl, benzisoxazolyl, benzothiazolyl, dibenzazepinyl, dibenzoxepinyl, dibenzothiazepinyl, dibenzothiepinyl, carbazolyl, and fluorenyl. In another embodiment, the polycyclic heteroaryl is a 7-13 member polycyclic heteroaryl group formed by covalently bonding a heteroaryl to a dioxanyl, for example, fusing a pyridinyl, pyridazinyl, pyrimidinyl, or pyrazinyl with a dioxanyl. In one particular embodiment, R¹ is dihydrobenzodioxinyl. In another particular embodiment, R¹ is dihydrodioxinopyridinyl. In yet another particular embodiment, R¹ is dihydrodioxinopyridazinyl. In yet another particular embodiment, R¹ is dihydrodioxinopyrimidinyl. In yet another particular embodiment, R¹ is dihydrodioxinopyrazinyl. In yet another embodiment, R¹ is dihydropyrrolopyridinyl. In still another embodiment, R¹ is tetrahydronaphthyridinyl. In still another embodiment, R¹ is tetrahydropyridopyridazinyl. In still another embodiment, R¹ is tetrahydropyridopyrazinyl. In still another embodiment, R¹ is tetrahydropyridopyrimidinyl. In still another embodiment, R¹ is benzodioxinyl.

In one aspect, R² can be selected from hydrogen, halo, hydroxyl, alkyl, alkenyl, alkynyl, alkoxy, acyl, acyloxy, carboxylic acid, ester, amine, amide, carbonate, carbamate, cyano, nitro, thioether, thioester, cycloalkyl, cycloheteroalkyl, aryl, heteroaryl, or heterocyclyl, each of which is optionally substituted with halo, hydroxyl, sulfhydryl, nitro, nitroso, cyano, azido, alkyl, alkenyl, cycloalkyl, cycloheteroalkyl, aryl, heteroaryl or heterocyclyl. In one embodiment, R² can be hydrogen, halo, cyano, or alkynyl.

In one aspect, each of R³ and R⁴ is independently selected from the group consisting of hydrogen, halo, hydroxyl, alkyl, alkenyl, alkynyl, alkoxy, cycloalkyl, and cycloheteroalkyl, any of which is optionally substituted with halo, hydroxyl, alkyl, or cycloalkyl.

In another aspect, R³ taken together with one or more atoms of R⁴ forms a cycloalkyl or cycloheteroalkyl, each of which is optionally substituted with halo, hydroxyl, alkyl, alkenyl, alkynyl, or alkoxy.

In one aspect, R⁵ is selected from the group consisting of halo, hydroxyl, alkyl, alkenyl, alkynyl, alkoxy, acyl, acyloxy, carboxylic acid, ester, amine, amide, carbonate, carbamate, cyano, nitro, thioether, thioester, cycloalkyl, cycloheteroalkyl, heterocyclyl, aryl, and heteroaryl, each of which is optionally substituted with halo, hydroxyl, sulfhydryl, nitro, nitroso, cyano, azido, alkyl, alkenyl, cycloalkyl, cycloheteroalkyl, heterocyclyl, aryl or heteroaryl.

In one embodiment, R⁵ is hydrogen.

In one embodiment, R⁵ is cyano.

In another embodiment, R⁵ is alkyl optionally substituted with halo, hydroxyl, sulfhydryl, nitro, nitroso, cyano, azido, alkyl, alkenyl, cycloalkyl, cycloheteroalkyl, heterocyclyl, aryl or heteroaryl. In one particular embodiment, R⁵ is methyl. In another particular embodiment, R⁵ is methyl substituted with phenyl. In another embodiment, R⁵ is ethyl.

In one embodiment, R⁵ can be in geometric isomerism. For example, R⁵ can be attached in a cis or trans conformation with respect to R¹.

In some embodiments, provided are compounds of Formula (I), and pharmaceutically acceptable salts thereof with the proviso that (1) R¹ is not any one of the group consisting of

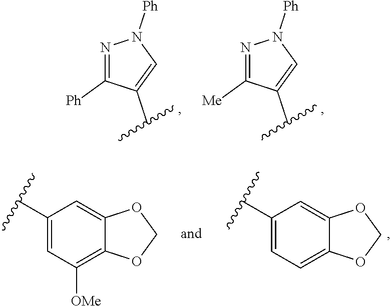 [SMILES: *c1cn(-c2ccccc2)nc1-c1ccccc1 ; *c1cn(-c2ccccc2)nc1C ; *c1cc(OC)c2c(c1)OCO2 ; *c1ccc2c(c1)OCO2]

and Y are C(O) and R², R³, R⁴ and R⁵ are all hydrogen; (2) R is not unsubstituted or substituted pyridinyl, when n is 1, X and Y are C(O), R² is halo, and R³, R⁴ and R⁵ are all hydrogen; and (3) R¹ is not unsubstituted or substituted pyrazinyl, when n is 1, X and Y are C(O), R² is halo, and R³, R⁴ and R⁵ are all hydrogen.

The present invention contemplates pharmaceutical compositions comprising a compound of Formula (I) and a pharmaceutically acceptable carrier. In certain embodiments, such pharmaceutical compositions are formulated for intravenous administration, subcutaneous administration, inhalation, oral administration, rectal administration, parenteral, intravitreal administration, intramuscular administration, intranasal administration, dermal administration, topical administration, otic administration, ophthalmic administration, buccal administration, tracheal administration, bronchial administration, or sublingual administration. In other embodiments, such pharmaceutical compositions are formulated as tablets, pills, solution, capsules, liquid, an inhalant, nasal spray solution, suppository, gel, emulsion, or ointment.

The present invention also contemplates methods for treating an autoimmune or inflammatory disease, comprising administering to a subject in need of such treatment a therapeutically effective amount of a compound of Formula (I), a pharmaceutically acceptable salt thereof, pharmaceutical compositions or medicaments thereof. In one aspect, the present invention provides methods of reducing inflammation in a subject in need thereof with the compound of Formula (I) or a pharmaceutically acceptable salt thereof.

### DETAILED DESCRIPTION

The present disclosure provides a group of 1-[(E)-3-(3,4,5-trimethoxyphenyl)prop-2-enoyl]-2,3-dihydropyridin-6-one (piperlongumine) derivatives and pharmaceutically acceptable salts thereof that are useful in reducing inflammation and/or treating an autoimmune or inflammatory disease. The present disclosure also provides pharmaceutical formulations comprising at least one of the compounds described herein with a pharmaceutically acceptable carrier, diluent or excipient therefor for use in a method disclosed herein.

### Definitions

As used herein, “a” or “an” means “at least one” or “one or more.”

As used herein, “or” means “and/or.”

As used herein, the term “alkyl” refers to saturated hydrocarbon groups in a straight, branched, or cyclic configuration or any combination thereof, and particularly contemplated alkyl groups include those having ten or less carbon atoms, especially 1-6 carbon atoms and lower alkyl groups having 1-4 carbon atoms. Exemplary alkyl groups are methyl, ethyl, propyl, isopropyl, butyl, sec-butyl, tert-butyl, pentyl, isopentyl, hexyl, cyclopropylmethyl, and the like. Alkyl groups can be unsubstituted, or they can be substituted to the extent that such substitution is chemically feasible. Typical substituents include, but are not limited to, halo, ═O, ═N—CN, ═N—OR^{a}, ═NR^{a}, —OR^{a}, —NR^{a}₂, —SR^{a}, —SO₂R^{a}, —SO₂NR^{a}₂, —NR^{a}SO₂R^{a}, —NR^{a}CONR^{a}₂, —NR^{a}COOR^{a}, —NR^{a}COR^{a}, —NO₂, —CN, —COOR^{a}, —CONR^{a}₂, —OOCR^{a}, —COR^{a}, and —R^{a}, wherein each R^{a} is independently H, C₁-C₈ alkyl, C₂-C₈ heteroalkyl, C₃-C₈ heterocyclyl, C₄-C₁₀ heterocycloalkyl, C₁-C₈ acyl, C₂-C₈ heteroacyl, C₂-C₈ alkenyl, C₂-C₈ heteroalkenyl, C₂-C₈ alkynyl, C₂-C₈ heteroalkynyl, C₆-C₁₀ aryl, or C₅-C₁₀ heteroaryl, and each R^{a} is optionally substituted with halo, ═O, ═N—CN, ═N—OR^{b}, ═NR^{b}, —OR^{b}, —NR^{b}₂, —SR^{b}, —SO₂R^{b}, —SO₂NR^{b}₂, —NR^{b}SO₂R, —NR^{b}CONR^{b}₂, —NR^{b}COOR^{b}, —NR^{b}COR^{b}, —NO₂, —CN, —COOR^{b}, —CONR^{b}₂, —OOCR^{b}, —COR^{b}, and —R^{b}, wherein each R^{b} is independently H, C₁-C₈ alkyl, C₂-C₈ heteroalkyl, C₃-C₈ heterocyclyl, C₄-C₁₀ heterocycloalkyl, C₁-C₈ acyl, C₂-C₈ heteroacyl, C₂-C₈ alkenyl, C₂-C₈ heteroalkenyl, C₂-C₈ alkynyl, C₂-C₈ heteroalkynyl, C₆-C₁₀ aryl, or C₅-C₁₀ heteroaryl. Alkyl, alkenyl and alkynyl groups can also be substituted by C₁-C₈ acyl, C₂-C₈ heteroacyl, C₆-C₁₀ aryl or C₅-C₁₀ heteroaryl, each of which can be substituted by the substituents that are appropriate for the particular group. Where a substituent group contains two R^{a} or R^{b} groups on the same or adjacent atoms (e.g., —NR^{b}₂, or —NR^{b}—C(O)R^{b}), the two R^{a} or R^{b} groups can optionally be taken together with the atoms in the substituent group to which are attached to form a ring having 5-8 ring members, which can be substituted as allowed for the R^{a} or R^{b} itself, and can contain an additional heteroatom (N, O or S) as a ring member.

As used herein, the term “alkenyl” refers to hydrocarbon chain having at least two carbon atoms and at least one carbon-carbon double bond and includes straight, branched, or cyclic alkenyl groups having two to ten carbon atoms. Non-limiting examples of “alkenyl” include ethenyl, propenyl, butenyl, pentenyl, and cyclic alkenyl groups. An alkenyl can be unsubstituted or substituted with one or more suitable substituents.

As used herein, the term “alkynyl” refers to unbranched and branched hydrocarbon moieties having at least two (preferably three) carbon atoms and at least one carbon-carbon triple bond and includes ethynyl, propynyl, butynyl, cyclopropylethynyl, and the like. An alkynyl can be unsubstituted or substituted with one or more suitable substituents.

As used herein, the term “alkoxy” refers to the alkyl groups above bound through oxygen, examples of which include methoxy, ethoxy, propyloxy, isopropoxy, tert-butoxy, methoxyethoxy, benzyloxy, allyloxy, and the like. In addition, alkoxy also refers to polyethers such as —O— (CH₂)₂O—CH₃, and the like. An alkoxy can be any hydrocarbon group connected through an oxygen atom wherein the hydrocarbon portion may have any number of carbon atoms, typically 1-10 carbon atoms, may further include a double or triple bond and may include one or two oxygen, sulfur or nitrogen atoms in the alkyl chains. An alkoxy can be unsubstituted or substituted with one or more suitable substituents, e.g., aryl, heteroaryl, cycloalkyl, and/or heterocyclyl.

As used herein, the term “cycloalkyl” refers to cyclic alkane in which a chain of carbon atoms of a hydrocarbon forms a ring, and includes a monocyclic or polycyclic hydrocarbon ring group, for example, cyclopropyl, cyclobutyl, cyclopentyl, cyclohexyl, cycloheptyl, cyclooctyl, cyclodecyl, adamantyl, norpinanyl, decalinyl, norbornyl, housanyl, and the like. Further, a cycloalkyl can also include one or two double bonds, which form the “cycloalkenyl” groups (e.g., cyclopropenyl, cyclobutenyl, cyclopentenyl, cyclohexenyl, cycloheptenyl, cyclooctenyl, norbornenyl, norbornadienyl, and the like). A cycloalkyl can also comprise one or more heteroatoms and referred to as “cycloheteroalkyl” and can include, for example, piperazinyl piperidinyl, morpholinyl, thiomorpholinyl, oxanyl, dioxanyl (e.g., 1,4-dioxanyl), thianyl, dithianyl, hexahydro-1,3,5-triazinyl, trioxanyl, trithianyl, pyrrolidinyl, imidazolidinyl, pyranyl, tetrahydropyranyl, pyrazolidinyl, oxolanyl, oxazolidinyl, thiolanyl, thiazolidinyl, pyrrolinyl, pyrazolinyl, imidazolinyl, tetrahydrofuranyl, and the like. A cycloalkyl or cycloheteroalkyl group can be unsubstituted or substituted with one or more suitable substituents.

As used herein, the term “hetero” refers to an atom of any element other than carbon or hydrogen. As used herein, the term “heteroatom” means nitrogen (N), oxygen (O), or sulfur (S).

As used herein, the term “heterocycle” or “heterocyclyl” encompasses all limitations of “cycloheteroalkyl” and “heteroaryl” groups in so far as chemically feasible. The term “heterocycle” or “heterocyclyl” refers to any compound in which a plurality of atoms forms a ring via a plurality of covalent bonds, wherein the ring includes at least one atom other than a carbon atom as a ring member. A heterocycle can be saturated, unsaturated, or partially unsaturated. An unsaturated heterocycle can be aromatic aryl. Non-limiting examples of a heterocyclic ring include 3-, 4-, 5-, 6-, 7-, 8- and 9-membered monocyclic rings containing one or more N, O, or S as the non-carbon member(s) and are as follows: (1) a saturated 3 atom heterocyclic ring can be, for example, aziridinyl, diaziridinyl, oxiranyl, dioxiranyl, oxaziridinyl, thiiranyl, or the like, and an unsaturated 3 atom heterocyclic ring can be, for example, azirinyl, oxirenyl, thiirenyl, diazirinyl, or the like; (2) a saturated 4 atom heterocyclic ring can be, for example, azetidinyl, diazetidinyl, oxetanyl, dioxetanyl, thietanyl, dithietanyl, or the like, and an unsaturated 4 atom heterocyclic ring can be, for example, azetyl, diazetyl, oxetyl, dioxetyl, thietyl, dithietyl, or the like; (3) a saturated 5 atom heterocyclic ring can be, for example, pyrrolidinyl, imidazolidinyl, pyrazolidinyl, oxolanyl, oxazolidinyl, thiolanyl, thiazolidinyl, or the like, and an unsaturated and partially unsaturated 5 atom heterocyclic ring can be, for example, pyrrolyl, pyrrolinyl, pyrazolyl, pyrazolinyl, imidazolyl, imidazolinyl, triazolyl, tetrazolyl, thiophenyl, thiazolyl, dithiazolyl, thiazolinyl, isothiazolyl, thiadiazolyl, furanyl, furazanyl, oxazolyl, isoxazolyl, oxadiazolyl, or the like; (4) a saturated 6 atom heterocyclic ring can be, for example, piperidinyl, piperazinyl, morpholinyl, thiomorpholinyl, oxanyl, dioxanyl (e.g., 1,4-dioxacyclohexane), thianyl, dithianyl, hexahydro-1,3,5-triazinyl, trioxanyl, trithianyl, or the like, and an unsaturated 6 atom heterocyclic ring can be, for example, pyridinyl, diazinyl (e.g., pyrimidinyl, or pyridazinyl), pyranyl, oxazinyl (e.g., 1,2-oxazinyl; 1,3-oxazinyl, or 1,4-oxazinyl), thiazinyl, 1,4-dioxinyl, dithiinyl, triazinyl (e.g., 1,2,3-triazinyl, 1,2,4-triazinyl, or 1,3,5-triazinyl), tetrazinyl, pentazinyl, thiopyranyl, or the like; (5) a saturated 7 atom heterocyclic ring can be, for example, azepanyl, diazepanyl, oxepanyl, thiepanyl, or the like, and an unsaturated 7 atom heterocyclic ring can be, for example, azepinyl, diazepinyl, oxepinyl, thiepinyl, thiazepinyl, or the like; (6) a saturated 8 atom heterocyclic ring can be, for example, azocanyl, oxocanyl, thiocanyl, or the like, and an unsaturated 8 atom heterocyclic ring can be, for example, azocinyl, oxocinyl, thiocinyl, or the like; and (7) a saturated 9 atom heterocyclic ring can be, for example, azonanyl, oxonanyl, thionanyl, or the like, and an unsaturated 9 atom heterocyclic ring can be, for example, azoninyl, oxoninyl, thioninyl, or the like. Further contemplated heterocycles may be fused, for example, covalently bound with two atoms on the first non-heterocyclic group (e.g., phenyl) to one or two heterocycles (e.g., 1,4-dioxanyl, 1,4-dioxinyl, and tetrahydropyranyl), or covalently bound with two atoms on the first heterocyclic ring (e.g., pyrrolyl, imidazolyl, thiazolyl, pyrimidinyl, and pyridinyl) to one or two nonheterocyclic or heterocyclic group (e.g., 1,4-dioxanyl, 1,4-dioxinyl, and morpholinyl), and taken together are thus termed “fused heterocycle” or “fused heterocyclic moieties” or “heteroaryl-fused-cycloheteroalkyl” as used herein. The fused heterocycle can be, for example, a saturated or unsaturated (e.g., aromatic) bicyclic or tricyclic compound. Non-limiting examples of fused heterocycle include dihydrobenzodioxinyl, dihydrodioxinopyridinyl, dihydrodioxinopyridazinyl, dihydrodioxinopyrimidinyl, dihydrodioxinopyrazinyl, dihydropyrrolopyridinyl, tetrahydronaphthyridinyl, tetrahydropyridopyridazinyl, tetrahydropyridopyrazinyl, tetrahydropyridopyrimidinyl, chromanyl, indolyl, purinyl, isoindolyl, quinolinyl, isoquinolinyl, quinoxalinyl, quinazolinyl, quinolizinyl, 1,8-naphthyridinyl, pyrido[3,2-d]pyrimidinyl, pyrido[4,3-d]pyrimidinyl, pyrido[3,4-b]pyrazinyl, pyrido[2,3-b]pyrazinyl, pteridinyl, acridinyl, cinnolinyl, phthalazinyl, benzimidazolyl, phenazinyl, phenoxazinyl, phenothiazinyl, phenoxathiinyl, benzazepinyl, benzodiazepinyl, benzofuranyl, dibenzofuranyl, isobenzofuranyl, benzothiophenyl, benzoxazinyl, quinolin-2(1H)-onyl, isoquinolin-1(2H)-onyl, indazolyl, benzoxazolyl, benzisoxazolyl, benzothiazolyl, dibenzazepinyl, dibenzoxepinyl, dibenzothiazepinyl, dibenzothiepinyl, carbazolyl, fluorenyl, and the like. Where the heterocyclic ring is aromatic, it can be also referred to herein as “heteroaryl” or “heteroaromatic” as described further below. A heterocyclic ring that is not aromatic can be substituted with any group suitable for alkyl group substituents described above.

As used herein, the term “aryl” refers to unsubstituted or substituted aromatic monocyclic or polycyclic groups, which may further include one or more non-carbon atoms. The term “aryl” also includes aromatic rings fused to non-aromatic carbocyclic ring, or to a heterocyclyl group having 1-7 heteroatoms. The term “aryl” may be interchangeably used with “aryl ring,” “aromatic group,” and “aromatic ring.” An aryl group may contain 1-9 heteroatom(s) that are generally referred to as “heteroaryl.” Heteroaryl groups typically have 4 to 14 atoms, 1 to 9 of which are independently selected from the group consisting of N, O, and S. In a 5-8 membered aromatic group, for example, a heteroaryl group can contain 1-4 heteroatoms. An aryl or heteroaryl can be unsubstituted or substituted with one or more suitable substituents.

An aryl or heteroaryl can be a mono- or polycyclic (e.g., bicyclic) aromatic group. Typical aryl groups include, for example, phenyl and naphthalenyl and the like. Typical heteroaryl groups include, for example, quinolinyl, pyrrolyl, pyrazolyl, imidazolyl, triazolyl, tetrazolyl, thiophenyl, thiazolyl, dithiazolyl, thiazolinyl, isothiazolyl, thiadiazolyl, furanyl, furazanyl, oxazolyl, isoxazolyl, oxadiazolyl, pyridinyl, diazinyl (e.g., pyrazinyl, pyrimidinyl, or pyridazinyl), triazinyl (e.g., 1,2,3-triazinyl, 1,2,4-triazinyl, or 1,3,5-triazinyl), pyranyl, oxazinyl (e.g., 1,2-oxazinyl; 1,3-oxazinyl, or 1,4-oxazinyl), thiazinyl, dioxinyl, dithiinyl, triazinyl, tetrazinyl, pentazinyl, thiopyranyl, azepinyl, diazepinyl, oxepinyl, thiepinyl, thiazepinyl, azocinyl, oxocinyl, thiocinyl, azoninyl, oxoninyl, thioninyl, indolyl, indazolyl, purinyl, isoindolyl, quinolinyl, isoquinolinyl, quinoxalinyl, acridinyl, quinazolinyl, cinnolinyl, phthalazinyl, benzimidazolyl, benzofuranyl, isobenzofuranyl, benzoxazolyl, benzisoxazolyl, benzothiazolyl, or the like. Polycyclic aryl or polycyclic heteroaryl groups can be formed by fusing (i.e., covalently bonding) 2 atoms on the first aryl or heteroaryl ring with at least one carbocyclic or heterocyclic group, and are thus termed “fused aryl” or “heteroaryl-fused-cycloheteroalkyl.”

As used herein, the term “heteroaryl-fused-cycloheteroalkyl” refers to a heterocyclyl moiety consisting of a monocyclic heteroaryl group, such as pyridyl or furanyl, fused to a cycloheteroalkyl group, in which the heteroaryl and cycloheteroalkyl parts are as defined herein. Exemplary heteroaryl-fused-heterocycloalkyl groups include dihydrodioxinopyridinyl, dihydrodioxinopyridazinyl, dihydrodioxinopyrimidinyl, dihydrodioxinopyrazinyl, dihydrodioxinotriazinyl, dihydropyrrolopyridinyl, dihydrofuranopyridinyl and dioxolopyridinyl. The heteroaryl-fused-heterocycloalkyl group may be attached to the remainder of the molecule by any available carbon or nitrogen atom.

Typical heteroaryl groups include 5 or 6 member monocyclic aromatic groups such as pyridyl, pyrimidyl, pyrazinyl, pyridazinyl, thienyl, furanyl, pyrrolyl, pyrazolyl, thiazolyl, oxazolyl, isothiazolyl, isoxazolyl, thiophenyl, triazolyl (1,2,4-triazolyl and 1,2,3-triazolyl), tetrazolyl, furazanyl, oxadiazolyl (1,2,5-oxadiazolyl and 1,2,3-oxadiazolyl), and imidazolyl and the fused bicyclic moieties formed by fusing one of heterocyclic groups with a phenyl ring or with any of the heteroaromatic monocyclic groups include indolyl, benzimidazolyl, indazolyl, benzotriazolyl, isoquinolyl, quinolyl, benzothiazolyl, benzofuranyl, pyrazolopyridyl, pyrazolopyrimidyl, quinazolinyl, quinoxalinyl, cinnolinyl, imidazopyrimidinyl, and the like.

As used herein, the term “monocyclic” refers to an unsubstituted or substituted single ring structure. As used herein, the terms “polycyclic” and “bicyclic” refer to an unsubstituted or substituted poly-ring structure that comprises at least two ring structures fused by any two adjacent atoms. A bicyclic ring can be an aryl or heteroaryl ring fused to an aromatic ring or a non-aromatic carbocyclic ring such as cycloalkyl or cycloheteroalkyl. A bicyclic ring can be also non-aromatic carbocyclic ring fused to another non-aromatic carbocyclic ring such as cycloalkyl or cycloheteroalkyl. Non-limiting examples of bicyclic rings include dihydrobenzodioxinyl, dihydrodioxinopyridinyl, dihydrodioxinopyridazinyl, dihydrodioxinopyrimidinyl, dihydrodioxinopyrazinyl, dihydropyrrolopyridinyl, tetrahydronaphthyridinyl, tetrahydropyridopyridazinyl, tetrahydropyridopyrazinyl, tetrahydropyridopyrimidinyl, chromanyl, decalinyl, purinyl, indolyl, isoindolyl, quinolyl, quinazolinyl, benzimidazolyl, imidazopyridyl, cinnolinyl, phthalazinyl, imidazopyrimidinyl, and the like. Any monocyclic or fused bicyclic system which has the characteristics of aromaticity in terms of electron distribution throughout the ring system is included in this definition. It also includes bicyclic groups where at least the ring which is directly attached to the remainder of the molecule has the characteristics of aromaticity.

Aryl and heteroaryl groups can be substituted where permitted. Suitable substituents include, but are not limited to, halo, R^{a}, —OR^{a}, —NR^{a}₂, —SR^{a}, —SO₂R^{a}, —SO₂NR^{a}₂, —NR^{a}SO₂R^{a}, —NR^{a}CONR^{a}₂, —NR^{a}COOR^{a}, —NR^{a}COR^{a}, —CN, —COOR^{a}, —CONR^{a}₂, —OOCR^{a}, —COR^{a}, and —NO₂, wherein each R^{a} is independently H, C₁-C₈ alkyl, C₂-C₈ heteroalkyl, C₃-C₈ heterocyclyl, C₄-C₁₀ heterocycloalkyl, C₁-C₈ acyl, C₂-C₈ heteroacyl, C₂-C₈ alkenyl, C₂-C₈ heteroalkenyl, C₂-C₈ alkynyl, C₂-C₈ heteroalkynyl, C₆-C₁₀ aryl, or C₅-C₁₀ heteroaryl, and each R^{a} is optionally substituted with halo, ═O, ═N—CN, ═N—OR^{b}, ═NR^{b}, —OR^{b}, —NR^{b}₂, —SR^{b}, —SO₂R^{b}, —SO₂NR^{b}₂, —NR^{b}SO₂R, —NR^{b}CONR^{b}₂, —NR^{b}COOR^{b}, —NR^{b}COR^{b}, —CN, —COOR^{b}, —CONR^{b}₂, —OOCR^{b}, —COR^{b}, and —NO₂, wherein each R^{b} is independently H, C₁-C₈ alkyl, C₂-C₈ heteroalkyl, C₃-C₈ heterocyclyl, C₄-C₁₀ heterocycloalkyl, C₁-C₈ acyl, C₂-C₈ heteroacyl, C₂-C₈ alkenyl, C₂-C₈ heteroalkenyl, C₂-C₈ alkynyl, C₂-C₈ heteroalkynyl, C₆-C₁₀ aryl, or C₅-C₁₀ heteroaryl. Alkyl, alkenyl and alkynyl groups can also be substituted by C₁-C₈ acyl, C₂-C₈ heteroacyl, C₆-C₁₀ aryl or C₅-C₁₀ heteroaryl, each of which can be substituted by the substituents that are appropriate for the particular group. Where a substituent group contains two R^{a} or R^{b} groups on the same or adjacent atoms (e.g., —NR^{b}₂, or —NR^{b}—C(O)R^{b}), the two R^{a} or R^{b} groups can optionally be taken together with the atoms in the substituent group to which are attached to form a ring having 5-8 ring members, which can be substituted as allowed for the R^{a} or R^{b} itself, and can contain an additional heteroatom (N, O or S) as a ring member.

The term “sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cycloalkenyl, SO₂-aryl, SO₂-substituted aryl, SO₂-heteroaryl, SO₂-substituted heteroaryl, SO₂-heterocyclic, and SO₂-substituted heterocyclic, wherein each alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

As used herein, the term “acyl” when used without the “substituted” modifier refers to the group —C(O)R, in which R is a hydrogen, alkyl, aryl, halides, aralkyl or heteroaryl, as those terms are defined herein.

As used herein, the term “acyloxy” refers a straight-chain or branched alkanoyl group having 1 to 6 carbon atoms, such as formyl, acetyl, propanoyl, butyryl, valeryl, pivaloyl and hexanoyl, and arylcarbonyl group described below, or a heteroarylcarbonyl group described below. The aryl moiety of the arylcarbonyl group means a group having 6 to 16 carbon atoms such as phenyl, biphenyl, naphthyl, or pyrenyl. The heteroaryl moiety of the heteroarylcarbonyl group contains at least one hetero atom from 0, N, and S, such as pyridyl, pyrimidyl, pyrroleyl, furyl, benzofuryl, thienyl, benzothienyl, imidazolyl, triazolyl, quinolyl, iso-quinolyl, benzoimidazolyl, thiazolyl, benzothiazolyl, oxazolyl, and indolyl.

As used herein, the term “carboxylic acid” refers to a group —C(O)OH.

As used herein, the term “ester,” as used herein, refers to a group —C(O)O—.

As used herein, the term “nitro” means —NO₂.

As used herein, the term “cyano” means —CN.

As used herein, the term “azido” means relating to a monovalent group containing —N₃.

As used herein, the term “sulfhydryl” means thiol, —SH.

As used herein, the term “amine” means primary, secondary and tertiary amines, —R—NH₂, —R—NH—R′, and —R—N—(R″)R′, respectively.

As used herein, the term “amide” means primary, secondary and tertiary amides, —R—C(O)NH₂, —R—C(O)NH—R′, and —R—C(O)NR′R″, respectively.

As used herein, the term “carbonate” means ester of carbonic acid, a group containing C(═O)(O—)₂.

As used herein, the term “carbamate” means a group containing NH₂COOH.

As used herein, the term “hydroxyl” means —OH.

As used herein, the terms “halo,” “halogen,” and “halide” mean fluoro (—F), chloro (—Cl), bromo (—Br), and iodo (—I).

As used herein, the term “haloalkyl” refers to any alkyl having one or more hydrogen atoms replaced by one or more halogen atoms. Non-limiting examples of haloalkyl include —CF₃, —CFH₂, —CF₂H, and the like.

As used herein, the term “arylalkyl” refers to any alkyl in which one or more hydrogen atoms are replaced by an aryl or heteroaryl group. Examples of arylalkyl include benzyl (C₆H₅CH₂—) and the like.

As used herein, the term “hydroxyalkyl” refers to any hydroxy derivative of alkyl and includes any alkyl having one or more hydrogen atoms replaced by a —OH group.

The term “haloalkyl” refers to an alkyl group as described above with one or more hydrogen atoms on the alkyl group substituted with a halo group. Examples of such groups include, without limitation, fluoroalkyl groups, such as fluoroethyl, difluoromethyl, trifluoromethyl, trifluoroethyl and the like.

The term “haloalkoxy” refers to the group alkyl-O— with one or more hydrogen atoms on the alkyl group substituted with a halo group (e.g., —F, —Cl, —Br, and —I) and include, for example, groups such as trifluoromethoxy and the like.

The term “substituted” as used herein refers to a replacement of a hydrogen atom of the unsubstituted group with a functional group, and particularly contemplated functional groups include nucleophilic groups (e.g., —NH₂, —OH, —SH, —CN, etc.), electrophilic groups (e.g., C(O)OR, C(X)OH, etc.), polar groups (e.g., —OH), non-polar groups (e.g., heterocycle, aryl, alkyl, alkenyl, alkynyl, etc.), ionic groups (e.g., —NH₃+), and halogens (e.g., —F, —Cl), NHCOR, NHCONH₂, OCH₂COOH, OCH₂CONH₂, OCH₂CONHR, NHCH₂COOH, NHCH₂CONH₂, NHSO₂R, OCH₂-heterocycles, PO₃H, SO₃H, amino acids, and all chemically reasonable combinations thereof. Moreover, the term “substituted” also includes multiple degrees of substitution, and where multiple substituents are disclosed or claimed, the substituted compound can be independently substituted by one or more of the disclosed or claimed substituent moieties.

Unless indicated otherwise, the nomenclature of substituents that are not explicitly defined herein are arrived at by naming the terminal portion of the functionality followed by the adjacent functionality toward the point of attachment. For example, the substituent “alkylaryloxycarbonyl” refers to the group (alkyl)-(aryl)-O—C(O)—.

As to any of the groups disclosed herein which contain one or more substituents, it is understood that such groups do not contain any substitution or substitution patterns which are sterically impractical and/or synthetically non-feasible. In addition, the subject compounds include all stereochemical isomers arising from the substitution of these compounds.

In addition to the disclosure herein, in a certain embodiment, a group that is substituted has 1 substituent, 1 or 2 substituents, 1, 2, or 3 substituents, or 1, 2, 3, or 4 substituents.

As used herein, the term “administration” or “administering” of the subject compound refers to providing a compound of the invention to a subject in need of treatment.

Unless clearly indicated otherwise, “a subject” as used herein intends a mammal, including but not limited to a primate, human, bovine, horse, feline, canine, or rodent. In one variation, the subject is a human.

As used herein, “treatment” or “treating” is an approach for obtaining beneficial or desired results including clinical results. Beneficial or desired results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, delaying the occurrence or recurrence of the disease, delay or slowing the progression of the disease, ameliorating the disease state, providing a remission (whether partial or total) of the disease, decreasing the dose of one or more other medications required to treat the disease, enhancing effect of another medication, delaying the progression of the disease, increasing the quality of life, and/or prolonging survival. The methods described herein contemplate any one or more of these aspects of treatment.

As used herein, the term “effective amount” intends such amount of a compound of the invention which should be effective in a given therapeutic form. As is understood in the art, an effective amount may be in one or more doses, i.e., a single dose or multiple doses may be required to achieve the desired treatment. An effective amount may be considered in the context of administering one or more therapeutic agents, and a single agent may be considered to be given in an effective amount if, in conjunction with one or more other agents, a desirable or beneficial result may be or is achieved. Suitable doses of any of the co-administered compounds may optionally be lowered due to the combined action (e.g., additive or synergistic effects) of the compounds.

A “therapeutically effective amount” refers to an amount of a compound or salt thereof sufficient to produce a desired therapeutic outcome.

As used herein, “unit dosage form” refers to physically discrete units, suitable as unit dosages, each unit containing a predetermined quantity of active ingredient calculated to produce the desired therapeutic effect in association with the required pharmaceutical carrier. Unit dosage forms may contain a single or a combination therapy.

As used herein, the term “acceptable” with respect to a formulation, composition or ingredient, as used herein, means having no persistent detrimental effect on the general health of the subject being treated.

The term “about” when referring to a number or a numerical range means that the number or numerical range referred to is an approximation within experimental variability (or within statistical experimental error), and thus the number or numerical range may vary from, for example, between 1% and 10% of the stated number or numerical range.

As used herein, the term “carrier” refers to chemical compounds or agents that facilitate the incorporation of a compound described herein into cells or tissues.

As used herein, the terms “comprise,” “have,” and “include” are open-ended linking verbs. Any forms or tenses of one or more of these verbs, such as “comprises,” “comprising,” “has,” “having,” “includes,” and “including” are open-ended. For example, any method that “comprises,” “has,” or “includes” one or more moieties is not limited to possessing only those one or more moieties and also covers other unlisted moieties.

### Derivatives of Piperlongumine

Disclosed herein is a class of compounds having Formula (I), pharmaceutical acceptable salts, protected derivatives, individual isomers and mixture of isomers thereof.

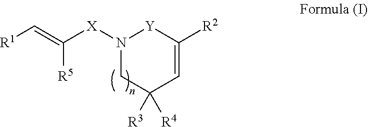

When n is an integer of 1 according to Formula (I), then
X or Y is independently C(O) or S(O)₂;
R¹ is selected from the group consisting of:
   i) a 3-9 member saturated or partially unsaturated cycloalkyl group having 0-5 heteroatom(s);
   ii) a 5 member monocyclic heteroaryl group having 2-3 heteroatoms;
   iii) a 6 member monocyclic heteroaryl group having 1-3 heteroatom(s), wherein said 6 member monocyclic heteroaryl group has:
   a) 1-3 heteroatom(s) when X or Y is S(O)₂ or when at least one of R², R³ and R⁴ is not hydrogen;
   b) two heteroatoms when said 6 member monocyclic heteroaryl group is pyridazinyl or pyrimidyl; or
   c) three heteroatoms when X or Y is independently C(O);
   iv) a 7-9 member monocyclic unsaturated cycloheteroalkyl group having 1-3 heteroatom(s);
   v) a 7-13 member polycyclic heteroaryl group having 1-5 heteroatom(s), wherein said 7-13 member heteroaryl group has:
   a) 1-5 heteroatom(s) when at least one of X or Y is S(O)₂ and at least one of R², R³ and R⁴ is not hydrogen; or
   b) 2-5 heteroatoms when X and Y are C(O);
   vi) a C₁-C₄ alkyl group; and
   vii) a phenyl group when at least one of R², R³ and R⁴ is —CN,
   wherein said heteroatom(s) contained in each cyclic group is independently selected from the group consisting of N, O, and S; and
   wherein each cyclic group is optionally substituted with halo, alkyl, alkenyl, alkynyl, alkoxy, acyl, acyloxy, carboxylic acid, ester, amine, amide, carbonate, carbamate, carbonyl, sulfonyl, cyano, nitro, thioether, thioester, cycloalkyl, cycloheteroalkyl, heterocyclyl, aryl, or heteroaryl, any of which is unsubstituted or substituted with halo, hydroxyl, sulfhydryl, nitro, nitroso, cyano, azido, alkyl, alkenyl, cycloalkyl, cycloheteroalkyl, heterocyclyl, aryl, or heteroaryl,
R² is selected from the group consisting of hydrogen, halo, hydroxyl, alkyl, alkenyl, alkynyl, alkoxy, acyl, acyloxy, carboxylic acid, ester, amine, amide, carbonate, carbamate, cyano, nitro, thioether, thioester, cycloalkyl, cycloheteroalkyl, heterocyclyl, aryl, and heteroaryl, each of which is optionally substituted with halo, hydroxyl, sulfhydryl, nitro, nitroso, cyano, azido, alkyl, alkenyl, cycloalkyl, cycloheteroalkyl, heterocyclyl, aryl or heteroaryl;
each of R³ and R⁴ is independently selected from the group consisting of hydrogen, halo, hydroxyl, alkyl, alkenyl, and alkynyl, any of which is optionally substituted with halo, hydroxyl, alkyl, or cycloalkyl, or R³ taken together with one or more atoms of R⁴ forms a cycloalkyl or cycloheteroalkyl, each of which is optionally substituted with halo, hydroxyl, alkyl, alkenyl, alkynyl, or alkoxy; and
R⁵ is selected from the group consisting of hydrogen, halo, hydroxyl, alkyl, alkenyl, alkynyl, alkoxy, acyl, acyloxy, carboxylic acid, ester, amine, amide, carbonate, carbamate, cyano, nitro, thioether, thioester, cycloalkyl, cycloheteroalkyl, heterocyclyl, aryl, and heteroaryl, each of which is optionally substituted with halo, hydroxyl, sulfhydryl, nitro, nitroso, cyano, azido, alkyl, alkenyl, cycloalkyl, cycloheteroalkyl, heterocyclyl, aryl or heteroaryl.

In some aspects, X is C(O) and Y is S(O)₂.

In some aspects, X is S(O)₂ and Y is C(O).

In some aspects, X is S(O)₂ and Y is S(O)₂.

In some aspects when X or Y is S(O)₂, R¹ can be a 3-9 member cycloalkyl group.

Particularly, R¹ can be a 3-9 member saturated or partially unsaturated cycloalkyl group having 0-5 heteroatom(s). For example, R¹ can be a 3-9 member saturated cycloalkyl group having no heteroatom selected from cyclopropyl, cyclobutyl, cyclopentyl, cyclohexyl, cycloheptyl, cyclooctyl, cyclodecyl, adamantyl, norpinanyl, decalinyl, norbornyl, or housanyl. In one particular embodiment, R¹ is cyclopropyl optionally substituted with halo, alkyl, alkenyl, alkynyl, alkoxy, acyl, acyloxy, carboxylic acid, ester, amine, amide, carbonate, carbamate, carbonyl, cyano, nitro, thioether, thioester, cycloalkyl, cycloheteroalkyl, aryl, heteroaryl, or heterocyclyl. In another embodiment, R¹ is cyclohexyl optionally substituted with halo, alkyl, alkenyl, alkynyl, alkoxy, acyl, acyloxy, carboxylic acid, ester, amine, amide, carbonate, carbamate, carbonyl, cyano, nitro, thioether, thioester, cycloalkyl, cycloheteroalkyl, aryl, heteroaryl, or heterocyclyl. In another embodiment, the 3-9 member cycloalkyl group can be a cycloalkenyl selected from the group consisting of cyclopropenyl, cyclobutenyl, cyclopentenyl, cyclohexenyl, cycloheptenyl, cyclooctenyl, norbornenyl, and norbornadienyl.

In one aspect, R¹ can be a 3-9 member cycloheteroalkyl group containing one or more heteroatoms, each heteroatom independently selected from N, O, or S. For example, R¹ can be a 3-9 member saturated cycloalkyl group having 1-5 heteroatom(s) such as aziridinyl, diaziridinyl, oxiranyl, dioxiranyl, oxaziridinyl, thiiranyl, azetidinyl, diazetidinyl, oxetanyl, dioxetanyl, thietanyl, dithietanyl, pyrrolidinyl, imidazolidinyl, pyrazolidinyl, tetrahydropyranyl, tetrahydrofuranyl, oxolanyl, oxazolidinyl, thiolanyl, thiazolidinyl, piperidinyl, piperazinyl, morpholinyl, thiomorpholinyl, oxanyl, dioxanyl, thianyl, dithianyl, hexahydro-1,3,5-triazinyl, trioxanyl, trithianyl, azepanyl, diazepanyl, oxepanyl, thiepanyl, azocanyl, oxocanyl, thiocanyl, azonanyl, oxonanyl, quinolizinyl, and thionanyl. In one embodiment, R¹ is piperidinyl optionally substituted with halo, alkyl, alkenyl, alkynyl, alkoxy, acyl, acyloxy, carboxylic acid, ester, amine, amide, carbonate, carbamate, carbonyl, cyano, nitro, thioether, thioester, cycloalkyl, cycloheteroalkyl, aryl, heteroaryl, or heterocyclyl.

In one aspect, R¹ can be a 3-9 member partially unsaturated cycloalkyl group having 1-5 heteroatom(s), e.g., pyranyl, pyrrolinyl, pyrazolinyl, imidazolinyl, pyridonyl, dihydropyranyl, dihydropyrrolyl, dihydroimidazolyl, dihydropyrimidinyl, dihydropyrazinyl, dihydrotriazinyl, dihydropyridinyl, dihydrooxadiazolyl, thiazolinyl, dioxinyl, oxazinyl, and oxazolyl. In a preferred embodiment, R¹ is pyridonyl optionally substituted with alkyl.

In one embodiment, the cycloalkyl or cycloheteroalkyl is optionally and independently substituted with halo, alkyl, alkenyl, alkynyl, alkoxy, acyl, acyloxy, carboxylic acid, ester, amine, amide, carbonate, carbamate, carbonyl, cyano, nitro, cycloalkyl, cycloheteroalkyl, aryl, heteroaryl, or heterocyclyl, any of which is optionally substituted with halo, hydroxyl, sulfhydryl, nitro, nitroso, cyano, azido, alkyl, alkenyl, cycloalkyl, cycloheteroalkyl, aryl, heteroaryl, or heterocyclyl.

In some embodiments when X or Y is S(O)₂, R¹ can be a 3-5 member monocyclic heterocyclyl group having 1-3 heteroatom(s), each heteroatom independently selected from N, O, or S. In one embodiment, the 3-5 member monocyclic heterocyclyl group can be an unsaturated 3-4 member monocyclic heterocyclyl group having 1-3 heteroatom(s), e.g., azirinyl, oxirenyl, thiirenyl, diazirinyl, azetyl, diazetyl, oxetyl, dioxetyl, thietyl, or dithietyl. In one embodiment, R¹ is a 5 member unsaturated monocyclic heteroaryl group having 2-3 heteroatoms. In a preferred embodiment, the 5 member monocyclic heteroaryl group is selected from the group consisting of pyrazolyl, imidazolyl, triazolyl (e.g., 1,2,3-triazolyl and 1,2,4-triazolyl), tetrazolyl, thiophenyl, thiazolyl, dithiazolyl, isothiazolyl, thiadiazolyl, furanyl, furazanyl, oxazolyl, isoxazolyl, and oxadiazolyl (e.g., 1,2,5-oxidiazolyl, 1,2,3-oxidiazolyl, and 1,2,4-oxidiazolyl), with the proviso that R¹ is not furanyl or thiophenyl when Y is S(O)₂. In one particular embodiment, R¹ is furazanyl. In yet another preferred embodiment, R¹ is 1,2,4-oxadiazolyl which is optionally substituted. In yet another preferred embodiment, R¹ is 1,2,3-triazolyl or 1,2,4-triazolyl, each of which is optionally substituted. In yet another preferred embodiment, R¹ is tetrazolyl. In yet another preferred embodiment, R¹ is imidazolyl optionally substituted. In yet another preferred embodiment, R¹ is pyrazonyl optionally substituted. The 5 member unsaturated monocyclic heteroaryl group can be substituted with halo, alkyl, alkenyl, alkynyl, alkoxy, acyl, acyloxy, carboxylic acid, ester, amine, amide, carbonate, carbamate, carbonyl, cyano, nitro, cycloalkyl, cycloheteroalkyl, heterocyclyl, aryl, or heteroaryl, any of which is optionally substituted with halo, hydroxyl, sulfhydryl, nitro, nitroso, cyano, azido, alkyl, alkenyl, cycloalkyl, cycloheteroalkyl, aryl, heteroaryl, or heterocyclyl.

In one aspect, R¹ can be a 6 member unsaturated monocyclic heteroaryl group having 1-3 heteroatom(s) with the proviso that R¹ is not pyridinyl or parazinyl when both X and Y are C(O). In one embodiment, the R¹ is a 6 member monocyclic heteroaryl group selected from the group consisting of pyridinyl, diazinyl (e.g., pyrazinyl, pyrimidinyl, and pyridazinyl), pyranyl, oxazinyl (e.g., 1,2-oxazinyl, 1,3-oxazinyl, and 1,4-oxazinyl), thiazinyl, dioxinyl, dithiinyl, triazinyl (e.g., 1,2,3-triazinyl, 1,2,4-triazinyl, and 1,3,5-triazinyl), tetrazinyl, pentazinyl, or thiopyranyl. In a preferred embodiment, the 6 member monocyclic heteroaryl group is pyrazinyl, pyridazinyl or pyrimidyl. In another preferred embodiment, the 6 member monocyclic heteroaryl group is triazinyl (e.g., 1,2,3-triazinyl, 1,2,4-triazinyl, and 1,3,5-triazinyl). The 6 member monocyclic heteroaryl group is optionally and independently substituted with halo, alkyl, alkenyl, alkynyl, alkoxy, acyl, acyloxy, carboxylic acid, ester, amine, amide, carbonate, carbamate, carbonyl, cyano, nitro, cycloalkyl, cycloheteroalkyl, aryl, heteroaryl, or heterocyclyl, any of which is optionally substituted with halo, hydroxyl, sulfhydryl, nitro, nitroso, cyano, azido, alkyl, alkenyl, cycloalkyl, cycloheteroalkyl, aryl, heteroaryl, or heterocyclyl.

In one aspect when X or Y is S(O)₂, R¹ can be a 7-9 member unsaturated monocyclic heterocyclyl group having 1-4 heteroatom(s). In one embodiment, the 7-9 member monocyclic unsaturated heterocyclyl group is selected from the group consisting of azepinyl, diazepinyl, oxepinyl, thiepinyl, thiazepinyl, azocinyl, oxocinyl, thiocinyl, azoninyl, oxoninyl, and thioninyl. The 7-9 member monocyclic unsaturated heterocyclyl group is optionally and independently substituted with halo, alkyl, alkenyl, alkynyl, alkoxy, acyl, acyloxy, carboxylic acid, ester, amine, amide, carbonate, carbamate, carbonyl, cyano, nitro, cycloalkyl, cycloheteroalkyl, heterocyclyl, aryl, or heteroaryl, any of which is optionally substituted with halo, hydroxyl, sulfhydryl, nitro, nitroso, cyano, azido, alkyl, alkenyl, cycloalkyl, cycloheteroalkyl, aryl, heteroaryl, or heterocyclyl.

In one aspect when X or Y is S(O)₂, R¹ is a 7-13 member polycyclic aryl group having 0-5 heteroatom(s). In one particular aspect, R¹ is a 7-13 member polycyclic aryl group having no heteroatom. In another particular aspect, R¹ is a 7-13 member polycyclic heteroaryl group having 1-5 heteroatom(s). In another particular aspect, the 7-13 member polycyclic heteroaryl group is formed by covalently bonding an aryl or a heteroaryl group to a heterocyclyl group. For example, dihydrobenzodioxinyl can be formed by covalently bonding a phenyl group to a heterocyclyl a group, e.g., dioxanyl. Similarly, a 7-13 member polycyclic heteroaryl group can be formed by covalently bonding a pyridinyl, pyridazinyl, pyrimidinyl, or pyrazinyl to a dioxanyl, leading to dihydrodioxinopyridinyl, dihydrodioxinopyridazinyl, dihydrodioxinopyrimidinyl, or dihydrodioxinopyrazinyl, respectively. The 7-13 member polycyclic aryl group having 1-5 heteroatom(s) is selected from the group consisting of dihydrobenzodioxinyl, dihydrodioxinopyridinyl, dihydrodioxinopyridazinyl, dihydrodioxinopyrimidinyl, dihydrodioxinopyrazinyl, dihydropyrrolopyridinyl, tetrahydronaphthyridinyl, tetrahydropyridopyridazinyl, tetrahydropyridopyrazinyl, tetrahydropyridopyrimidinyl, chromanyl, indolyl, purinyl, isoindolyl, quinolinyl, isoquinolinyl, quinoxalinyl, quinazolinyl, quinolizinyl, 1,8-naphthyridinyl, pyrido[3,2-d]pyrimidinyl, pyrido[4,3-d]pyrimidinyl, pyrido[3,4-b]pyrazinyl, pyrido[2,3-b]pyrazinyl, pteridinyl, acridinyl, cinnolinyl, phthalazinyl, benzimidazolyl, phenazinyl, phenoxazinyl, phenothiazinyl, phenoxathiinyl, benzazepinyl, benzodiazepinyl, benzofuranyl, dibenzofuranyl, isobenzofuranyl, benzothiophenyl, benzoxazinyl, quinolin-2(1H)-onyl, isoquinolin-1(2H)-onyl, indazolyl, benzoxazolyl, benzisoxazolyl, benzothiazolyl, dibenzazepinyl, dibenzoxepinyl, dibenzothiazepinyl, dibenzothiepinyl, carbazolyl, and fluorenyl, with the proviso that R¹ is not indolyl or imidazopyridinyl when Y is S(O)₂ or that R¹ is not quinolinyl when both X and Y are C(O). In one embodiment, the polycyclic heteroaryl is a 7-13 member polycyclic heteroaryl group formed by covalently bonding a heteroaryl group to a cycloheteroalkyl group, for example, fusing pyridinyl, pyridazinyl, pyrimidinyl, or pyrazinyl with dioxanyl. In one embodiment, R¹ is dihydrobenzodioxinyl. In one embodiment, R¹ is dihydrodioxinopyridinyl. In one embodiment, R¹ is dihydrodioxinopyridazinyl. In one embodiment, R¹ is dihydrodioxinopyrimidinyl. In one embodiment, R¹ is dihydrodioxinopyrazinyl. In one embodiment, R¹ is dihydropyrrolopyridinyl. In one embodiment, R¹ is tetrahydronaphthyridinyl.

In one embodiment, R¹ is tetrahydropyridopyridazinyl. In one embodiment, R¹ is tetrahydropyridopyrazinyl. In one embodiment, R¹ is tetrahydropyridopyrimidinyl. In one embodiment, R¹ is benzodioxinyl. The 7-9 member polycyclic unsaturated heteroaryl group is optionally substituted with halo, alkyl, alkenyl, alkynyl, alkoxy, acyl, acyloxy, carboxylic acid, ester, amine, amide, carbonate, carbamate, carbonyl, cyano, nitro, cycloalkyl, cycloheteroalkyl, heterocyclyl, aryl, or heteroaryl, any of which is optionally substituted with halo, hydroxyl, sulfhydryl, nitro, nitroso, cyano, azido, alkyl, alkenyl, cycloalkyl, cycloheteroalkyl, aryl, heteroaryl, or heterocyclyl.

In one aspect, R² is selected from hydrogen, halo, hydroxyl, alkyl, alkenyl, alkynyl, alkoxy, acyl, acyloxy, carobxylic acid, ester, amine, amide, carbonate, carbamate, cyano, nitro, thioether, thioester, cycloalkyl, cycloheteroalkyl, heterocyclyl, aryl, or heteroaryl, each of which is optionally substituted with halo, hydroxyl, sulfhydryl, nitro, nitroso, cyano, azido, alkyl, alkenyl, cycloalkyl, cycloheteroalkyl, heterocyclyl, aryl or heteroaryl. In one embodiment, R² can be hydrogen, halo, cyano, or alkynyl. In one particular embodiment, R² is hydrogen. In another particular embodiment, R² is halo (—F, —Cl, —Br, or —I). In another particular embodiment, R² is cyano. In another particular embodiment, R² is alkynyl, optionally substituted with halo, hydroxyl, sulfhydryl, nitro, nitroso, cyano, azido, alkyl, alkenyl, cycloalkyl, cycloheteroalkyl, heterocyclyl, aryl or heteroaryl. In a preferred embodiment, R² is alkynyl substituted with halo, cycloalkyl, aryl or heteroaryl.

In one aspect, R³ is selected from hydrogen, halo, hydroxyl, alkyl, alkenyl, or alkynyl, any of which is optionally substituted with halo, hydroxyl, alkyl, or cycloalkyl.

In one aspect, R⁴ is hydrogen, halo, hydroxyl, alkyl, alkenyl, or alkynyl, any of which is optionally substituted with halo, hydroxyl, alkyl, or cycloalkyl.

In one aspect, R³ is taken together with one or more atoms of R⁴ forms a cycloalkyl or cycloheteroalkyl, each of which are optionally and independently substituted with halo, hydroxyl, alkyl, alkenyl, alkynyl, alkoxy, acyl, acyloxy, carboxylic acid, ester, amine, amide, carbonate, carbamate, cyano, nitro, thioether, thioester, cycloalkyl, cycloheteroalkyl, aryl, heteroaryl, or heterocyclyl.

In one aspect, R⁵ is selected from the group consisting of halo, hydroxyl, alkyl, alkenyl, alkynyl, alkoxy, acyl, acyloxy, carboxylic acid, ester, amine, amide, carbonate, carbamate, cyano, nitro, thioether, thioester, cycloalkyl, cycloheteroalkyl, heterocyclyl, aryl, and heteroaryl, each of which is optionally substituted with halo, hydroxyl, sulfhydryl, nitro, nitroso, cyano, azido, alkyl, alkenyl, cycloalkyl, cycloheteroalkyl, heterocyclyl, aryl or heteroaryl.

In one embodiment, R⁵ is hydrogen.

In one embodiment, R⁵ is cyano.

In another embodiment, R⁵ is alkyl optionally substituted with halo, hydroxyl, sulfhydryl, nitro, nitroso, cyano, azido, alkyl, alkenyl, cycloalkyl, cycloheteroalkyl, heterocyclyl, aryl or heteroaryl. In one particular embodiment, R⁵ is methyl. In another particular embodiment, R⁵ is methyl substituted with phenyl. In another embodiment, R⁵ is ethyl.

In one embodiment, R⁵ can be in geometric isomerism. For example, R⁵ can be attached in a cis or trans conformation with respect to R¹.

In some aspects when X and Y are C(O) and n is an integer of 1 according to Formula (I), R¹ can be a saturated or partially unsaturated 3-9 member cycloalkyl group having 0-5 heteroatom(s). For example, in one aspect, R¹ can be a 3-9 member saturated cycloalkyl group having no heteroatom selected from cyclopropyl, cyclobutyl, cyclopentyl, cyclohexyl, cycloheptyl, cyclooctyl, cyclodecyl, adamantyl, norpinanyl, decalinyl, norbornyl, or housanyl. In another embodiment, the 3-9 member cycloalkyl is a cycloalkenyl group selected from the group consisting of cyclopropenyl, cyclobutenyl, cyclopentenyl, cyclohexenyl, cycloheptenyl, cyclooctenyl, norbornenyl, and norbornadienyl. In one embodiment, R¹ is cyclopropyl optionally substituted with halo, alkyl, alkenyl, alkynyl, alkoxy, acyl, acyloxy, carboxylic acid, ester, amine, amide, carbonate, carbamate, carbonyl, cyano, nitro, thioether, thioester, cycloalkyl, cycloheteroalkyl, aryl, heteroaryl, or heterocyclyl. In another embodiment, R¹ is cyclohexyl optionally substituted with halo, alkyl, alkenyl, alkynyl, alkoxy, acyl, acyloxy, carboxylic acid, ester, amine, amide, carbonate, carbamate, carbonyl, cyano, nitro, thioether, thioester, cycloalkyl, cycloheteroalkyl, aryl, heteroaryl, or heterocyclyl.

In another aspect when X and Y are C(O) and n is an integer of 1 according to Formula (I), R¹ can be a 3-9 member cycloheteroalkyl group containing one or more heteroatoms, each heteroatom independently selected from N, O, or S. For example, R¹ can be a 3-9 member saturated cycloalkyl group having 1-5 heteroatom(s) such as aziridinyl, diaziridinyl, oxiranyl, dioxiranyl, oxaziridinyl, thiiranyl, azetidinyl, diazetidinyl, oxetanyl, dioxetanyl, thietanyl, dithietanyl, pyrrolidinyl, imidazolidinyl, pyrazolidinyl, tetrahydropyranyl, tetrahydrofuranyl, oxolanyl, oxazolidinyl, thiolanyl, thiazolidinyl, piperidinyl, piperazinyl, morpholinyl, thiomorpholinyl, oxanyl, dioxanyl, thianyl, dithianyl, hexahydro-1,3,5-triazinyl, trioxanyl, trithianyl, azepanyl, diazepanyl, oxepanyl, thiepanyl, azocanyl, oxocanyl, thiocanyl, azonanyl, oxonanyl, quinolizinyl, and thionanyl. In one embodiment, R¹ is piperidinyl optionally substituted with halo, alkyl, alkenyl, alkynyl, alkoxy, acyl, acyloxy, carboxylic acid, ester, amine, amide, carbonate, carbamate, carbonyl, cyano, nitro, thioether, thioester, cycloalkyl, cycloheteroalkyl, aryl, heteroaryl, or heterocyclyl.

In another aspect when X and Y are C(O) and n is an integer of 1 according to Formula (I), R¹ can be a 3-9 member partially unsaturated cycloalkyl group having 1-5 heteroatom(s), e.g., pyranyl, pyrrolinyl, pyrazolinyl, imidazolinyl, pyridonyl, dihydropyranyl, dihydropyrrolyl, dihydroimidazolyl, dihydropyrimidinyl, dihydropyrazinyl, dihydrotriazinyl, dihydropyridinyl, dihydrooxadiazolyl, thiazolinyl, dioxinyl, oxazinyl, and oxazolyl. In a preferred embodiment, R¹ is pyridonyl optionally substituted with alkyl.

In another aspect when X and Y are C(O) and n is an integer of 1 according to Formula (I), the cycloalkyl or cycloheteroalkyl is optionally and independently substituted with halo, alkyl, alkenyl, alkynyl, alkoxy, acyl, acyloxy, carboxylic acid, ester, amine, amide, carbonate, carbamate, carbonyl, cyano, nitro, cycloalkyl, cycloheteroalkyl, aryl, heteroaryl, or heterocyclyl, any of which is optionally substituted with halo, hydroxyl, sulfhydryl, nitro, nitroso, cyano, azido, alkyl, alkenyl, cycloalkyl, cycloheteroalkyl, aryl, heteroaryl, or heterocyclyl.

In another aspect when X and Y are C(O) and n is an integer of 1 according to Formula (I), R¹ can be a 3-5 member monocyclic heterocyclyl group having 1-3 heteroatom(s), each heteroatom independently selected from N, O, or S. In one embodiment, the 3-5 member monocyclic heterocyclyl group can be an unsaturated 3-4 member monocyclic heterocyclyl group having 1-3 heteroatom(s), e.g., azirinyl, oxirenyl, thiirenyl, diazirinyl, azetyl, diazetyl, oxetyl, dioxetyl, thietyl, or dithietyl. In one embodiment, R¹ is a 5 member unsaturated monocyclic heteroaryl group having 2-3 heteroatoms. In one embodiment, R¹ is a 5 member unsaturated monocyclic heteroaryl group having 3 heteroatoms. In a preferred embodiment, the 5 member monocyclic heteroaryl group is selected from the group consisting of pyrazolyl, imidazolyl, triazolyl (e.g., 1,2,3-triazolyl and 1,2,4-triazolyl), tetrazolyl, thiophenyl, thiazolyl, dithiazolyl, isothiazolyl, thiadiazolyl, furanyl, furazanyl, oxazolyl, isoxazolyl, and oxadiazolyl (e.g., 1,2,5-oxidiazolyl, 1,2,3-oxidiazolyl, and 1,2,4-oxidiazolyl), with the proviso that R¹ is not furanyl or thiophenyl. In one particular embodiment, R¹ is furazanyl. In yet another preferred embodiment, R¹ is 1,2,4-oxadiazolyl which is optionally substituted. In yet another preferred embodiment, R¹ is 1,2,3-triazolyl or 1,2,4-triazolyl, each of which is optionally substituted. In yet another preferred embodiment, R¹ is tetrazolyl. In yet another preferred embodiment, R¹ is imidazolyl optionally substituted. In yet another preferred embodiment, R¹ is pyrazonyl optionally substituted. The 5 member unsaturated monocyclic heteroaryl group can be substituted with halo, alkyl, alkenyl, alkynyl, alkoxy, acyl, acyloxy, carboxylic acid, ester, amine, amide, carbonate, carbamate, carbonyl, cyano, nitro, cycloalkyl, cycloheteroalkyl, heterocyclyl, aryl, or heteroaryl, any of which is optionally substituted with halo, hydroxyl, sulfhydryl, nitro, nitroso, cyano, azido, alkyl, alkenyl, cycloalkyl, cycloheteroalkyl, aryl, heteroaryl, or heterocyclyl.

In another aspect when X and Y are C(O) and n is an integer of 1 according to Formula (I), R¹ is a 6 member monocyclic heteroaryl group having 1-3 heteroatom(s) with the proviso that R¹ is not pyridinyl or parazinyl. In one embodiment, the R¹ is a 6 member monocyclic heteroaryl group selected from the group consisting of pyrimidinyl, pyridazinyl, pyranyl, oxazinyl (e.g., 1,2-oxazinyl, 1,3-oxazinyl, and 1,4-oxazinyl), thiazinyl, dioxinyl, dithiinyl, triazinyl (e.g., 1,2,3-triazinyl, 1,2,4-triazinyl, and 1,3,5-triazinyl), tetrazinyl, pentazinyl, or thiopyranyl. In a preferred embodiment, the 6 member monocyclic heteroaryl group is pyridazinyl or pyrimidyl. In another preferred embodiment, the 6 member monocyclic heteroaryl group is triazinyl (e.g., 1,2,3-triazinyl, 1,2,4-triazinyl, and 1,3,5-triazinyl). The 6 member monocyclic heteroaryl group is optionally and independently substituted with halo, alkyl, alkenyl, alkynyl, alkoxy, acyl, acyloxy, carboxylic acid, ester, amine, amide, carbonate, carbamate, carbonyl, cyano, nitro, cycloalkyl, cycloheteroalkyl, aryl, heteroaryl, or heterocyclyl, any of which is optionally substituted with halo, hydroxyl, sulfhydryl, nitro, nitroso, cyano, azido, alkyl, alkenyl, cycloalkyl, cycloheteroalkyl, aryl, heteroaryl, or heterocyclyl.

In another aspect when X and Y are C(O) and n is an integer of 1 according to Formula (I), R¹ can be a 7-9 member unsaturated monocyclic heterocyclyl group having 1-4 heteroatom(s). In one embodiment, the 7-9 member monocyclic unsaturated heterocyclyl group is selected from the group consisting of azepinyl, diazepinyl, oxepinyl, thiepinyl, thiazepinyl, azocinyl, oxocinyl, thiocinyl, azoninyl, oxoninyl, and thioninyl. The 7-9 member monocyclic unsaturated heterocyclyl group is optionally and independently substituted with halo, alkyl, alkenyl, alkynyl, alkoxy, acyl, acyloxy, carboxylic acid, ester, amine, amide, carbonate, carbamate, carbonyl, cyano, nitro, cycloalkyl, cycloheteroalkyl, heterocyclyl, aryl, or heteroaryl, any of which is optionally substituted with halo, hydroxyl, sulfhydryl, nitro, nitroso, cyano, azido, alkyl, alkenyl, cycloalkyl, cycloheteroalkyl, aryl, heteroaryl, or heterocyclyl.

In another aspect when X and Y are C(O) and n is an integer of 1 according to Formula (I), R¹ is a 7-13 member polycyclic aryl group having 0-5 heteroatom(s). In one embodiment, R¹ is a 7-13 member polycyclic aryl group having no heteroatom. In another embodiment, R¹ is a 7-13 member polycyclic heteroaryl group having 1-5 heteroatom(s). In one particular embodiment, the 7-13 member polycyclic heteroaryl group is formed by covalently bonding an aryl or a heteroaryl group to a heterocyclyl group. For example, dihydrobenzodioxinyl can be formed by covalently bonding a phenyl group to a heterocyclyl a group, e.g., dioxanyl. Similarly, a 7-13 member polycyclic heteroaryl group can be formed by covalently bonding a pyridinyl, pyridazinyl, pyrimidinyl, or pyrazinyl to a dioxanyl, leading to dihydrodioxinopyridinyl, dihydrodioxinopyridazinyl, dihydrodioxinopyrimidinyl, or dihydrodioxinopyrazinyl, respectively. The 7-13 member polycyclic aryl group having 1-5 heteroatom(s) is selected from the group consisting of dihydrobenzodioxinyl, dihydrodioxinopyridinyl, dihydrodioxinopyridazinyl, dihydrodioxinopyrimidinyl, dihydrodioxinopyrazinyl, dihydropyrrolopyridinyl, tetrahydronaphthyridinyl, tetrahydropyridopyridazinyl, tetrahydropyridopyrazinyl, tetrahydropyridopyrimidinyl, chromanyl, indolyl, purinyl, isoindolyl, quinoxalinyl, quinazolinyl, quinolizinyl, 1,8-naphthyridinyl, pyrido[3,2-d]pyrimidinyl, pyrido[4,3-d]pyrimidinyl, pyrido[3,4-b]pyrazinyl, pyrido[2,3-b]pyrazinyl, pteridinyl, acridinyl, cinnolinyl, phthalazinyl, benzimidazolyl, phenazinyl, phenoxazinyl, phenothiazinyl, phenoxathiinyl, benzazepinyl, benzodiazepinyl, benzofuranyl, dibenzofuranyl, isobenzofuranyl, benzothiophenyl, benzoxazinyl, quinolin-2(1H)-onyl, isoquinolin-1(2H)-onyl, indazolyl, benzoxazolyl, benzisoxazolyl, benzothiazolyl, dibenzazepinyl, dibenzoxepinyl, dibenzothiazepinyl, dibenzothiepinyl, carbazolyl, and fluorenyl, with the proviso that R¹ is not quinolinyl. In one embodiment, the polycyclic heteroaryl is a 7-13 member polycyclic heteroaryl group formed by covalently bonding a heteroaryl group to a cycloheteroalkyl group, for example, fusing pyridinyl, pyridazinyl, pyrimidinyl, or pyrazinyl with dioxanyl. In one embodiment, R¹ is dihydrobenzodioxinyl. In one embodiment, R¹ is dihydrodioxinopyridinyl. In one embodiment, R¹ is dihydrodioxinopyridazinyl. In one embodiment, R¹ is dihydrodioxinopyrimidinyl. In one embodiment, R¹ is dihydrodioxinopyrazinyl. In one embodiment, R¹ is dihydropyrrolopyridinyl. In one embodiment, R¹ is tetrahydronaphthyridinyl.

In one embodiment, R¹ is tetrahydropyridopyridazinyl. In one embodiment, R¹ is tetrahydropyridopyrazinyl. In one embodiment, R¹ is tetrahydropyridopyrimidinyl. In one embodiment, R¹ is benzodioxinyl. The 7-9 member polycyclic unsaturated heteroaryl group is optionally substituted with halo, alkyl, alkenyl, alkynyl, alkoxy, acyl, acyloxy, carboxylic acid, ester, amine, amide, carbonate, carbamate, carbonyl, cyano, nitro, cycloalkyl, cycloheteroalkyl, heterocyclyl, aryl, or heteroaryl, any of which is optionally substituted with halo, hydroxyl, sulfhydryl, nitro, nitroso, cyano, azido, alkyl, alkenyl, cycloalkyl, cycloheteroalkyl, aryl, heteroaryl, or heterocyclyl.

In another aspect when X and Y are C(O) and n is an integer of 1 according to Formula (I), R¹ can be a C₁-C₄ alkyl group. In some embodiments, R¹ can be a C₁-C₃ alkyl group. In one embodiment, R¹ is a branched C₁-C₄ alkyl group (e.g., tert-butyl).

In one aspect when X and Y are C(O) and n is an integer of 1 according to Formula (I), R² is selected from hydrogen, halo, hydroxyl, alkyl, alkenyl, alkynyl, alkoxy, acyl, acyloxy, carboxylic acid, ester, amine, amide, carbonate, carbamate, cyano, nitro, thioether, thioester, cycloalkyl, cycloheteroalkyl, heterocyclyl, aryl, or heteroaryl, each of which is optionally substituted with halo, hydroxyl, sulfhydryl, nitro, nitroso, cyano, azido, alkyl, alkenyl, cycloalkyl, cycloheteroalkyl, heterocyclyl, aryl or heteroaryl. In one embodiment, R² is hydrogen. In another embodiment, R² is halo (—F, —Cl, —Br, or —I). In another embodiment, R² is cyano. In one embodiment, R² is alkynyl, optionally substituted with halo, hydroxyl, sulfhydryl, nitro, nitroso, cyano, azido, alkyl, alkenyl, cycloalkyl, cycloheteroalkyl, heterocyclyl, aryl or heteroaryl. In a preferred embodiment, R² is alkynyl substituted with halo, cycloalkyl, aryl or heteroaryl.

In one aspect when X and Y are C(O) and n is an integer of 1 according to Formula (I), R³ is selected from hydrogen, halo, hydroxyl, alkyl, alkenyl, or alkynyl, any of which is optionally substituted with halo, hydroxyl, alkyl, or cycloalkyl.

In one aspect when X and Y are C(O) and n is an integer of 1 according to Formula (I), R⁴ is hydrogen, halo, hydroxyl, alkyl, alkenyl, or alkynyl, any of which is optionally substituted with halo, hydroxyl, alkyl, or cycloalkyl.

In one aspect when X and Y are C(O) and n is an integer of 1 according to Formula (I), R³ is taken together with one or more atoms of R⁴ forms a cycloalkyl or cycloheteroalkyl, each of which are optionally and independently substituted with halo, hydroxyl, alkyl, alkenyl, alkynyl, alkoxy, acyl, acyloxy, carboxylic acid, ester, amine, amide, carbonate, carbamate, cyano, nitro, thioether, thioester, cycloalkyl, cycloheteroalkyl, heterocyclyl, aryl, and heteroaryl.

In one aspect when X and Y are C(O) and n is an integer of 1 according to Formula (I), R⁵ is selected from the group consisting of halo, hydroxyl, alkyl, alkenyl, alkynyl, alkoxy, acyl, acyloxy, carboxylic acid, ester, amine, amide, carbonate, carbamate, cyano, nitro, thioether, thioester, cycloalkyl, cycloheteroalkyl, heterocyclyl, aryl, and heteroaryl, each of which is optionally substituted with halo, hydroxyl, sulfhydryl, nitro, nitroso, cyano, azido, alkyl, alkenyl, cycloalkyl, cycloheteroalkyl, heterocyclyl, aryl or heteroaryl.

In one embodiment, R⁵ is hydrogen.

In one embodiment, R⁵ is cyano.

In another embodiment, R⁵ is alkyl optionally substituted with halo, hydroxyl, sulfhydryl, nitro, nitroso, cyano, azido, alkyl, alkenyl, cycloalkyl, cycloheteroalkyl, heterocyclyl, aryl or heteroaryl. In one particular embodiment, R⁵ is methyl. In another particular embodiment, R⁵ is methyl substituted with phenyl. In another embodiment, R⁵ is ethyl.

In one embodiment, R⁵ can be in geometric isomerism. For example, R⁵ can be attached in a cis or trans conformation with respect to R¹.

The heteroatom(s) contained in each cyclic group described above is independently selected from the group consisting of N, O, and S; and each cyclic group is optionally substituted with halo, alkyl, alkenyl, alkynyl, alkoxy, acyl, acyloxy, carboxylic acid, ester, amine, amide, carbonate, carbamate, carbonyl, cyano, nitro, thioether, thioester, cycloalkyl, cycloheteroalkyl, heterocyclyl, aryl, or heteroaryl, any of which is unsubstituted or substituted with halo, hydroxyl, sulfhydryl, nitro, nitroso, cyano, azido, alkyl, alkenyl, cycloalkyl, cycloheteroalkyl, aryl, heteroaryl, or heterocyclyl.

The present disclosure also contemplates compounds of Formula (I) having n that is an integer of 0 or 2. When n is an integer of 0 or 2, X or Y can be independently C(O) or S(O)₂.

In some aspects, X is C(O) and Y is C(O).

In other aspects, X is C(O) and Y is S(O)₂.

In other aspects, X is S(O)₂ and Y is C(O).

In yet other aspects, X is S(O)₂ and Y is S(O)₂.

In some aspects when n is an integer of 0 or 2, and X or Y is independently C(O) or S(O)₂, R¹ can be selected from the group consisting of branched C₁-C₄ alkyl (e.g., tert-butyl), C₃-C₉ cycloalkyl, C₃-C₉ cycloheteroalkyl, and C₃-C₁₃ heterocyclyl having 1-5 heteroatom(s), each of the heteroatom independently selected from N, O, or S. The R¹ group can be optionally substituted with halo, alkyl, alkenyl, alkynyl, alkoxy, acyl, acyloxy, carboxylic acid, ester, amine, amide, carbonate, carbamate, carbonyl, sulfonyl, cyano, nitro, thioether, thioester, cycloalkyl, cycloheteroalkyl, heterocyclyl, aryl, or heteroaryl, any of which can be unsubstituted or further substituted with halo, hydroxyl, sulfhydryl, nitro, nitroso, cyano, azido, alkyl, alkenyl, cycloalkyl, cycloheteroalkyl, heterocyclyl, aryl or heteroaryl. In one embodiment when n is an integer of 0 or 2 and X or Y is independently C(O) or S(O)₂, R¹ can be a branched C₁-C₃ alkyl.

In one particular embodiment when n is an integer of 0 or 2 and X or Y is independently C(O) or S(O)₂, R¹ is tert-butyl.

In one aspect when n is an integer of 0 or 2 and X or Y is independently C(O) or S(O)₂, R¹ can be a 3-9 member cycloalkyl group. Particularly, R¹ can be a 3-9 member saturated or partially unsaturated cycloalkyl group having 0-5 heteroatom(s).

For example, R¹ can be a 3-9 member saturated cycloalkyl group having no heteroatom selected from cyclopropyl, cyclobutyl, cyclopentyl, cyclohexyl, cycloheptyl, cyclooctyl, cyclodecyl, adamantyl, norpinanyl, decalinyl, norbornyl, or housanyl. In one another embodiment, R¹ is cyclopropyl optionally substituted with halo, alkyl, alkenyl, alkynyl, alkoxy, acyl, acyloxy, carboxylic acid, ester, amine, amide, carbonate, carbamate, carbonyl, cyano, nitro, thioether, thioester, cycloalkyl, cycloheteroalkyl, aryl, heteroaryl, or heterocyclyl. In another embodiment, R¹ is cyclohexyl optionally substituted with halo, alkyl, alkenyl, alkynyl, alkoxy, acyl, acyloxy, carboxylic acid, ester, amine, amide, carbonate, carbamate, carbonyl, cyano, nitro, thioether, thioester, cycloalkyl, cycloheteroalkyl, aryl, heteroaryl, or heterocyclyl. In another embodiment, the 3-9 member cycloalkyl group can be a cycloalkenyl selected from the group consisting of cyclopropenyl, cyclobutenyl, cyclopentenyl, cyclohexenyl, cycloheptenyl, cyclooctenyl, norbornenyl, and norbornadienyl.

In another aspect when n is an integer of 0 or 2 and X or Y is independently C(O) or S(O)₂, R¹ can be a 3-9 member cycloheteroalkyl group containing one or more heteroatoms, each heteroatom independently selected from N, O, or S. For example, R¹ can be a 3-9 member saturated cycloheteroalkyl group having 1-5 heteroatom(s) such as aziridinyl, diaziridinyl, oxiranyl, dioxiranyl, oxaziridinyl, thiiranyl, azetidinyl, diazetidinyl, oxetanyl, dioxetanyl, thietanyl, dithietanyl, pyrrolidinyl, imidazolidinyl, pyrazolidinyl, tetrahydropyranyl, tetrahydrofuranyl, oxolanyl, oxazolidinyl, thiolanyl, thiazolidinyl, piperidinyl, piperazinyl, morpholinyl, thiomorpholinyl, oxanyl, dioxanyl, thianyl, dithianyl, hexahydro-1,3,5-triazinyl, trioxanyl, trithianyl, azepanyl, diazepanyl, oxepanyl, thiepanyl, azocanyl, oxocanyl, thiocanyl, azonanyl, oxonanyl, quinolizinyl, and thionanyl. In one embodiment, R¹ is piperidinyl optionally substituted with halo, alkyl, alkenyl, alkynyl, alkoxy, acyl, acyloxy, carboxylic acid, ester, amine, amide, carbonate, carbamate, carbonyl, cyano, nitro, thioether, thioester, cycloalkyl, cycloheteroalkyl, aryl, heteroaryl, or heterocyclyl.

In one aspect when n is an integer of 0 or 2 and X or Y is independently C(O) or S(O)₂, R¹ can be a 3-9 member partially unsaturated cycloalkyl group having 1-5 heteroatom(s), e.g., pyranyl, pyrrolinyl, pyrazolinyl, imidazolinyl, pyridonyl, dihydropyranyl, dihydropyrrolyl, dihydroimidazolyl, dihydropyrimidinyl, dihydropyrazinyl, dihydrotriazinyl, dihydropyridinyl, dihydrooxadiazolyl, thiazolinyl, dioxinyl, oxazinyl, and oxazolyl. In a preferred embodiment, R¹ is pyridonyl optionally substituted with alkyl.

In one aspect when n is an integer of 0 or 2 and X or Y is independently C(O) or S(O)₂, the cycloalkyl or cycloheteroalkyl is optionally and independently substituted with halo, alkyl, alkenyl, alkynyl, alkoxy, acyl, acyloxy, carboxylic acid, ester, amine, amide, carbonate, carbamate, carbonyl, cyano, nitro, cycloalkyl, cycloheteroalkyl, aryl, heteroaryl, or heterocyclyl, any of which is optionally substituted with halo, hydroxyl, sulfhydryl, nitro, nitroso, cyano, azido, alkyl, alkenyl, cycloalkyl, cycloheteroalkyl, aryl, heteroaryl, or heterocyclyl.

In one aspect when n is an integer of 0 or 2 and X or Y is independently C(O) or S(O)₂, R¹ can be a 3-5 member monocyclic heterocyclyl group having 1-3 heteroatom(s), each heteroatom independently selected from N, O, or S. In one embodiment, the 3-5 member monocyclic heterocyclyl group can be an unsaturated 3-4 member monocyclic heterocyclyl group having 1-3 heteroatom(s), e.g., azirinyl, oxirenyl, thiirenyl, diazirinyl, azetyl, diazetyl, oxetyl, dioxetyl, thietyl, or dithietyl. In one embodiment, R¹ is a 5 member unsaturated monocyclic heteroaryl group having 2-3 heteroatoms. In a preferred embodiment, the 5 member monocyclic heteroaryl group is selected from the group consisting of pyrazolyl, imidazolyl, triazolyl (e.g., 1,2,3-triazolyl and 1,2,4-triazolyl), tetrazolyl, thiophenyl, thiazolyl, dithiazolyl, isothiazolyl, thiadiazolyl, furanyl, furazanyl, oxazolyl, isoxazolyl, and oxadiazolyl (e.g., 1,2,5-oxidiazolyl, 1,2,3-oxidiazolyl, and 1,2,4-oxidiazolyl), with the proviso that R¹ is not furanyl or thiophenyl when Y is S(O)₂. In one particular embodiment, R¹ is furazanyl. In yet another preferred embodiment, R¹ is 1,2,4-oxadiazolyl which is optionally substituted. In yet another preferred embodiment, R¹ is 1,2,3-triazolyl or 1,2,4-triazolyl, each of which is optionally substituted. In yet another preferred embodiment, R¹ is tetrazolyl. In yet another preferred embodiment, R¹ is imidazolyl optionally substituted. In yet another preferred embodiment, R¹ is pyrazonyl optionally substituted. The 5 member unsaturated monocyclic heteroaryl group can be substituted with halo, alkyl, alkenyl, alkynyl, alkoxy, acyl, acyloxy, carboxylic acid, ester, amine, amide, carbonate, carbamate, carbonyl, cyano, nitro, cycloalkyl, cycloheteroalkyl, heterocyclyl, aryl, or heteroaryl, any of which is optionally substituted with halo, hydroxyl, sulfhydryl, nitro, nitroso, cyano, azido, alkyl, alkenyl, cycloalkyl, cycloheteroalkyl, aryl, heteroaryl, or heterocyclyl.

In one aspect when n is an integer of 0 or 2 and X or Y is independently C(O) or S(O)₂, R¹ can be a 6 member unsaturated monocyclic heteroaryl group having 1-3 heteroatom(s) with the proviso that R¹ is not pyridinyl or parazinyl when both X and Y are C(O). In one embodiment, the R¹ is a 6 member monocyclic heteroaryl group selected from the group consisting of pyridinyl, diazinyl (e.g., pyrazinyl, pyrimidinyl, and pyridazinyl), pyranyl, oxazinyl (e.g., 1,2-oxazinyl, 1,3-oxazinyl, and 1,4-oxazinyl), thiazinyl, dioxinyl, dithiinyl, triazinyl (e.g., 1,2,3-triazinyl, 1,2,4-triazinyl, and 1,3,5-triazinyl), tetrazinyl, pentazinyl, or thiopyranyl. In a preferred embodiment, the 6 member monocyclic heteroaryl group is pyrazinyl, pyridazinyl or pyrimidyl. In another preferred embodiment, the 6 member monocyclic heteroaryl group is triazinyl (e.g., 1,2,3-triazinyl, 1,2,4-triazinyl, and 1,3,5-triazinyl). The 6 member monocyclic heteroaryl group is optionally and independently substituted with halo, alkyl, alkenyl, alkynyl, alkoxy, acyl, acyloxy, carboxylic acid, ester, amine, amide, carbonate, carbamate, carbonyl, cyano, nitro, cycloalkyl, cycloheteroalkyl, aryl, heteroaryl, or heterocyclyl, any of which is optionally substituted with halo, hydroxyl, sulfhydryl, nitro, nitroso, cyano, azido, alkyl, alkenyl, cycloalkyl, cycloheteroalkyl, aryl, heteroaryl, or heterocyclyl.

In one aspect when n is an integer of 0 or 2 and X or Y is independently C(O) or S(O)₂, R¹ can be a 7-9 member unsaturated monocyclic heterocyclyl group having 1-4 heteroatom(s). In one embodiment, the 7-9 member monocyclic unsaturated heterocyclyl group is selected from the group consisting of azepinyl, diazepinyl, oxepinyl, thiepinyl, thiazepinyl, azocinyl, oxocinyl, thiocinyl, azoninyl, oxoninyl, and thioninyl. The 7-9 member monocyclic unsaturated heterocyclyl group is optionally and independently substituted with halo, alkyl, alkenyl, alkynyl, alkoxy, acyl, acyloxy, carboxylic acid, ester, amine, amide, carbonate, carbamate, carbonyl, cyano, nitro, cycloalkyl, cycloheteroalkyl, heterocyclyl, aryl, or heteroaryl, any of which is optionally substituted with halo, hydroxyl, sulfhydryl, nitro, nitroso, cyano, azido, alkyl, alkenyl, cycloalkyl, cycloheteroalkyl, aryl, heteroaryl, or heterocyclyl.

In one aspect when n is an integer of 0 or 2 and X or Y is independently C(O) or S(O)₂, R¹ is a 7-13 member polycyclic aryl group having 0-5 heteroatom(s). In one embodiment, R¹ is a 7-13 member polycyclic aryl group having no heteroatom. In another embodiment, R¹ is a 7-13 member polycyclic heteroaryl group having 1-5 heteroatom(s). In another embodiment, the 7-13 member polycyclic heteroaryl group is formed by covalently bonding an aryl or a heteroaryl group to a heterocyclyl group. For example, dihydrobenzodioxinyl can be formed by covalently bonding a phenyl group to a heterocyclyl a group, e.g., dioxanyl. Similarly, a 7-13 member polycyclic heteroaryl group can be formed by covalently bonding a pyridinyl, pyridazinyl, pyrimidinyl, or pyrazinyl to a dioxanyl, leading to dihydrodioxinopyridinyl, dihydrodioxinopyridazinyl, dihydrodioxinopyrimidinyl, or dihydrodioxinopyrazinyl, respectively. The 7-13 member polycyclic aryl group having 1-5 heteroatom(s) is selected from the group consisting of dihydrobenzodioxinyl, dihydrodioxinopyridinyl, dihydrodioxinopyridazinyl, dihydrodioxinopyrimidinyl, dihydrodioxinopyrazinyl, dihydropyrrolopyridinyl, tetrahydronaphthyridinyl, tetrahydropyridopyridazinyl, tetrahydropyridopyrazinyl, tetrahydropyridopyrimidinyl, chromanyl, indolyl, purinyl, isoindolyl, quinolinyl, isoquinolinyl, quinoxalinyl, quinazolinyl, quinolizinyl, 1,8-naphthyridinyl, pyrido[3,2-d]pyrimidinyl, pyrido[4,3-d]pyrimidinyl, pyrido[3,4-b]pyrazinyl, pyrido[2,3-b]pyrazinyl, pteridinyl, acridinyl, cinnolinyl, phthalazinyl, benzimidazolyl, phenazinyl, phenoxazinyl, phenothiazinyl, phenoxathiinyl, benzazepinyl, benzodiazepinyl, benzofuranyl, dibenzofuranyl, isobenzofuranyl, benzothiophenyl, benzoxazinyl, quinolin-2(1H)-onyl, isoquinolin-1(2H)-onyl, indazolyl, benzoxazolyl, benzisoxazolyl, benzothiazolyl, dibenzazepinyl, dibenzoxepinyl, dibenzothiazepinyl, dibenzothiepinyl, carbazolyl, and fluorenyl. In one embodiment, the polycyclic heteroaryl is a 7-13 member polycyclic heteroaryl group formed by covalently bonding a heteroaryl group to a cycloheteroalkyl group, for example, fusing pyridinyl, pyridazinyl, pyrimidinyl, or pyrazinyl with dioxanyl. In one particular embodiment, R¹ is dihydrobenzodioxinyl. In one particular embodiment, R¹ is dihydrodioxinopyridinyl. In one particular embodiment, R¹ is dihydrodioxinopyridazinyl. In one particular embodiment, R¹ is dihydrodioxinopyrimidinyl. In one particular embodiment, R¹ is dihydrodioxinopyrazinyl. In one particular embodiment, R¹ is dihydropyrrolopyridinyl. In one particular embodiment, R¹ is tetrahydronaphthyridinyl. In one particular embodiment, R¹ is tetrahydropyridopyridazinyl. In one particular embodiment, R¹ is tetrahydropyridopyrazinyl. In one particular embodiment, R¹ is tetrahydropyridopyrimidinyl. In one particular embodiment, R¹ is benzodioxinyl. The 7-9 member polycyclic unsaturated heteroaryl group is optionally substituted with halo, alkyl, alkenyl, alkynyl, alkoxy, acyl, acyloxy, carboxylic acid, ester, amine, amide, carbonate, carbamate, carbonyl, cyano, nitro, cycloalkyl, cycloheteroalkyl, heterocyclyl, aryl, or heteroaryl, any of which is optionally substituted with halo, hydroxyl, sulfhydryl, nitro, nitroso, cyano, azido, alkyl, alkenyl, cycloalkyl, cycloheteroalkyl, aryl, heteroaryl, or heterocyclyl.

In some aspects when n is an integer of 0 or 2 and X or Y is independently C(O) or S(O)₂, R² can be selected from the group consisting of hydrogen, halo, hydroxyl, alkyl, alkenyl, alkynyl, alkoxy, acyl, acyloxy, carboxylic acid, ester, amine, amide, carbonate, carbamate, cyano, nitro, thioether, thioester, cycloalkyl, cycloheteroalkyl, heterocyclyl, aryl, and heteroaryl, each of which may optionally be substituted with halo, hydroxyl, sulfhydryl, nitro, nitroso, cyano, azido, alkyl, alkenyl, cycloalkyl, cycloheteroalkyl, heterocyclyl, aryl or heteroaryl; and

In some aspects when n is an integer of 0 or 2 and X or Y is independently C(O) or S(O)₂, each of R³ and R⁴ is independently selected from the group consisting of hydrogen, halo, hydroxyl, alkyl, alkenyl, alkynyl, alkoxy, cycloalkyl, and cycloheteroalkyl, any of which can optionally be substituted with halo, hydroxyl, alkyl, or cycloalkyl.

In some aspects when n is an integer of 0 or 2 and X or Y is independently C(O) or S(O)₂, R₃ taken together with one or more atoms of R⁴ forms a cycloalkyl or cycloheteroalkyl, each of which is optionally substituted with halo, hydroxyl, alkyl, alkenyl, alkynyl, or alkoxy.

In one aspect when n is an integer of 0 or 2 and X or Y is independently C(O) or S(O)₂, R⁵ is selected from the group consisting of halo, hydroxyl, alkyl, alkenyl, alkynyl, alkoxy, acyl, acyloxy, carboxylic acid, ester, amine, amide, carbonate, carbamate, cyano, nitro, thioether, thioester, cycloalkyl, cycloheteroalkyl, heterocyclyl, aryl, and heteroaryl, each of which is optionally substituted with halo, hydroxyl, sulfhydryl, nitro, nitroso, cyano, azido, alkyl, alkenyl, cycloalkyl, cycloheteroalkyl, heterocyclyl, aryl or heteroaryl.

In one embodiment, R⁵ is hydrogen.

In another embodiment, R⁵ is cyano.

In yet another embodiment, R⁵ is alkyl optionally substituted with halo, hydroxyl, sulfhydryl, nitro, nitroso, cyano, azido, alkyl, alkenyl, cycloalkyl, cycloheteroalkyl, heterocyclyl, aryl or heteroaryl. In one particular embodiment, R⁵ is methyl. In another particular embodiment, R⁵ is methyl substituted with phenyl. In another embodiment, R⁵ is ethyl.

In one embodiment, R⁵ can be in geometric isomerism. For example, R⁵ can be attached in a cis or trans conformation with respect to R¹.

The present invention also contemplates at least one of X or Y is S(O)₂ and at least one of R², R³ and R⁴ is not hydrogen according to Formula (I), wherein n can be any integer of 1, 2, or 3.

In some aspects, at least one of X or Y is S(O)₂ and at least one of R², R³ and R⁴ is not hydrogen. In some aspects, X are Y are independently S(O)₂ and at least one of R², R³ and R⁴ is not hydrogen,

In one aspect when at least one of X or Y is S(O)₂ and at least one of R², R³ and R⁴ is not hydrogen, R¹ can be selected from the group consisting of branched C₁-C₄ alkyl (e.g., tert-butyl), C₃-C₉ cycloalkyl, C₃-C₉ cycloheteroalkyl, and C₃-C₁₃ heterocyclyl having 1-5 heteroatom(s), each of the heteroatom independently selected from N, O, or S. The R¹ group can be optionally substituted with halo, alkyl, alkenyl, alkynyl, alkoxy, acyl, acyloxy, carboxylic acid, ester, amine, amide, carbonate, carbamate, carbonyl, cyano, nitro, thioether, thioester, cycloalkyl, cycloheteroalkyl, heterocyclyl, aryl, or heteroaryl, any of which can be unsubstituted or further substituted with halo, hydroxyl, sulfhydryl, nitro, nitroso, cyano, azido, alkyl, alkenyl, cycloalkyl, cycloheteroalkyl, heterocyclyl, aryl or heteroaryl. In one embodiment when n is an integer of 0 or 2 and X or Y is independently C(O) or S(O)₂, R¹ can be a branched C₁-C₃ alkyl. In one particular embodiment when n is an integer of 0 or 2 and X or Y is independently C(O) or S(O)₂, R¹ is tert-butyl.

In one aspect when at least one of X or Y is S(O)₂ and at least one of R², R³ and R⁴ is not hydrogen, R¹ can be a 3-9 member cycloalkyl group. Particularly, R¹ can be a 3-9 member saturated or partially unsaturated cycloalkyl group having 0-5 heteroatom(s).

For example, R¹ can be a 3-9 member saturated cycloalkyl group having no heteroatom selected from cyclopropyl, cyclobutyl, cyclopentyl, cyclohexyl, cycloheptyl, cyclooctyl, cyclodecyl, adamantyl, norpinanyl, decalinyl, norbornyl, or housanyl. In one another embodiment, R¹ is cyclopropyl optionally substituted with halo, alkyl, alkenyl, alkynyl, alkoxy, acyl, acyloxy, carboxylic acid, ester, amine, amide, carbonate, carbamate, carbonyl, cyano, nitro, thioether, thioester, cycloalkyl, cycloheteroalkyl, aryl, heteroaryl, or heterocyclyl. In another embodiment, R¹ is cyclohexyl optionally substituted with halo, alkyl, alkenyl, alkynyl, alkoxy, acyl, acyloxy, carboxylic acid, ester, amine, amide, carbonate, carbamate, carbonyl, cyano, nitro, thioether, thioester, cycloalkyl, cycloheteroalkyl, aryl, heteroaryl, or heterocyclyl. In another embodiment, the 3-9 member cycloalkyl group can be a cycloalkenyl selected from the group consisting of cyclopropenyl, cyclobutenyl, cyclopentenyl, cyclohexenyl, cycloheptenyl, cyclooctenyl, norbornenyl, and norbornadienyl.

In one aspect when at least one of X or Y is S(O)₂ and at least one of R², R³ and R⁴ is not hydrogen, R¹ can be a 3-9 member cycloheteroalkyl group containing one or more heteroatoms, each heteroatom independently selected from N, O, or S. For example, R¹ can be a 3-9 member saturated cycloheteroalkyl group having 1-5 heteroatom(s) such as aziridinyl, diaziridinyl, oxiranyl, dioxiranyl, oxaziridinyl, thiiranyl, azetidinyl, diazetidinyl, oxetanyl, dioxetanyl, thietanyl, dithietanyl, pyrrolidinyl, imidazolidinyl, pyrazolidinyl, tetrahydropyranyl, tetrahydrofuranyl, oxolanyl, oxazolidinyl, thiolanyl, thiazolidinyl, piperidinyl, piperazinyl, morpholinyl, thiomorpholinyl, oxanyl, dioxanyl, thianyl, dithianyl, hexahydro-1,3,5-triazinyl, trioxanyl, trithianyl, azepanyl, diazepanyl, oxepanyl, thiepanyl, azocanyl, oxocanyl, thiocanyl, azonanyl, oxonanyl, quinolizinyl, and thionanyl. In one embodiment, R¹ is piperidinyl optionally substituted with halo, alkyl, alkenyl, alkynyl, alkoxy, acyl, acyloxy, carboxylic acid, ester, amine, amide, carbonate, carbamate, carbonyl, cyano, nitro, thioether, thioester, cycloalkyl, cycloheteroalkyl, aryl, heteroaryl, or heterocyclyl.

In one aspect when at least one of X or Y is S(O)₂ and at least one of R², R³ and R⁴ is not hydrogen, R¹ can be a 3-9 member partially unsaturated cycloalkyl group having 1-5 heteroatom(s), e.g., pyranyl, pyrrolinyl, pyrazolinyl, imidazolinyl, pyridonyl, dihydropyranyl, dihydropyrrolyl, dihydroimidazolyl, dihydropyrimidinyl, dihydropyrazinyl, dihydrotriazinyl, dihydropyridinyl, dihydrooxadiazolyl, thiazolinyl, dioxinyl, oxazinyl, and oxazolyl. In a preferred embodiment, R¹ is pyridonyl optionally substituted with alkyl.

In one aspect when at least one of X or Y is S(O)₂ and at least one of R², R³ and R⁴ is not hydrogen, the cycloalkyl or cycloheteroalkyl is optionally and independently substituted with halo, alkyl, alkenyl, alkynyl, alkoxy, acyl, acyloxy, carboxylic acid, ester, amine, amide, carbonate, carbamate, carbonyl, cyano, nitro, cycloalkyl, cycloheteroalkyl, aryl, heteroaryl, or heterocyclyl, any of which is optionally substituted with halo, hydroxyl, sulfhydryl, nitro, nitroso, cyano, azido, alkyl, alkenyl, cycloalkyl, cycloheteroalkyl, aryl, heteroaryl, or heterocyclyl.

In one aspect when at least one of X or Y is S(O)₂ and at least one of R², R³ and R⁴ is not hydrogen, R¹ can be a 3-5 member monocyclic heterocyclyl group having 1-3 heteroatom(s), each heteroatom independently selected from N, O, or S. In one embodiment, the 3-5 member monocyclic heterocyclyl group can be an unsaturated 3-4 member monocyclic heterocyclyl group having 1-3 heteroatom(s), e.g., azirinyl, oxirenyl, thiirenyl, diazirinyl, azetyl, diazetyl, oxetyl, dioxetyl, thietyl, or dithietyl. In one embodiment, R¹ is a 5 member unsaturated monocyclic heteroaryl group having 2-3 heteroatoms. In a preferred embodiment, the 5 member monocyclic heteroaryl group is selected from the group consisting of pyrazolyl, imidazolyl, triazolyl (e.g., 1,2,3-triazolyl and 1,2,4-triazolyl), tetrazolyl, thiophenyl, thiazolyl, dithiazolyl, isothiazolyl, thiadiazolyl, furanyl, furazanyl, oxazolyl, isoxazolyl, and oxadiazolyl (e.g., 1,2,5-oxidiazolyl, 1,2,3-oxidiazolyl, and 1,2,4-oxidiazolyl). In one particular embodiment, R¹ is furazanyl. In yet another preferred embodiment, R¹ is 1,2,4-oxadiazolyl which is optionally substituted. In yet another preferred embodiment, R¹ is 1,2,3-triazolyl or 1,2,4-triazolyl, each of which is optionally substituted. In yet another preferred embodiment, R¹ is tetrazolyl. In yet another preferred embodiment, R¹ is imidazolyl optionally substituted. In yet another preferred embodiment, R¹ is pyrazonyl optionally substituted. The 5 member unsaturated monocyclic heteroaryl group can be substituted with halo, alkyl, alkenyl, alkynyl, alkoxy, acyl, acyloxy, carboxylic acid, ester, amine, amide, carbonate, carbamate, carbonyl, cyano, nitro, cycloalkyl, cycloheteroalkyl, heterocyclyl, aryl, or heteroaryl, any of which is optionally substituted with halo, hydroxyl, sulfhydryl, nitro, nitroso, cyano, azido, alkyl, alkenyl, cycloalkyl, cycloheteroalkyl, aryl, heteroaryl, or heterocyclyl.

In one aspect when at least one of X or Y is S(O)₂ and at least one of R², R³ and R⁴ is not hydrogen, R¹ can be a 6 member unsaturated monocyclic heteroaryl group having 1-3 heteroatom(s). In one embodiment, the R¹ is a 6 member monocyclic heteroaryl group selected from the group consisting of pyridinyl, diazinyl (e.g., pyrazinyl, pyrimidinyl, and pyridazinyl), pyranyl, oxazinyl (e.g., 1,2-oxazinyl, 1,3-oxazinyl, and 1,4-oxazinyl), thiazinyl, dioxinyl, dithiinyl, triazinyl (e.g., 1,2,3-triazinyl, 1,2,4-triazinyl, and 1,3,5-triazinyl), tetrazinyl, pentazinyl, or thiopyranyl. In a preferred embodiment, the 6 member monocyclic heteroaryl group is pyrazinyl, pyridazinyl or pyrimidyl. In another preferred embodiment, the 6 member monocyclic heteroaryl group is triazinyl (e.g., 1,2,3-triazinyl, 1,2,4-triazinyl, and 1,3,5-triazinyl). The 6 member monocyclic heteroaryl group is optionally and independently substituted with halo, alkyl, alkenyl, alkynyl, alkoxy, acyl, acyloxy, carboxylic acid, ester, amine, amide, carbonate, carbamate, carbonyl, cyano, nitro, cycloalkyl, cycloheteroalkyl, aryl, heteroaryl, or heterocyclyl, any of which is optionally substituted with halo, hydroxyl, sulfhydryl, nitro, nitroso, cyano, azido, alkyl, alkenyl, cycloalkyl, cycloheteroalkyl, aryl, heteroaryl, or heterocyclyl.

In one aspect when at least one of X or Y is S(O)₂ and at least one of R², R³ and R⁴ is not hydrogen, R¹ can be a 7-9 member unsaturated monocyclic heterocyclyl group having 1-4 heteroatom(s). In one embodiment, the 7-9 member monocyclic unsaturated heterocyclyl group is selected from the group consisting of azepinyl, diazepinyl, oxepinyl, thiepinyl, thiazepinyl, azocinyl, oxocinyl, thiocinyl, azoninyl, oxoninyl, and thioninyl. The 7-9 member monocyclic unsaturated heterocyclyl group is optionally and independently substituted with halo, alkyl, alkenyl, alkynyl, alkoxy, acyl, acyloxy, carboxylic acid, ester, amine, amide, carbonate, carbamate, carbonyl, cyano, nitro, cycloalkyl, cycloheteroalkyl, heterocyclyl, aryl, or heteroaryl, any of which is optionally substituted with halo, hydroxyl, sulfhydryl, nitro, nitroso, cyano, azido, alkyl, alkenyl, cycloalkyl, cycloheteroalkyl, aryl, heteroaryl, or heterocyclyl.

In one aspect when at least one of X or Y is S(O)₂ and at least one of R², R³ and R⁴ is not hydrogen, R¹ is a 7-13 member polycyclic aryl group having 0-5 heteroatom(s). In one embodiment, R¹ is a 7-13 member polycyclic aryl group having no heteroatom. In another embodiment, R¹ is a 7-13 member polycyclic heteroaryl group having 1-5 heteroatom(s). In another embodiment, the 7-13 member polycyclic heteroaryl group is formed by covalently bonding an aryl or a heteroaryl group to a heterocyclyl group. For example, dihydrobenzodioxinyl can be formed by covalently bonding a phenyl group to a heterocyclyl a group, e.g., dioxanyl. Similarly, a 7-13 member polycyclic heteroaryl group can be formed by covalently bonding a pyridinyl, pyridazinyl, pyrimidinyl, or pyrazinyl to a dioxanyl, leading to dihydrodioxinopyridinyl, dihydrodioxinopyridazinyl, dihydrodioxinopyrimidinyl, or dihydrodioxinopyrazinyl, respectively. The 7-13 member polycyclic aryl group having 1-5 heteroatom(s) is selected from the group consisting of dihydrobenzodioxinyl, dihydrodioxinopyridinyl, dihydrodioxinopyridazinyl, dihydrodioxinopyrimidinyl, dihydrodioxinopyrazinyl, dihydropyrrolopyridinyl, tetrahydronaphthyridinyl, tetrahydropyridopyridazinyl, tetrahydropyridopyrazinyl, tetrahydropyridopyrimidinyl, chromanyl, indolyl, purinyl, isoindolyl, quinolinyl, isoquinolinyl, quinoxalinyl, quinazolinyl, quinolizinyl, 1,8-naphthyridinyl, pyrido[3,2-d]pyrimidinyl, pyrido[4,3-d]pyrimidinyl, pyrido[3,4-b]pyrazinyl, pyrido[2,3-b]pyrazinyl, pteridinyl, acridinyl, cinnolinyl, phthalazinyl, benzimidazolyl, phenazinyl, phenoxazinyl, phenothiazinyl, phenoxathiinyl, benzazepinyl, benzodiazepinyl, benzofuranyl, dibenzofuranyl, isobenzofuranyl, benzothiophenyl, benzoxazinyl, quinolin-2(1H)-onyl, isoquinolin-1(2H)-onyl, indazolyl, benzoxazolyl, benzisoxazolyl, benzothiazolyl, dibenzazepinyl, dibenzoxepinyl, dibenzothiazepinyl, dibenzothiepinyl, carbazolyl, and fluorenyl. In one embodiment, the polycyclic heteroaryl is a 7-13 member polycyclic heteroaryl group formed by covalently bonding a heteroaryl group to a cycloheteroalkyl group, for example, fusing pyridinyl, pyridazinyl, pyrimidinyl, or pyrazinyl with dioxanyl. In one particular embodiment, R¹ is dihydrobenzodioxinyl. In one particular embodiment, R¹ is dihydrodioxinopyridinyl. In one particular embodiment, R¹ is dihydrodioxinopyridazinyl. In one particular embodiment, R¹ is dihydrodioxinopyrimidinyl. In one particular embodiment, R¹ is dihydrodioxinopyrazinyl. In one particular embodiment, R¹ is dihydropyrrolopyridinyl. In one particular embodiment, R¹ is tetrahydronaphthyridinyl. In one particular embodiment, R¹ is tetrahydropyridopyridazinyl. In one particular embodiment, R¹ is tetrahydropyridopyrazinyl. In one particular embodiment, R¹ is tetrahydropyridopyrimidinyl. In one particular embodiment, R¹ is benzodioxinyl. The 7-9 member polycyclic unsaturated heteroaryl group is optionally substituted with halo, alkyl, alkenyl, alkynyl, alkoxy, acyl, acyloxy, carboxylic acid, ester, amine, amide, carbonate, carbamate, carbonyl, cyano, nitro, cycloalkyl, cycloheteroalkyl, heterocyclyl, aryl, or heteroaryl, any of which is optionally substituted with halo, hydroxyl, sulfhydryl, nitro, nitroso, cyano, azido, alkyl, alkenyl, cycloalkyl, cycloheteroalkyl, aryl, heteroaryl, or heterocyclyl.

In another aspect, R¹ can be a 3-13 member substituted or unsubstituted heterocyclyl group having 1-5 heteroatom(s) independently selected from the group consisting of N, O, and S, such as: a) a 3-9 member monocyclic heterocycle group selected from pyridyl, pyrimidyl, pyrazinyl, pyridazinyl, thienyl, furanyl, pyrrolyl, pyrazolyl, thiazolyl, oxazolyl, isothiazolyl, isoxazolyl, thiophenyl, triazolyl (1,2,4-triazolyl and 1,2,3-triazolyl), tetrazolyl, oxadiazolyl (1,2,5-oxadiazolyl and 1,2,3-oxadiazolyl), and imidazolyl; and b) a 7-13 member polycyclic heterocycle group selected from dihydrobenzodioxinyl, dihydrodioxinopyridinyl, dihydrodioxinopyridazinyl, dihydrodioxinopyrimidinyl, dihydrodioxinopyrazinyl, dihydropyrrolopyridinyl, tetrahydronaphthyridinyl, tetrahydropyridopyridazinyl, tetrahydropyridopyrazinyl, tetrahydropyridopyrimidinyl, chromanyl, indolyl, purinyl, isoindolyl, quinolinyl, isoquinolinyl, quinoxalinyl, quinazolinyl, quinolizinyl, 1,8-naphthyridinyl, pyrido[3,2-d]pyrimidinyl, pyrido[4,3-d]pyrimidinyl, pyrido[3,4-b]pyrazinyl, pyrido[2,3-b]pyrazinyl, pteridinyl, acridinyl, cinnolinyl, phthalazinyl, benzimidazolyl, phenazinyl, phenoxazinyl, phenothiazinyl, phenoxathiinyl, benzazepinyl, benzodiazepinyl, benzofuranyl, dibenzofuranyl, isobenzofuranyl, benzothiophenyl, benzoxazinyl, quinolin-2(1H)-onyl, isoquinolin-1(2H)-onyl, indazolyl, benzoxazolyl, benzisoxazolyl, benzothiazolyl, dibenzazepinyl, dibenzoxepinyl, dibenzothiazepinyl, dibenzothiepinyl, carbazolyl, or fluorenyl.

In one aspect when X or Y is independently S(O)₂ and at least one of R², R³ and R⁴ is not hydrogen, R² can be selected from the group consisting of hydrogen, halo, hydroxyl, alkyl, alkenyl, alkynyl, alkoxy, acyl, acyloxy, carboxylic acid, ester, amine, amide, carbonate, carbamate, cyano, nitro, thioether, thioester, cycloalkyl, cycloheteroalkyl, heterocyclyl, aryl, and heteroaryl, each of which may optionally be substituted with halo, hydroxyl, sulfhydryl, nitro, nitroso, cyano, azido, alkyl, alkenyl, cycloalkyl, cycloheteroalkyl, heterocyclyl, aryl or heteroaryl; each of R³ and R⁴ is independently selected from the group consisting of hydrogen, halo, hydroxyl, alkyl, alkenyl, alkynyl, alkoxy, cycloalkyl, and cycloheteroalkyl, any of which can optionally be substituted with halo, hydroxyl, alkyl, or cycloalkyl, or R³ taken together with one or more atoms of R⁴ forms a cycloalkyl or cycloheteroalkyl, each of which is optionally substituted with halo, hydroxyl, alkyl, alkenyl, alkynyl, or alkoxy.

In one aspect when X or Y is independently S(O)₂ and at least one of R², R³ and R⁴ is not hydrogen, R⁵ is selected from the group consisting of halo, hydroxyl, alkyl, alkenyl, alkynyl, alkoxy, acyl, acyloxy, carboxylic acid, ester, amine, amide, carbonate, carbamate, cyano, nitro, thioether, thioester, cycloalkyl, cycloheteroalkyl, heterocyclyl, aryl, and heteroaryl, each of which is optionally substituted with halo, hydroxyl, sulfhydryl, nitro, nitroso, cyano, azido, alkyl, alkenyl, cycloalkyl, cycloheteroalkyl, heterocyclyl, aryl or heteroaryl.

In one embodiment, R⁵ is hydrogen.

In another embodiment, R⁵ is cyano.

In yet another embodiment, R⁵ is alkyl optionally substituted with halo, hydroxyl, sulfhydryl, nitro, nitroso, cyano, azido, alkyl, alkenyl, cycloalkyl, cycloheteroalkyl, heterocyclyl, aryl or heteroaryl. In one particular embodiment, R⁵ is methyl. In another particular embodiment, R⁵ is methyl substituted with phenyl. In another embodiment, R⁵ is ethyl.

In one embodiment, R⁵ can be in geometric isomerism. For example, R⁵ can be attached in a cis or trans conformation with respect to R¹.

In some embodiments, provided are compounds of Formula (I), and pharmaceutically acceptable salts thereof with the proviso that (1) R¹ is not any one of the group consisting of

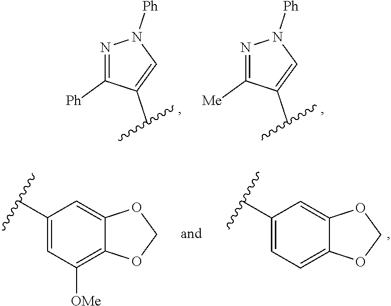 [SMILES: *c1cn(-c2ccccc2)nc1-c1ccccc1 ; *c1cn(-c2ccccc2)nc1C ; *c1cc(OC)c2c(c1)OCO2 ; *c1ccc2c(c1)OCO2]

when n is 1, X and Y are C(O) and R², R³, R⁴ and R⁵ are all hydrogen; (2) R¹ is not unsubstituted or substituted pyridinyl, when n is 1, X and Y are C(O), R² is halo, and R³, R⁴ and R⁵ are all hydrogen; and (3) R¹ is not unsubstituted or substituted pyrazinyl, when n is 1, X and Y are C(O), R² is halo, and R³, R⁴ and R⁵ are all hydrogen.

In some embodiments of the compound of formula (I), and pharmaceutically acceptable salts thereof, where applicable, the compound has one or more of the structural features as discussed herein. In one aspect, a compound of formula (I), or pharmaceutically acceptable salts thereof, is provided, wherein the compound has all of the structural features.

In the descriptions herein, it is understood that every description, variation, embodiment or aspect of a moiety may be combined with every description, variation, embodiment or aspect of other moieties the same as if each and every combination of descriptions is specifically and individually listed.

The compounds of the current disclosure, or their pharmaceutically acceptable salts may contain one or more asymmetric centers and may thus give rise to enantiomers, diastereomers, and other stereoisomeric forms that are defined, in terms of absolute stereochemistry, as (R)- or (S)- or, as (D)- or (L)- for amino acids. The present invention is meant to include all such possible isomers, as well as their racemic and optically pure forms. A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposeable mirror images of one another. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers.

When desired, the (R)- and (S)-isomers of the compounds of the present disclosure, if present, may be resolved by methods known to those skilled in the art, for example by formation of diastereoisomeric salts or complexes which may be separated, for example, by crystallization; via formation of diasteroisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. Alternatively, a specific enantiomer may be synthesized by asymmetric synthesis using optically active reagents, substrates, catalysts or solvents, or by converting one enantiomer to the other by asymmetric transformation.

Compounds may be dosed in their enantiomerically pure form. In some examples, the compound has an enantiomeric excess greater than about 50%, 60%, 70%, 80%, 90%, 95%, 96%, 97%, 98%, or 99%. Compounds may be dosed in their diasteriomerically pure form. In some examples, the compound has a diasteriomeric excess greater than about 50%, 60%, 70%, 80%, 90%, 95%, 96%, 97%, 98%, or 99%.

Compounds may have stereocenters in the R-configuration. Compounds may have stereocenters in the S-configuration.

Some compounds may exhibit polymorphism. It is to be understood that the present disclosure encompasses any racemic, optically-active, polymorphic, or stereoisomeric form, or mixtures thereof, of a compound of the disclosure, which possesses the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase).

The present invention provided herein contemplates pharmaceutical compositions comprising a compound of Formula (I). A pharmaceutical composition (e.g., for oral administration or for injection, infusion, subcutaneous delivery, intramuscular delivery, intraperitoneal delivery, sublingual delivery, or other method) may be in the form of a liquid. A liquid pharmaceutical composition may include, for example, one or more of the following: a sterile diluent such as water, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral composition can be enclosed in ampoules, disposable syringes or multiple dose vials made of glass or plastic. The use of physiological saline is preferred, and an injectable pharmaceutical composition is preferably sterile. A liquid pharmaceutical composition may be delivered orally.

For oral formulations, at least one of the compounds or agents described herein can be used alone or in combination with appropriate additives to make tablets, powders, granules or capsules, and if desired, with diluents, buffering agents, moistening agents, preservatives, coloring agents, and flavoring agents. The compounds may be formulated with a buffering agent to provide for protection of the compound from low pH of the gastric environment and/or an enteric coating. A compound included in a pharmaceutical composition may be formulated for oral delivery with a flavoring agent, e.g., in a liquid, solid or semi-solid formulation and/or with an enteric coating. In some cases, the compounds of this disclosure may be solubilized and encapsulated (e.g., in a liposome or a biodegradable polymer), or used in the form of microcrystals coated with an appropriate nontoxic lipid.

A pharmaceutical composition comprising any one of the compounds or agents described herein may be formulated for sustained or slow release (also called timed release or controlled release). Such compositions may generally be prepared using well known technology and administered by, for example, oral, rectal, intradermal, or subcutaneous implantation, or by implantation at the desired target site. Sustained-release formulations may contain the compound dispersed in a carrier matrix and/or contained within a reservoir surrounded by a rate controlling membrane. Excipients for use within such formulations are biocompatible, and may also be biodegradable; preferably the formulation provides a relatively constant level of active component release. Non-limiting examples of excipients include water, alcohol, glycerol, chitosan, alginate, chondroitin, Vitamin E, mineral oil, and dimethyl sulfoxide (DMSO). The amount of compound contained within a sustained release formulation depends upon the site of implantation, the rate and expected duration of release, and the nature of the condition, disease or disorder to be treated or prevented.

The present disclosure further provides salts of any compound described herein. The term “salt” refers to salts derived from a variety of organic and inorganic counter ions well known in the art. Salts include, for example, acid-addition salts and base-addition salts. The acid that is added to a compound to form an acid-addition salt can be an organic acid or an inorganic acid. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. A base that is added to a compound to form a base-addition salt can be an organic base or an inorganic base. In some cases, a salt can be a metal salt. In some cases, a salt can be an ammonium salt. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like.

Acid addition salts can arise from the addition of an acid to a compound described herein. In some cases, the acid can be organic. In some cases, the acid can be inorganic. Non-limiting examples of suitable acids include hydrochloric acid, hydrobromic acid, hydroiodic acid, nitric acid, nitrous acid, sulfuric acid, sulfurous acid, a phosphoric acid, nicotinic acid, isonicotinic acid, lactic acid, salicylic acid, 4-aminosalicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, citric acid, oxalic acid, maleic acid, hydroxymaleic acid, methylmaleic acid, glycolic acid, malic acid, cinnamic acid, mandelic acid, 2-phenoxybenzoic acid, 2-acetoxybenzoic acid, embonic acid, phenylacetic acid, N-cyclohexylsulfamic acid, methanesulfonic acid, ethanesulfonic acid, benzenesulfonic acid, p-toluenesulfonic acid, 2-hydroxyethanesulfonic acid, ethane-1,2-disulfonic acid, 4-methylbenzenesulfonic acid, naphthalene-2-sulfonic acid, naphthalene-1,5-disulfonic acid, 2-phosphoglyceric acid, 3-phosphoglyceric acid, glucose-6-phosphoric acid, and an amino acid.

Non-limiting examples of suitable acid addition salts include a hydrochloride salt, a hydrobromide salt, a hydroiodide salt, a nitrate salt, a nitrite salt, a sulfate salt, a sulfite salt, a phosphate salt, a hydrogen phosphate salt, a dihydrogen phosphate salt, a carbonate salt, a bicarbonate salt, a nicotinate salt, an isonicotinate salt, a lactate salt, a salicylate salt, a 4-aminosalicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a citrate salt, an oxalate salt, a maleate salt, a hydroxymaleate salt, a methylmaleate salt, a glycolate salt, a malate salt, a cinnamate salt, a mandelate salt, a 2-phenoxybenzoate salt, a 2-acetoxybenzoate salt, an embonate salt, a phenylacetate salt, an N-cyclohexylsulfamate salt, a methanesulfonate salt, an ethanesulfonate salt, a benzenesulfonate salt, a p-toluenesulfonate salt, a 2-hydroxyethanesulfonate salt, an ethane-1,2-disulfonate salt, a 4-methylbenzenesulfonate salt, a naphthalene-2-sulfonate salt, a naphthalene-1,5-disulfonate salt, a 2-phosphoglycerate salt, a 3-phosphoglycerate salt, a glucose-6-phosphate salt, and an amino acid salt.

Metal salts can arise from the addition of an inorganic base to a compound described herein. The inorganic base consists of a metal cation paired with a basic counterion, such as, for example, hydroxide, carbonate, bicarbonate, or phosphate. The metal can be an alkali metal, alkaline earth metal, transition metal, or main group metal. Non-limiting examples of suitable metals include lithium, sodium, potassium, caesium, cerium, magnesium, manganese, iron, calcium, strontium, cobalt, titanium, aluminium, copper, cadmium, and zinc.

Non-limiting examples of suitable metal salts include a lithium salt, a sodium salt, a potassium salt, a caesium salt, a cerium salt, a magnesium salt, a manganese salt, an iron salt, a calcium salt, a strontium salt, a cobalt salt, a titanium salt, an aluminium salt, a copper salt, a cadmium salt, and a zinc salt.

Ammonium salts can arise from the addition of ammonia or an organic amine to a compound described herein. Non-limiting examples of suitable organic amines include triethyl amine, diisopropyl amine, ethanol amine, diethanol amine, triethanol amine, morpholine, N-methylmorpholine, piperidine, N-methylpiperidine, N-ethylpiperidine, dibenzyl amine, piperazine, pyridine, pyrrazole, pipyrrazole, imidazole, pyrazine, pipyrazine, ethylenediamine, N,N′-dibenzylethylene diamine, procaine, chloroprocaine, choline, dicyclohexyl amine, and N-methylglucamine. Non-limiting examples of suitable ammonium salts can be a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzyl amine salt, a piperazine salt, a pyridine salt, a pyrrazole salt, a pipyrrazole salt, an imidazole salt, a pyrazine salt, a pipyrazine salt, an ethylene diamine salt, an N,N′-dibenzylethylene diamine salt, a procaine salt, a chloroprocaine salt, a choline salt, a dicyclohexyl amine salt, and a N-methylglucamine salt.

The present disclosure further provides a pharmaceutically acceptable carrier or a pharmaceutically acceptable excipient of any compound described herein. A pharmaceutically acceptable carrier or excipient may include any and all solvents, dispersion media, coatings, emulsions, isotonic and absorption delaying agents and the like. The use of such media and agents for pharmaceutically active substances is well known in the art. Except insofar as any conventional media or agent is incompatible with an active compound of Formula (I), its use in the therapeutic compositions of the disclosure is contemplated. Supplementary active ingredients can also be incorporated into the compositions. The pharmaceutically acceptable excipient can also include vehicles and carriers capable of being co-administered with a compound to facilitate the performance of its intended function. Any other conventional carrier suitable for use with the multi-binding compounds also falls within the scope of the present disclosure.

In making the compositions of this disclosure, the active ingredient can be diluted by an excipient. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyethylene glycol (PEG), polyvinylpyrrolidone, cellulose, water, sterile saline, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

In some cases, the pharmaceutical compositions described herein may comprise an excipient that can provide long term preservation, bulk up a formulation that contains potent active ingredients, facilitate drug absorption, reduce viscosity, add flavoring, or enhance the solubility of the pharmaceutical composition. Non-limiting examples of excipients can include anti-adherents, binders (e.g., sucrose, lactose, starches, cellulose, gelatin, or PEG), coatings (e.g., hydroxypropyl methylcellulose or gelatin), disintegrants, dyes, flavors (e.g., mint, peach, raspberry, or vanilla), glidants, lubricants, preservatives (e.g., acids, esters, phenols, mercurial compounds, or ammonium compounds), sorbents, or vehicles (e.g., petroleum or mineral oil).

### Methods of Treatment

The compounds and compositions described herein can be administered to a subject in need of treatment for a cell proliferation disorder such as cancer, particularly cancers selected from leukemia, lymphoma, osteosarcoma, melanoma, breast cancer, renal cancer, prostate cancer, gastric cancer, colorectal cancer, liver cancer, thyroid cancer, head and neck cancer, ovarian cancer, pancreatic cancer, neuronal cancer, lung cancer, or uterine cancer. The subject is typically a mammal diagnosed as being in need of treatment for one or more of such proliferative disorders, and frequently the subject is a human. The methods comprise administering an effective amount of at least one compound of the invention; optionally the compound may be administered in combination with one or more additional therapeutic agents, particularly the therapeutic agents known to be useful for treating the cancer or proliferative disorder afflicting the particular subject.

A subject of this disclosure may have any type of cancer. Examples of cancer can include, but are not limited to, adrenal cancer, anal cancer, bile duct cancer, bladder cancer, cancer of the blood, bone cancer, a brain tumor, breast cancer, cancer of the cardiovascular system, cervical cancer, colon cancer, cancer of the digestive system, cancer of the endocrine system, endometrial cancer, esophageal cancer, eye cancer, gallbladder cancer, a gastrointestinal tumor, kidney cancer, laryngeal cancer, leukemia, liver cancer, lung cancer, lymphoma, mesothelioma, cancer of the muscular system, myelodysplastic syndrome, myeloma, nasal cavity cancer, nasopharyngeal cancer, cancer of the nervous system, cancer of the lymphatic system, oral cancer, oropharyngeal cancer, ovarian cancer, pancreatic cancer, penile cancer, pituitary tumors, prostate cancer, cancer of the reproductive system, cancer of the respiratory system, a sarcoma, salivary gland cancer, skeletal system cancer, skin cancer, small intestine cancer, stomach cancer, testicular cancer, thymus cancer, thyroid cancer, bladder cancer, or vaginal cancer. The term ‘lymphoma’ may refer to any type of lymphoma including B-cell lymphoma (e.g., diffuse large B-cell lymphoma, follicular lymphoma, small lymphocytic lymphoma, mantle cell lymphoma, marginal zone B-cell lymphoma, Burkitt lymphoma, lymphoplasmacytic lymphoma, hairy cell leukemia, or primary central nervous system lymphoma) or a T-cell lymphoma (e.g., precursor T-lymphoblastic lymphoma, or peripheral T-cell lymphoma). The term “leukemia” may refer to any type of leukemia including acute leukemia or chronic leukemia. Types of leukemia include acute myeloid leukemia, chronic myeloid leukemia, acute lymphocytic leukemia, acute undifferentiated leukemia, or chronic lymphocytic leukemia.

Examples of cancer include cancers that cause solid tumors as well as cancers that do not cause solid tumors. Furthermore, any of the cancers mentioned herein may be a primary cancer (e.g., a cancer that is named after the part of the body where it first started to grow) or a secondary or metastatic cancer (e.g., a cancer that has originated from another part of the body).

A patient at risk of cancer may be at risk because of a particular condition such as a pre-cancerous condition. Pre-cancerous conditions include but are not limited to actinic keratosis, Barrett's esophagus, atrophic gastritis, ductal carcinoma in situ, dyskeratosis congenita, sideropenic dysphagia, lichen planus, oral submucous fibrosis, solar elastosis, cervical dysplasia, leukoplakia, and erythroplakia). In some cases, a patient may be at risk of cancer because of cell or tissue dysplasia (e.g., an abnormal change in cell number, abnormal change in cell shape, abnormal change in cell size, or abnormal change in cell pigmentation).

The compounds, pharmaceutically acceptable salt thereof, and compositions described herein can be administered to a subject in need of treatment for an autoimmune or inflammatory disease. The compounds, pharmaceutically acceptable salt thereof, and compositions described herein can be administered to a subject in need of reducing inflammation. In some embodiments, the inflammation is chronic inflammation. In some embodiments, the inflammation is acute inflammation. In some embodiments, the inflammation is a symptom of an autoimmune or inflammatory disease. In some embodiments, the compound, pharmaceutically acceptable salt thereof, or composition is administered to the individual according to a dosage and/or method of administration described herein.

Exemplary autoimmune or inflammatory diseases include, but are not limited to acute coronary syndrome, acute inflammation, acute respiratory distress syndrome (ARDS), allergic rhinitis, allergy, alopecia areata, Alzheimer's disease, ankylosing spondylitis (AS), antiphospholipid syndrome, asthma, atherosclerosis, atopic allergy, atopic dermatitis, autoimmune Addison's disease, autoimmune chronic active hepatitis, autoimmune hemolytic anemia, autoimmune hepatitis, autoimmune inner ear disease (AIED), autoimmune lymphoproliferative syndrome (ALPS), autoimmune thrombocytopenia, Behcet's disease, bronchial asthma, cardiomyopathy, celiac spruedermatitis hepetiformis, chronic fatigue immune dysfunction syndrome (CFIDS), chronic inflammation, chronic inflammatory demyelinating polyneuropathy (CIPD), chronic obstructive pulmonary disease, cicatricial pemphigold, cold agglutinin disease, contact dermatitis, crest syndrome, Crohn's Disease, Degos' disease, dermatomyositis, type-1 diabetes mellitus, diabetic retinopathy, discoid lupus, eczema, endometriosis, eosinophilic pneumonia, erythema multiforme, essential mixed cryoglobulinemia, fibromyalgia-fibromyositis, food hypersensitivity, glomerulonephritis, gluten-sensitive enteropathy, graft rejection, graft versus host disease, granulomatous hepatitis, Graves' disease, Guillain-Barre syndrome, Hashimoto's thyroiditis, hemolytic anemias, hepatobiliary diseases, hidradenitis suppurativa, hypersensitivity pneumonitis, idiopathic demyelinating polyneuropathy or Guillain-Barre syndrome, idiopathic inflammatory myopathies, idiopathic pulmonary fibrosis, idiopathic thrombocytopenia purpura (ITP), IgA nephropathy, immune mediated renal disease, inflammatory bowel disease (IBD), insulin-dependent diabetes mellitus, ischemic reperfusion, juvenile chronic arthritis, juvenile idiopathic arthritis, juvenile rheumatoid arthritis, Meniere's disease, mixed connective tissue disease, multiple sclerosis, myasthenia gravis, inflammatory autoimmune myositis, osteoarthritis, pemacious anemia, plaque psoriasis (PP), polyarteritis nodosa, polychondritis, polyglandular syndromes, polymyalgia rheumatica, polymyositis, primary agammaglobulinemia, primary biliary cirrhosis, progressive systemic sclerosis, psoriasis, psoriatic arthritis (PsA), Raynaud's phenomena, Reiter's syndrome, rheumatic fever, rheumatoid arthritis (RA), sarcoidosis, scleroderma, progressive systemic sclerosis (PSS), sclerosing cholangitis, sepsis, Sjogren's syndrome, spondyloarthropathy, stiffman syndrome, stroke, systemic lupus erythematosus, systemic sclerosis, systemic vasculitis, Takayasu arteritis, temporal arteritis/giant cell arteritis, thyroiditis, transplant rejection, ulcerative colitis (UC), urticaria, uveitis, vasculitis, ventilator induced lung injury, vitiligo granulomatosis, Wegener's granulomatosis, or Whipple's disease.

In some embodiments, the autoimmune or inflammatory disease is rheumatoid arthritis (RA), systemic lupus erythematosus, inflammatory bowel disease, type 1 diabetes mellitus, Guillain-Barre syndrome, psoriasis, chronic inflammatory demyelinating polyneuropathy, Graves' disease, Hashimoto's thyroiditis, myasthenia gravis, vasculitis, or transplant rejection.

In some embodiments, the autoimmune or inflammatory disease is rheumatoid arthritis (RA) Rheumatoid arthritis (RA) is a chronic systemic autoimmune inflammatory disease that typically involves the synovial membrane of multiple joints with resultant injury to the articular cartilage. RA can be difficult to diagnose, because symptoms may mimic those of other diseases. Degeneration of joints can be detected by imaging the joints, in many cases over time. Methods of imaging joints are known, such as X-Ray, MRI, and ultrasound tests. Blood tests, such as detection and/or measurements of erythrocyte sedimentation rate (ESR), C-reactive protein (CRP), rheumatoid factor, and/or anti-cyclic citrullinated peptide (anti-CCP) antibodies, can aid the diagnosis of RA. Accordingly, in some embodiments, the subject has one or more markers of RA. In some embodiments, the one or more markers of RA are selected from: degenerating joints, elevated erythrocyte sedimentation rate (ESR), elevated C-reactive protein (CRP), elevated rheumatoid factor, and/or elevated anti-cyclic citrullinated peptide (anti-CCP) antibodies.

RA can be categorized as mild, moderate, or severe. Mild RA or stage 1 RA generally involves the initial inflammation in the joint capsule and swelling of synovial tissue. The swelling causes the symptoms of joint pain, swelling, and stiffness. Moderate RA or stage 2 RA is generally a stage wherein the inflammation of the synovial tissue becomes severe enough that it creates cartilage damage. In this stage, symptoms of loss of mobility and decreased joint range of motion become more frequent. Severe RA generally involves bone deformities and can further be divided into stage 3 and stage 4. Stage 3 generally involves inflammation in the synovium that is destroying the bone and the cartilage of the joint. Potential symptoms of this stage include increased pain and swelling and a further decrease in mobility and even muscle strength. Physical deformities of the joint may start to develop as well. Stage 4 generally includes cessation of the inflammatory process and the joints stop functioning altogether. Pain, swelling, stiffness and loss of mobility are still the primary symptoms in this stage.

In some embodiments, provided herein are methods to treat mild, moderate, or severe RA. In some embodiments, provided herein are methods to treat mild to moderate RA. In some embodiments, provided herein are methods to treat moderate to severe RA. In some embodiments, provided herein are methods to treat mild RA. In some embodiments, provided herein are methods to treat moderate RA. In some embodiments, provided herein are methods to treat severe RA. In some embodiments, provided herein are methods to treat stage 1-4 RA. In some embodiments, provided herein are methods to treat stage 1-2 RA. In some embodiments, provided herein are methods to treat stage 2-4 RA. In some embodiments, provided herein are methods to treat stage 2-3 RA. In some embodiments, provided herein are methods to treat stage 3-4 RA. In some embodiments, provided herein are methods to treat stage 1 RA. In some embodiments, provided herein are methods to treat stage 2 RA. In some embodiments, provided herein are methods to treat stage 3 RA. In some embodiments, provided herein are methods to treat stage 4 RA.

The compounds of the present disclosure or their pharmaceutically acceptable salts are generally administered in a therapeutically effective amount. The amount of the compound actually administered to a subject may be determined by a physician or caregiver, in the light of the relevant circumstances, including the condition to be treated, the chosen route of administration, the compound administered and its relative activity, the age, weight, the response of the individual patient, the severity of the patient's symptoms, and the like. Thus, the therapeutically effective amount may vary, for example, it may vary depending upon the subject's condition, the weight and age of the subject, the severity of the disease condition, the manner of administration and the like.

The effective amount of the compound may in one aspect be a dose of between about 0.01 and about 100 mg/kg, such as about 10-100 mg/kg, about 20-100 mg/kg, about 30-100 mg/kg, about 40-100 mg/kg, about 50-100 mg/kg, about 20-60 mg/kg, about 30-60 mg/kg, about 40-60 mg/kg. Effective amounts or doses of the compounds of the invention may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

Compounds of the present disclosure or a pharmaceutically acceptable salt thereof can be administered, e.g., intravenously, orally, subcutaneously, at a dose of, for example, about 0.05 mg per kg, 0.06 mg per kg, about 0.07 mg per kg, about 0.08 mg per kg, about 0.09 mg per kg, about 0.1 mg per kg, about 0.2 mg per kg, about 0.3 mg per kg, about 0.4 mg per kg, about 0.5 mg per kg, about 0.6 mg per kg, about 0.7 mg per kg, about 0.8 mg per kg, about 0.9 mg per kg, about 1.0 mg per kg, about 1.5 mg per kg, about 2.0, mg per kg about 3.0 mg per kg, about 4.0 mg per kg, about 5.0 mg per kg, about 6.0 mg per kg, about 7.0 mg per kg, about 8.0 mg per kg, about 9.0 mg per kg, about 10 mg per kg, about 11 mg per kg, about 12 mg per kg, about 13 mg per kg, about 14 mg per kg, about 15 mg per kg, about 16 mg per kg, about 17 mg per kg, about 18 mg per kg, about 19 mg per kg, about 20 mg per kg, about 21 mg per kg, about 22 mg per kg, about 23 mg per kg, about 24 mg per kg, about 25 mg per kg, about 26 mg per kg, about 27, mg per kg about 28 mg per kg, about 29 mg per kg, about 30 mg per kg, about 31 mg per kg, about 32 mg per kg, about 33 mg per kg, about 34 mg per kg, about 35 mg per kg, about 40 mg per kg, about 43 mg per kg, about 45 mg per kg, about 48 mg per kg, about 50 mg per kg, about 55 mg per kg, or about 60 mg per kg of the subject.

In one embodiment, compounds of the present disclosure or a pharmaceutically acceptable salt thereof can be administered intravenously at a dose of, for example, 0.05 mg per kg, 0.06 mg per kg, about 0.07 mg per kg, about 0.08 mg per kg, about 0.09 mg per kg, about 0.1 mg per kg, about 0.2 mg per kg, about 0.3 mg per kg, about 0.4 mg per kg, about 0.5 mg per kg, 0.6 mg per kg, about 0.7 mg per kg, about 0.8 mg per kg, about 0.9 mg per kg, about 1.0 mg per kg, about 1.5 mg per kg, about 2.0 mg per kg, about 5 mg per kg, about 6 mg per kg, about 7 mg per kg, about 8 mg per kg, about 9 mg per kg, about 10 mg per kg, about 11 mg per kg, about 12 mg per kg, about 13 mg per kg, about 14 mg per kg, about 15 mg per kg, about 16 mg per kg, about 17 mg per kg, about 18 mg per kg, about 19 mg per kg, about 20 mg per kg, about 21 mg per kg, about 22 mg per kg, about 23 mg per kg, about 24 mg per kg, about 25 mg per kg, about 26 mg per kg, about 27 mg per kg, about 28 mg per kg, about 29 mg per kg, about 30 mg per kg, about 31 mg per kg, about 32 mg per kg, about 33 mg per kg, about 34 mg per kg, about 35 mg per kg, about 40 mg per kg, about 43 mg per kg, about 45 mg per kg, about 48 mg per kg, about 50 mg per kg, about 55 mg per kg, or about 60 mg per kg of the subject.

In one embodiment, compounds of the present disclosure or a pharmaceutically acceptable salt thereof can be administered orally at a dose of, for example, 0.05 mg per kg, 0.06 mg per kg, about 0.07 mg per kg, about 0.08 mg per kg, about 0.09 mg per kg, about 0.1 mg per kg, about 0.2 mg per kg, about 0.3 mg per kg, about 0.4 mg per kg, about 0.5 mg per kg, 0.6 mg per kg, about 0.7 mg per kg, about 0.8 mg per kg, about 0.9 mg per kg, about 1.0 mg per kg, about 1.5 mg per kg, about 2.0 mg per kg, about 5 mg per kg, about 6 mg per kg, about 7 mg per kg, about 8 mg per kg, about 9 mg per kg, about 10 mg per kg, about 11 mg per kg, about 12 mg per kg, about 13 mg per kg, about 14 mg per kg, about 15 mg per kg, about 16 mg per kg, about 17 mg per kg, about 18 mg per kg, about 19 mg per kg, about 20 mg per kg, about 21 mg per kg, about 22 mg per kg, about 23 mg per kg, about 24 mg per kg, about 25 mg per kg, about 26 mg per kg, about 27 mg per kg, about 28 mg per kg, about 29 mg per kg, about 30 mg per kg, about 31 mg per kg, about 32 mg per kg, about 33 mg per kg, about 34 mg per kg, about 35 mg per kg, about 40 mg per kg, about 43 mg per kg, about 45 mg per kg, about 48 mg per kg, about 50 mg per kg, about 55 mg per kg, or about 60 mg per kg of the subject.

In one embodiment, compounds of the present disclosure or a pharmaceutically acceptable salt thereof can be administered subcutaneously at a dose of, for example, 0.05 mg per kg, 0.06 mg per kg, about 0.07 mg per kg, about 0.08 mg per kg, about 0.09 mg per kg, about 0.1 mg per kg, about 0.2 mg per kg, about 0.3 mg per kg, about 0.4 mg per kg, about 0.5 mg per kg, 0.6 mg per kg, about 0.7 mg per kg, about 0.8 mg per kg, about 0.9 mg per kg, about 1.0 mg per kg, about 1.5 mg per kg, about 2.0 mg per kg, about 5 mg per kg, about 6 mg per kg, about 7 mg per kg, about 8 mg per kg, about 9 mg per kg, about 10 mg per kg, about 11 mg per kg, about 12 mg per kg, about 13 mg per kg, about 14 mg per kg, about 15 mg per kg, about 16 mg per kg, about 17 mg per kg, about 18 mg per kg, about 19 mg per kg, about 20 mg per kg, about 21 mg per kg, about 22 mg per kg, about 23 mg per kg, about 24 mg per kg, about 25 mg per kg, about 26 mg per kg, about 27 mg per kg, about 28 mg per kg, about 29 mg per kg, about 30 mg per kg, about 31 mg per kg, about 32 mg per kg, about 33 mg per kg, about 34 mg per kg, about 35 mg per kg, about 40 mg per kg, about 43 mg per kg, about 45 mg per kg, about 48 mg per kg, about 50 mg per kg, about 55 mg per kg, or about 60 mg per kg of the subject.

Any of the methods provided herein may in one aspect comprise administering to an individual a pharmaceutical composition that contains an effective amount of a compound provided herein or a salt thereof and a pharmaceutically acceptable excipient.

A compound or composition of the invention may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

The compounds of the current disclosure may be administered by any of the accepted modes of administration of agents having similar utilities, for example, by cutaneous, oral, topical, intradermal, intrathecal, intravenous, subcutaneous, intramuscular, intra-articular, intraspinal or spinal, nasal, epidural, rectal, vaginal, or transdermal/transmucosal routes. The most suitable route will depend on the nature and severity of the condition being treated. Intravenous administration may be a route of administration for compounds of this disclosure. Oral administration may be a route of administration for compounds of this disclosure. Subcutaneous, intradermal and percutaneous injections can be routes for the compounds of this disclosure. Sublingual administration may be a route of administration for compounds of this disclosure. In a particular example, the pharmaceutical composition provided herein may be administered to a human patient orally. In another particular example, the pharmaceutical composition provided herein may be administered to a human patient intravenously.

In some embodiments, the individual is a mammal. In some embodiments, the individual is a primate, bovine, ovine, porcine, equine, canine, feline, or rodent. In some embodiments, the individual is a human. In some embodiments, the individual has or is suspected of having any of the diseases disclosed herein. In some embodiments, the individual is a risk for developing any of the diseases disclosed herein.

In some embodiments, the individual is human. In some embodiments, the human is at least about or is about any of 21, 25, 30, 35, 40, 45, 50, 55, 60, 65, 70, 75, 80, or 85 years old. In some embodiments, the human is a child. In some embodiments, the human is less than about or about any of 21, 18, 15, 12, 10, 8, 6, 5, 4, 3, 2, or 1 years old.

Also provided herein are uses of a compound described herein or a pharmaceutically acceptable salt thereof, or a pharmaceutical composition described herein, in the manufacture of a medicament. In some embodiments, the manufacture of a medicament is for the treatment of a disease described herein. In some embodiments, the manufacture of a medicament is for reducing inflammation. In some embodiments, the manufacture of a medicament is for treating inflammation.

### Combination Therapy

The disclosure provided herein describes methods to treat cancer in a subject by administering to the subject at least one compound of the present disclosure. The methods disclosed herein can further comprise administering to the subject a combination of a compound of Formula (I) or a salt thereof and at least one additional anticancer agent wherein the combined composition may be administered as a co-formulation or separately.

The disclosure also provided herein describes methods to reduce inflammation and/or treat autoimmune or inflammatory diseases in a subject by administering to the subject at least one compound of the present disclosure. The methods disclosed herein can further comprise administering to the subject a combination of a compound of Formula (I) or a salt thereof and at least one additional agent. The combination may be administered as a co-formulation or separately.

In certain particular embodiments, more than one compound of the current disclosure may be administered at a time to the subject. In some embodiments, two compounds of the current disclosure in combination may act synergistically or additively, and either compound may be used in a lesser amount than if administered alone.

In certain embodiments, compounds disclosed herein and/or pharmaceutical compositions thereof can be used with additional therapeutic agent(s) in combination therapy. The compounds disclosed herein and/or pharmaceutical compositions thereof and the therapeutic agent can act additively or, more preferably, synergistically. In some embodiments, compounds disclosed herein and/or pharmaceutical compositions thereof are administered concurrently with the administration of another therapeutic agent. For example, compounds disclosed herein and/or pharmaceutical compositions thereof may be administered together with another therapeutic agent. In other embodiments, compounds disclosed herein and/or pharmaceutical compositions thereof are administered prior or subsequent to administration of other therapeutic agents. The additional therapeutic agent(s) used in combination therapy can be an anticancer agent such as etoposide, cisplatin, oxaliplatin, gemcitabine, irinotecan, anthracycline, and taxol.

In certain embodiments, compounds disclosed herein and/or pharmaceutical compositions thereof can be used with a second agent. The compounds disclosed herein and/or pharmaceutical compositions thereof and the second agent can act additively or synergistically. In some embodiments, the method disclosed herein comprises sequentially administering, concurrently administering, or simultaneously administering a compound described herein, or a pharmaceutically acceptable salt thereof, or a pharmaceutical composition described herein; and a second agent.

In certain embodiments, the method disclosed herein comprises administering a compound described herein, or a pharmaceutically acceptable salt thereof, or a pharmaceutical composition described herein; and a second agent with a time separation of about 15 minutes or less, such as about any of 10, 5, or 1 minutes or less. In certain embodiments, the method disclosed herein comprises administering a compound described herein, or a pharmaceutically acceptable salt thereof, or a pharmaceutical composition described herein; and a second agent are administered with a time separation of about 15 minutes or more, such as about any of 20, 30, 40, 50, 60, or more minutes. Either a compound described herein, or a pharmaceutically acceptable salt thereof, or a pharmaceutical composition described herein; or a second agent may be administered first. In certain embodiments, the method disclosed herein comprises administering a compound described herein, or a pharmaceutically acceptable salt thereof, or a pharmaceutical composition described herein; and a second agent simultaneously.

In some embodiments, the second agent is an agent useful in the treatment of a disease disclosed herein and/or reducing inflammation. In some embodiments, the second agent is a nonsteroidal anti-inflammatory drug (NSAID), a disease-modifying antirheumatic drug (DMARD), a biologic, or a corticosteroid.

In some embodiments, the second agent is a NSAID. NSAIDs are a class of agents that specifically exclude steroids and are believed to work by inhibiting the activity of one or more cyclooxygenase enzymes. Non-limiting examples of NSAIDs include aspirin, diflunisal, salsalate, ibuprofen, dexibuprofen, naproxen, fenoprofen, ketoprofen, dexketoprofen, flurbiprofen, oxaprozin, loxoprofen, indomethacin, tolmetin, sulindac, etodolac, ketorolac, diclofenac, aceclofenac, nabumetone, piroxicam, meloxicam, tenoxicam, droxicam, lornoxicam, phenylbutazone, mefenamic acid, meclofenamic acid, flufenamic acid, tolfenamic acid, celecoxib, etoricoxib, and nimesulide.

In some embodiments, the second agent is a DMARD. DMARDs are a class of agents that slow the progression of joint damage from rheumatoid arthritis. Non-limiting examples of DMARDs include methotrexate, hydroxychloroquine, leflunomide, sulfasalazine, cyclophosphamine, azathioprine, cyclosporine, and Janus kinase (JAK) inhibitors such as tofacitinib, baricitinib, or ruxolitinib.

In some embodiments, the second agent is a biologic, such as an anti-inflammatory biologic. Biologics are a class of agents that are manufactured in, extracted from, or semisynthesized from biological sources. Non-limiting examples of biologics include abatacept, adalimumab, adalimumab-atto, anakinra, baricitinib, certolizumab, etanercept, etanercept-szzs, golimumab, infliximab, infliximab-dyyb, rituximab, sarilumab, tocilizumab, tofacitinib, and biosimilars thereof.

In some embodiments, the second agent is a corticosteroid. Corticosteroids are a class of cortisone-like drugs. Non-limiting examples of corticosteroids include prednisone, prednisolone, or methylprednisolone.

### Articles of Manufacture and Kits

The present disclosure further provides articles of manufacture comprising a compound described herein or a salt thereof, a composition described herein, or one or more unit dosages described herein in suitable packaging. In certain embodiments, the article of manufacture is for use in any of the methods described herein. Suitable packaging is known in the art and includes, for example, vials, vessels, ampules, bottles, jars, flexible packaging and the like. An article of manufacture may further be sterilized and/or sealed.

The present disclosure further provides kits for carrying out the methods of the invention, which comprises one or more compounds described herein or a composition comprising a compound described herein. The kits may employ any of the compounds disclosed herein. In one variation, the kit employs a compound described herein or a salt thereof. The kits may be used for any one or more of the uses described herein, and, accordingly, may contain instructions for the treatment any disease or described herein, for example for the treatment of cancer.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound as disclosed herein and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to a subject.

The present invention is further illustrated by the following examples which should not be construed as further limiting. The contents of all references, patents and published patent applications cited throughout this application, as well as the Figures provided herein, are expressly incorporated herein by reference in their entirety.

### EXAMPLES

Derivatives of piperlongumine were designed and synthesized (see Table 1) using the synthesis methods described further below.

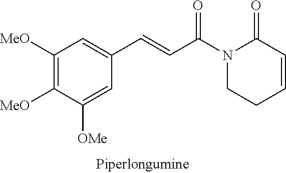 [SMILES: COc1cc(/C=C/C(=O)N2CCC=CC2=O)cc(OC)c1OC]

### General Information

¹H NMR spectra and ¹³C NMR spectra were recorded on a Varian 400 MHz or Bruker Avance III500 MHz spectrometers. Spectra are referenced to residual chloroform (δ 7.26, ¹H), DMSO (δ 2.54, ¹H) or methanol (δ 3.34, ¹H) unless otherwise noted. Chemical shifts are reported in ppm (δ); multiplicities are indicated by s (singlet), d (doublet), t (triplet), q (quartet), quint (quintet), sext (sextet), m (multiplet) and br (broad). Coupling constants, J, are reported in Hertz. Silica gel chromatography was performed using a Teledyne Isco CombiFlash® Rf+ instrument using Hi-Purit Silica Flash Cartridges (National Chromatography Inco) or RediSep Rf Gold C18 Cartridges (Teledyne Isco). Analytical HPLC was performed on a Waters ACQUITY UPLC with a photodiode array detector using and a Waters ACQUITY BEH Shield RPC18 (2.1×50 mm, 1.7 μm) column. Analytical LCMS was performed on a Waters ACQUITY UPLC with a Waters 3100 mass detector. Chiral HPLC was performed on a Waters Alliance e2695 with a photodiode array detector using Daicel Chiralpak® AD-H, Chiralpak® IA, Chiralpak® IB, Chiralpak® IC, Chiralcel® OD-H or Chiralcel® OJ-H columns. Optical rotations were obtained on a Jasco P-2000 digital polarimeter and are reported as [α]_{D}^{T} temperature (T), concentration (c=g/100 mL) and solvent. Commercially available reagents and solvents were used as received unless otherwise indicated.

TABLE 1

| | | | MOLP-8 | DU4475 | HCT116 |
|---|---|---|---|---|---|
| | | | IC₅₀ | IC₅₀ | IC₅₀ |
| No. | Structure | Name | (μM) | (μM) | (μM) |
| 1 | 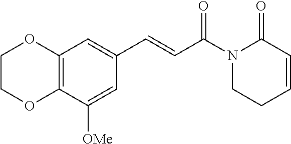 [SMILES: COc1cc(/C=C/C(=O)N2CCC=CC2=O)cc2c1OCCO2] | (E)-1-(3-(8- methoxy-2,3- dihydrobenzo[b][1, 4]dioxin-6- yl)acryloyl)-5,6- dihydropyridin- 2(1H)-one | 0.74 | 0.83 | 1.9 |
| 2 | 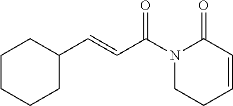 [SMILES: O=C1C=CCCN1C(=O)/C=C/C1CCCCC1] | (E)-1-(3- cyclohexylacryloyl)- 5,6- dihydropyridin- 2(1H)-one | 1.05 | 0.95 | 2.6 |
| 3 | 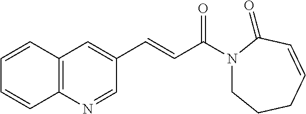 [SMILES: O=C1C=CCCCN1C(=O)/C=C/c1cnc2ccccc2c1] | (E)-1-(3-(quinolin- 3-yl)acryloyl)- 1,5,6,7-tetrahydro- 2H-azepin-2-one | 0.36 | 0.76 | 1.7 |
| 4 | 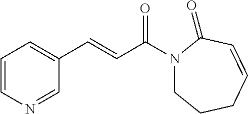 [SMILES: O=C1C=CCCCN1C(=O)/C=C/c1cccnc1] | (E)-1-(3-(pyridin-3- yl)acryloyl)- 1,5,6,7-tetrahydro- 2H-azepin-2-one | 0.52 | 0.59 | 1.9 |
| 5 | 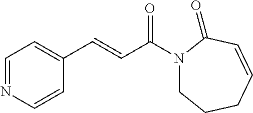 [SMILES: O=C1C=CCCCN1C(=O)/C=C/c1ccncc1] | (E)-1-(3-(pyridin-4- yl)acryloyl)- 1,5,6,7-tetrahydro- 2H-azepin-2-one | 0.49 | 0.57 | 2.0 |
| 6 | 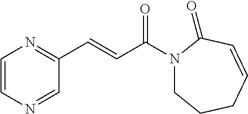 [SMILES: O=C1C=CCCCN1C(=O)/C=C/c1cnccn1] | (E)-1-(3-(pyrazin- 2-yl)acryloyl)- 1,5,6,7-tetrahydro- 2H-azepin-2-one | 0.44 | 0.56 | 2.4 |
| 7 | 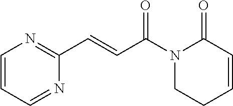 [SMILES: O=C1C=CCCN1C(=O)/C=C/c1ncccn1] | (E)-1-(3- (pyrimidin-2- yl)acryloyl)-5,6- dihydropyridin- 2(1H)-one | 0.57 | 0.49 | 1.5 |
| 8 | 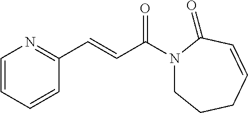 [SMILES: O=C1C=CCCCN1C(=O)/C=C/c1ccccn1] | (E)-1-(3-(pyridin-2- yl)acryloyl)- 1,5,6,7-tetrahydro- 2H-azepin-2-one | 0.41 | 0.50 | 1.5 |
| 9 | 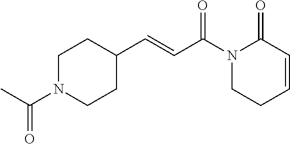 [SMILES: CC(=O)N1CCC(/C=C/C(=O)N2CCC=CC2=O)CC1] | (E)-1-(3-(1- acetylpiperidin-4- yl)acryloyl)-5,6- dihydropyridin- 2(1H)-one | 0.97 | 0.92 | 5.7 |
| 10 | 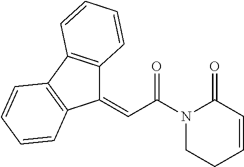 [SMILES: O=C1C=CCCN1C(=O)C=C1c2ccccc2-c2ccccc21] | 1-(2-(9H-fluoren-9- ylidene)acetyl)-5,6- dihydropyridin- 2(1H)-one | 2.12 | 0.95 | 1.9 |
| 11 | 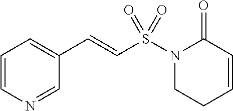 [SMILES: O=C1C=CCCN1S(=O)(=O)/C=C/c1cccnc1] | (E)-1-((2-(pyridin- 3- yl)vinyl)sulfonyl)- 5,6-dihydropyridin- 2(1H)-one | 0.50 | 0.58 | 1.9 |
| 12 | 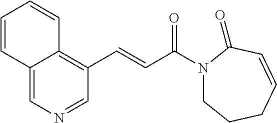 [SMILES: O=C1C=CCCCN1C(=O)/C=C/c1cncc2ccccc12] | (E)-1-(3- (isoquinolin-4- yl)acryloyl)- 1,5,6,7-tetrahydro- 2H-azepin-2-one | 0.89 | 0.60 | 0.96 |
| 13 | 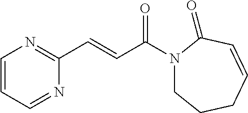 [SMILES: O=C1C=CCCCN1C(=O)/C=C/c1ncccn1] | (E)-1-(3- (pyrimidin-2- yl)acryloyl)- 1,5,6,7-tetrahydro- 2H-azepin-2-one | 0.51 | 0.67 | 1.3 |
| 14 | 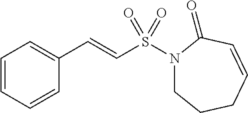 [SMILES: O=C1C=CCCCN1S(=O)(=O)/C=C/c1ccccc1] | (E)-1- (styrylsulfonyl)- 1,5,6,7-tetrahydro- 2H-azepin-2-one | 3.51 | 3.04 | 7.0 |
| 15 | 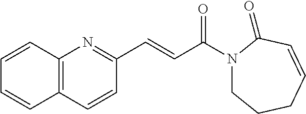 [SMILES: O=C1C=CCCCN1C(=O)/C=C/c1ccc2ccccc2n1] | (E)-1-(3-(quinolin- 2-yl)acryloyl)- 1,5,6,7-tetrahydro- 2H-azepin-2-one | 0.5 | 0.5 | 0.88 |
| 16 | 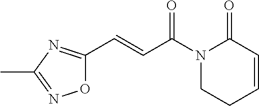 [SMILES: Cc1noc(/C=C/C(=O)N2CCC=CC2=O)n1] | (E)-1-(3-(3-methyl- 1,2,4-oxadiazol-5- yl)acryloyl)-5,6- dihydropyridin- 2(1H)-one | 0.66 | 0.75 | 0.96 |
| 17 | 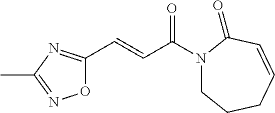 [SMILES: Cc1noc(/C=C/C(=O)N2CCCC=CC2=O)n1] | (E)-1-(3-(3-methyl- 1,2,4-oxadiazol-5- yl)acryloyl)- 1,5,6,7-tetrahydro- 2H-azepin-2-one | 0.33 | 1.16 | 5.93 |
| 18 | 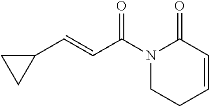 [SMILES: O=C1C=CCCN1C(=O)/C=C/C1CC1] | (E)-1-(3- cyclopropylacryloyl)- 5,6- dihydropyridin- 2(1H)-one | 0.68 | 2.05 | 14.2 |
| 19 | 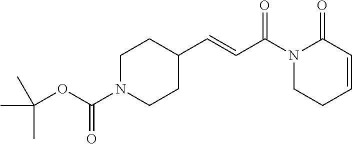 [SMILES: CC(C)(C)OC(=O)N1CCC(/C=C/C(=O)N2CCC=CC2=O)CC1] | tert-butyl (E)-4-(3- oxo-3-(6-oxo-3,6- dihydropyridin- 1(2H)-yl)prop-1- en-1-yl)piperidine- 1-carboxylate | 0.41 | 0.82 | 1.97 |
| 20 | 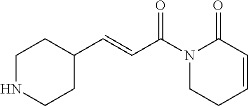 [SMILES: O=C1C=CCCN1C(=O)/C=C/C1CCNCC1] | (E)-1-(3-(piperidin- 4-yl)acryloyl)-5,6- dihydropyridin- 2(1H)-one | 0.69 | 1.32 | 2.13 |
| 21 | 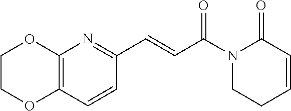 [SMILES: O=C1C=CCCN1C(=O)/C=C/c1ccc2c(n1)OCCO2] | (E)-1-(3-(2,3- dihydro- [1,4]dioxino[2,3- b]pyridin-6- yl)acryloyl)-5,6- dihydropyridin- 2(1H)-one | 0.68 | 0.53 | 3.16 |
| 22 | 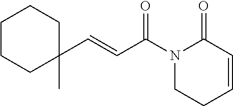 [SMILES: CC1(/C=C/C(=O)N2CCC=CC2=O)CCCCC1] | (E)-1-(3-(1- methylcyclohexyl) acryloyl)-5,6- dihydropyridin- 2(1H)-one | 2.21 | 1.84 | 3.85 |
| 23 | 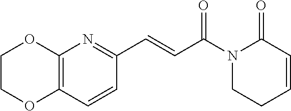 [SMILES: O=C1C=CCCN1C(=O)/C=C/c1ccc2c(n1)OCCO2] | (E)-1-(3-(2,3- dihydro- [1,4]dioxino[2,3- b]pyridin-6- yl)acryloyl)-5,6- dihydropyridin- 2(1H)-one | 0.7 | 0.5 | 3.16 |
| 24 | 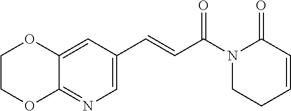 [SMILES: O=C1C=CCCN1C(=O)/C=C/c1cnc2c(c1)OCCO2] | (E)-1-(3-(2,3- dihydro- [1,4]dioxino[2,3- b]pyridin-7- yl)acryloyl)-5,6- dihydropyridin- 2(1H)-one | 0.32 | 0.68 | 1.9 |
| 25 | 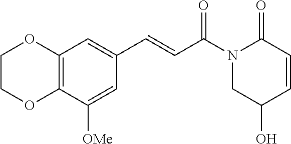 [SMILES: COc1cc(/C=C/C(=O)N2CC(O)C=CC2=O)cc2c1OCCO2] | 5-hydroxy-1-[(2E)- 3-(8-methoxy-2,3- dihydro-1,4- benzodioxin-6- yl)prop-2-enoyl]- 5,6-dihydropyridin- 2(1H)-one | 0.37 | 1.77 | 4.61 |
| 26 | 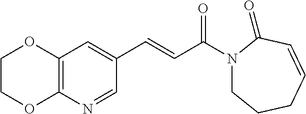 [SMILES: O=C1C=CCCCN1C(=O)/C=C/c1cnc2c(c1)OCCO2] | (E)-1-(3-(2,3- dihydro- [1,4]dioxino[2,3- b]pyridin-7- yl)acryloyl)-6,7- dihydro-1H-azepin- 2(5H)-one | 0.17 | 0.63 | 3.42 |
| 27 | 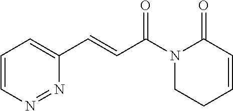 [SMILES: O=C1C=CCCN1C(=O)/C=C/c1cccnn1] | (E)-1-(3-(pyridazin- 3-yl)acryloyl)-5,6- dihydropyridin- 2(1H)-one | 0.76 | 0.95 | 1.9 |
| 28 | 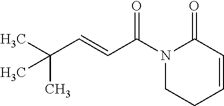 | (E)-1-(4,4- dimethylpent-2- enoyl)-5,6- dihydropyridin- 2(1H)-one | 0.49 | 1.38 | 3.53 |
| 29 | 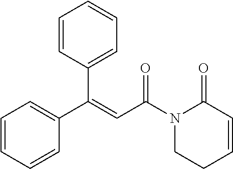 [SMILES: O=C1C=CCCN1C(=O)C=C(c1ccccc1)c1ccccc1] | 1-(3,3- diphenylacryloyl)- 5,6-dihydropyridin- 2(1H)-one | 2.7 | 1.72 | 6.4 |
| 30 | 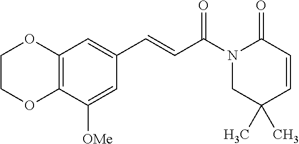 [SMILES: COc1cc(/C=C/C(=O)N2CC(C)(C)C=CC2=O)cc2c1OCCO2] | (E)-1-(3-(8- methoxy-2,3- dihydrobenzo[b][1, 4]dioxin-6- yl)acryloyl)-5,5- dimethyl-5,6- dihydropyridin- 2(1H)-one | 2.8 | 5.5 | 9.9 |
| 31 | 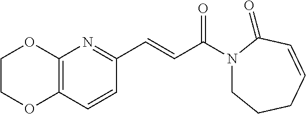 [SMILES: O=C1C=CCCCN1C(=O)/C=C/c1ccc2c(n1)OCCO2] | (E)-1-(3-(2,3- dihydro- [1,4]dioxino[2,3- b]pyridin-6- yl)acryloyl)- 1,5,6,7- tetrahydroazepin-2- one | 0.27 | 0.56 | 1.55 |
| 32 | 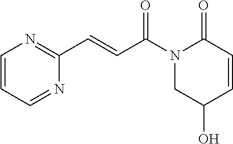 [SMILES: O=C1C=CC(O)CN1C(=O)/C=C/c1ncccn1] | 5-hydroxy-1-[(2E)- 3-(pyrimidin-2- yl)prop-2-enoyl]- 5,6-dihydropyridin- 2(1H)-one | 0.38 | 1.49 | 7.2 |
| 33 | 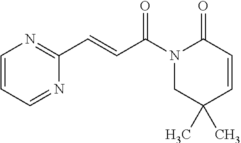 [SMILES: CC1(C)C=CC(=O)N(C(=O)/C=C/c2ncccn2)C1] | (E)-5,5-dimethyl-1- (3-(pyrimidin-2- yl)acryloyl)-5,6- dihydropyridin- 2(1H)-one | 0.56 | 1.3 | 4.07 |
| 34 | 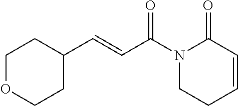 [SMILES: O=C1C=CCCN1C(=O)/C=C/C1CCOCC1] | 1-[(2E)-3- (tetrahydro-2H- pyran-4-yl)prop-2- enoyl]-5,6- dihydropyridin- 2(1H)-one | 0.51 | 1.43 | 3.8 |
| 35 | 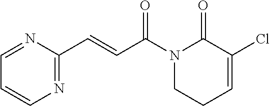 [SMILES: O=C(/C=C/c1ncccn1)N1CCC=C(Cl)C1=O] | (E)-3-chloro-1-(3- (pyrimidin-2- yl)acryloyl)-5,6- dihydropyridin- 2(1H)-one | 0.08 | 0.21 | 0.38 |
| 36 | 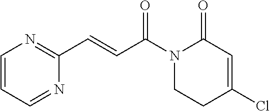 [SMILES: O=C1C=C(Cl)CCN1C(=O)/C=C/c1ncccn1] | (E)-4-chloro-1-(3- (pyrimidin-2- yl)acryloyl)-5,6- dihydropyridin- 2(1H)-one | N/A | N/A | 2.97 |
| 37 | 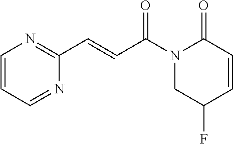 [SMILES: O=C1C=CC(F)CN1C(=O)/C=C/c1ncccn1] | (E)-5-fluoro-1-(3- (pyrimidin-2- yl)acryloyl)-5,6- dihydropyridin- 2(1H)-one | 0.11 | 0.42 | 0.87 |
| 38 | 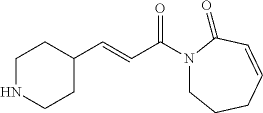 [SMILES: O=C1C=CCCCN1C(=O)/C=C/C1CCNCC1] | (E)-1-(3-(piperidin- 4-yl)acryloyl)-6,7- dihydro-1H-azepin- 2(5H)-one | 0.23 | 1.03 | 0.92 |
| 39 | 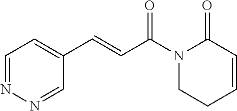 [SMILES: O=C1C=CCCN1C(=O)/C=C/c1ccnnc1] | (E)-1-(3-(pyridazin- 4-yl)acryloyl)-5,6- dihydropyridin- 2(1H)-one | 0.3 | 0.65 | N/A |
| 40 | 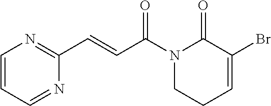 [SMILES: O=C(/C=C/c1ncccn1)N1CCC=C(Br)C1=O] | 3-bromo-1-[(2E)-3- (pyrimidin-2- yl)prop-2-enoyl]- 5,6-dihydropyridin- 2(1H)-one | 0.04 | 0.1 | 0.36 |
| 41 | 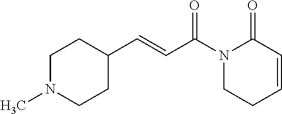 [SMILES: CN1CCC(/C=C/C(=O)N2CCC=CC2=O)CC1] | (E)-1-(3-(1- methylpiperidin-4- yl)acryloyl)-5,6- dihydropyridin- 2(1H)-one | N/A | N/A | 1.24 |
| 42 | 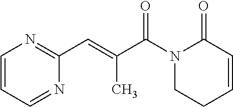 [SMILES: C/C(=C\c1ncccn1)C(=O)N1CCC=CC1=O] | (E)-1-(2-methyl-3- (pyrimidin-2- yl)acryloyl)-5,6- dihydropyridin- 2(1H)-one | N/A | N/A | 2.2 |
| 43 | 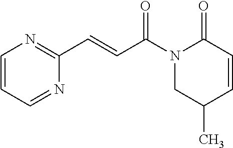 [SMILES: CC1C=CC(=O)N(C(=O)/C=C/c2ncccn2)C1] | (E)-5-methyl-1-(3- (pyrimidin-2- yl)acryloyl)-5,6- dihydropyridin- 2(1H)-one | 0.28 | 0.53 | 1.27 |
| 44 | 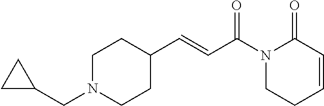 [SMILES: O=C1C=CCCN1C(=O)/C=C/C1CCN(CC2CC2)CC1] | (E)-1-(3-(1- (cyclopropylmethyl) piperidin-4- yl)acryloyl)-5,6- dihydropyridin- 2(1H)-one | 0.49 | 1.05 | 1.35 |
| 45 | 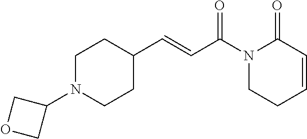 [SMILES: O=C1C=CCCN1C(=O)/C=C/C1CCN(C2COC2)CC1] | (E)-1-(3-(1- (oxetan-3- yl)piperidin-4- yl)acryloyl)-5,6- dihydropyridin- 2(1H)-one | 0.72 | 1.35 | 3.24 |
| 46 | 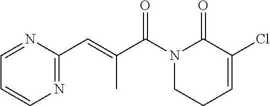 [SMILES: C/C(=C\c1ncccn1)C(=O)N1CCC=C(Cl)C1=O] | (E)-3-chloro-1-(2- methyl-3- (pyrimidin-2- yl)acryloyl)- 5,6- dihydropyridin- 2(1H)-one | N/A | N/A | 0.97 |
| 47 | 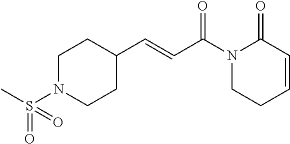 [SMILES: CS(=O)(=O)N1CCC(/C=C/C(=O)N2CCC=CC2=O)CC1] | 1-{(2E)-3-[1- (methylsulfonyl) piperidin-4-yl]prop- 2-enoyl}-5,6- dihydropyridin- 2(1H)-one | N/A | N/A | 3.14 |
| 48 | 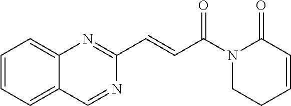 [SMILES: O=C1C=CCCN1C(=O)/C=C/c1ncc2ccccc2n1] | (E)-1-(3- (quinazolin-2- yl)acryloyl)-5,6- dihydropyridin- 2(1H)-one | 0.29 | 0.35 | N/A |
| 49 | 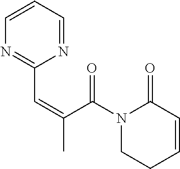 [SMILES: C/C(=C/c1ncccn1)C(=O)N1CCC=CC1=O] | 1-[(2E)-3-(4,5- dihydropyrimidin- 2-yl)-2-methylprop- 2-enoyl]-5,6- dihydropyridin- 2(1H)-one | 6.11 | 5.06 | N/A |
| 50 | 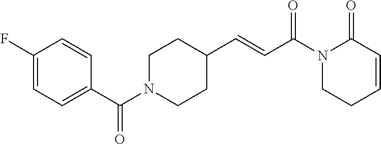 [SMILES: O=C(c1ccc(F)cc1)N1CCC(/C=C/C(=O)N2CCC=CC2=O)CC1] | (E)-1-(3-(1-(4- fluorobenzoyl)piper- idin-4-yl)acryloyl)- 5,6-dihydropyridin- 2(1H)-one | 0.41 | 0.5 | N/A |
| 51 | 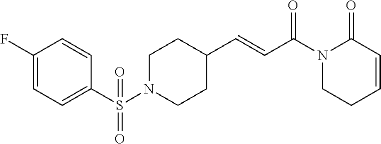 [SMILES: O=C1C=CCCN1C(=O)/C=C/C1CCN(S(=O)(=O)c2ccc(F)cc2)CC1] | (E)-1-(3-(1-(4- fluorophenylsulfon- yl)piperidin-4- yl)acryloyl)-5,6- dihydropyridin- 2(1H)-one | 0.56 | 0.57 | N/A |
| 52 | 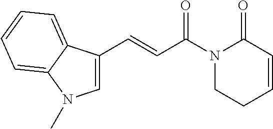 [SMILES: Cn1cc(/C=C/C(=O)N2CCC=CC2=O)c2ccccc21] | 1-[(2E)-3-(1- methyl-1H-indol-3- yl)prop-2-enoyl]- 5,6-dihydropyridin- 2(1H)-one | 1.00 | 1.52 | N/A |
| 53 | 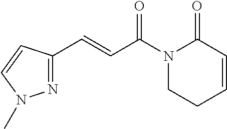 [SMILES: Cn1ccc(/C=C/C(=O)N2CCC=CC2=O)n1] | 1-[(2E)-3-(1- methyl-1H-pyrazol- 3-yl)prop-2-enoyl]- 5,6-dihydropyridin- 2(1H)-one | 0.56 | 0.73 | 2.12 |
| 54 | 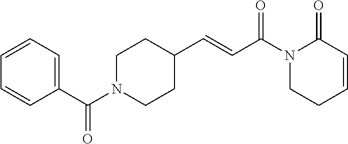 [SMILES: O=C(c1ccccc1)N1CCC(/C=C/C(=O)N2CCC=CC2=O)CC1] | 1-[(2E)-3-(1- benzoylpiperidin-4- yl)prop-2-enoyl]- 5,6-dihydropyridin- 2(1H)-one | N/A | N/A | 1.4 |
| 55 | 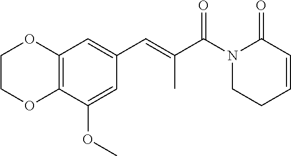 [SMILES: COc1cc(/C=C(\C)C(=O)N2CCC=CC2=O)cc2c1OCCO2] | 1-[(2E)-3-(8- methoxy-2,3- dihydro-1,4- benzodioxin-6-yl)- 2-methylprop-2- enoyl]-5,6- dihydropyridin- 2(1H)-one | 0.58 | 0.83 | 1.96 |
| 56 | 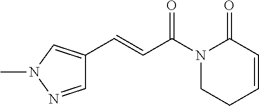 [SMILES: Cn1cc(/C=C/C(=O)N2CCC=CC2=O)cn1] | (E)-1-(3-(1-methyl- 1H-pyrazol-4- yl)acryloyl)-5,6- dihydropyridin- 2(1H)-one | 0.60 | 1.38 | 3.6 |
| 57 | 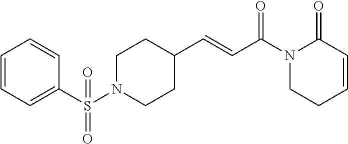 [SMILES: O=C1C=CCCN1C(=O)/C=C/C1CCN(S(=O)(=O)c2ccccc2)CC1] | (E)-1-(3-(1- (phenylsulfonyl) piperidin-4- yl)acryloyl)-5,6- dihydropyridin- 2(1H)-one | 0.68 | 0.53 | 1.36 |
| 58 | 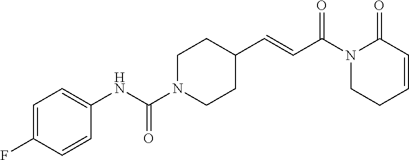 [SMILES: O=C(Nc1ccc(F)cc1)N1CCC(/C=C/C(=O)N2CCC=CC2=O)CC1] | N-(4-fluorophenyl)- 4-[(1E)-3-oxo-3-(6- oxo-3,6- dihydropyridin- 1(2H)-yl)prop-1- en-1-yl]piperidine- 1-carboxamide | 0.38 | 0.44 | 1.1 |
| 59 | 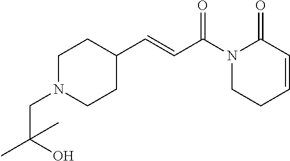 [SMILES: CC(C)(O)CN1CCC(/C=C/C(=O)N2CCC=CC2=O)CC1] | 1-{(2E)-3-[1-(2- hydroxy-2- methylpropyl)piperi- din-4-yl]prop-2- enoyl}-5,6- dihydropyridin- 2(1H)-one | 0.39 | 0.84 | N/A |
| 60 | 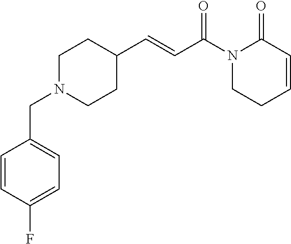 [SMILES: O=C1C=CCCN1C(=O)/C=C/C1CCN(Cc2ccc(F)cc2)CC1] | 1-{3-[1-(4- fluorobenzyl)piperi- din-4-yl]prop-2- enoyl}-5,6- dihydropyridin- 2(1H)-one | 0.34 | 0.59 | N/A |
| 61 | 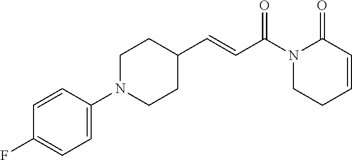 [SMILES: O=C1C=CCCN1C(=O)/C=C/C1CCN(c2ccc(F)cc2)CC1] | 1-{3-[1-(4- fluorophenyl)piperi- din-4-yl]prop-2- enoyl}-5,6- dihydropyridin- 2(1H)-one | 0.68 | 0.95 | N/A |
| 62 | 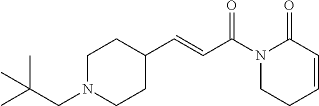 [SMILES: CC(C)(C)CN1CCC(/C=C/C(=O)N2CCC=CC2=O)CC1] | (E)-1-(3-(1- neopentylpiperidin- 4-yl)acryloyl)-5,6- dihydropyridin- 2(1H)-one | 0.31 | 0.58 | 1.73 |
| 63 | 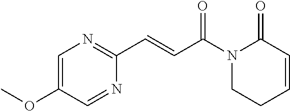 [SMILES: COc1cnc(/C=C/C(=O)N2CCC=CC2=O)nc1] | (E)-1-(3-(5- methoxypyrimidin- 2-yl)acryloyl)-5,6- dihydropyridin- 2(1H)-one | 0.29 | 0.48 | 2.11 |
| 64 | 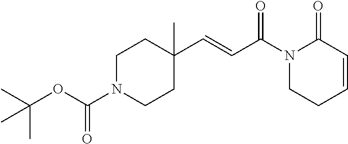 [SMILES: CC1(/C=C/C(=O)N2CCC=CC2=O)CCN(C(=O)OC(C)(C)C)CC1] | tert-butyl (E)-4- mcthyl-4-(3-oxo-3- (6-oxo-3,6- dihydropyridin- 1(2H)-yl)prop-1- en-1-yl)piperidine- 1-carboxylate | N/A | N/A | 4.32 |
| 65 | 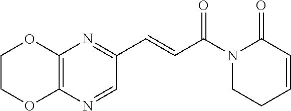 [SMILES: O=C1C=CCCN1C(=O)/C=C/c1cnc2c(n1)OCCO2] | (E)-1-(3-(2,3- dihydro- [1,4]dioxino[2,3- b]pyrazin-6- yl)acryloyl)-5,6- dihydropyridin- 2(1H)-one | N/A | N/A | 2.25 |
| 66 | 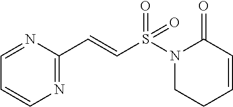 [SMILES: O=C1C=CCCN1S(=O)(=O)/C=C/c1ncccn1] | 1-{[(E)-2- (pyrimidin-2- yl)ethenyl]sulfonyl}- 5,6- dihydropyridin- 2(1H)-one | 0.21 | 0.95 | 3.11 |
| 67 | 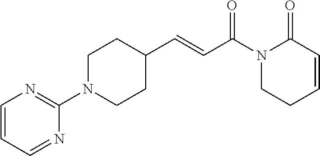 [SMILES: O=C1C=CCCN1C(=O)/C=C/C1CCN(c2ncccn2)CC1] | 1-{(2E)-3- [1- (pyrimidin- 2- yl)piperidin- 4-yl]prop-2- enoyl}-5,6- dihydropyridin- 2(1H)- one | 0.76 | 0.96 | 3.4 |
| 68 | 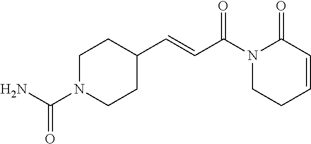 [SMILES: NC(=O)N1CCC(/C=C/C(=O)N2CCC=CC2=O)CC1] | (E)-4-(3-oxo-3-(6- oxo-3,6- dihydropyridin- 1(2H)-yl)prop-1- en-1-yl)piperidine- 1-carboxamide | 0.67 | 1.55 | 7.38 |
| 69 | 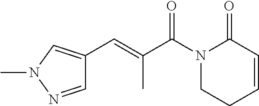 [SMILES: C/C(=C\c1cnn(C)c1)C(=O)N1CCC=CC1=O] | 1-[(2E)-2-methyl-3- (1-methyl-1H- pyrazol-4-yl)prop- 2-enoyl]-5,6- dihydropyridin- 2(1H)-one | 1.98 | 4.01 | 14 |
| 70 | 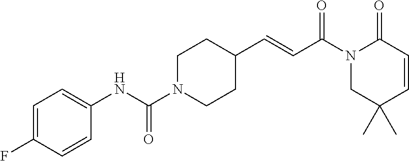 [SMILES: CC1(C)C=CC(=O)N(C(=O)/C=C/C2CCN(C(=O)Nc3ccc(F)cc3)CC2)C1] | (E)-4-(3-(3,3- dimethyl-6-oxo- 3,6-dihydropyridin- 1(2H)-yl)-3- oxoprop-1-en-1-yl)- N-(4- fluorophenyl)piperi- dine-1-carboxamide | 1.35 | 1.75 | 7.06 |
| 71 | 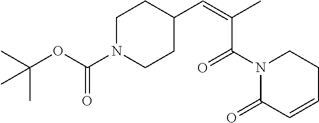 [SMILES: C/C(=C/C1CCN(C(=O)OC(C)(C)C)CC1)C(=O)N1CCC=CC1=O] | (Z)-tert-butyl 4-(2- methyl-3-oxo-3-(2- oxo-5,6- dihydropyridin- 1(2H)-yl)prop-1- enyl)piperidine-1- carboxylate | 3.91 | 4.67 | 13.6 |
| 72 | 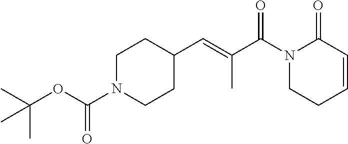 [SMILES: C/C(=C\C1CCN(C(=O)OC(C)(C)C)CC1)C(=O)N1CCC=CC1=O] | tert-butyl (E)-4-(2- methyl-3-oxo-3-(6- oxo-3,6- dihydropyridin- 1(2H)-yl)prop-1- en-1-yl)piperidine- 1-carboxylate | 2.67 | 4.43 | 10.3 |
| 73 | 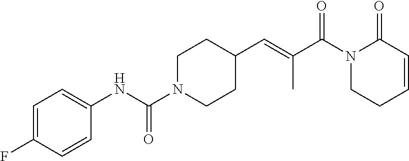 [SMILES: C/C(=C\C1CCN(C(=O)Nc2ccc(F)cc2)CC1)C(=O)N1CCC=CC1=O] | (E)-N-(4- fluorophenyl)-4-(2- methyl-3-oxo-3-(6- oxo-3,6- dihydropyridin- 1(2H)-yl)prop-1- en-1-yl)piperidine- 1-carboxamide | 0.95 | 1.62 | 4.28 |
| 74 | 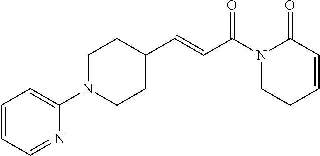 [SMILES: O=C1C=CCCN1C(=O)/C=C/C1CCN(c2ccccn2)CC1] | (E)-(3-(1- (pyridin-2- yl)piperidin-4- yl)acryloyl)-5,6- dihydropyridin- 2(1H)-one | 0.66 | 1 | 2.58 |
| 75 | 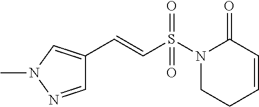 [SMILES: Cn1cc(/C=C/S(=O)(=O)N2CCC=CC2=O)cn1] | 1-{[(E)-2-(1- methyl-1H-pyrazol- 4- yl)ethenyl]sulfonyl}- 5,6- dihydropyridin- 2(1H)-one | 0.86 | 1.6 | 4.62 |
| 76 | 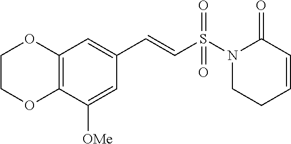 [SMILES: COc1cc(/C=C/S(=O)(=O)N2CCC=CC2=O)cc2c1OCCO2] | 1-{[(E)-2-(8- methoxy-2,3- dihydro-1,4- benzodioxin-6- yl)ethenyl]sulfonyl}- 5,6- dihydropyridin- 2(1H)-one | 0.563 | 1 | 1.85 |
| 77 | 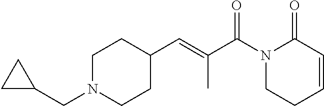 [SMILES: C/C(=C\C1CCN(CC2CC2)CC1)C(=O)N1CCC=CC1=O] | (E)-1-(3-(1- (cyclopropylmethyl) piperidin-4-yl)-2- methylacryloyl)- 5,6-dihydropyridin- 2(1H)-one | 3.34 | 5.44 | 7.5 |
| 78 | 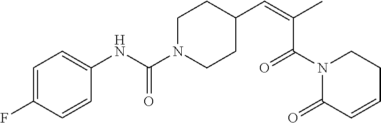 [SMILES: C/C(=C/C1CCN(C(=O)Nc2ccc(F)cc2)CC1)C(=O)N1CCC=CC1=O] | (Z)-1-(3-(1-(4- fluorophenyl)piperi- din-4-yl)-2- methylacryloyl)- 5,6-dihydropyridin- 2(1H)-one | 0.95 | 1.53 | 4.85 |
| 79 | 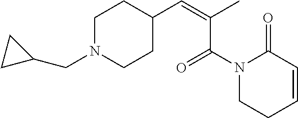 [SMILES: C/C(=C/C1CCN(CC2CC2)CC1)C(=O)N1CCC=CC1=O] | (Z)-1-(3-(1- (cyclopropylmethyl) piperidin-4-yl)-2- methylacryloyl)- 5,6-dihydropyridin- 2(1H)-one | 1.03 | 1.91 | 2.69 |
| 80 | 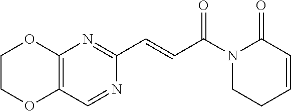 [SMILES: O=C1C=CCCN1C(=O)/C=C/c1ncc2c(n1)OCCO2] | (E)-1-(3-(6,7- dihydro- [1,4]dioxino[2,3- d]pyrimidin-2- yl)acryloyl)-5,6- dihydropyridin- 2(1H)-one | 1.15 | 1.8 | 3.21 |
| 81 | 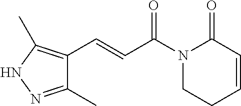 [SMILES: Cc1n[nH]c(C)c1/C=C/C(=O)N1CCC=CC1=O] | (E)-1-(3-(3,5- dimethyl-1H- pyrazol-4- yl)acryloyl)-5,6- dihydropyridin- 2(1H)-one | 1.49 | 2.7 | 5.1 |
| 82 | 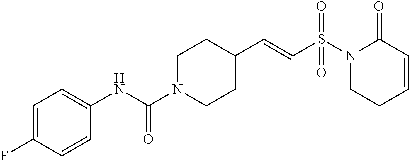 [SMILES: O=C(Nc1ccc(F)cc1)N1CCC(/C=C/S(=O)(=O)N2CCC=CC2=O)CC1] | (E)-N-(4- fluorophenyl)-4-(2- ((6-oxo-3,6- dihydropyridin- 1(2H)- yl)sulfonyl)vinyl) piperidine-1- carboxamide | 0.36 | 0.8 | 2.25 |
| 83 | 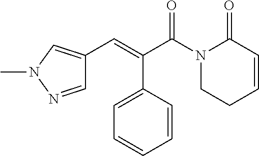 [SMILES: Cn1cc(/C=C(/C(=O)N2CCC=CC2=O)c2ccccc2)cn1] | 1-[(2E)-3-(1- methyl-1H-pyrazol- 4-yl)-2-phenylprop- 2-enoyl]-5,6- dihydropyridin- 2(1H)-one | 2.75 | 4.84 | 11.1 |
| 84 | 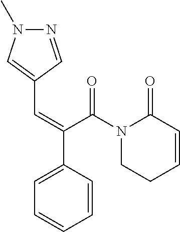 [SMILES: Cn1cc(/C=C(\C(=O)N2CCC=CC2=O)c2ccccc2)cn1] | (Z)-1-(3-(1-methyl- 1H-pyrazol-4-yl)-2- phenylacryloyl)- 5,6-dihydropyridin- 2(1H)-one | 1.55 | 1.63 | 3.71 |
| 85 | 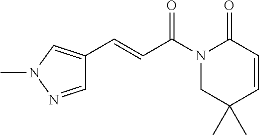 [SMILES: Cn1cc(/C=C/C(=O)N2CC(C)(C)C=CC2=O)cn1] | (E)-5,5-dimethyl-1- (3-(1-methyl-1H- pyrazol-4- yl)acryloyl)-5,6- dihydropyridin- 2(1H)-one | 4.6 | 6.79 | 31 |
| 86 | 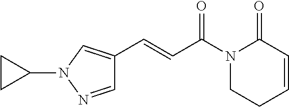 [SMILES: O=C1C=CCCN1C(=O)/C=C/c1cnn(C2CC2)c1] | (E)-1-(3-(1- cyclopropyl-1H- pyrazol-4- yl)acryloyl)-5,6- dihydropyridin- 2(1H)-one | 0.85 | 1.67 | 3.83 |
| 87 | 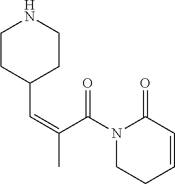 [SMILES: C/C(=C/C1CCNCC1)C(=O)N1CCC=CC1=O] | (Z)-1-(2-methyl-3- (piperidin-4- yl)acryloyl)-5,6- dihydropyridin- 2(1H)-one 2,2,2- trifluoroacetate | 3.66 | 5.53 | 10.1 |
| 88 | 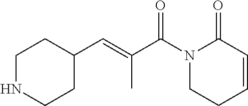 [SMILES: C/C(=C\C1CCNCC1)C(=O)N1CCC=CC1=O] | (E)-1-(2-methyl-3- (piperidin-4- yl)acryloyl)-5,6- dihydropyridin- 2(1H)-one 2,2,2- trifluoroacetate | 3.78 | 6.56 | 7.06 |
| 89 | 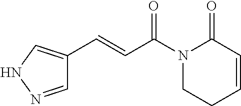 [SMILES: O=C1C=CCCN1C(=O)/C=C/c1cn[nH]c1] | (E)-1-(3-(1H- pyrazol-4- yl)acryloyl)-5,6- dihydropyridin- 2(1H)-one | 3.58 | 8.41 | 2.7 |
| 90 | 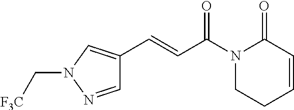 [SMILES: O=C1C=CCCN1C(=O)/C=C/c1cnn(CC(F)(F)F)c1] | 1 11-{(2E)-3-[1- (2,2,2- trifluoroethyl)-1H- pyrazol-4-yl]prop- 2-enoyl}-5,6- dihydropyridin- 2(1H)-one | 0.44 | 1.47 | 7.07 |
| 91 | 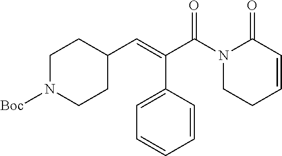 [SMILES: CC(C)(C)OC(=O)N1CCC(/C=C(/C(=O)N2CCC=CC2=O)c2ccccc2)CC1] | (E)-tert-butyl 4-(3- oxo-3-(2-oxo-5,6- dihydropyridin- 1(2H)-yl)-2- phenylprop-1- enyl)piperidine-1- carboxylate | 3.2 | 4.14 | 9.16 |
| 92 | 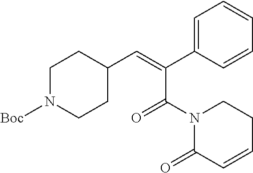 [SMILES: CC(C)(C)OC(=O)N1CCC(/C=C(\C(=O)N2CCC=CC2=O)c2ccccc2)CC1] | (Z)-tert-butyl 4-(3- oxo-3-(2-oxo-5,6- dihydropyridin- 1(2H)-yl)-2- phenylprop-1- enyl)piperidine-1- carboxylate | 4.32 | 4.75 | 9.72 |
| 93 | 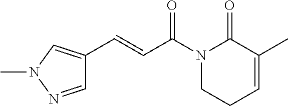 | (E)-3-methyl-1-(3- (1-methyl-1H- pyrazol-4- yl)acryloyl)-5,6- dihydropyridin- 2(1H)-one | N/A | N/A | 88.3 |
| 94 | 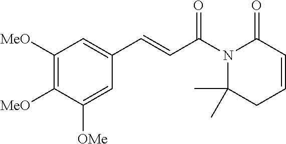 [SMILES: COc1cc(/C=C/C(=O)N2C(=O)C=CCC2(C)C)cc(OC)c1OC] | (E)-6,6-dimethyl-1- (3-(3,4,5- trimethoxyphenyl) acryloyl)-5,6- dihydropyridin- 2(1H)-one | N/A | N/A | 3.15 |
| 95 | 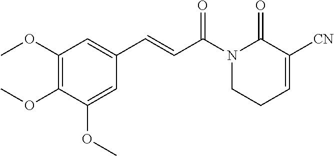 [SMILES: COc1cc(/C=C/C(=O)N2CCC=C(C#N)C2=O)cc(OC)c1OC] | (E)-2-oxo-1-(3- (3,4,5- trimethoxyphenyl) acryloyl)-1,2,5,6- tetrahydropyridine- 3-carbonitrile | N/A | N/A | N/A |
| 96 | 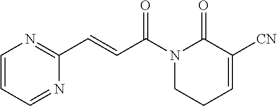 [SMILES: N#CC1=CCCN(C(=O)/C=C/c2ncccn2)C1=O] | (E)-2-oxo-1-(3- (pyrimidin-2- yl)acryloyl)- 1,2,5,6- tetrahydropyridine- 3-carbonitrile | N/A | N/A | N/A |
| 97 | 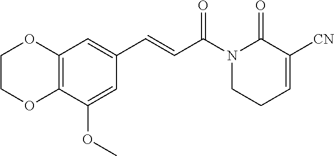 [SMILES: COc1cc(/C=C/C(=O)N2CCC=C(C#N)C2=O)cc2c1OCCO2] | (E)-1-(3-(8- methoxy-2,3- dihydrobenzo[b][1, 4]dioxin-6- yl)acryloyl)-2-oxo- 1,2,5,6- tetrahydropyridine- 3-carbonitrile | N/A | N/A | N/A |
| 98 | 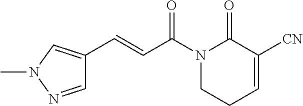 [SMILES: Cn1cc(/C=C/C(=O)N2CCC=C(C#N)C2=O)cn1] | (E)-1-(3-(1-methyl- 1H-pyrazol-4- yl)acryloyl)-2-oxo- 1,2,5,6- tetrahydropyridine- 3-carbonitrile | N/A | N/A | N/A |

### Example 1

### Preparation of (E)-1-(3-(8-methoxy-2,3-dihydrobenzo[b][1,4]dioxin-6-yl)acryloyl)-5,6-dihydropyridin-2(1H)-one

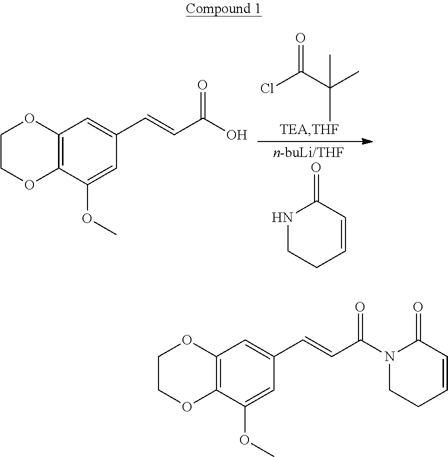

To a solution of (E)-3-(8-methoxy-2,3-dihydrobenzo[b][1,4]dioxin-6-yl)acrylic acid (0.2 g, 0.847 mmol) in dry THF was added triethylamine (0.12 mL, 0.932 mmol, 1.1 eq) and pivaloyl chloride (0.11 mL, 0.932 mmol, 1.1 eq) the reaction mixture was stirred at 0° C. for 45 min. Progress was monitored by TLC. After completion, the mixture was filtered and the filtrate was used directly for the next step. To a stirred solution of 5,6-dihydropyridin-2(1H)-one (0.1 g, 1.03 mmol, 1.0 eq) in dry THF at −78° C. was added n-BuLi (2.5 M in hexane, 0.45 mL, 1.13 mmol, 1.1 eq) and the mixture was allowed to stir at the same temperature for 30 min. To this solution was added a solution of (E)-3-(8-methoxy-2,3-dihydrobenzo[b][1,4]dioxin-6-yl)acrylic pivalic anhydride (0.29 g, 0.927 mmol, 0.9 eq). The mixture was allowed to stir at −78° C. for 1 h. Progress was monitored by TLC and LCMS. After 1 h, the reaction mixture was brought to 0° C., diluted with water (30 mL) and extracted with ethyl acetate (3×30 mL). The combined organic layers were washed with brine, dried over anhydrous Na₂SO₄, filtered and evaporated to dryness under vacuum. The crude material was purified by reversed phase HPLC to afford the title compound (58 mg, 21.8%).

LCMS: 316 [M+1]⁺

¹H NMR: (400 MHz, CDCl₃) δ 7.62 (d, 1H), 7.39 (d, 1H), 6.90-6.95 (m, 1H), 6.75 (d, 2H), 6.02 (d, 1H), 4.35 (t, 2H), 4.25 (t, 2H), 4.01 (t, 2H), 3.88 (s, 3H), 2.41-2.46 (m, 2H).

### Example 2

### Preparation of (E)-1-(3-cyclohexylacryloyl)-5,6-dihydropyridin-2(1H)-one

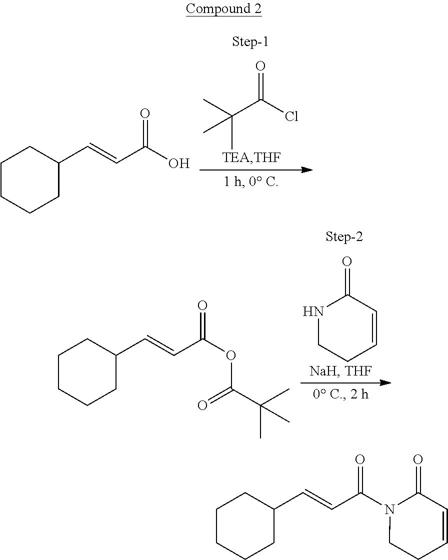

### Step-1

To a stirred solution of (E)-3-cyclohexylacrylic acid (400 mg, 2.59 mmol) in THF (5 mL) at 0° C. was added triethylamine (0.55 mL, 3.89 mmol, 1.5 eq.) and pivaloyl chloride (0.35 mL, 2.85 mmol, 1.1 eq.) and the reaction mixture was allowed to stir at 0° C. for 1 h. Progress was monitored by TLC. After completion, the reaction mixture was filtered and the filtrate was carried to the next step without further purification.

LCMS: 239 [M+1]⁺

### Step-2

To the stirred solution of 5,6-dihydropyridin-2(1H)-one (200 mg, 2.06 mmol) in THF (5 mL) at 0° C. was added NaH (174 mg, 3.08 mmol, 1.5 eq) and the mixture was stirred at 0° C. for 30 mins. To this was added (E)-3-cyclohexylacrylic pivalic anhydride (540 mg, 2.27 mmol, 1.1 eq) and the resulting mixture was allowed to stir at 0° C. for 2 h. Progress was monitored by TLC. After completion, the reaction mixture was diluted with water (20 mL) and extracted with EtOAc (2×100 mL). The combined organic layers were washed with brine, dried over Na₂SO₄, filtered and concentrated under reduced pressure. The crude product was purified by reversed phase HPLC to afford the title compound (43 mg, 9.0%) as a white solid.

LCMS: 234 [M+1]⁺

¹H NMR: (400 MHz, CDCl₃) δ 6.98 (m, 1H), 6.82 (m, 1H), 6.78 (d, 1H), 6.0 (d, 1H), 3.98 (t, 2H), 2.42 (d, 2H), 2.19 (m, 1H), 1.78 (t, 4H), 1.67 (d, 1H), 1.36-1.10 (m, 5H).

### Example 3

### Preparation of (E)-1-(3-(quinolin-3-yl)acryloyl)-1,5,6,7-tetrahydro-2H-azepin-2-one

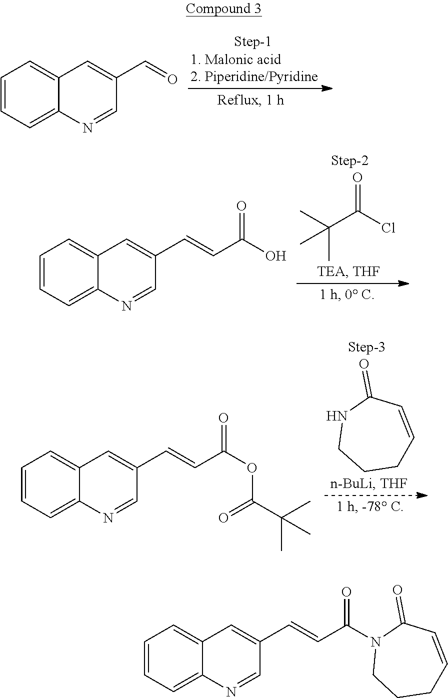

### Step-1

To the stirred solution of quinoline-3-carbaldehyde (350 mg, 2.23 mmol) in pyridine (10 mL) and piperidine (2 mL) at room temperature was added malonic acid (487 mg, 4.68 mmol, 2.1 eq). The reaction mixture was heated to 110° C. and allowed to proceed for 1 h. Progress of the reaction was monitored by TLC. After completion, the reaction mixture was diluted with 1N HCl (20 mL) and extracted with EtOAc (2×100 mL). The combined organic layers were washed with water, dried over Na₂SO₄, filtered and concentrated under reduced pressure to obtain (E)-3-(quinolin-3-yl)acrylic acid (210 mg, 47%) which was used without further purification.

LCMS: 200 [M+1]⁺

### Step-2

To a stirred solution of (E)-3-(quinolin-3-yl)acrylic acid (150 mg, 0.75 mmol, 1.0 eq) in THF (5 mL) at 0° C. was added triethylamine (0.16 mL, 1.13 mmol, 1.5 eq) and pivaloyl chloride (0.1 mL, 0.83 mmol, 1.1 eq) and the reaction mixture was allowed to stir at 0° C. for 1 h. Progress was monitored by TLC. After completion, the reaction mixture was filtered and the filtrate was carried to the next step without further purification.

LCMS: 284 [M+1]⁺

### Step-3

To the stirred solution of 1,5,6,7-tetrahydro-2H-azepin-2-one (75 mg, 0.67 mmol) in THF (5 mL) at −78° C. was added n-BuLi (2.5 M in hexane, 0.35 mL, 0.88 mmol, 1.3 eq) and the resulting mixture was stirred at −78° C. for 30 min. To this was added pivalic (E)-3-(quinolin-3-yl)acrylic anhydride (210 mg, 0.74 mmol, 1.1 eq) and the mixture was stirred at −70° C. for 2 h. Progress was monitored by TLC. After completion, the reaction mixture was diluted with water (20 mL) and extracted with EtOAc (2×100 mL). The combined organic layers were washed with brine, dried over Na₂SO₄, filtered and concentrated under reduced pressure. The crude product was purified by reversed phase HPLC to afford the title compound (10 mg, 5.1%) as a white solid.

LCMS: 293 [M+1]⁺

¹H NMR: (400 MHz, CDCl₃) δ 9.10 (s, 1H), 8.32 (s, 1H), 8.11 (d, 1H), 7.87 (m, 2H), 7.75 (t, 1H), 7.66 (d, 1H), 7.58 (t, 1H), 6.6-6.54 (m, 1H), 6.15-6.12 (d, 1H), 4.05 (t, 2H), 2.45 (q, 2H), 2.04 (m, 2H).

### Example 4

### Preparation of (E)-1-(3-(pyridin-3-yl)acryloyl)-1,5,6,7-tetrahydro-2H-azepin-2-one

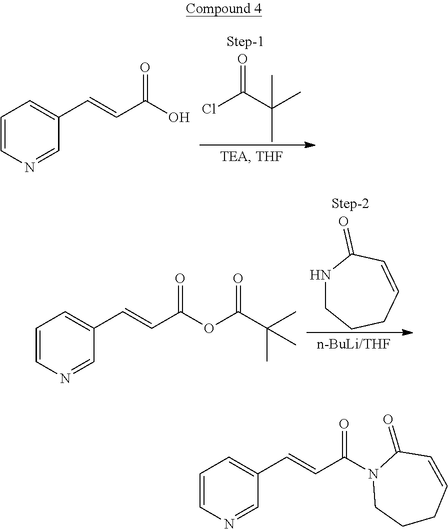

### Step-1

To a solution of (E)-3-(pyridin-3-yl)acrylic acid (0.2 g, 1.34 mmol) in dry THF (10 mL) was added triethylamine (0.18 mL, 1.47 mmol, 1.1 eq) followed by pivaloyl chloride 2 (0.18 mL, 1.47 mmol, 1.1 eq) and the mixture was stirred at 0° C. for 45 min. Progress was monitored by TLC. After completion, reaction mixture was filtered and the filtrate was carried to the next step without further purification.

### Step-2

To a stirred solution of 1,5,6,7-tetrahydro-2H-azepin-2-one (0.1 g, 0.89 mmol) in dry THF at −78° C. was added n-BuLi (2.5 M in hexane, 0.45 mL, 0.98 mmol, 1.1 eq) and the mixture was allowed to stir at the same temperature for 30 min. To this mixture was added a solution of pivalic (E)-3-(pyridin-3-yl)acrylic anhydride (0.187 g, 0.80 mmol, 0.9 eq) in THF (10 mL) and the resulting mixture was allowed to stir at −78° C. for 1 h. Progress was monitored by TLC and LCMS. After completion, the reaction mixture was warmed to 0° C., diluted with water (30 mL) and extracted with EtOAc (3×30 mL). The combined organic layers were washed with brine (30 mL), dried over anhydrous Na₂SO₄, filtered and concentrated. The crude material was purified by reversed phase HPLC to afford the title compound (60 mg, 18.8%).

LCMS: 243 [M+1]⁺

¹H NMR: (500 MHz, CDCl₃) δ 8.82-8.74 (m, 1H), 8.61-8.56 (m, 1H), 7.90 (dt, 1H), 7.72 (d, 1H), 7.51 (d, 1H), 7.31 (dd, 1H), 6.56 (dt, 1H), 6.11 (dd, 1H), 4.06-3.92 (m, 2H), 2.49-2.33 (m, 2H), 2.07-1.92 (m, 2H).

### Example 5

### Preparation of (E)-1-(3-(pyridin-4-yl)acryloyl)-1,5,6,7-tetrahydro-2H-azepin-2-one

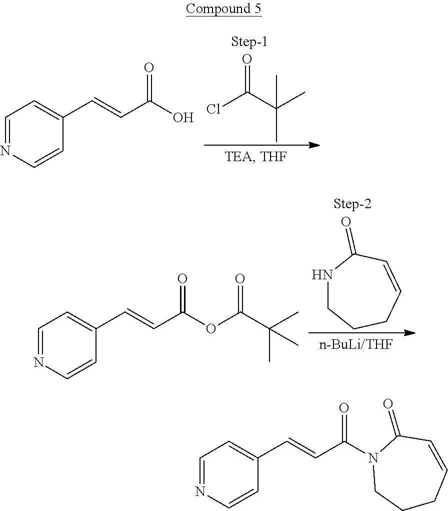

### Step-1

To a solution of (E)-3-(pyridin-4-yl)acrylic acid (0.2 g, 1.34 mmol) in dry THF (10 mL) was added triethylamine (0.18 mL, 1.47 mmol, 1.1 eq) followed by pivaloyl chloride (0.18 mL, 1.47 mmol, 1.1 eq.) and the reaction mixture was stirred at 0° C. for 45 min. Progress was monitored by TLC. After completion, the reaction mixture was filtered and the filtrate was used directly for the next step.

### Step-2

To a stirred solution of 1,5,6,7-tetrahydro-2H-azepin-2-one (0.1 g, 0.89 mmol) in dry THF (10 mL) at −78° C. was added n-BuLi (2.5 M in hexane, 0.45 mL, 0.98 mmol, 1.1 eq) and the mixture was allowed to stir at the same temperature for 30 min. To this mixture was added a solution of pivalic (E)-3-(pyridin-4-yl)acrylic anhydride (0.187 g, 0.80 mmol, 0.9 eq.) in THF (10 mL) and the resulting mixture was allowed to stir at −78° C. for 1 h. Progress was monitored by TLC and LCMS. After completion, the reaction mixture was warmed to 0° C., diluted with water (30 mL) and extracted with EtOAc (3×30 mL). The combined organic layers were washed with brine, dried over anhydrous Na₂SO₄, filtered and concentrated. The crude material was purified by reversed phase HPLC to afford the title compound (80 mg, 25.1%).

LCMS: 243 [M+1]⁺

¹H NMR: (500 MHz, CDCl₃) δ 8.63 (d, 2H), 7.65-7.53 (m, 2H), 7.43-7.38 (m, 2H), 6.57 (dt, 1H), 6.12 (dt, 1H), 4.03-3.97 (m, 2H), 2.47-2.39 (m, 2H), 2.06-1.97 (m, 2H).

### Example 6

### Preparation of (E)-1-(3-(pyrazin-2-yl)acryloyl)-1,5,6,7-tetrahydro-2H-azepin-2-one

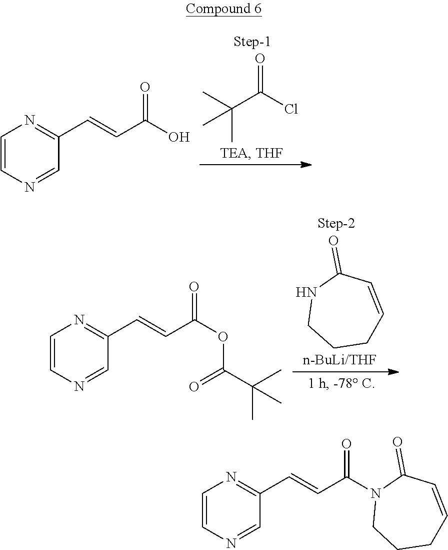

### Step-1

To a stirred solution of (E)-3-(pyrazin-2-yl)acrylic acid (150 mg, 0.99 mmol) in THF (5 mL) at 0° C. was added triethylamine (0.2 mL, 1.50 mmol, 1.5 eq) followed by pivaloyl chloride (0.13 mL, 1.10 mmol, 1.1 eq.) and the reaction mixture was allowed to stir at 0° C. for 1 h. Progress was monitored by TLC. After completion, the reaction mixture was filtered and the filtrate was carried to next step without any further purification.

LCMS: 235 [M+1]⁺

### Step-2

To the stirred solution of 1,5,6,7-tetrahydro-2H-azepin-2-one (100 mg, 0.90 mmol) in THF (5 mL) at −78° C. was added n-BuLi (0.47 mL, 1.17 mmol, 1.3 eq) and the reaction mixture was stirred at −78° C. for 30 min. To this mixture was added a solution of pivalic (E)-3-(pyrazin-2-yl)acrylic anhydride (211 mg, 0.90 mmol, 1.1 eq.) in THF (5 mL) and the resulting mixture was stirred at −78° C. for 2 h. Progress was monitored by TLC. After completion, the reaction mixture was diluted with water (20 mL) and extracted with EtOAc (2×100 mL). The combined organic layers were washed with brine, dried over Na₂SO₄, filtered and concentrated under reduced pressure. The crude material was purified by reversed phase HPLC to afford the title compound (21 mg, 9.6%) as white solid.

LCMS: 244 [M+1]⁺

¹H NMR: (500 MHz, CDCl₃) δ 8.69 (d, 1H), 8.60 (t, 1H), 8.51 (d, 1H), 7.89 (d, 1H), 7.69 (d, 1H), 6.57 (d, 1H), 6.12 (d, 1H), 4.01 (t, 2H), 2.43 (q, 2H), 2.06-1.96 (m, 2H).

### Example 7

### Preparation of (E)-1-(3-(pyrimidin-2-yl)acryloyl)-5,6-dihydropyridin-2(1H)-one

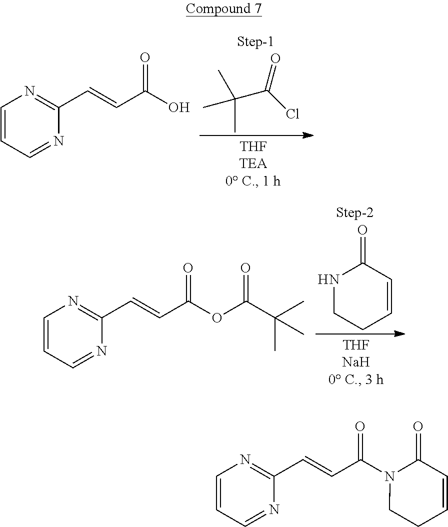

### Step-1

To a stirred solution of (E)-3-(pyrimidin-2-yl)prop-2-enoic acid (150 mg, 1.0 mmol) in THF (8 mL) at 0° C. was added triethylamine (0.1 mL, 1.1 mmol) followed by pivaloyl chloride (0.1 mL, 1.1 mmol) and the resulting mixture was stirred at the same temperature for 1 h. Progress was monitored by TLC. After completion, the reaction mixture was filtered and the filtrate was carried to next step without any further purification.

### Step-2

To a stirred solution of 5,6-dihydropyridin-2(1H)-one (80 mg, 0.82 mmol) in THF (5 mL) was added NaH (39 mg, 0.98 mmol, 1.2 eq) at 0° C. and the resulting mixture was allowed to stir at the same temperature for 30 min. To this mixture was added a solution of 2,2-dimethylpropanoic (E)-3-(pyrimidin-2-yl)prop-2-enoic anhydride (200 mg, 1.1 mmol, 1.3 eq) in THF (8 mL) and the resulting mixture was allowed to stir at the same temperature for 3 h. Progress was monitored by TLC and LCMS. After completion, the reaction mixture was diluted with ice cold water (20 mL) and extracted with EtOAc (3×50 mL). The combined organic layers were washed with water (2×25 mL), dried over anhydrous Na₂SO₄ and filtered. Removal of solvent under reduce pressure afforded an oily residue which was purified by reversed phase HPLC to afford the title compound (30 mg, 13%).

LCMS: 230 [M+1]⁺

¹H NMR: (500 MHz, CDCl₃) δ 8.76 (d, 2H), 8.07 (d, 1H), 7.65 (d, 1H), 7.19 (t, 1H), 6.96 (dt, 1H), 6.05 (d, 1H), 4.05 (t, 2H), 2.50 (tdd, 2H).

### Example 8

### Preparation of (E)-1-(3-(pyridin-2-yl)acryloyl)-1,5,6,7-tetrahydro-2H-azepin-2-one

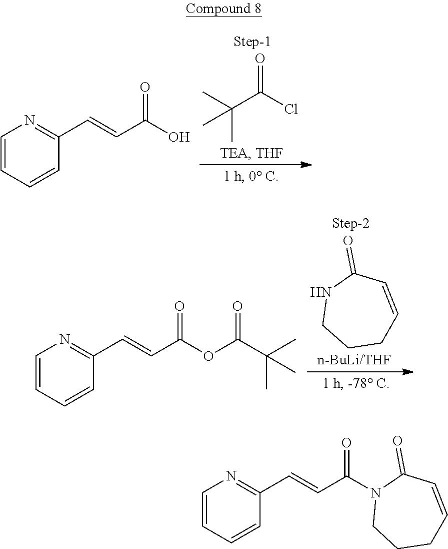

### Step-1

To a stirred solution of (E)-3-(pyridin-2-yl)acrylic acid (150 mg, 1.01 mmol) in THF (5 mL) at 0° C. was added triethylamine (0.21 mL, 1.51 mmol, 1.5 eq) followed by pivaloyl chloride (0.13 mL, 1.11 mmol, 1.1 eq) and the reaction mixture was allowed to stir at 0° C. for 1 h. Progress was monitored by TLC. After completion, the reaction mixture was filtered and the filtrate was carried to next step without any further purification.

LCMS: 234 [M+1]⁺

### Step-2

To a stirred solution of 1,5,6,7-tetrahydro-2H-azepin-2-one (100 mg, 0.90 mmol) in THF (5 mL) at −78° C. was added n-BuLi (2.5 M in hexane, 0.47 mL, 1.17 mmol, 1.3 eq) and the reaction mixture was stirred at −78° C. for 30 min. To this reaction mixture was added pivalic (E)-3-(pyridin-2-yl)acrylic anhydride (210 mg, 0.90 mmol, 1.1 eq) and stirred at −78° C. for 2 h. Progress was monitored by TLC. After completion, the reaction mixture was diluted with water (20 mL) and extracted with EtOAc (2×100 mL). The combined organic layers were washed with brine, dried over Na₂SO₄, filtered and concentrated. The crude material was purified by reversed phase HPLC to afford the title compound (28 mg, 12.8%) as a white solid.

LCMS: 243 [M+1]⁺

¹H NMR: (500 MHz, CDCl₃) δ 8.64 (d, 1H), 7.82-7.66 (m, 3H), 7.46 (d, 1H), 7.25-7.21 (m, 1H), 6.55 (d, 1H), 6.11 (d, 1H), 4.00 (t, 2H), 2.41 (q, 2H), 2.04-1.92 (m, 2H).

### Example 9

### Preparation of (E)-1-(3-(1-acetylpiperidin-4-yl)acryloyl)-5,6-dihydropyridin-2(1H)-one

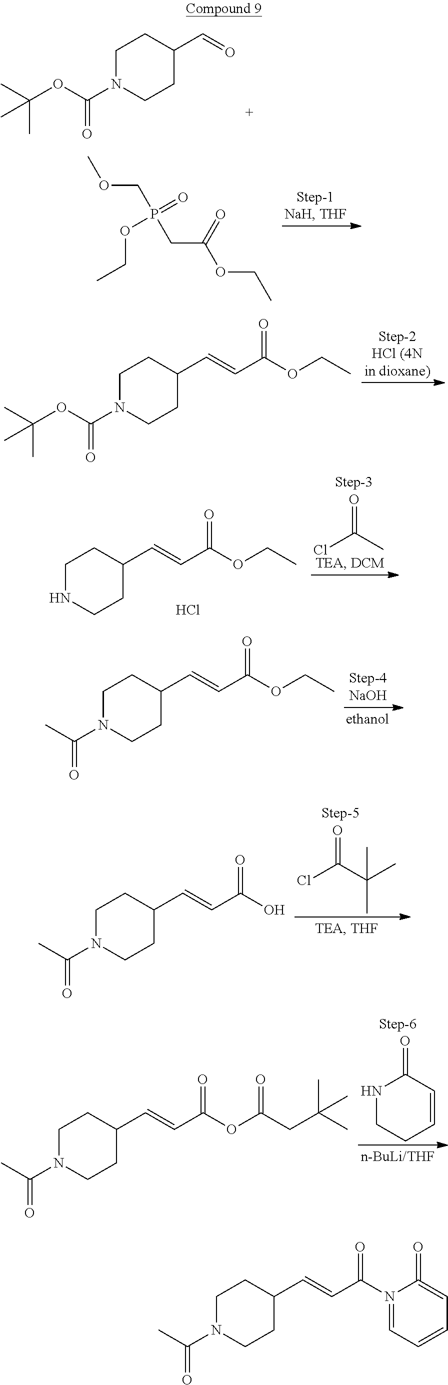

### Step-1

To a suspension of sodium hydride (60% in mineral oil, 1.03 g, 25.8 mmol, 1.1 eq) in THF (50 mL) was added ethyl 2-(diethoxyphosphoryl)acetate (10.51 g, 46.9 mmol, 2.0 eq) dropwise at 0° C. and the mixture was allowed to stir at the same temperature for 30 min. To this mixture was added a solution of tert-butyl 4-formylpiperidine-1-carboxylate (5.0 g, 23.5 mmol) in THF (5 mL) and the resulting mixture was allowed to stir at room temperature for 1 h. Progress was monitored by TLC. After completion, the mixture was diluted with saturated aqueous NH₄Cl (100 mL) and extracted with EtOAc (3×50 mL). The combined organic layers were washed with brine, dried over anhydrous Na₂SO₄, filtered and evaporated to dryness under vacuum. The crude product was purified by CombiFlash to afford tert-butyl (E)-4-(3-ethoxy-3-oxoprop-1-en-1-yl)piperidine-1-carboxylate (3.5 g, 52.7%).

LCMS: 284 [M+1]⁺

### Step-2

A solution of tert-butyl(E)-4-(3-ethoxy-3-oxoprop-1-en-1-yl)piperidine-1-carboxylate (1.50 g, 7.18 mmol) in 4N HCl in dioxane (30 mL) was stirred at room temperature for 4 h. Progress was monitored by TLC. After completion, the reaction mixture was evaporated under reduce pressure, triturated with Et₂O to afford ethyl (E)-3-(piperidin-4-yl)acrylate hydrochloride (0.85 g, 88%).

LCMS: 184 [M+1]⁺

### Step-3

To a stirred solution of ethyl (E)-3-(piperidin-4-yl)acrylate hydrochloride (0.85 g, 4.37 mmol) and triethylamine (1.22 mL, 8.74 mmol, 2.0 eq) in CH₂Cl₂ at 0° C. was added acetyl chloride (0.37 mL, 5.24 mmol, 1.2 eq) dropwise. The ice bath was removed and the mixture was stirred at room temperature for 4 h. Progress was monitored by TLC and LCMS. After completion, the reaction mixture was diluted with saturated aqueous NH₄Cl (30 mL) and extracted with EtOAc (3×30 mL). The combined organic layers were washed with brine, dried over anhydrous Na₂SO₄, filtered and evaporated under vacuum. The crude product was purified by CombiFlash to afford ethyl (E)-3-(1-acetylpiperidin-4-yl)acrylate (0.56 g, 57%) as a pale yellow oil.

LCMS: 226 [M+1]⁺

### Step-4

To a solution of ethyl (E)-3-(1-acetylpiperidin-4-yl)acrylate (0.56 g, 2.48 mmol) in EtOH (200 mL) was added a solution of NaOH (0.298 g, 7.44 mmol) in water (20 mL) and the reaction mixture was stirred at 70° C. for 4 h. The mixture was concentrated under vacuum and then acidified with 2N HCl to pH 3. The mixture was then diluted saturated aqueous NH₄Cl (30 mL) and extracted with CH₂Cl₂ (3×30 mL). The combined organic layers were washed with brine (50 mL), dried over anhydrous Na₂SO₄ and filtered. The solvent was removed under reduced pressure to obtain (E)-3-(1-acetylpiperidin-4-yl)acrylic acid (0.32 g, 65%) as crystalline solid.

LCMS: 198 [M+1]⁺

### Step-5

To a solution of (E)-3-(1-acetylpiperidin-4-yl)acrylic acid (0.3 g, 1.52 mmol) in dry THF was added triethylamine (0.21 mL, 1.67 mmol, 1.1 eq) followed by pivaloyl chloride (0.2 mL, 1.67 mmol, 1.1 eq) and the reaction mixture was stirred at 0° C. for 45 min. Progress was monitored by TLC. After completion, the reaction mixture was filtered used directly for the next step.

### Step-6

To a stirred solution of 5,6-dihydropyridin-2(1H)-one (0.1 g, 1.03 mmol) in dry THF at −78° C. was added n-BuLi (2.5 M in hexane, 0.45 mL, 1.13 mmol, 1.1 eq) and the mixture was allowed to stir at the same temperature for 30 min. To this solution was added a solution of (E)-3-(1-acetylpiperidin-4-yl)acrylic (tert-butyl carbonic) anhydride (0.275 g, 0.92 mmol, 0.9 eq) in THF and the reaction mixture was allowed to stir at −78° C. for 1 h. Progress was monitored by TLC and LCMS. After completion, the reaction mixture was brought to 0° C., diluted with water (30 mL) and extracted with EtOAc (3×30 mL). The combined organic layers were washed with brine, dried over anhydrous Na₂SO₄, filtered and evaporated to dryness under vacuum. The crude material which was purified by reversed phase HPLC to afford the title compound (0.014 g, 15%).

LCMS: 277 [M+1]⁺

¹H NMR: (400 MHz, CDCl₃) δ 6.88-6.97 (m, 2H), 6.85 (d, 1H), 6.00 (d, 1H), 4.50-4.45 (m, 1H), 4.45-4.02 (m, 2H), 3.90-3.80 (m, 1H), 3.18-3.08 (m, 1H), 2.50-2.35 (m, 2H), 2.10 (s, 3H), 190-1.25 (m, 6H).

### Example 10

### Preparation of 1-(2-(9H-fluoren-9-ylidene)acetyl)-5,6-dihydropyridin-2(1H)-one

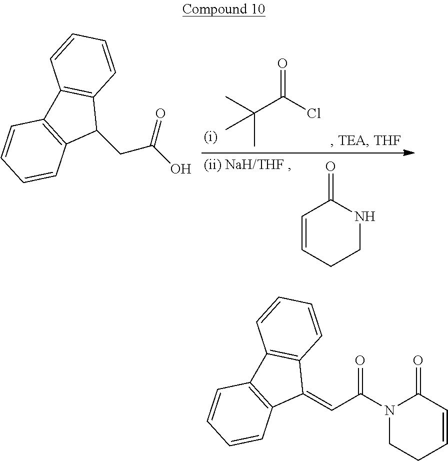

To a stirred solution of 9H-fluoren-9-ylideneacetic acid (180 mg, 0.80 mmol) in THF (5 mL) at 0° C. was triethylamine (0.1 mL, 0.96 mmol, 1.2 eq) dropwise followed by the addition of pivaloyl chloride (115 mg, 1.2 mmol, 1.2 eq) and the reaction mixture was allowed to stir at same temperature for 1 h. Progress was monitored by TLC and the reaction mixture was used directly in the next step. To a solution of 5,6-dihydropyridin-2(1H)-one (100 mg, 1.030 mmol) in THF (5 mL) at 0° C. was added NaH (49 mg, 1.23 mmol, 1.2 eq) and the reaction mixture was allowed to stir at the same temperature for 30 min. To this solution at 0° C. was added the mixed anhydride solution through a syringe filter and the resulting mixture was allowed to stir as 0° C. for 1 h. Progress was monitored by TLC and LCMS. After completion, the reaction mixture was diluted with water (50 mL) and extracted with EtOAc (2×40 mL). The combined organic layers were washed with water (3×20 mL), dried over anhydrous Na₂SO₄ and filtered. The solvent was removed under reduced pressure and the crude product was purified by reversed phase HPLC to afford the title compound (28 mg, 9%).

LCMS: 302 [M+1]⁺

¹H NMR: (400 MHz, CDCl₃) δ 8.34 (d, 1H), 7.70 (d, 2H), 7.63 (dd, 1H), 7.44 (d, 2H), 7.37 (dt, 1H), 7.27 (d, 1H), 7.24 (d, 1H), 6.98 (d, 1H), 6.05 (d, 1H), 4.18-4.08 (m, 2H), 2.50-2.25 (m, 2H).

### Example 11

### Preparation of (E)-1-((2-(pyridin-3-yl)vinyl)sulfonyl)-5,6-dihydropyridin-2(1H)-one

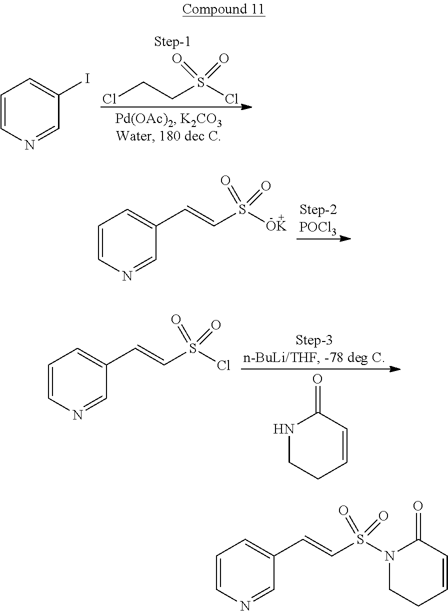

### Step-1

To a suspension of anhydrous K₂CO₃ (402 mg, 2.91 mmol, 3 eq), palladium(II) acetate (11 mg, 0.049 mmol, 0.02 eq) and 3-iodopyridine (200 mg, 0.97 mmol) in water (5 mL) in a 30 mL microwave vial with a magnetic stir bar was added 2-chloroethanesulfonyl chloride (0.1 mL 0.97 mmol) dropwise. The vial was sealed and heated to 180° C. by microwave irradiation for 10 min. The vial was cooled to room temperature and to it was added a fresh portion of palladium(II) acetate (11 mg, 0.049 mmol, 0.02 eq). The vial was then resealed and heated under same conditions for an additional 10 min. Progress was monitored by TLC. After completion, the reaction mixture was diluted with water (50 mL) and the mixture was filtered through a bed of Celite. The filtrate was lyophilized to afford crude potassium (E)-2-(pyridin-3-yl)ethenesulfonate (500 mg) which was used in the next step without further purification.

### Step-2

A mixture of (E)-2-(pyridin-3-yl)ethenesulfonate (500 mg) and POCl₃ (10 mL) was allowed to stir at 80° C. for 1 h. Progress was monitored by TLC. After completion, the reaction mixture was cooled to room temperature and POCl₃ was evaporated under reduced pressure to give an oily residue. The residue was dissolved in EtOAc (50 mL). The solution was washed water (3×50 mL), dried over anhydrous Na₂SO₄ and filtered. Removal of the solvent under reduced pressure afforded (E)-2-(pyridin-3-yl)ethenesulfonyl chloride (250 mg) which was used in the next step without further purification.

### Step-3

To solution of 5,6-dihydropyridin-2(1H)-one (100 mg, 1.03 mmol) in THF (5 mL) at −78° C. was added n-BuLi (2.5 M in THF, 0.5 mL, 1.03 mmol, 1.2 eq) dropwise and the reaction mixture was allowed to stir at the same temperature for 30 min. To this solution was added a solution of (E)-2-(pyridin-3-yl)ethenesulfonyl chloride (209 mg, 1.2 eq) in THF (5 mL) and the reaction mixture was allowed to stir at −78° C. for 2 h. Progress was monitored by TLC and LCMS. After completion, the reaction mixture was diluted with water (30 mL) and extracted with EtOAc (3×50 mL). The combined organic layers were dried over anhydrous Na₂SO₄ and filtered. Removal of the solvent under reduced pressure afforded a crude oil which was purified by reversed phase HPLC to provide the title compound (12 mg, 4.4%).

LCMS: 265 [M+1]⁺

¹H NMR: (400 MHz, CDCl₃) δ 8.77 (s, 1H), 8.66 (d, 1H), 7.85 (d, 1H), 7.67 (d, 1H), 7.40-7.35 (m, 1H), 7.30 (d, 1H), 6.90-6.80 (m, 1H), 6.00 (d, 1H), 4.05-3.98 (m, 2H), 2.62-2.50 (m, 2H).

### Example 12

### Preparation of (E)-1-(3-(isoquinolin-4-yl)acryloyl)-1,5,6,7-tetrahydro-2H-azepin-2-one

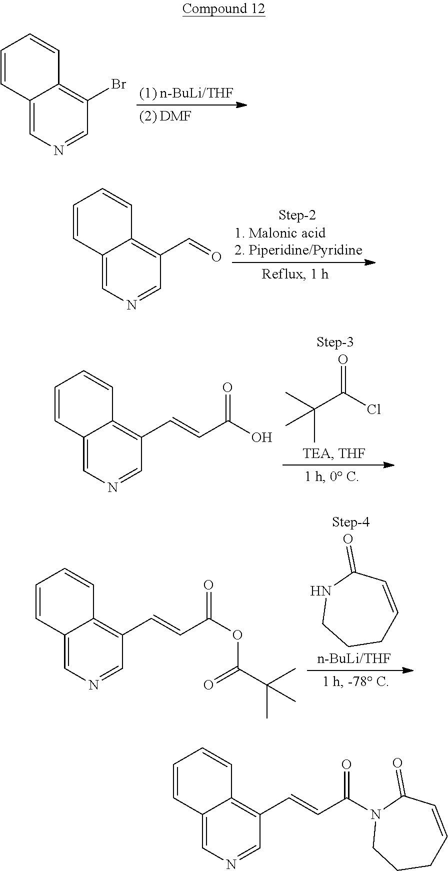

### Step-1

To a solution of 4-bromoisoquinoline (1 g, 4.80 mmol) in a solution of THF-diethyl ether (60 mL, 2:1) at −78° C. was added n-BuLi (2.5 Min THF, 3.8 mL, 9.62 mmol) dropwise and the mixture was allowed to stir at the same temperature for 1 h. To this solution was added a solution of DMF (0.9 mL, 12 mmol. 2.5 eq) in THF (5 mL) dropwise and the resulting mixture was allowed to stir at −78° C. for 2 h. Progress was monitored by TLC and LCMS. After completion, the reaction mixture was diluted with saturated, aqueous NH₄Cl (50 mL) and extracted with EtOAc (3×100 mL). The combined organic layers were washed with brine (100 mL), dried over anhydrous Na₂SO₄ and filtered. Removal of solvent gave the crude product which was purified by CombiFlash to afford isoquinoline-4-carbaldehyde (553 mg, 73%).

LCMS: 158 [M+1]⁺

### Step-2

To the stirred solution of isoquinoline-4-carbaldehyde (530 mg, 3.37 mmol) in pyridine (15 mL) and piperidine (3 mL) at room temperature was added malonic acid (737 mg, 7.08 mmol, 2.1 eq) and the reaction mixture was stirred at 110° C. for 1 h. Progress was monitored by TLC.

After completion, the reaction mixture was diluted with 1N HCl (50 mL) and extracted with EtOAc (2×200 mL). The combined organic layers were washed with water, dried over anhydrous Na₂SO₄, filtered and concentrated under reduced pressure. The crude material was purified by CombiFlash to afford (E)-3-(isoquinolin-4-yl)acrylic acid (433 mg, 65%).

LCMS: 200 [M+1]⁺

### Step-3

To a stirred solution of (E)-3-(isoquinolin-4-yl)acrylic acid (200 mg, 1.00 mmol) in THF (20 mL) at 0° C. was added triethylamine (0.21 mL, 1.50 mmol, 1.5 eq) followed by pivaloyl chloride (0.14 mL, 1.11 mmol, 1.1 eq) and the reaction mixture was allowed to stir at 0° C. for 1 h. Progress was monitored by TLC. After completion, the reaction mixture was filtered and the filtrate was carried to the next step without any further purification.

LCMS: 284 [M+1]⁺

### Step-4

To the stirred solution of 1,5,6,7-tetrahydro-2H-azepin-2-one (100 mg, 0.90 mmol) in THF (15 mL) at −78° C. was added n-BuLi (2.5 M in hexane, 0.47 mL, 1.17 mmol, 1.3 eq) and the reaction mixture was stirred at the same temperature for 30 min. To this mixture was added a solution of (E)-3-(isoquinolin-4-yl)acrylic pivalic anhydride (280 mg, 0.99 mmol, 1.1 eq) in THF (20 mL) and the resulting solution was allowed to stir at −78° C. for 2 h. Progress was monitored by TLC and LCMS. After completion, the reaction mixture was diluted with water (20 mL) and extracted with EtOAc (3×50 mL). The combined organic layers were washed with brine, dried over Na₂SO₄, filtered and concentrated under reduced pressure. The crude product was purified by CombiFlash to afford the title compound (21 mg, 7%) as a sticky solid.

LCMS: 293 [M+1]⁺

¹H NMR: (400 MHz, CDCl₃) δ 9.23 (s, 1H), 8.80 (s, 1H), 8.41 (d, 1H), 8.23 (d, 1H), 8.01 (d, 1H), 7.80 (t, 1H), 7.65 (t, 1H), 7.58 (d, 1H), 6.60-6.50 (m, 1H), 6.17 (d, 1H), 4.00-4.10 (m, 2H), 2.50-2.40 (m, 2H), 2.10-2.00 (m, 2H).

### Example 13

### Preparation of (E)-1-(3-(pyrimidin-2-yl)acryloyl)-1,5,6,7-tetrahydro-2H-azepin-2-one

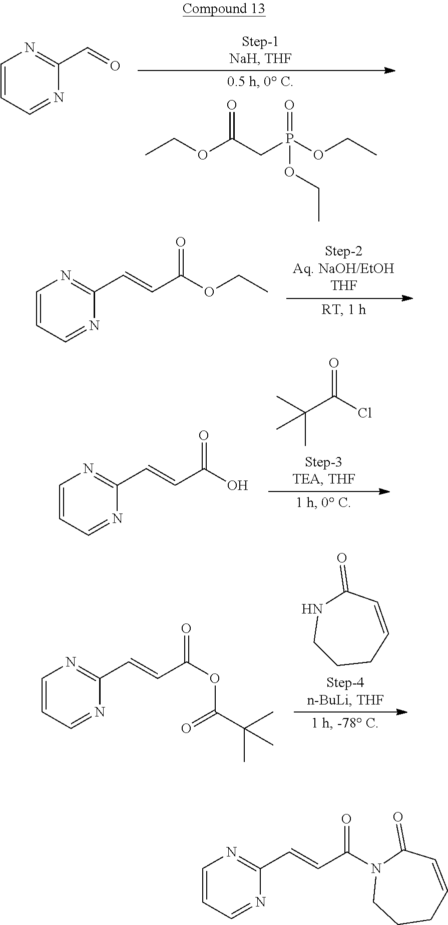

### Step-1

To a suspension of NaH (60% in mineral oil, 178 mg, 4.45 mmol, 1.2 eq) in THF (15 mL) at 0° C. was added ethyl 2-(diethoxyphosphoryl)acetate (0.82 mL, 4.07 mmol, 1.1 eq) dropwise and the reaction mixture was allowed to stir at the same temperature for 20 min. To this mixture was added a solution of pyrimidine-2-carbaldehyde (400 mg, 3.70 mmol) in THF (10 mL) and the reaction mixture was allowed to stir at 0° C. for 30 min. Progress was monitored by TLC and LCMS. After completion, the reaction mixture was diluted with water (50 mL) and extracted with EtOAc (3×50 mL). The combined organic layers were washed with brine (20 mL) and dried over anhydrous Na₂SO₄ and filtered. Removal of solvent gave (E)-ethyl 3-(pyrimidin-2-yl)acrylate (520 mg, 79%) which was used in the next step without further purification.

LCMS: 179 [M+1]⁺

### Step-2

To the stirred solution of (E)-ethyl 3-(pyrimidin-2-yl)acrylate (500 mg, 2.81 mmol) in EtOH-THF (1:1) was added aqueous 5N NaOH (3 mL) dropwise at 0° C. and the reaction mixture was stirred at room temperature for 1 h. Progress was monitored by TLC. After completion, the reaction mixture was diluted with 3N HCl (20 mL) and extracted with EtOAc (2×100 mL). The combined organic layers were washed with water, dried over Na₂SO₄, filtered and concentrated under reduced pressure to afford (E)-3-(pyrimidin-2-yl)acrylic acid (370 mg, 88%) as white solid.

LCMS: 151 [M+1]⁺

### Step-3

To a stirred solution of (E)-3-(pyrimidin-2-yl)acrylic acid (150 mg, 0.99 mmol) in THF (5 mL) at 0° C. were added triethylamine (0.2 mL, 1.50 mmol, 1.5 eq) and pivaloyl chloride (0.13 mL, 1.10 mmol, 1.1 eq) and the reaction mixture was allowed to stir at 0° C. for 1 h. Progress was monitored by TLC. After completion, the reaction mixture was filtered and the filtrate was carried to next step without any further purification.

LCMS: 235 [M+1]⁺

### Step-4

To a stirred solution of 6,7-dihydro-1H-azepin-2(5H)-one (100 mg, 0.90 mmol) in THF (5 mL) at −78° C. was added n-BuLi (2.5 M in hexane, 0.47 mL, 1.17 mmol, 1.3 eq) and the reaction mixture was stirred at same temperature for 30 min. To this mixture was added a solution of pivalic (E)-3-(pyrimidin-2-yl)acrylic anhydride (211 mg, 0.90 mmol, 1.1 eq) in THF (5 mL) and the resulting mixture was stirred at −78° C. for 2 h. Progress was monitored by TLC and LCMS. After completion, the reaction mixture was diluted with water (20 mL) and extracted with EtOAc (2×100 mL). The combined organic layers were washed with brine, dried over Na₂SO₄, filtered and concentrated under reduced pressure. The crude material was purified by CombiFlash to afford the title compound (48 mg, 22%) as a semi-solid.

LCMS: 244 [M+1]⁺

¹H NMR: (400 MHz, CDCl₃) δ 8.78 (d, 2H), 8.01 (d, 1H), 7.65 (d, 1H), 7.20 (t, 1H), 6.65-6.50 (m, 1H), 6.12 (d, 1H), 4.03-3.96 (m, 2H), 2.46-2.38 (m, 2H), 2.05-1.98 (m, 2H).

### Example 14

### Preparation of (E)-1-(styrylsulfonyl)-1,5,6,7-tetrahydro-2H-azepin-2-one

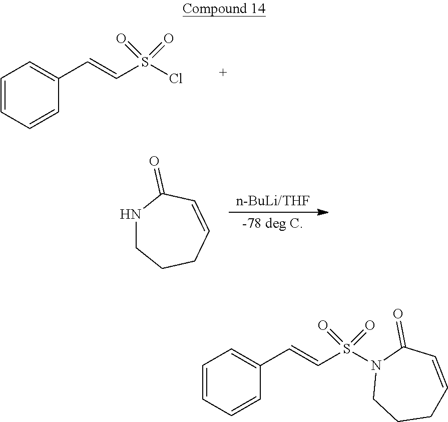 [SMILES: O=S(=O)(Cl)/C=C/c1ccccc1 ; O=C1C=CCCCN1 ; O=C1C=CCCCN1S(=O)(=O)/C=C/c1ccccc1]

To a solution of 6,7-dihydro-1H-azepin-2(5H)-one (100 mg, 0.90 mmol) in THF (10 mL) at −78° C. was added n-BuLi (2.5 M in THF, 0.47 mL, 1.17 mmol, 1.3 eq) and the reaction mixture was allowed to stir at the same temperature for 30 min. To this was added a solution of (E)-2-phenylethenesulfonyl chloride (201 mg, 1.1 eq) in THF (5 mL) and the reaction mixture was allowed to stir at −78° C. for 2 h. Progress was monitored by TLC and LCMS. After completion, the reaction mixture was diluted with water (30 mL) and extracted with EtOAc (3×50 mL). The combined organic layers were dried over anhydrous sodium sulfate. Removal of solvent gave a crude oil which was purified by reversed phase HPLC to afford the title compound (12 mg, 4%) as a semi-solid.

LCMS: 278 [M+1]⁺

¹H NMR: (400 MHz, CDCl₃) δ 7.63 (d, 1H), 7.56-7.46 (m, 2H), 7.45-7.38 (m, 3H), 7.27 (d, 1H), 6.55-6.45 (m, 1H), 6.00 (d, 1H), 3.92-3.81 (m, 2H), 250-2.40 (m, 2H), 2.12-2.02 (m, 2H).

### Example 15

### Preparation of (E)-1-(3-(quinolin-2-yl)acryloyl)-1,5,6,7-tetrahydro-2H-azepin-2-one

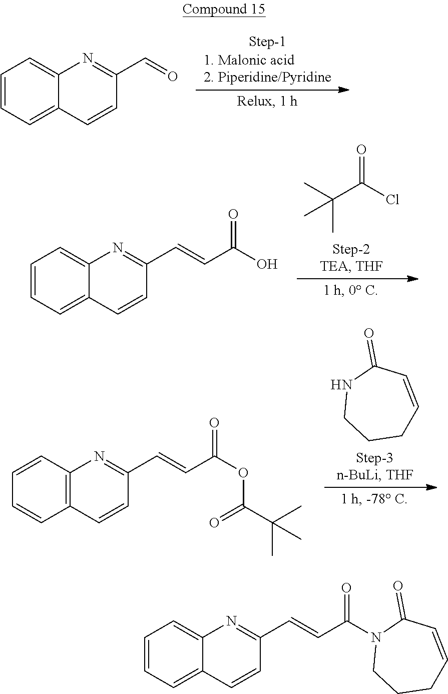

### Step-1

To the stirred solution of quinoline-2-carbaldehyde (2 g, 12.7 mmol) in pyridine (27 mL) and piperidine (5 mL) at room temperature was added malonic acid (2.78 g, 26.7 mmol, 2.1 eq) and the reaction mixture was stirred at 110° C. for 1 h. Progress was monitored by TLC. After completion, the reaction mixture was diluted with 1N HCl (50 mL) and extracted with EtOAc (2×200 mL). The combined organic layers were washed with water, dried over Na₂SO₄, filtered and concentrated under reduced pressure. The crude product was purified by CombiFlash to afford (E)-3-(quinolin-2-yl)acrylic acid (470 mg, 18.5%).

LCMS: 200 [M+1]⁺

### Step-2

To a stirred solution of (E)-3-(quinolin-2-yl)acrylic acid (200 mg, 1.00 mmol) in THF (5 mL) at 0° C. was added triethylamine (0.21 mL, 1.50 mmol, 1.5 eq) and pivaloyl chloride (0.14 mL, 1.11 mmol, 1.1 eq) and the reaction mixture was allowed to stir at 0° C. for 1 h. Progress was monitored by TLC. After completion, the reaction mixture was filtered and the filtrate was carried to next step without any further purification.

LCMS: 284 [M+1]⁺

### Step-3

To the stirred solution of 1,5,6,7-tetrahydro-2H-azepin-2-one (100 mg, 0.90 mmol) in THF (5 mL) at −78° C. was added n-BuLi (2.5 M in hexane, 0.47 mL, 1.17 mmol, 1.3 eq) and the reaction mixture was stirred at the same temperature for 30 min. To this reaction mixture was added a solution of pivalic (E)-3-(quinolin-2-yl)acrylic anhydride (280 mg, 0.99 mmol, 1.1 eq) in THF and the resulting mixture was allowed to stir at −78° C. for 2 h. Progress was monitored by TLC and LCMS. After completion, the reaction mixture was diluted with water (20 mL) and extracted with EtOAc (2×100 mL). The combined organic layers were washed with brine, dried over Na₂SO₄, filtered and concentrated under reduced pressure. The crude material was purified by reversed phase HPLC to afford the title compound (17 mg, 6.4%) as a white solid.

LCMS: 293 [M+1]⁺

¹H NMR: (400 MHz, CDCl₃) δ 8.19 (d, 1H), 8.16 (d, 1H), 7.95-7.85 (m, 2H), 7.80 (d, 1H), 7.78-7.65 (m, 2H), 7.55 (t, 1H), 6.61-6.65 (m, 1H), 6.17 (d, 1H), 4.08-3.98 (m, 2H), 2.45-2.38 (m, 2H), 2.10-1.98 (m, 2H).

### Example 16

### Preparation of (E)-1-(3-(3-methyl-1,2,4-oxadiazol-5-yl)acryloyl)-5,6-dihydropyridin-2(1H)-one

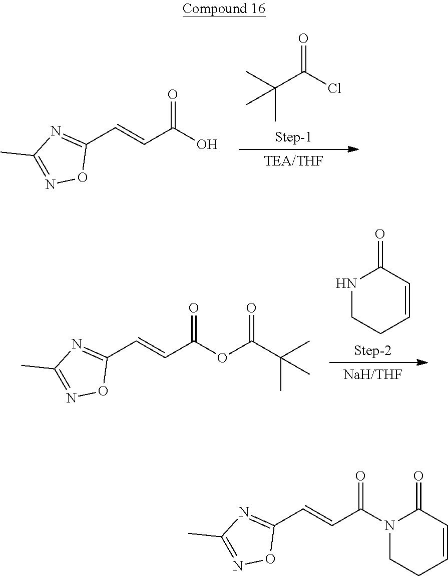

### Step-1

To a stirred solution of (E)-3-(1,2,4-oxadiazol-5-yl)acrylic acid (250 mg, 1.62 mmol) in THF (10 mL) at 0° C. was added triethylamine (0.23 mL, 1.78 mmol, 1.1 eq) followed by pivaloyl chloride (0.23 mL, 1.78 mmol, 1.1 eq) and the reaction mixture was allowed to stir at 0° C. for 1 h. Progress was monitored by TLC. After completion, the mixture was filtered and the filtrate was used in the next step without any further purification.

### Step-2

To the stirred solution of 5,6-dihydropyridin-2(1H)-one (143 mg, 1.48 mmol) in THF (10 mL) at 0° C. was added NaH (60% in mineral oil, 118 mg, 3.24 mmol, 2 eq) and the reaction mixture was stirred 0° C. for 30 minutes. To this was added a solution of (E)-3-(3-methyl-1,2,4-oxadiazol-5-yl)acrylic pivalic anhydride in THF (10 mL) and the reaction mixture was allowed to stir at 0° C. for 2 h. Progress was monitored by TLC. After completion, the mixture was diluted with water (20 mL) and extracted with EtOAc (3×50 mL). The combined organic layers were washed with brine, dried over Na₂SO₄, filtered and concentrated under reduced pressure. The crude material was purified by CombiFlash to afford the title compound (35 mg, 10%).

LCMS: 234 [M+1]⁺

¹H NMR: (400 MHz, CDCl₃) δ 7.90 (d, 1H), 7.38 (d, 1H), 7.05-6.95 (m, 1H), 6.05 (d, 1H), 4.08-4.00 (m, 2H), 2.52-2.45 (m, 2H), 2.41 (s, 3H).

### Example 17

### Preparation of (E)-1-(3-(3-methyl-1,2,4-oxadiazol-5-yl)acryloyl)-1,5,6,7-tetrahydro-2H-azepin-2-one

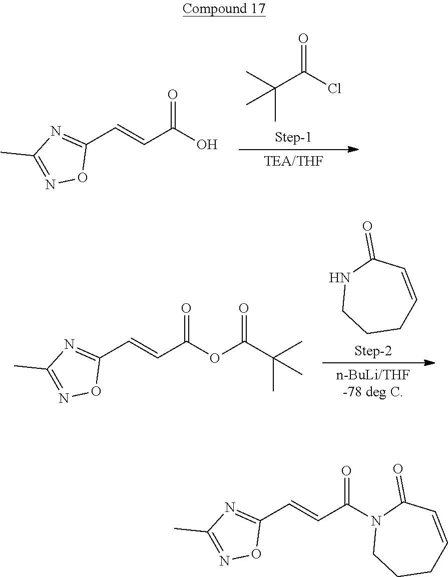

### Step-1

To a stirred solution of (E)-3-(1,2,4-oxadiazol-5-yl)acrylic acid (250 mg, 1.62 mmol) in THF (10 mL) at 0° C. was added triethylamine (0.23 mL, 1.78 mmol, 1.1 eq) followed by pivaloyl chloride (0.23 mL, 1.78 mmol, 1.1 eq) and the reaction mixture was allowed to stir at 0° C. for 1 h. Progress was monitored by TLC. After completion, the mixture was filtered and the filtrate was used in the next step without any further purification.

### Step-2

To the stirred solution of 6,7-dihydro-1H-azepin-2(5H)-one (100 mg, 0.90 mmol) in THF (10 mL) at −78° C. was added n-BuLi (2.5 M in hexane, 0.44 mL, 1.1 mmol, 1.2 eq) and the reaction mixture was stirred −78° C. for 30 minutes. To this reaction mixture was added a solution of (E)-3-(3-methyl-1,2,4-oxadiazol-5-yl)acrylic pivalic anhydride in THF (10 mL) and the reaction mixture was allowed to stir at 0° C. for 2 h. Progress was monitored by TLC. After completion, the reaction mixture was diluted with water (20 mL) and extracted with EtOAc (3×50 mL). The combined organic layers were washed with brine, dried over Na₂SO₄, filtered and concentrated under reduced pressure. The crude material was purified by CombiFlash by CombiFlash to obtain the title compound (80 mg, 36%).

LCMS: 248 [M+1]⁺

¹H NMR: (400 MHz, CDCl₃) δ 7.82 (d, 1H), 7.40 (d, 1H), 6.65-6.56 (m, 1H), 6.10 (d, 1H), 4.03-3.95 (m, 2H), 2.45-2.37 (m, 2H), 2.42 (s, 3H), 2.06-1.95 (m, 2H).

### Example 18

### Preparation of (E)-1-(3-cyclopropylacryloyl)-5,6-dihydropyridin-2(1H)-one

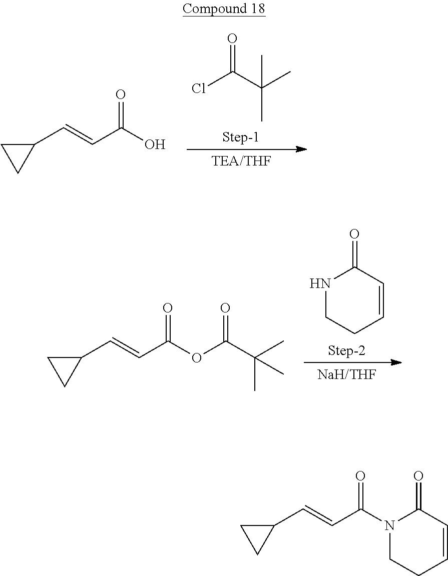

### Step-1

To a solution of (2E)-3-cyclopropylprop-2-enoic acid (300 mg, 2.67 mmol) in dry THF (10 mL) was added triethylamine (0.41 mL, 2.94 mmol, 1.1 eq) followed by pivaloyl chloride (0.35 mL, 2.94 mmol, 1.1 eq) and the reaction mixture was stirred at 0° C. for 45 min. Progress was monitored by TLC. After completion, the reaction mixture was filtered and the filtrate was used directly for the next step.

### Step-2

To a stirred solution of 5,6-dihydropyridin-2(1H)-one (150 mg, 1.54 mmol) in dry THF (10 mL) at 0° C. was added NaH (60% in mineral oil, 68 mg, 1.70 mmol, 1.1 eq.) and the mixture was allowed to stir at the same temperature for 30 min. To this solution was added a solution of (2E)-3-cyclopropylprop-2-enoic 2,2-dimethylpropanoic anhydride (333 mg, 1.70 mmol, 1.1 eq.) in THF (10 mL) and the resulting mixture was allowed to stir at 0° C. for 1 h. Progress was monitored by TLC and LCMS. After completion, the reaction mixture was brought to RT, diluted with water (30 mL) and extracted with EtOAc (3×30 mL). The combined organic layers were washed with brine (30 mL), dried over anhydrous Na₂SO₄, filtered and evaporated to dryness. The crude material was purified by CombiFlash to afford the title compound (22 mg, 15%).

LCMS: 192 [M+1]⁺

¹H NMR: (400 MHz, CDCl₃) δ 6.97 (d, 1H), 6.92-6.82 (m, 1H), 656-6.43 (m, 1H), 6.00 (d, 1H), 4.04-3.91 (m, 2H), 2.43-2.37 (m, 2H), 1.68-1.58 (m, 1H), 2.00-1.92 (m, 2H), 0.66-0.60 (m, 2H).

### Example 19

### Preparation of (E)-1-(3-(piperidin-4-yl)acryloyl)-5,6-dihydropyridin-2(1H)-one and tert-butyl (E)-4-(3-oxo-3-(6-oxo-3,6-dihydropyridin-1(2H)-yl)prop-1-en-1-yl)piperidine-1-carboxylate

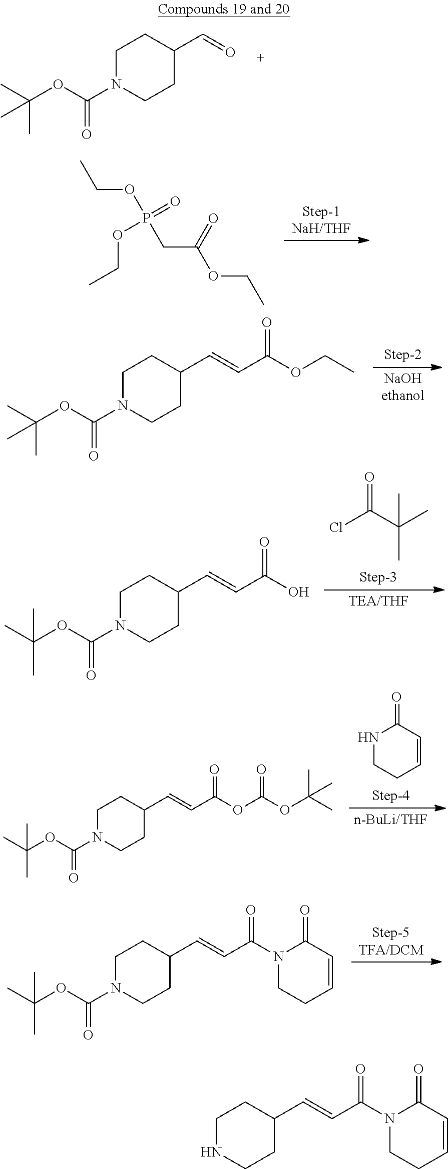

### Step-1

To a suspension of NaH (60% in mineral oil, 1.03 g, 25.8 mmol, 1.1 eq.) in THF (50 mL) was added ethyl 2-(diethoxyphosphoryl)acetate (10.51 g, 46.9 mmol, 2 eq.) dropwise at 0° C. and the mixture was allowed to stir at the same temperature for 30 min. To this mixture was added a solution of tert-butyl 4-formylpiperidine-1-carboxylate (5 g, 23.5 mmol) in THF (5 mL) and the resulting mixture was allowed to stir at room temperature for 1 h. Progress was monitored by TLC. After completion, the mixture was diluted with saturated aqueous NH₄Cl (100 mL) and extracted with EtOAc (3×50 mL). The combined organic layers were washed with brine, dried over anhydrous Na₂SO₄, filtered and evaporated. The crude material was purified by CombiFlash to afford tert-butyl (E)-4-(3-ethoxy-3-oxoprop-1-en-1-yl)piperidine-1-carboxylate (3.5 g, 52.7%).

LCMS: 284 [M+1]⁺

### Step-2

To a solution of tert-butyl (E)-4-(3-ethoxy-3-oxoprop-1-en-1-yl)piperidine-1-carboxylate (1 g, 3.53 mmol) in EtOH (20 mL) was added a solution of NaOH (2 M, 1.6 mL) in water and the reaction mixture was stirred at 70° C. for 4 h. The mixture was concentrated under vacuum and then acidified with 2N HCl to pH 3. The mixture was then diluted with saturated aqueous NH₄Cl (30 mL) and extracted with CH₂Cl₂ (3×30 mL). The combined organic layers were washed with brine (50 mL), dried over anhydrous Na₂SO₄ and filtered. Removal of solvent under reduced pressure afforded (E)-3-(1-(tert-butoxycarbonyl) piperidin-4-yl)acrylic acid (0.43 g, 47%) as crystalline solid.

LCMS: 256 [M+1]⁺

### Step-3

To a solution of (E)-3-(1-(tert-butoxycarbonyl) piperidin-4-yl)acrylic acid (0.4 g, 1.56 mmol) in dry THF was added triethylamine (0.24 mL, 1.72 mmol, 1.1 eq.) followed by pivaloyl chloride (0.21 mL, 1.72 mmol, 1.1 eq.) and the reaction mixture was stirred at 0° C. for 45 min. Progress was monitored by TLC. After completion, the mixture was filtered and the filtrate was used directly for the next step.

### Step-4

To a stirred solution of 5,6-dihydropyridin-2(1H)-one (0.18 g, 1.85 mmol) in dry THF at −78° C. was added n-BuLi (2.5 M in hexane, 0.81 mL, 2.04 mmol, 1.1 eq.) and the mixture was allowed to stir at the same temperature for 30 min. To this solution was added a solution of (E)-3-(1-(tert-butoxycarbonyl)piperidin-4-yl)acrylic(tert-butyl carbonic) anhydride in THF and the reaction mixture was allowed to stir at −78° C. for 1 h. Progress was monitored by TLC and LCMS. After completion, the mixture was brought to 0° C., diluted with water (30 mL) and extracted with EtOAc (3×30 mL). The combined organic layers were washed with brine, dried over anhydrous Na₂SO₄, filtered and evaporated to dryness. The crude material was purified by reversed phase HPLC to afford tert-butyl (E)-4-(3-oxo-3-(6-oxo-3,6-dihydropyridin-1(2H)-yl)prop-1-en-1-yl)piperidine-1-carboxylate (0.160 g, 30.6%).

LCMS: 335 [M+1]⁺

### Step-5

To a solution of tert-butyl (E)-4-(3-oxo-3-(6-oxo-3,6-dihydropyridin-1(2H)-yl)prop-1-en-1-yl)piperidine-1-carboxylate (0.16 g, 0.48 mmol) in CH₂Cl₂ (10 mL) was added trifluoroacetic acid (3.0 mL) at 0° C. and the resulting mixture was stirred at room temperature for 2 h. Progress was monitored by TLC. After completion, the mixture was evaporated under reduce pressure and the crude material was purified by reversed phase HPLC to afford the title compound (23 mg, 20.5%).

LCMS: 235 [M+1]⁺

¹H NMR: (400 MHz, DMSO-d₆) δ 8.25-8.62 (br s, 2H), 7.12-7.03 (m, 1H), 6.80-6.67 (m, 2H), 5.93 (d, 1H), 3.87-3.79 (m, 2H), 3.40-3.20 (m, 2H), 2.96-2.80 (m, 2H), 2.60-2.38 (m, 3H), 1.96-1.80 (m, 2H), 1.59-1.40 (m, 2H).

### Example 20

### Preparation of (E)-1-(3-(2,3-dihydro-[1,4]dioxino[2,3-b]pyridin-6-yl)acryloyl)-5,6-dihydropyridin-2(1H)-one

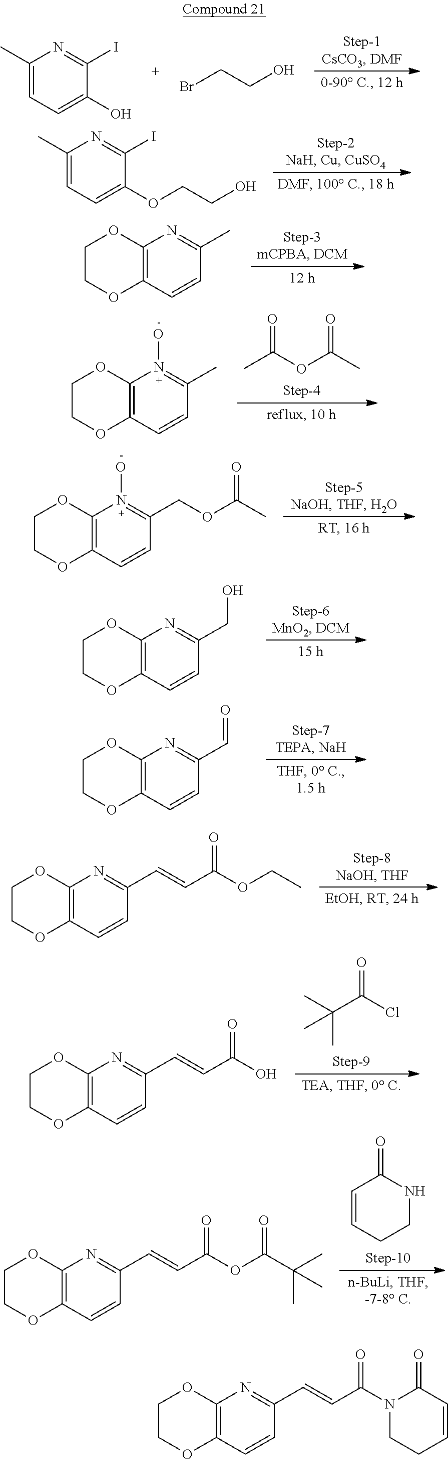

### Step-1

To a solution of 2-iodo-6-methylpyridin-3-ol (10 g, 42.5 mmol) in DMF (250 mL) at room temperature was added Cs₂CO₃ (16.6 g, 51 mmol, 1.2 eq.) followed by 2-bromoethanol (5.99 mL, 85 mmol, 2 eq.) dropwise. The resulting mixture was stirred at 90° C. overnight. Progress was monitored by TLC. After completion, the reaction mixture was cooled to room temperature, diluted with ice cold water (300 mL) and extracted with CH₂Cl₂ (3×300 mL). The combined organic layers were washed with brine, dried over anhydrous Na₂SO₄ and filtered. Removal of solvent under reduced pressure afforded 2-(2-iodo-6-methylpyridin-3-yloxy)ethanol (9 g, 76.1%) which was used without further purification.

LCMS: 279 [M+1]⁺

### Step-2

To a stirred solution of 2-(2-iodo-6-methylpyridin-3-yloxy)ethanol (6 g, 32.2 mmol) in anhydrous DMF (200 mL) at 0° C. was added NaH (60% in mineral oil, 0.928 g, 38.3 mmol, 1.2 eq.), Cu (0.81 g, 12.9 mmol, 0.4 eq.) and CuSO₄ (3.07 g, 19.3 mmol, 0.6 eq.). The resulting mixture was stirred at 100° C. overnight. Progress was monitored by TLC. After completion, the reaction mixture was cooled to room temperature, diluted with ice cold water (300 mL) and extracted with CH₂Cl₂ (3×300 mL). The combined organic layers were washed with brine, dried over anhydrous Na₂SO₄ and filtered. Removal of solvent under reduced pressure afforded the crude material which was purified by CombiFlash to afford 6-methyl-2,3-dihydro-[1,4]dioxino[2,3-b]pyridine (0.95 g, 19.5%).

LCMS: 152 [M+1]⁺

### Step-3

To a stirred solution of 6-methyl-2,3-dihydro-[1,4]dioxino[2,3-b]pyridine (0.95 g, 6.27 mmol) in anhydrous DMF (200 mL) at 0° C. was added m-CPBA (1.29 g, 7.5 mmol, 1.2 eq.) portion-wise. After addition the resulting mixture was stirred at room temperature overnight. Progress was monitored by TLC. After completion, the solvent was removed under reduced pressure to afford crude 6-methyl-2,3-dihydro-[1,4]dioxino[2,3-b]pyridine 5-oxide (0.95 g, 90%) which was used without further purification.

LCMS: 168 [M+1]⁺

### Step-4

A stirred solution of 6-methyl-2,3-dihydro-[1,4]dioxino[2,3-b]pyridine 5-oxide (0.95 g, 5.68 mmol) in acetic anhydride (20 mL) was refluxed for 10 h. Progress was monitored by TLC.

After completion, the volatiles were removed under reduced pressure to afford crude 6-(acetoxymethyl)-2,3-dihydro-[1,4]dioxino[2,3-b]pyridine 5-oxide (4 g) which used without further purification.

LCMS: 227 [M+1]⁺

### Step-5

To a solution of 6-(acetoxymethyl)-2,3-dihydro-[1,4]dioxino[2,3-b]pyridine 5-oxide (4 g, 19.1 mmol) in a mixture of THF and water (1:1, 20 mL) was added NaOH (1.35 g, 38.3 mmol, 2 eq). The resulting mixture was stirred at room temperature for 12 h. Progress was monitored by TLC. After the completion, the solvent was removed under reduced pressure to afford (2,3-dihydro-[1,4]dioxino[2,3-b]pyridin-6-yl)methanol (1.1 g) which was used without further purification.

LCMS: 168 [M+1]⁺

### Step-6

To a solution of (2,3-dihydro-[1,4]dioxino[2,3-b]pyridin-6-yl)methanol (0.3 g, 1.79 mmol) in CH₂Cl₂ (10 mL) was added activated MnO₂ (0.31 g, 3.59 mmol, 2 eq.) and the resulting slurry was stirred at room temperature for 15 h. Progress was monitored by TLC. After completion, the slurry was filtered through a bed of Celite. The Celite-bed was washed with CH₂Cl₂ (10 mL) and the filtrate was concentrated under reduced pressure to afforded 2,3-dihydro-[1,4]dioxino[2,3-b]pyridine-6-carbaldehyde (0.25 g) which was used without further purification.

LCMS: 166 [M+1]⁺

### Step-7

To a stirred suspension of NaH (60% in mineral oil, 47 mg, 1.96 mmol, 1.3 eq.) in anhydrous THF (10 mL) at 0° C. was added triethylphosphonoacetate (0.36 mL, 1.81 mmol, 1.2 eq.) and the mixture was stirred at the same temperature for 10 min. To this mixture was added a solution of 2,3-dihydro-[1,4]dioxino[2,3-b]pyridine-6-carbaldehyde (0.25 g, 1.51 mmol) in THF (10 mL). The resulting mixture was stirred at 0° C. 1.5 h. Progress was monitored by TLC. After completion, the reaction mixture was diluted with brine (50 mL) and extracted with EtOAc (3×50 mL). The combined organic layers were dried over anhydrous Na₂SO₄ and filtered. Removal of solvent under reduced pressure afforded a crude material which was purified by CombiFlash to afford of (E)-ethyl 3-(2,3-dihydro-[1,4]dioxino[2,3-b]pyridin-6-yl)acrylate (0.2 g, 56.2%).

LCMS: 236 [M+1]⁺

### Step-8

To a solution of (E)-ethyl 3-(2,3-dihydro-[1,4]dioxino[2,3-b]pyridin-6-yl)acrylate (0.2 g, 0.851 mmol) in a mixture of THF:EtOH (1:1, 10 mL) was added a solution of NaOH (0.34 g, 8.51 mmol, 10 eq.) in water (1 mL). The resulting mixture was stirred at room temperature for 2 h. Progress was monitored by TLC. After completion, the reaction mixture was neutralized with 2 M aqueous HCl and extracted with EtOAc (3×50 mL). The combined organic layers were dried over Na₂SO₄, filtered and concentrated to afforded a crude material. Trituration with Et₂O and pentane afforded (E)-3-(2,3-dihydro-[1,4]dioxino[2,3-b]pyridin-6-yl)acrylic acid (0.1 g, 56.8%).

LCMS: 208 [M+1]⁺

### Step-9

To a stirred solution of (E)-3-(2,3-dihydro-[1,4]dioxino[2,3-b]pyridin-6-yl)acrylic acid (0.1 g, 0.48 mmol) in THF (15 mL) at 0° C. was added triethylamine (0.07 mL, 0.579 mmol, 1.2 eq.) followed by pivaloyl chloride (0.07 mL, 0.579 mmol, 1.2 eq.) and the resulting mixture was stirred at 0° C. for 2 h. Progress was monitored by TLC. After completion, the reaction mixture was used directly in the next step.

### Step-10

To a stirred solution of 5,6-dihydropyridin-2(1H)-one (0.08 g, 0.71 mmol, 2.1 eq.) in THF (10 mL) at −78° C. was added n-BuLi (0.3 mL, 0.75 mmol, 2.2 eq.) and the resulting mixture was stirred at the same temperature for 30 min. To this mixture was added a solution of (E)-3-(2,3-dihydro-[1,4]dioxino[2,3-b]pyridin-6-yl)acrylic pivalic anhydride in THF (15 mL) dropwise and the reaction mixture was stirred at −78° C. for 2 h. Progress was monitored by TLC.

After completion, the reaction mixture was diluted with an aqueous NH₄Cl solution (50 mL) and extracted with EtOAc (3×50 mL). The combined organic layers were dried over Na₂SO₄ and filtered. Removal of the solvent provided crude material which was purified by reversed phase HPLC to afford the title compound (5 mg, 5%).

LCMS: 287 [M+1]⁺

¹H NMR: (400 MHz, CDCl₃) δ 7.64 (d, 1H), 7.57 (d, 1H), 7.16 (d, 1H), 7.08 (d, 1H), 6.96-6.89 (m, 1H), 6.03 (d, 1H), 4.45-4.40 (m, 2H), 4.28-4.20 (m, 2H), 4.03-3.96 (m, 2H), 2.46-2.40 (m, 2H).

### Example 21

### Preparation of (E)-1-(3-(1-methylcyclohexyl)acryloyl)-5,6-dihydropyridin-2(1H)-one

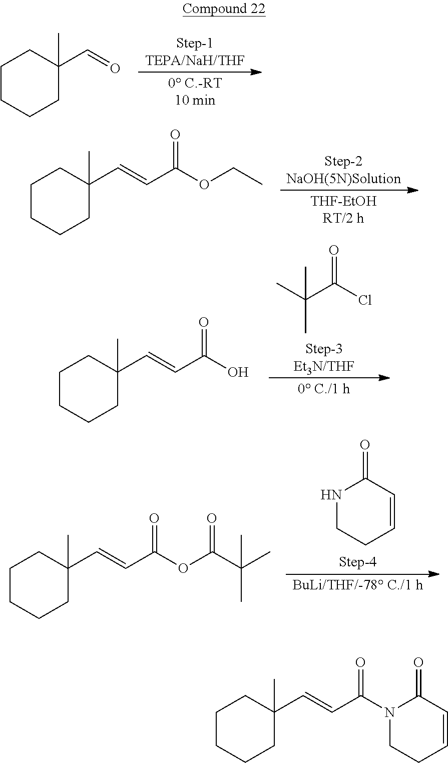

### Step-1

To a solution of triethyl phosphonoacetate (TEPA) (1.95 g, 8.7 mmol, 1.1 eq.) in THF (8 mL) was added NaH (60% in mineral oil, 0.377 g, 9.5 mmol, 1.2 eq.) at 0° C. The mixture was stirred for 5 min. To this mixture was then added a solution of 1-methylcyclohexane-1-carbaldehyde (1 g, 7.9 mmol) in THF (2 mL) dropwise at 0° C. and the reaction mixture was stirred for 5 min. Progress was monitored by TLC. After completion, the reaction mixture was diluted with water (20 mL) and extracted with EtOAc (3×50 mL). The combined organic layers were washed with brine, dried over Na₂SO₄, filtered and concentrated under reduced pressure to obtain ethyl (E)-3-(1-methylcyclohexyl)acrylate (1.4 g, 93.3%) which was used without purification.

LCMS: 197 [M+1]⁺

### Step-2

To the stirred solution of ethyl (E)-3-(1-methylcyclohexyl)acrylate (1 g, 5.0 mmol) in 1:1 THF-EtOH (10 mL) at room temperature was added 5N aqueous NaOH (10.2 mL, 50 mol, 10 eq.) and the mixture was stirred for 2 h. Progress was monitored by TLC. After completion, the reaction mixture was diluted with 1N HCl (15 mL) and extracted with EtOAc (2×100 mL). The combined organic layers were washed with water, dried over anhydrous Na₂SO₄, filtered and concentrated under to obtain (E)-3-(1-methylcyclohexyl)acrylic acid (0.33 g, 38.5%) which was used without purification.

LCMS: 169 [M+1]⁺

### Step-3

To a stirred solution of (E)-3-(1-methylcyclohexyl)acrylic acid (330 mg, 1.96 mmol) in THF (5 mL) at 0° C. was added triethylamine (0.3 mL, 2.16 mmol, 1.1 eq) followed by pivaloyl chloride (0.26 mL, 2.16 mmol, 1.1 eq.) and the reaction mixture was allowed to stir at 0° C. for 1 h. Progress was monitored by TLC. After completion, the reaction mixture was filtered and carried to next step without any further purification.

LCMS: 251 [M+1]⁺

### Step-4

To the stirred solution of 5,6-dihydropyridin-2(1H)-one (200 mg, 2.06 mmol) in THF (10 mL) at −78° C. was added n-BuLi (2.5 M in hexane, 0.98 mL, 2.47 mmol, 1.2 eq.) and the reaction mixture was stirred at the same temperature for 30 min. To this reaction mixture was added a solution of (E)-3-(1-methylcyclohexyl)acrylic pivalic anhydride (572 mg, 2.49 mmol, 1.1 eq.) in THF (10 mL) and the resulting mixture was allowed to stir at −78° C. for 2 h. Progress was monitored by TLC and LCMS. After completion, the reaction mixture was diluted with water (20 mL) and extracted with EtOAc (3×50 mL). The combined organic layers were washed with brine, dried over Na₂SO₄, filtered and concentrated. The crude material was purified by CombiFlash to afford the title compound (120 mg, 23.6%) as an oil.

LCMS: 248 [M+1]⁺

¹H NMR: (400 MHz, CDCl₃) δ 6.98 (d, 1H), 6.95-6.85 (m, 1H), 6.77 (d, 1H), 6.00 (d, 1H), 4.01-3.90 (m, 2H), 2.45-2.38 (m, 2H), 1.65-1.30 (m, 10H).

### Example 22

### Preparation of (E)-1-(3-(2,3-dihydro-[1,4]dioxino[2,3-b]pyridin-6-yl)acryloyl)-5,6-dihydropyridin-2(1H)-one

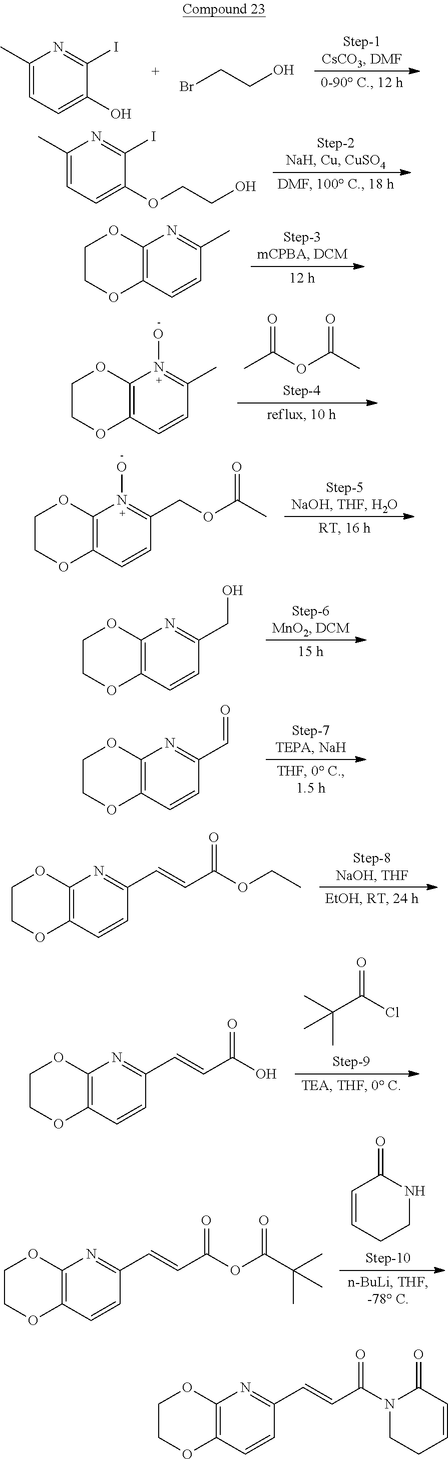

### Step-1

To a solution of 2-iodo-6-methylpyridin-3-ol (10 g, 42.5 mmol, 1 eq.) in DMF (250 mL) at room temperature was added Cs₂CO₃ (16.57 g, 51 mmol, 1.2 eq.) followed by 2-bromoethanol (5.99 mL, 85 mmol, 2 eq.) dropwise. The resulting mixture was stirred at 90° C. overnight. Progress of reaction was monitored by TLC. After the completion, reaction mixture was cooled to room temperature, diluted with ice cold water (300 mL) and extracted with DCM (3×300 mL). Combined organic layer was washed with brine and dried over anhydrous sodium sulphate. Removal of solvent under reduced pressure afforded 2-(2-iodo-6-methylpyridin-3-yloxy)ethanol (9 g, 76.14%).

LCMS: 279 [M+1]⁺

### Step-2

To a stirred solution of 2-(2-iodo-6-methylpyridin-3-yloxy)ethanol (6 g, 32.2 mmol, 1 eq.) in anhydrous DMF (200 mL) at 0° C. were added NaH (0.928 g, 38.25 mmol, 1.2 eq.), Cu (0.81 g, 12.9 mmol, 0.4 eq.) and CuSO₄ (3.07 g, 19.3 mmol, 0.6 eq.) and the resulting mixture stirred at 100° C. overnight. Progress of reaction was monitored by TLC. After the completion, reaction mixture was cooled to room temperature, diluted with ice cold water (300 mL) and extracted with DCM (3×300 mL). Combined organic layer was washed with brine solution and dried over anhydrous sodium sulphate. Removal of solvent under reduced pressure afforded crude which was purified by Combi-Flash on silica gel chromatography using methanol-dichloromethane (0-10%) as eluents to afford 6-methyl-2,3-dihydro-[1,4]dioxino[2,3-b]pyridine (0.950 g, 19.50%).

LCMS: 152[M+1]⁺

### Step-3

To a stirred solution of 6-methyl-2,3-dihydro-[1,4]dioxino[2,3-b]pyridine (0.950 g, 6.27 mmol, 1 eq.) in anhydrous DMF (200 mL) at 0° C. was added m-CPBA (1.29 g, 7.5 mmol, 1.2 eq.) portionwise. After addition the resulting mixture was stirred at room temperature overnight. Progress of reaction was monitored by TLC. After completion, solvent was removed under reduced pressure to afford crude 6-methyl-2,3-dihydro-[1,4]dioxino[2,3-b]pyridine 5-oxide (0.950 g, 90%) which was used in the next step without further purification.

LCMS: 168[M+1]⁺

### Step-4

To a stirred solution of 6-methyl-2,3-dihydro-[1,4]dioxino[2,3-b]pyridine 5-oxide (0.950 g, 5.68 mmol, 1 eq.) in acetic anhydride (20 mL) and the mixture was reflux for 10 h. Progress of reaction was monitored by TLC. After completion, solvent was removed under reduced pressure to afford crude 6-(acetoxymethyl)-2,3-dihydro-[1,4]dioxino[2,3-b]pyridine 5-oxide (4 g) which used in next step without further purification.

LCMS: 227[M+1]⁺

### Step-5

To a solution of 6-(acetoxymethyl)-2,3-dihydro-[1,4]dioxino[2,3-b]pyridine 5-oxide (4 g, 19.13 mmol, 1 eq.) in a mixture of THF and water (1:1, 20 mL) was added with NaOH (1.35 g, 38.27 mmol, 2 eq.). The resulting mixture was stir at room temperature for 12 h. Progress of reaction was monitored by TLC. After the completion, solvent was removed under reduced pressure to afford (2,3-dihydro-[1,4]dioxino[2,3-b]pyridin-6-yl)methanol (1.1 g crude).

LCMS: 168[M+1]⁺

### Step-6

To a solution of (2,3-dihydro-[1,4]dioxino[2,3-b]pyridin-6-yl)methanol (0.300 g, 1.79 mmol, 1 eq.) in dichloromethane (10 mL) was added activated MnO₂ (0.31 g, 3.59 mmol, 2 eq.) and the resulting slurry was stirred at room temperature for 15 h. Progress of reaction was monitored by TLC. After the completion, the slurry was filtered through celite-bed. The celite-bed was washed with dichloromethane (10 mL) and filtrate was removed under reduced pressure to afford afforded crude 2,3-dihydro-[1,4]dioxino[2,3-b]pyridine-6-carbaldehyde (0.25 g crude) which was used in the next step without further purification.

LCMS: 166[M+1]⁺

### Step-7

To a stirred suspension of NaH (0.047 g, 1.96 mmol, 1.3 eq.) in anhydrous THF (10 mL) at 0° C. were added triethylphosphono acetate (0.36 mL, 1.81 mmol, 1.2 eq.) and the mixture was stirred at the same temperature for 10 minutes. To this mixture was added a solution of 2,3-dihydro-[1,4]dioxino[2,3-b]pyridine-6-carbaldehyde (0.25 g, 1.51 mmol, 1 eq.) in THF (10 mL).

The resulting mixture was stir for at 0° C. 1.5 h. Progress of reaction was monitored by TLC. After the completion, reaction mixture was diluted with brine (50 mL), extracted with ethyl acetate (3×50 mL). Combined organic layer was dried over Na₂SO₄. Removal of solvent under reduced pressure afforded crude which was purified by Combi-Flash on silica gel using ethyl acetate-hexane (0-50%) as eluents to afford of (E)-ethyl 3-(2,3-dihydro-[1,4]dioxino[2,3-b]pyridin-6-yl)acrylate (0.200 g, 56.17%).

LCMS: 236[M+1]⁺

### Step-8

To a solution of (E)-ethyl 3-(2,3-dihydro-[1,4]dioxino[2,3-b]pyridin-6-yl)acrylate (0.2 g, 0.851 mmol, 1 eq.) in a mixture of THF-ethanol (1:1, 10 mL) was added a solution of NaOH (0.34 g, 8.51 mmol, 10 eq.) in water (1 mL). The resulting mixture was stirred at room temperature for 2 h. Progress of reaction was monitored by TLC. After completion, reaction mixture was neutralized with 2M aq. HCl and extracted with acetate (3×50 mL). Combined organic layer was dried over sodium sulphate, removal of solvent under reduced pressure afforded crude which was purified by trituration with diethyl ether and pentane to afford (E)-3-(2,3-dihydro-[1,4]dioxino[2,3-b]pyridin-6-yl)acrylic acid (0.1 g, 56.81%).

LCMS: 208[M+1]⁺

### Step-9

To a stirred solution of (E)-3-(2,3-dihydro-[1,4]dioxino[2,3-b]pyridin-6-yl)acrylic acid (0.1 g, 0.48 mmol, 1 eq.) in THF (15 mL) at 0° C. was added triethylamine (0.07 mL, 0.579 mmol, 1.2 eq.) followed by pivaloyl chloride (0.07 mL, 0.579 mmol, 1.2 eq.) and the resulting mixture was stirred stir at 0° C. for 2 h. Progress of reaction was monitored by TLC. After the completion, reaction mixture was used as such in the next reaction.

### Step-10

To a stirred solution of 5,6-dihydropyridin-2(1H)-one (0.08 g, 0.71 mmol, 2.1 eq.) in THF (10 mL) at −78° C. was added n-BuLi (0.3 mL, 0.75 mmol, 2.2 eq.) and the resulting mixture was stirred at the same temperature for 30 minutes. To this mixture was added a solution of (E)-3-(2,3-dihydro-[1,4]dioxino[2,3-b]pyridin-6-yl)acrylic pivalic anhydride in THF (15 mL) dropwise and the reaction mixture was stirred at −78° C. for 2 h. Progress of reaction was monitored by TLC. After completion, reaction mixture was diluted with ammonium chloride solution (50 mL) and extracted with ethyl acetate (3×50 mL). Combined organic layer was dried over sodium sulphate. Removal of solvent under reduced pressure crude which was purified by reversed phase HPLC to afford (E)-1-(3-(2,3-dihydro-[1,4]dioxino[2,3-b]pyridin-6-yl)acryloyl)-5,6-dihydropyridin-2(1H)-one (5 mg, 5%)

LCMS: 287[M+1]⁺

¹H NMR (400 MHz, CDCl₃) δ 7.64 (d, 1H), 7.57 (d, 1H), 7.16 (d, 1H), 7.08 (d, 1H), 6.96-6.89 (m, 1H), 6.03 (d, 1H), 4.45-4.40 (m, 2H), 4.28-4.20 (m, 2H), 4.03-3.96 (m, 2H), 2.46-2.40 (m, 2H).

### Example 23

### Preparation of (E)-1-(3-(2,3-dihydro-[1,4]dioxino[2,3-b]pyridin-7-yl)acryloyl)-5,6-dihydropyridin-2(1H)-one

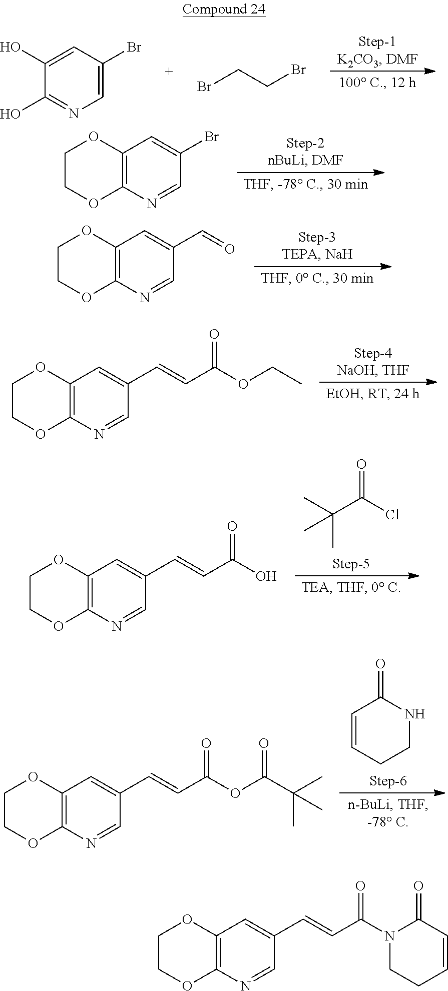

### Step-1

To a solution of 5-bromopyridine-2,3-diol (10 g, 53.19 mmol, 1 eq.) in DMF (250 mL) at room temperature was added K₂CO₃ (21 g, 159.57 mmol, 3 eq.) followed by 1,2-dibromoethane (5.5 mL, 63.82 mmol, 1.2 eq.) dropwise. The resulting mixture was stirred at 100° C. overnight. Progress of reaction was monitored by TLC. After the completion, reaction mixture was cooled to room temperature, diluted with ice cold water (300 mL) and extracted with DCM (3×300 mL). Combined organic layer was washed with brine and dried over anhydrous sodium sulphate. Removal of solvent under reduced pressure afforded crude was purified by Combi-Flash on silica gel using methanol-dichloromethane (0-50%) as eluents to afford 7-bromo-2,3-dihydro-[1,4]dioxino[2,3-b]pyridine (2.9 g, 25.26%).

LCMS: 215 [M+1]⁺

### Step-2

To a solution of 7-bromo-2,3-dihydro-[1,4]dioxino[2,3-b]pyridine (0.500 g, 2.31 mmol, 1 eq.) in THF (15 mL) at −78° C. was added n-BuLi (1.43 mL, 0.50 mmol, 1.6 eq.) and the resulting mixture was allowed stirred at the same temperature for 30 minutes. To this solution was added a solution of DMF (0.43 mL, 5.54 mmol, 2.4 eq.) in THF (4 mL) and reaction mixture was allowed to stir at the same temperature for 2 h. Progress of reaction was monitored by TLC. After the completion, the reaction mixture was brought to 0° C., diluted with saturated NH₄Cl solution (50 mL) and extracted with ethyl acetate (3×50 mL). Combined organic layer dried over Na₂SO₄. Removal of solvent under reduced pressure afforded 2,3-dihydro-[1,4]dioxino[2,3-b]pyridine-7-carbaldehyde (0.35 g, 92.1%) which was used in the next step without further purification.

LCMS: 166 [M+1]⁺

### Step-3

To a solution of triethyl phosphonoacetate (0.618 g, 2.76 mmol, 1.3 eq.) in THF (30 mL) at 0° C. was added NaH (0.127 g, 3.18 mmol, 1.5 eq.) and the reaction mixture was allowed stirred at the same temperature for 10 minute. To this mixture was added a solution of 2,3-dihydro-[1,4]dioxino[2,3-b]pyridine-7-carbaldehyde (0.35 g, 2.76 mmol, 1 eq.) in THF (1 mL) and reaction mixture was allowed to stir at the same temperature for 30 minutes. Progress of reaction was monitored by TLC. After the completion, the reaction mixture was diluted with brine solution (50 mL) and extracted with ethyl acetate (3×50 mL). Combined organic layer was dried over Na₂SO₄. Removal of solvent under reduced pressure afforded (E)-ethyl 3-(2,3-dihydro-[1,4]dioxino[2,3-b]pyridin-7-yl)acrylate (0.45 g, 90.36%) which was used in the next step without further purification.

LCMS: 236 [M+1]⁺

### Step-4

To a solution of (E)-ethyl 3-(2,3-dihydro-[1,4]dioxino[2,3-b]pyridin-6-yl)acrylate (0.45 g, 1.91 mmol, 1 eq.) in a mixture of THF-ethanol (1:1, 10 mL) was added 5N aq. NaOH solution (3.82 mL, 19.1 mmol, 10 eq.) and mixture was stirred at room temperature overnight. Progress of reaction was monitored by TLC. After completion, reaction mixture was diluted with water (30 mL) and extracted with ethyl acetate (30 mL). Aqueous layer was acidified up to pH 6 and extracted with ethyl acetate (3×50 mL). Combined organic layer was washed with brine and dried over sodium sulphate. Removal of solvent under reduced pressure afforded crude (E)-3-(2,3-dihydro-[1,4]dioxino[2,3-b]pyridin-7-yl)acrylic acid (0.340 g, 39.6%) which was used in the next step without further purification.

LCMS: 208[M+1]⁺

### Step-5

To a stirred solution of (E)-3-(2,3-dihydro-[1,4]dioxino[2,3-b]pyridin-7-yl)acrylic acid (0.1 g, 0.48 mmol, 1 eq.) in THF (15 mL) at 0° C. was added triethylamine (0.07 mL, 0.579 mmol, 1.2 eq.) followed by pivaloyl chloride (0.07 mL, 0.579 mmol, 1.2 eq.) and the resulting mixture was stirred stir at 0° C. for 2 h. Progress of reaction was monitored by TLC. After the completion, reaction mixture was used as such in the next reaction.

### Step-06

To a stirred solution of 5,6-dihydropyridin-2(1H)-one (0.07 g, 0.72 mmol, 1.5 eq.) in THF (10 mL) at −78° C. was added n-BuLi (0.3 mL, 0.75 mmol, 2.2 eq.) and the resulting mixture was stirred at the same temperature for 30 minutes. To this mixture was added a solution of (E)-3-(2,3-dihydro-[1,4]dioxino[2,3-b]pyridin-7-yl)acrylic pivalic anhydride in THF (15 mL) dropwise and the reaction mixture was stirred at −78° C. for 2 h. Progress of reaction was monitored by TLC. After completion, reaction mixture was diluted with ammonium chloride solution (50 mL) and extracted with ethyl acetate (3×50 mL). Combined organic layer was dried over sodium sulphate. Removal of solvent under reduced pressure crude which was purified by reversed phase HPLC to afford (E)-1-(3-(2,3-dihydro-[1,4]dioxino[2,3-b]pyridin-7-yl)acryloyl)-5,6-dihydropyridin-2(1H)-one (7 mg, 5%).

LCMS 287[M+1]⁺

¹H NMR (400 MHz, CD₃OD) δ 7.98 (s, 1H), 7.80 (d, 1H), 7.79 (s, 1H), 7.38 (d, 1H), 7.10-6.98 (m, 1H), 4.50-4.42 (m, 2H), 4.32-4.28 (m, 2H), 3.95-4.03 (m, 2H), 2.47-2.40 (m, 2H).

### Example 24

### Preparation of 5-hydroxy-1-[(2E)-3-(8-methoxy-2,3-dihydro-1,4-benzodioxin-6-yl)prop-2-enoyl]-5,6-dihydropyridin-2(1H)-one

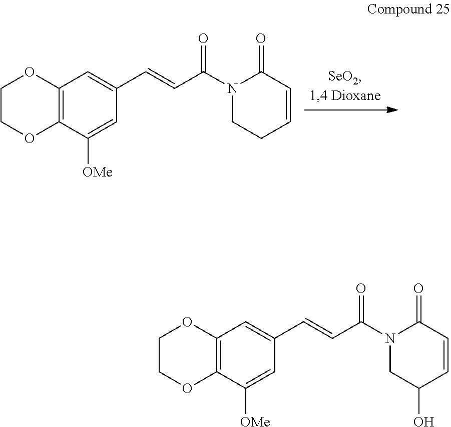 [SMILES: COc1cc(/C=C/C(=O)N2CCC=CC2=O)cc2c1OCCO2 ; COc1cc(/C=C/C(=O)N2CC(O)C=CC2=O)cc2c1OCCO2]

To the stirred solution of 1-[(2E)-3-(8-methoxy-2,3-dihydro-1,4-benzodioxin-6-yl)prop-2-enoyl]-5,6-dihydropyridin-2(H)-one (300 mg, 0.95 mmol, 1 eq.) in 1,4 dioxane (6.0 mL) was added selenium dioxide (314 mg, 2.85 mmol, 3 eq.) portionwise. The reaction mixture was stirred at 120° C. in microwave for 2 h. Progress of the reaction was monitored by TLC and LCMS. After 40% completion, reaction mixture was diluted with water (20 mL) and extracted with ethyl acetate (2×50 mL). Combined organic layer was washed with brine, dried over Na₂SO₄ and concentrated under reduced pressure to obtain crude which was purified by reversed phase HPLC to afford 5-hydroxy-1-[(2E)-3-(8-methoxy-2,3-dihydro-1,4-benzodioxin-6-yl)prop-2-enoyl]-5,6-dihydropyridin-2(1H)-one (50 mg, 15.87%) as yellow solid.

LCMS: 332 [M+1]⁺

¹H NMR (400 MHz, DMSO-d₆) δ 7.49 (d, 1H), 7.25 (d, 1H), 6.97 (dd, 1H), 6.86 (s, 1H) 6.81 (s, 1H), 5.96 (d, 1H), 5.59 (d, 1H), 4.38 (brs., 1H), 4.25 (brs, 3H), 3.90 (d, 1H), 3.84-3.71 (m, 2H).

### Example 25

### Preparation of (E)-1-(3-(2,3-dihydro-[1,4]dioxino[2,3-b]pyridin-7-yl)acryloyl)-6,7-dihydro-1H-azepin-2(5H)-one

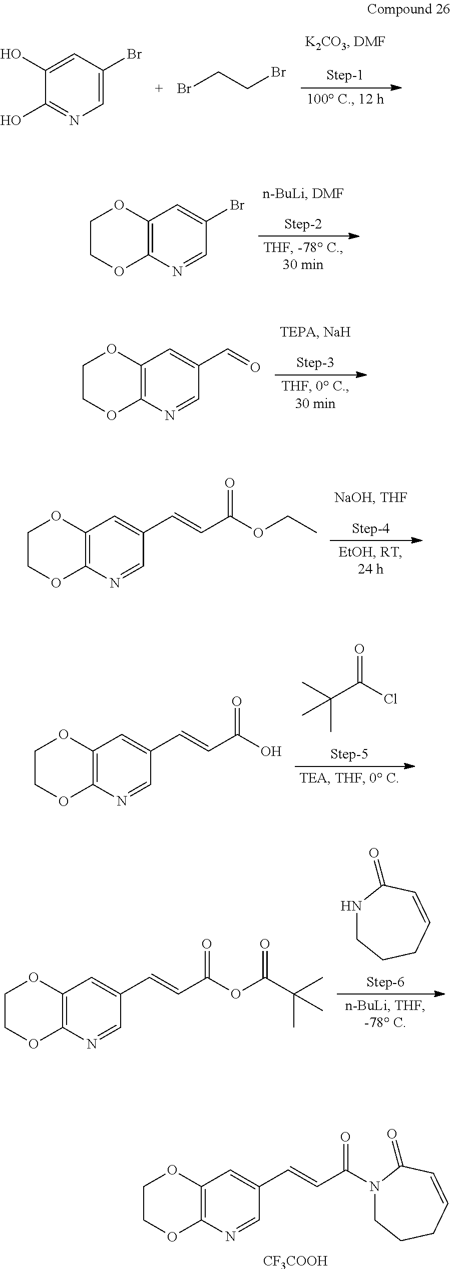

### Step-1

To a solution of 5-bromopyridine-2,3-diol (10 g, 53.19 mmol, 1 eq.) in DMF (250 mL) at room temperature was added K₂CO₃ (21 g, 159.57 mmol, 3 eq.) followed by 1,2-dibromoethane (5.5 mL, 63.82 mmol, 1.2 eq.) dropwise. The resulting mixture was stirred at 100° C. overnight. Progress of reaction was monitored by TLC. After the completion, reaction mixture was cooled to room temperature, diluted with ice cold water (300 mL) and extracted with DCM (3×300 mL). Combined organic layer was washed with brine and dried over anhydrous sodium sulphate. Removal of solvent under reduced pressure afforded crude was purified by Combi-Flash on silica gel using methanol-dichloromethane (0-50%) as eluents to afford 7-bromo-2,3-dihydro-[1,4]dioxino[2,3-b]pyridine (2.9 g, 25.26%).

LCMS: 215 [M+1]⁺

### Step-2

To a solution of 7-bromo-2,3-dihydro-[1,4]dioxino[2,3-b]pyridine (0.500 g, 2.31 mmol, 1 eq.) in THF (15 mL) at −78° C. was added n-BuLi (1.43 mL, 0.50 mmol, 1.6 eq.) and the resulting mixture was allowed stirred at the same temperature for 30 minutes. To this solution was added a solution of DMF (0.43 mL, 5.54 mmol, 2.4 eq.) in THF (4 mL) and reaction mixture was allowed to stir at the same temperature for 2 h. Progress of reaction was monitored by TLC. After completion, the reaction mixture was brought to 0° C., diluted with saturated NH₄Cl solution (50 mL) and extracted with ethyl acetate (3×50 mL). The combined organic layer was dried over Na₂SO₄. Removal of solvent under reduced pressure afforded 2,3-dihydro-[1,4]dioxino[2,3-b]pyridine-7-carbaldehyde (0.35 g, 92.1%) which was used in the next step without further purification.

LCMS: 166 [M+1]⁺

### Step-3

To a solution of triethyl phosphonoacetate (0.618 g, 2.76 mmol, 1.3 eq.) in THF (30 mL) at 0° C. was added NaH (0.127 g, 3.18 mmol, 1.5 eq.) and the reaction mixture was allowed stirred at the same temperature for 10 minutes. To this mixture was added a solution of 2,3-dihydro-[1,4]dioxino[2,3-b]pyridine-7-carbaldehyde (0.35 g, 2.76 mmol, 1 eq.) in THF (1 mL) and reaction mixture was allowed to stir at the same temperature for 30 minutes. Progress of reaction was monitored by TLC. After the completion, the reaction mixture was diluted with brine solution (50 mL) and extracted with ethyl acetate (3×50 mL). Combined organic layer was dried over Na₂SO₄. Removal of solvent under reduced pressure afforded (E)-ethyl 3-(2,3-dihydro-[1,4]dioxino[2,3-b]pyridin-7-yl)acrylate (0.45 g, 90.36%) which was used in the next step without further purification.

LCMS: 236 [M+1]⁺

### Step-4

To a solution of (E)-ethyl 3-(2,3-dihydro-[1,4]dioxino[2,3-b]pyridin-6-yl)acrylate (0.45 g, 1.91 mmol, 1 eq.) in a mixture of THF-ethanol (1:1, 10 mL) was added 5N aq. NaOH solution (3.82 mL, 19.1 mmol, 10 eq.) and mixture was stirred at room temperature overnight. Progress of reaction was monitored by TLC. After completion, reaction mixture was diluted with water (30 mL) and extracted with ethyl acetate (30 mL). Aqueous layer was acidified up to pH 6 and extracted with ethyl acetate (3×50 mL). Combined organic layer was washed with brine and dried over sodium sulphate. Removal of solvent under reduced pressure afforded crude (E)-3-(2,3-dihydro-[1,4]dioxino[2,3-b]pyridin-7-yl)acrylic acid (0.340 g, 39.6%) which was used in the next step without further purification.

LCMS: 208[M+1]⁺

### Step-5

To a stirred solution of (E)-3-(2,3-dihydro-[1,4]dioxino[2,3-b]pyridin-7-yl)acrylic acid (0.1 g, 0.48 mmol, 1 eq.) in THF (15 mL) at 0° C. was added triethylamine (0.07 mL, 0.579 mmol, 1.2 eq.) followed by pivaloyl chloride (0.07 mL, 0.579 mmol, 1.2 eq.) and the resulting mixture was stirred stir at 0° C. for 2 h. Progress of reaction was monitored by TLC. After the completion, reaction mixture was used as such in the next reaction.

### Step-6

To a stirred solution of 6,7-dihydro-1H-azepin-2(5H)-one (0.08 g, 0.71 mmol, 1.5 eq.) in THF (10 mL) at −78° C. was added n-BuLi (0.3 mL, 0.75 mmol, 2.2 eq.) and the resulting mixture was stirred at the same temperature for 30 minutes. To this mixture was added a solution of (E)-3-(2,3-dihydro-[1,4]dioxino[2,3-b]pyridin-7-yl)acrylic pivalic anhydride in THF (15 mL) dropwise and the reaction mixture was stirred at −78° C. for 2 h. Progress of reaction was monitored by TLC. After completion, reaction mixture was diluted with ammonium chloride solution (50 mL) and extracted with ethyl acetate (3×50 mL). Combined organic layer was dried over sodium sulphate. Removal of solvent under reduced pressure crude which was purified by reversed phase HPLC to afford (E)-1-(3-(2,3-dihydro-[1,4]dioxino[2,3-b]pyridin-7-yl)acryloyl)-6,7-dihydro-1H-azepin-2(5H)-one (11 mg, 6%) as TFA salt.

LCMS: 301[M+1]⁺

¹H NMR (400 MHz, DMSO-d₆) δ 8.01 (s, 1H), 7.60 (s, 1H), 7.37 (d, 1H), 7.23 (d, 1H), 6.65-6.55 (m, 1H), 6.03 (m, 1H), 4.45 (brs, 2H), 4.26 (brs, 2H), 3.90-3.80 (m, 2H), 2.41-2.30 (m, 2H), 1.90-1.80 (m, 2H).

### Example 26

### Preparation of (E)-1-(3-(pyridazin-3-yl)acryloyl)-5,6-dihydropyridin-2(1H)-one

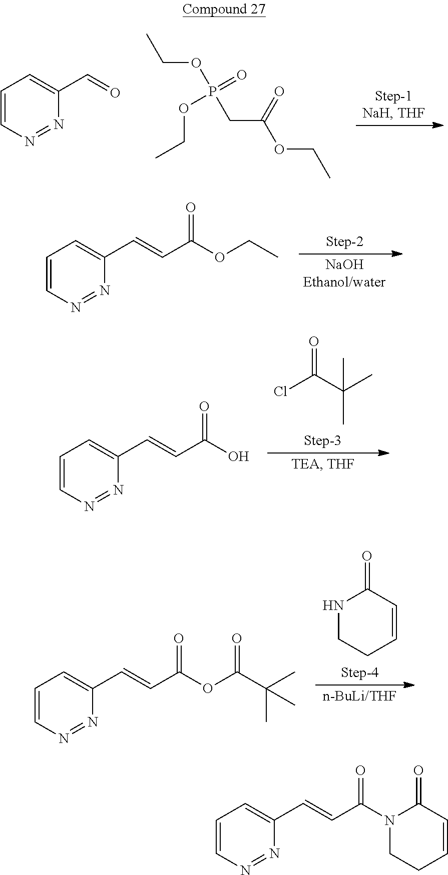

### Step-1

To a suspension of sodium hydride (60% in mineral oil, 0.203 g, 5.09 mmol, 1.1 eq.) in THF (50 mL) was added ethyl 2-(diethoxyphosphoryl)acetate (2.07 g, 9.25 mmol, 2.0 eq.) dropwise at 0° C. and the mixture was allowed to stir at the same temperature for 30 minutes. To this mixture was added a solution of pyridazine-3-carbaldehyde (0.5 g, 4.62 mmol, 1.0 eq.) in THF (5 mL) and the resulting mixture was allowed to stir at room temperature for 1 h. Progress of reaction was monitored by TLC. After completion, reaction mixture was diluted with saturated aq. NH₄Cl (100 mL) and extracted with ethyl acetate (3×50 ml). Combined organic layer was washed with brine, dried over anhydrous Na₂SO₄ and evaporated to dryness under vacuum to afford crude which was purified by Combi-Flash to using ethyl acetate-hexane system as eluent to afford ethyl (E)-3-(pyridazin-3-yl)acrylate (0.42 g, 50.97%).

LCMS: 179 [M+1]⁺

### Step-2

To a solution of ethyl (E)-3-(pyridazin-3-yl)acrylate (0.4 g, 2.24 mmol, 1.0 eq.) in ethanol (200 mL) was added a solution of sodium hydroxide (2M, 2.0 ml) in water (10 mL) and the reaction mixture was stirred at 70° C. for 3 h. Reaction mixture was concentrated under vacuum and then acidified with 2N HCl to make pH 3. The mixture was diluted saturated aq. NH₄Cl (25 mL) and extracted with dichloromethane (3×25 ml). The combined organic layer was washed with brine solution (50 mL) and dried over anhydrous Na₂SO₄. Removal of solvent under reduced pressure to obtain (E)-3-(pyridazin-3-yl)acrylic acid (0.29 g, 86%) as crystalline solid.

LCMS: 151 [M+1]⁺

### Step-3

To a solution of (E)-3-(pyridazin-3-yl)acrylic acid (0.29 g, 1.93 mmol, 1.0 eq.) in dry THF was added triethylamine (0.27 ml, 2.12 mmol, 1.1 eq.) followed by pivaloyl chloride (0.26 ml, 2.12 mmol, 1.1 eq.) and the reaction mixture was stirred at 0° C. for 45 minutes. Progress of reaction was monitored by TLC. After completion, reaction mixture was filtered and the filtrate was used directly for the next step.

### Step-4

To a stirred solution of 5,6-dihydropyridin-2(1H)-one (0.18 g, 1.85 mmol, 1.0 eq.) in dry THF at −78° C. was added n-butyllithium (2.5 M in hexane, 0.81 ml, 2.04 mmol, 1.1 eq.) and the mixture was allowed to stir at the same temperature for 30 minutes. To this solution was added a solution of (E)-pivalic (E)-3-(pyridazin-3-yl)acrylic anhydride (0.390 g, 1.67 mmol, 0.9 eq.) in THF and the reaction mixture was allowed to stir at −78° C. for 1 h. Progress of reaction was monitored by TLC and LCMS. After completion, reaction mixture was brought to 0° C., diluted with water (30 mL) and extracted with ethyl acetate (3×30 ml). Combined organic layer was washed with brine, dried over anhydrous Na₂SO₄ and evaporated to dryness under vacuum to afford crude which was purified by reversed phase HPLC to afford (E)-1-(3-(pyridazin-3-yl)acryloyl)-5,6-dihydropyridin-2(1H)-one (0.015 g, 3.52%).

LCMS: 230 [M+1]⁺

¹H NMR (400 MHz, DMSO-d₆) δ 9.21 (d, 1H), 8.02 (d, 1H), 7.85 (d, 1H), 7.76 (dd, 1H), 7.65 (d, 1H), 7.09-7.18 (m, 1H), 6.00 (d, 1H), 3.93 (t, 2H).

### Example 27

### Preparation of (E)-1-(4,4-dimethylpent-2-enoyl)-5,6-dihydropyridin-2(1H)-one

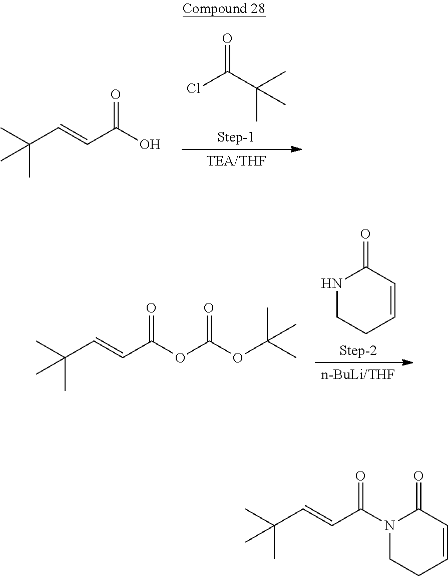

### Step-1

To a solution of (E)-4,4-dimethylpent-2-enoic acid (0.3 g, 2.34 mmol, 1.0 eq.) in dry THF (10 mL) was added triethylamine (0.33 ml, 2.57 mmol, 1.1 eq.) followed by pivaloyl chloride 2 (0.30 mL, 2.57 mmol, 1.1 eq.) and the reaction mixture was stirred at 0° C. for 45 minutes. Progress of reaction was monitored by TLC. After completion, reaction mixture was filtered and the filtrate was used directly for the next step.

### Step-2

To a stirred solution of 5,6-dihydropyridin-2(1H)-one (0.100 g, 1.03 mmol, 1.0 eq.) in dry THF at −78° C. was added n-butyllithium (2.5 M in hexane, 0.45 ml, 1.13 mmol, 1.1 eq.) and the mixture was allowed to stir at the same temperature for 30 minutes. To this solution was added a solution of (E)-(tert-butyl carbonic) (E)-4,4-dimethylpent-2-enoic anhydride (0.221 g, 0.92 mmol, 1.1 eq.) in THF (10 mL) and the reaction mixture was allowed to stir at −78° C. for 1 h. Progress of reaction was monitored by TLC and LCMS. After completion, reaction mixture was brought to 0° C., diluted with water (30 mL) and extracted with ethyl acetate (3×30 ml). Combined organic layer was washed with brine, dried over anhydrous Na₂SO₄ and evaporated to dryness under vacuum to afford crude which was purified by reversed phase HPLC to afford (E)-1-(4,4-dimethylpent-2-enoyl)-5,6-dihydropyridin-2(1H)-one (0.011 g, 6%).

LCMS: 208 ([M+1]⁺)

¹H NMR (500 MHz, CDCl₃) δ 7.02 (d, 1H), 6.84-6.96 (m, 1H), 6.78 (d, 1H), 6.00 (d, 1H), 3.98 (t, 2H), 2.44 (d, 2H), 1.11 (s, 9H).

### Example 28

### Preparation of 1-(3,3-diphenylacryloyl)-5,6-dihydropyridin-2(1H)-one

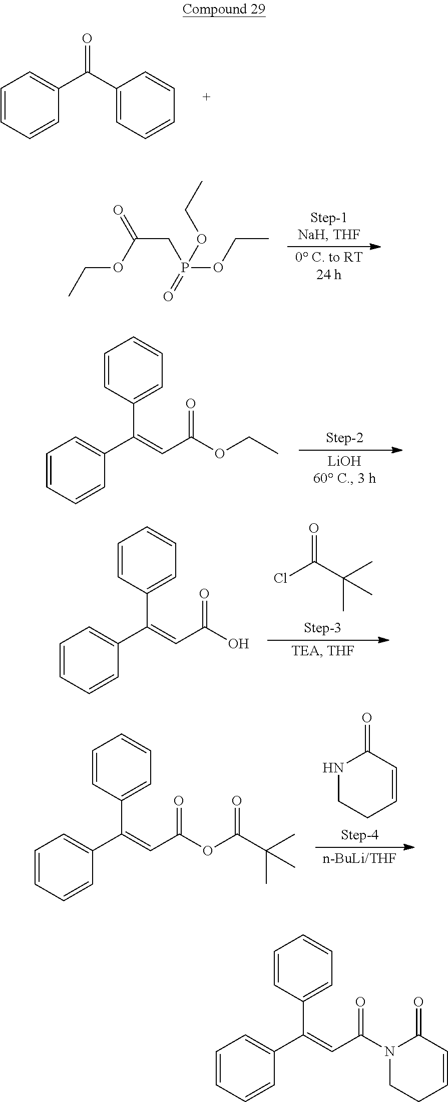

### Step-1

To a suspension of sodium hydride (60% in mineral oil, 0.439 g, 10.98 mmol, 2.0 eq.) in THF (50 mL) was added ethyl 2-(diethoxyphosphoryl)acetate (2.08 mL, 10.98 mmol, 2.0 eq.) dropwise at 0° C. and the mixture was allowed to stir at the same temperature for 30 minutes. To this mixture was added a solution of diphenylmethanone (1.0 g, 5.49 mmol, 1.0 eq.) in THF (5 mL) and the resulting mixture was allowed to stir at room temperature for 24 h. Progress of reaction was monitored by TLC. After completion, reaction mixture was diluted with saturated aq. NH₄Cl (100 mL) and extracted with ethyl acetate (3×50 ml). Combined organic layer was washed with brine, dried over anhydrous Na₂SO₄ and evaporated to dryness under vacuum to afford crude which was purified by Combi-Flash using ethyl acetate-hexane system as eluent to afford ethyl 3,3-diphenylprop-2-enoate (0.53 g, 38.4%).

LCMS: 253 [M+1]⁺

### Step-2

To a solution of ethyl 3,3-diphenylprop-2-enoate (0.530 g, 2.10 mmol, 1.0 eq.) in ethanol (100 mL) was added a solution of lithium hydroxide (0.2 g) in water (2 mL) and the reaction mixture was stirred at 70° C. for 3 h. Progress of reaction was monitored by TLC. After completion, reaction mixture was concentrated under vacuum, acidified with 1N HCl (50 mL) and extracted with ethyl acetate (3×50 ml). Combined organic layer was washed with brine solution (50 mL) and dried over anhydrous Na₂SO₄. Removal of solvent under reduced pressure afforded 3,3-diphenylprop-2-enoic acid (0.450 g, 95.54%) as crystalline solid.

LCMS: 225 [M+1]⁺

### Step-3

To a solution of 3,3-diphenylprop-2-enoic acid (0.45 g, 2.00 mmol, 1.0 eq.) in dry THF (10 mL) was added triethylamine (0.34 ml, 2.4 mmol, 1.2 eq.) followed by pivaloyl chloride (0.284 ml, 2.4 mmol, 1.2 eq.) and the reaction mixture was stirred at 0° C. for 45 minutes. Progress of reaction was monitored by TLC. After completion, reaction mixture was filtered and the filtrate was used directly for the next step.

### Step-4

To a stirred solution of 5,6-dihydropyridin-2(1H)-one (0.2 g, 2.05 mmol, 1.0 eq.) in dry THF at −78° C. was added n-butyllithium (2.5 M in hexane, 0.9 ml, 2.26 mmol, 1.1 eq.) and the mixture was allowed to stir at the same temperature for 30 minutes. To this solution was added a solution of 2,2-dimethylpropanoic 3,3-diphenylprop-2-enoic anhydride (0.698 g, 2.26 mmol, 1.1 eq.) in THF and the reaction mixture was allowed to stir at −78° C. for 1 h. Progress of reaction was monitored by TLC and LCMS. After completion, reaction mixture was brought to 0° C., diluted with water (30 mL) and extracted with ethyl acetate (3×50 mL). Combined organic layer was washed with brine, dried over anhydrous Na₂SO₄ and evaporated to dryness under vacuum to afford crude which was purified by combi flash to afford 1-(3,3-diphenylacryloyl)-5,6-dihydropyridin-2(1H)-one (0.22 g, 32.06%).

LCMS: 304 [M+1]⁺

¹H NMR (400 MHz, CDCl₃) δ 7.39-7.19 (m, 10H), 6.90 (s, 1H), 6.85-7.75 (m, 1H), 5.97 (d, 1H), 3.80 (t, 2H), 2.25-2.18 (m, 2H).

### Example 29

### Preparation of (E)-1-(3-(8-methoxy-2,3-dihydrobenzo[b][1,4]dioxin-6-yl)acryloyl)-5,5-dimethyl-5,6-dihydropyridin-2(1H)-one

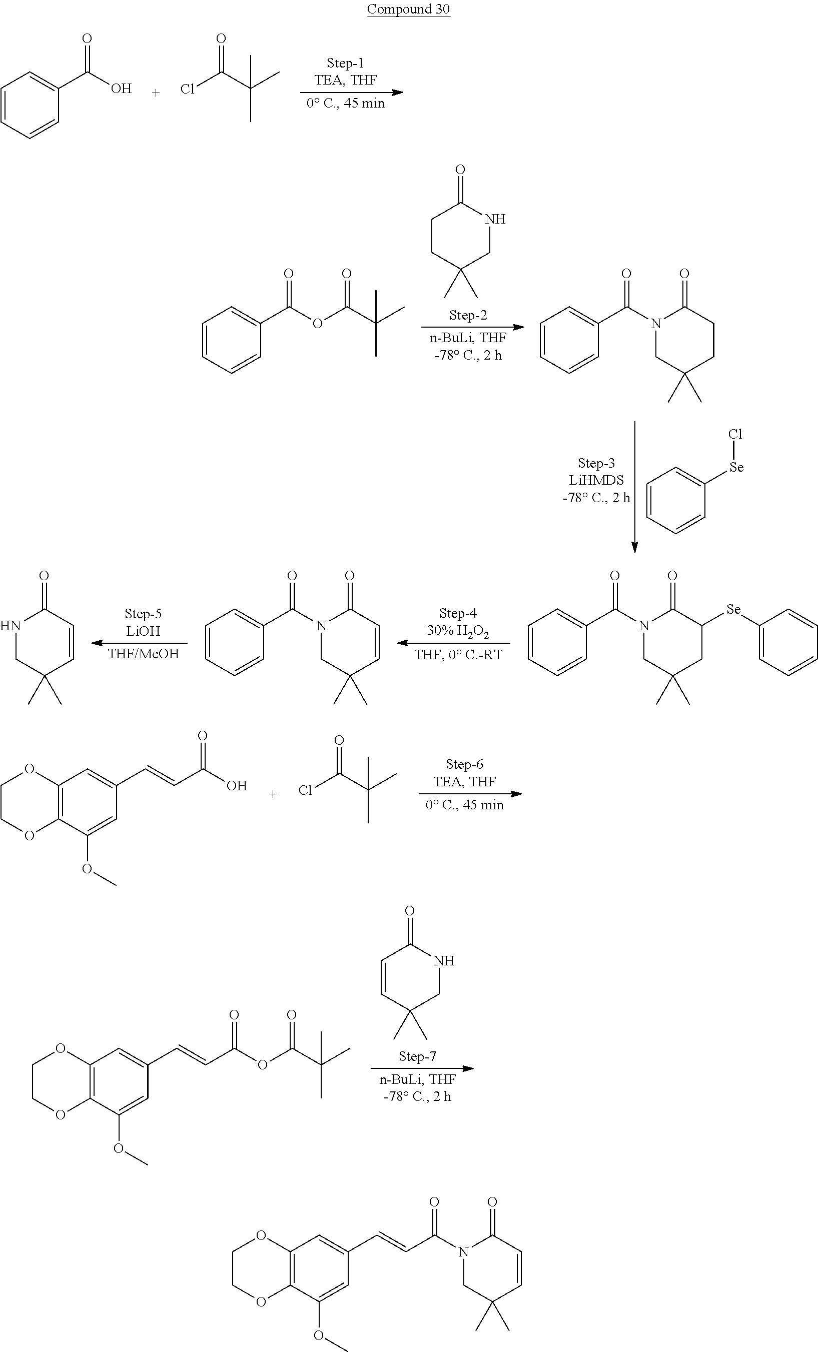

### Step-1

To a solution of benzoic acid (1.15 g, 9.41 mmol, 1.0 eq.) in dry THF was added triethylamine (1.50 mL, 11.30 mmol, 1.2 eq.) and pivaloyl chloride (1.39 mL, 11.30 mmol, 1.2 eq.) and the reaction mixture was stirred at 0° C. for 45 minutes. Progress of reaction was monitored by TLC. After completion, reaction mixture was filtered and the filtrate was used directly for the next step.

### Step-2

To a stirred solution of 5,5-dimethylpiperidin-2-one (1 g, 7.48 mmol, 0.8 eq.) in dry THF at −78° C. was added n-butyllithium (2.5 M in hexane, 3 mL, 7.50 mmol, 0.8 eq.) and the mixture was allowed to stir at the same temperature for 30 minutes. To this solution was added benzoic pivalic anhydride (1.93 g, 9.35 mmol, 1 eq.). Reaction mixture was allowed to stir at −78° C. for 90 minutes. Progress of reaction was monitored by TLC and LCMS. After completion, reaction mixture was brought to 0° C., diluted with water (40 mL) and extracted with ethyl acetate (3×50 mL). Combined organic layer was washed with brine, dried over anhydrous Na₂SO₄ and evaporated to dryness under vacuum to afford crude which was purified by CombiFlash on silica gel using ethyl acetate-hexane system as eluent to afford 1-benzoyl-5,5-dimethylpiperidin-2-one (0.550 g, 25.46%).

### Step-3

To a stirred solution of 1-benzoyl-5,5-dimethylpiperidin-2-one (0.3 g, 1.29 mmol, 1 eq.) in dry THF at −78° C. was added LiHMDS (1 M in THF, 2.59 mL, 2.59 mmol, 2 eq.) and the mixture was allowed to stir at the same temperature for 30 minutes. To this solution was added phenyl hypochloroselenoite (0.496 g, 2.59 mmol, 2 eq.). Reaction mixture was allowed to stir at −78° C. for 90 min. Progress of reaction was monitored by TLC and LCMS. Reaction mixture was brought to 0° C., diluted with water (35 mL) and extracted with ethyl acetate (3×40 mL). Combined organic layer was washed with brine, dried over anhydrous Na₂SO₄ and evaporated to dryness under vacuum to afford crude which was purified by CombiFlash on silica gel using ethyl acetate-hexane system as eluent to afford 1-benzoyl-5,5-dimethyl-3 (phenylselanyl)piperidin-2-one (0.3 g, 60%).

### Step-4

To a stirred solution of 1-benzoyl-5,5-dimethyl-3-(phenylselanyl)piperidin-2-one (0.3 g, 0.77 mmol, 1 eq.) in dry THF at 0° C. was added 30% aq. H₂O₂ (0.4 mL, 3.88 mmol, 5 eq.) and the mixture was allowed to stir at the same temperature for 10 minutes followed by stirring at room temperature for 20 min. Progress of reaction was monitored by TLC and LCMS. Reaction mixture was diluted with water (20 mL) and extracted with ethyl acetate (3×25 mL). Combined organic layer was washed with brine, dried over anhydrous Na₂SO₄ and evaporated to dryness under vacuum to afford crude 1-benzoyl-5,5-dimethyl-5,6-dihydropyridin-2(1H)-one (0.250 g crude) which is used in next step without purification.

### Step-5

To a stirred solution of 1-benzoyl-5,5-dimethyl-5,6-dihydropyridin-2(1H)-one (0.230 g, 1.00 mmol, 1 eq.) in mixture of MeOH (5 mL) and THF (5 mL) and the mixture was allowed to stir at room temperature for 10 minutes followed by addition of LiOH (0.144 g, 6.01 mmol, 6 eq.). Reaction mixture was allowed to at same temperature for 1 h. Progress of reaction was monitored by TLC and LCMS. Reaction mixture was diluted with water (20 mL) and extracted with ethyl acetate (3×35 mL). Combined organic layer was washed with brine, dried over anhydrous Na₂SO₄ and evaporated to dryness under vacuum to afford crude 5,5-dimethyl-5,6-dihydropyridin-2(1H)-one (0.11 g, 88%) which was used in the next step without purification.

### Step-6

To a solution of (E)-3-(8-methoxy-2,3-dihydrobenzo[b][1,4]dioxin-6-yl)acrylic acid (0.205 g, 0.869 mmol, 1.0 eq.) in dry THF was added triethylamine (0.14 mL, 1.04 mmol, 1.2 eq.) and pivaloyl chloride (0.14 mL, 1.04 mmol, 1.2 eq.) and the reaction mixture was stirred at 0° C. for 45 minutes. Progress of reaction was monitored by TLC. After completion, reaction mixture was filtered and the filtrate was used directly for the next step.

### Step-7

To a stirred solution of 5,5-dimethyl-5,6-dihydropyridin-2(1H)-one (0.110 g, 0.867 mmol, 1 eq.) in dry THF at −78° C. was added n-butyllithium (2.5 M in hexane, 0.38 mL, 0.953 mmol, 1.1 eq.) and the mixture was allowed to stir at the same temperature for 30 minutes. To this solution was added (E)-3-(8-methoxy-2,3-dihydrobenzo[b][1,4]dioxin-6-yl)acrylic pivalic anhydride (0.277 g, 0.867 mmol, 1 eq.). Reaction mixture was allowed to stir at −78° C. for 90 min. Progress of reaction was monitored by TLC and LCMS. Reaction mixture was brought to 0° C., diluted with water (35 mL) and extracted with ethyl acetate (3×40 mL). Combined organic layer was washed with brine, dried over anhydrous Na₂SO₄ and evaporated to dryness under vacuum to afford crude which was purified by reversed phase HPLC to afford (E)-1-(3-(8-methoxy-2,3-dihydrobenzo[b][1,4]dioxin-6-yl)acryloyl)-5,5-dimethyl-5,6-dihydropyridin-2(1H)-one (20 mg, 6.75%).

LCSM: 344 [M+1]⁺

¹H NMR (400 MHz, CDCl₃) δ 7.62 (d, 1H), 7.42 (d, 1H), 6.80 (s, 1H), 6.75 (s, 1H), 6.62 (d, 1H), 5.87 (d, 1H), 4.39-4.31 (m, 2H), 4.30-4.25 (m, 2H), 3.93 (s, 3H), 3.79 (s, 3H), 1.18 (s, 6H).

### Example 30

### Preparation of (E)-1-(3-(2,3-dihydro-[1,4]dioxino[2,3-b]pyridin-6-yl)acryloyl)-1,5,6,7-tetrahydroazepin-2-one

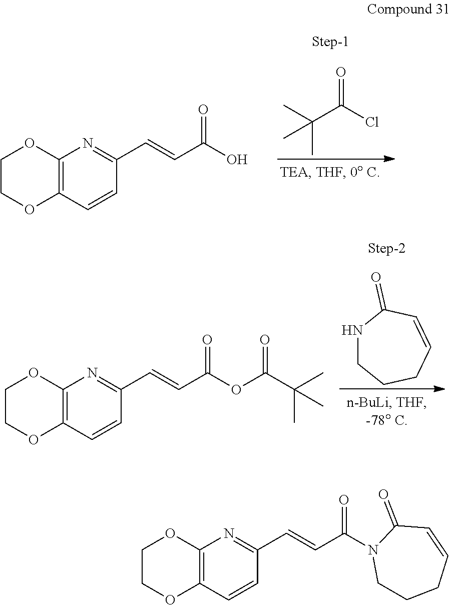

### Step-1

To a stirred solution of (E)-3-(2,3-dihydro-[1,4]dioxino[2,3-b]pyridin-6-yl)acrylic acid (0.7 g, 3.38 mmol, 1 eq.) in THF (15 mL) at 0° C. was added triethylamine (0.5 mL, 4.05 mmol, 1.2 eq.) followed by pivaloyl chloride (0.5 mL, 4.05 mmol, 1.2 eq.) and the resulting mixture was stirred stir at 0° C. for 2 h. Progress of reaction was monitored by TLC. After the completion, reaction mixture was used as such in the next reaction.

### Step-2

To a stirred solution of 1,5,6,7-tetrahydroazepin-2-one (0.4 g, 1.37 mmol, 1.5 eq.) in THF (15 mL) at −78° C. was added n-BuLi (1.2 mL, 3.02 mmol, 2.2 eq.) and the resulting mixture was stirred at the same temperature for 30 minutes. To this mixture was added a solution of (E)-3-(2,3-dihydro-[1,4]dioxino[2,3-b]pyridin-6-yl)acrylic pivalic anhydride in THF (15 mL) dropwise and the reaction mixture was stirred at −78° C. for 2 h. Progress of reaction was monitored by TLC. After completion, reaction mixture was diluted with ammonium chloride solution (50 mL) and extracted with ethyl acetate (3×50 mL). Combined organic layer was dried over sodium sulphate. Removal of a solvent under reduced pressure crude which was purified by reversed phase HPLC to afford (E)-1-(3-(2,3-dihydro-[1,4]dioxino[2,3-b]pyridin-6-yl)acryloyl)-1,5,6,7-tetrahydroazepin-2-one (20 mg, 4.8%).

LCMS: 301[M+1]⁺

¹H NMR (400 MHz, CDCl₃) δ 7.59 (s, 2H), 7.17 (d, 2H), 7.03 (d, 2H), 6.60-6.48 (m, 1H), 6.08 (d, 1H), 4.45-4.40 (m, 2H), 4.25-4.20 (m, 2H), 3.99-3.92 (m, 2H), 2.42-2.35 (m, 2H), 2.20-1.92 (m, 2H).

### Example 31

### Preparation of 5-hydroxy-1-[(2E)-3-(pyrimidin-2-yl)prop-2-enoyl]-5,6-dihydropyridin-2(1H)-one

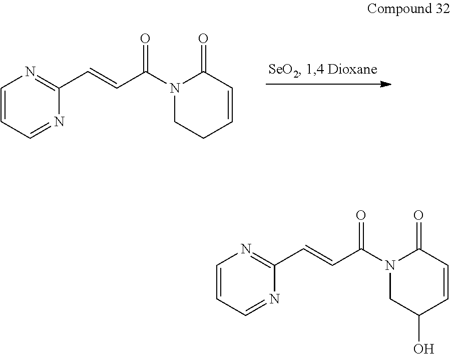 [SMILES: O=C1C=CCCN1C(=O)/C=C/c1ncccn1 ; O=C1C=CC(O)CN1C(=O)/C=C/c1ncccn1]

To the stirred solution of 1-[(2E)-3-(pyrimidin-2-yl)prop-2-enoyl]-5,6-dihydropyridin-2(1H)-one (500 mg, 2.18 mmol, 1 eq.) in 1,4 dioxane (15 mL) was added selenium dioxide (720 mg, 6.54 mmol, 3 eq.) portionwise. The reaction mixture was stirred at 120° C. in microwave for 2 h. Progress of the reaction was monitored by TLC and LCMS. After 40% completion, reaction mixture was diluted with water (20 mL) and extracted with ethyl acetate (2×50 mL). Combined organic layer was washed with brine, dried over Na₂SO₄ and concentrated under reduced pressure to obtain crude which was purified by reversed phase HPLC to afford 5-hydroxy-1-[(2E)-3-(pyrimidin-2-yl)prop-2-enoyl]-5,6-dihydropyridin-2(1H)-one (100 mg, 18.87%) as white solid.

LCMS: 246 [M+1]⁺

¹H NMR (400 MHz, CDCl₃) δ 8.87 (d, 2H), 8.00 (d, 1H), 7.47 (t, 1H), 7.39 (d, 1H), 7.10-6.95 (m, 1H), 5.99 (d, 1H), 5.62 (d, 1H), 4.40 (brs, 1H), 4.00-3.82 (m, 2H).

### Example 32

### Preparation of (E)-5,5-dimethyl-1-(3-(pyrimidin-2-yl)acryloyl)-5,6-dihydropyridin-2(1H)-one

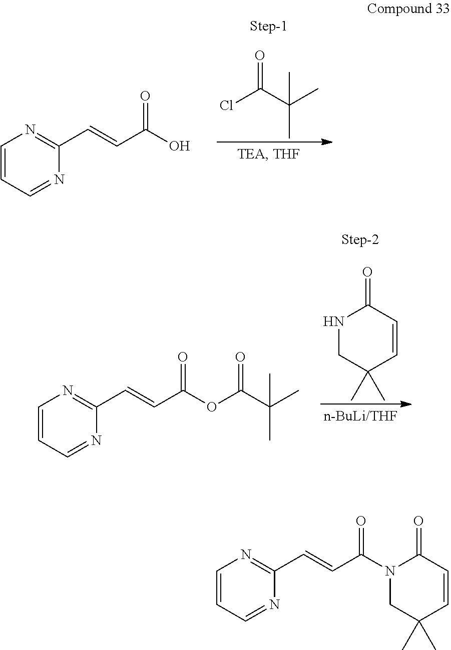

### Step-1

To a solution of (E)-3-(pyrimidin-2-yl)acrylic acid (0.150 g, 0.99 mmol, 0.99 eq.) in dry THF was added triethylamine (0.16 mL, 1.198 mmol, 1.2 eq.) and pivaloyl chloride (0.14 mL, 1.198 mmol, 1.2 eq.) the reaction mixture was stirred at 0° C. for 45 minutes. Progress of reaction was monitored by TLC. After completion, reaction mixture was filtered and the filtrate was used directly for the next step.

### Step-2

To a stirred solution of 5,5-dimethyl-5,6-dihydropyridin-2(1H)-one (0.1 g, 0.80 mmol, 0.8 eq.) in dry THF at −78° C. was added n-butyllithium (2.5 M in hexane, 0.3 mL, 0.80 mmol, 0.8 eq.) and the mixture was allowed to stir at the same temperature for 30 minutes. To this solution was added (E)-pivalic (E)-3-(pyrimidin-2-yl)acrylic anhydride (0.234 g, 1.00 mmol, 1 eq.).

Reaction mixture was allowed to stir at −78° C. for 1 h. Progress of reaction was monitored by TLC and LCMS. Reaction mixture was brought to 0° C., diluted with water (30 mL) and extracted with ethyl acetate (3×30 mL). Combined organic layer was washed with brine, dried over anhydrous Na₂SO₄ and evaporated to dryness under vacuum to afford crude which was purified by Combi-Flash to afford (E)-5,5-dimethyl-1-(3-(pyrimidin-2-yl)acryloyl)-5,6-dihydropyridin-2(1H)-one (80 mg, 34.18%).

LCMS: 258 [M+1]⁺

¹H NMR (400 MHz, CDCl₃) δ 8.78 (d, 2H), 8.11 (d, 1H), 7.67 (d, 1H), 7.19 (t, 1H), 6.65 (d, 1H), 5.88 (d, 1H), 3.80 (s, 2H), 1.19 (s, 6H).

### Example 33

### Preparation of 1-[(2E)-3-(tetrahydro-2H-pyran-4-yl)prop-2-enoyl]-5,6-dihydropyridin-2(1H)-one

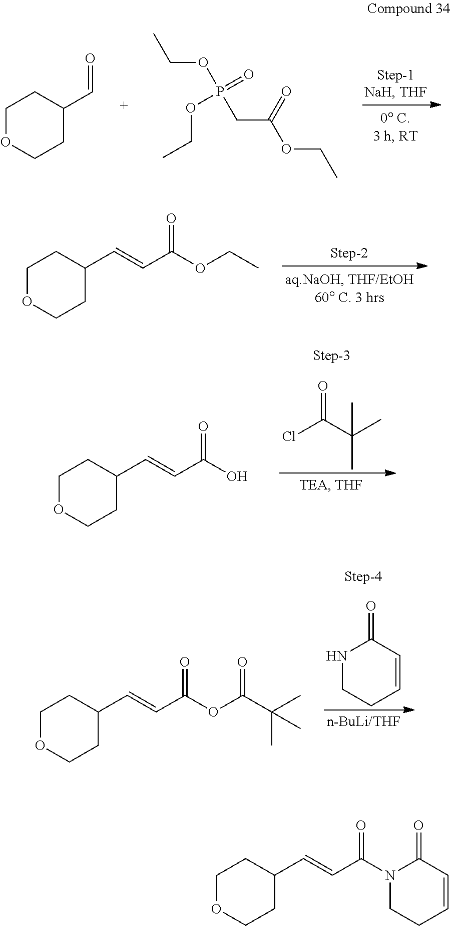

### Step-1

To a suspension of sodium hydride (60% in mineral oil, 0.44 g, 10.72 mmol, 1.2 eq.) in THF (20 mL) was added ethyl 2-(diethoxyphosphoryl)acetate (2.1 g, 9.38 mmol, 1.05 eq.) dropwise at 0° C. and the mixture was allowed to stir at the same temperature for 30 minutes. To this mixture was added a solution of tetrahydro-2H-pyran-4-carbaldehyde (1.0 g, 0.93 mmol, 1.0 eq.) in THF (5 mL) and the resulting mixture was allowed to stir at room temperature for 2 h. Progress of reaction was monitored by TLC. After completion, reaction mixture was diluted with saturated aq. NH₄Cl (100 mL) and extracted with ethyl acetate (3×50 mL). Combined organic layer was washed with brine, dried over anhydrous Na₂SO₄ and evaporated to dryness under vacuum to afford crude which was purified by Combi-Flash to using ethyl acetate-hexane system as eluent to afford ethyl (2E)-3-(tetrahydro-2H-pyran-4-yl)prop-2-enoate (1.2 g, 74.53%).

LCMS: 185 [M+1]⁺

### Step-2

To a solution of ethyl (2E)-3-(tetrahydro-2H-pyran-4-yl)prop-2-enoate (1.2 g, 2.10 mmol, 1.0 eq.) in ethanol:THF (1:1, 20 mL) was added sodium hydroxide (1.5 g) in water (6.0 mL) and the reaction mixture was stirred at room temperature for 3 h. Reaction mixture was concentrated under vacuum and then acidified with 1N HCL. The mixture was diluted with water (100 mL) and extracted with ethyl acetate (3×50 mL). The combined organic layer was washed with brine solution (50 mL) and dried over anhydrous Na₂SO₄. Removal of solvent under reduced pressure to obtain (2E)-3-(tetrahydro-2H-pyran-4-yl)prop-2-enoic acid (0.80 g, 79.20%).

LCMS: 157 [M+1]⁺

### Step-3

To a solution of (2E)-3-(tetrahydro-2H-pyran-4-yl)prop-2-enoic acid (0.20 g, 1.28 mmol, 1.0 eq.) in dry THF (20 mL) was added triethylamine (0.21 mL, 1.53 mmol, 1.2 eq.) followed by pivaloyl chloride (0.189 mL, 1.53 mmol, 1.2 eq.) and the reaction mixture was stirred at 0° C. for 45 minutes. Progress of reaction was monitored by TLC. After completion, reaction mixture was filtered and the filtrate was used directly for the next step.

### Step-4

To a stirred solution of 5,6-dihydropyridin-2(1H)-one (0.109 g, 1.12 mmol, 0.9 eq.) in dry THF at −78° C. was added n-butyllithium (2.5 M in hexane, 0.45 mL, 1.1 eq.) and the mixture was allowed to stir at the same temperature for 30 minutes. To this solution was added a solution of 2,2-dimethylpropanoic (2E)-3-(tetrahydro-2H-pyran-4-yl)prop-2-enoic anhydride (0.300 g, 1.24 mmol, 1.0 eq.) in THF and the reaction mixture was allowed to stir at −78° C. for 1 h. Progress of reaction was monitored by TLC and LCMS. After completion, reaction mixture was brought to 0° C., diluted with water (30 mL) and extracted with ethyl acetate (3×50 mL). Combined organic layer was washed with brine, dried over anhydrous Na₂SO₄ and evaporated to dryness under vacuum to afford crude which was purified by Combi-flash to afford 1-[(2E)-3-(tetrahydro-2H-pyran-4-yl)prop-2-enoyl]-5,6-dihydropyridin-2(1H)-one (0.05 g, 17.06%).

LCMS: 236 [M+1]⁺

¹H NMR (400 MHz, CDCl₃) δ 6.98-6.90 (m, 2H), 6.83 (d, 1H), 6.00 (d, 1H), 4.10-3.90 (m, 4H), 3.50-3.38 (m, 2H), 2.50-2.38 (m, 3H), 1.78-1.65 (m, 2H), 1.62-1.50 (m, 2H).

### Example 34

### (E)-3-chloro-1-(3-(pyrimidin-2-yl)acryloyl)-5,6-dihydropyridin-2(1H)-one

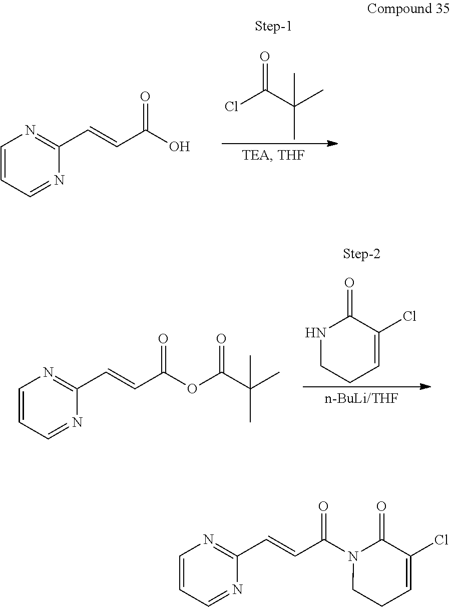

### Step-1

To a solution of (E)-3-(pyrimidin-2-yl)acrylic acid (0.126 g, 0.839 mmol, 1 eq.) in dry THF was added triethylamine (0.14 mL, 1.00 mmol, 1.2 eq.) and pivaloyl chloride (0.1 mL, 0.755 mmol, 0.9 eq.) the reaction mixture was stirred at 0° C. for 45 minutes. Progress of reaction was monitored by TLC. After completion, reaction mixture was filtered and the filtrate was used directly for the next step.

### Step-2

To a stirred solution of 3-chloro-5,6-dihydropyridin-2(1H)-one (0.1 g, 0.755 mmol, 0.9 eq.) in dry THF at −78° C. was added n-butyllithium (1.8 M in hexane, 0.41 mL, 0.755 mmol, 0.9 eq.) and the mixture was allowed to stir at the same temperature for 30 minutes. To this solution was added (E)-pivalic (E)-3-(pyrimidin-2-yl)acrylic anhydride (0.196 g, 0.839 mmol, 1 eq.).

Reaction mixture was allowed to stir at −78° C. for 1 h. Progress of reaction was monitored by TLC and LCMS. Reaction mixture was brought to 0° C., diluted with water (30 mL) and extracted with ethyl acetate (3×25 mL). Combined organic layer was washed with brine, dried over anhydrous Na₂SO₄ and evaporated to dryness under vacuum to afford crude which was purified by Combi-Flash to afford (E)-3-chloro-1-(3-(pyrimidin-2-yl)acryloyl)-5,6-dihydropyridin-2(1H)-one (55 mg, 25%).

LCMS: 264[M+1]⁺

¹H NMR (400 MHz, CDCl₃) δ 8.78 (d, 1H), 8.08 (d, 1H), 7.65 (d, 1H), 7.20 (t, 1H), 7.10 (t, 1H), 4.09 (t, 2H), 2.61-2.55 (m, 2H).

### Example 35

### Preparation of (E)-4-chloro-1-(3-(pyrimidin-2-yl)acryloyl)-5,6-dihydropyridin-2(1H)-one

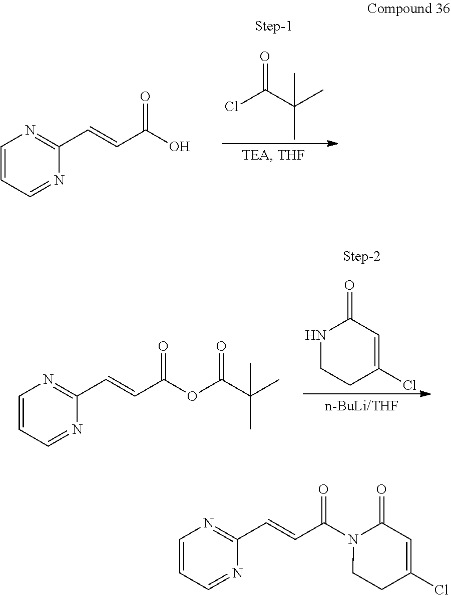

### Step-1

To a solution of (E)-3-(pyrimidin-2-yl)acrylic acid (0.126 g, 0.839 mmol, 1 eq.) in dry THF was added triethylamine (0.14 mL, 1.00 mmol, 1.2 eq.) and pivaloyl chloride (0.1 mL, 0.755 mmol, 0.9 eq.) the reaction mixture was stirred at 0° C. for 45 minutes. Progress of reaction was monitored by TLC. After completion, reaction mixture was filtered and the filtrate was used directly for the next step.

### Step-2

To a stirred solution of 4-chloro-5,6-dihydropyridin-2(1H)-one (0.1 g, 0.755 mmol, 0.9 eq.) in dry THF at −78° C. was added n-butyllithium (1.8 M in hexane, 0.41 mL, 0.755 mmol, 0.9 eq.) and the mixture was allowed to stir at the same temperature for 30 minutes. To this solution was added (E)-pivalic (E)-3-(pyrimidin-2-yl)acrylic anhydride (0.196 g, 0.839 mmol, 1 eq.). Reaction mixture was allowed to stir at −78° C. for 1 h. Progress of reaction was monitored by TLC and LCMS. Reaction mixture was brought to 0° C., diluted with water (30 mL) and extracted with ethyl acetate (3×25 ml). Combined organic layer was washed with brine, dried over anhydrous Na₂SO₄ and evaporated to dryness under vacuum to afford crude which was purified by CombiFlash to afford (E)-4-chloro-1-(3-(pyrimidin-2-yl)acryloyl)-5,6-dihydropyridin-2(1H)-one (120 mg, 61.22%).

LCMS: 264[M+1]⁺

¹H NMR (400 MHz, CDCl₃) δ 8.79 (d, 2H), 8.07 (d, 1H), 7.65 (d, 1H), 7.20 (t, 1H), 6.22 (s, 1H), 4.18-4.08 (m, 2H), 2.85-2.75 (m, 2H).

### Example 36

### Preparation of (E)-5-fluoro-1-(3-(pyrimidin-2-yl)acryloyl)-5,6-dihydropyridin-2(1H)-one

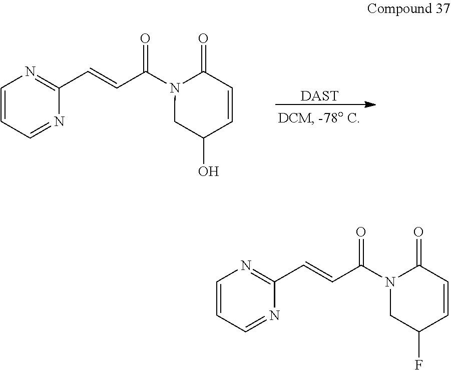

To a stirred solution of (E)-5-hydroxy-1-(3-(pyrimidin-2-yl)acryloyl)-5,6-dihydropyridin-2(1H)-one (0.07 g, 0.285 mmol, 1 eq.) in DCM (8 mL) was added DAST (0.15 mL, 1.14 mmol, 4 eq.) and the reaction mixture was stirred at −78° C. for 1 h. Progress of reaction was monitored by TLC. Reaction mixture was brought to room temperature, diluted with ammonium chloride solution (15 mL) and extracted using ethyl acetate (2×20 mL). Combined organic layer was washed with brine and dried over anhydrous sodium sulphate. Removal of solvent under reduced pressure afforded crude which was purified by Combi-Flash on silica gel using ethyl acetate-hexane system as eluent to afford (E)-5-fluoro-1-(3-(pyrimidin-2-yl)acryloyl)-5,6-dihydropyridin-2(1H)-one (15 mg, 21.42%).

LCMS: 248 [M+1]⁺

¹H NMR (CDCl₃, 400 MHz) δ (8.88 (d, 2H), 8.10 (d, 1H), 7.70 (d, 2H), 7.20 (t, 1H), 6.99-6.90 (m 1H), 6.30-6.20 (m, 1H), 5.35-5.28 (m, 1H), 4.60-4.43 (m, 1H), 4.10-3.90 (m, 1H).

### Example 37

### Preparation (E)-1-(3-(piperidin-4-yl)acryloyl)-6,7-dihydro-1H-azepin-2(5H)-one

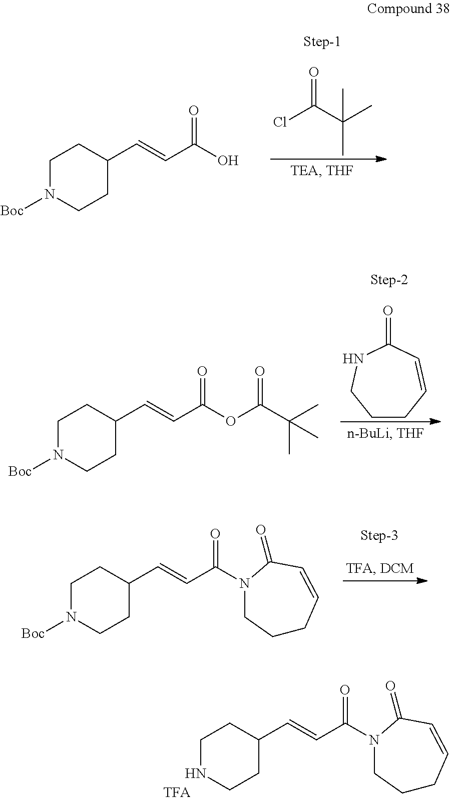

### Step-1

To a solution of (2E)-3-[1-(tert-butoxycarbonyl)piperidin-4-yl]prop-2-enoic acid (0.90 g, 3.52 mmol, 1.0 eq.) in dry THF (20 mL) was added triethylamine (0.60 mL, 4.23 mmol, 1.2 eq.) followed by pivaloyl chloride (0.509 mL, 4.23 mmol, 1.2 eq.) and the reaction mixture was stirred at 0° C. for 45 minutes. Progress of reaction was monitored by TLC. After completion, reaction mixture was filtered and the filtrate was used directly for the next step.

### Step-2

To a stirred solution of 1,5,6,7-tetrahydro-2H-azepin-2-one (0.324 g, 2.91 mmol, 0.9 eq.) in dry THF at −78° C. was added n-butyllithium (2.5 M in hexane, 1.16 mL, 1.1 eq.) and the mixture was allowed to stir at the same temperature for 30 minutes. To this solution was added a solution of anhydride (1.1 g, 3.24 mmol, 1.0 eq.) in THF and the reaction mixture was allowed to stir at −78° C. for 1 h. Progress of reaction was monitored by TLC and LCMS. After completion, reaction mixture was brought to 0° C., diluted with water (30 mL) and extracted with ethyl acetate (3×50 mL). Combined organic layer was washed with brine, dried over anhydrous Na₂SO₄ and evaporated to dryness under vacuum to afford crude which was purified by Combi-flash to afford (0.8 g, 71.42%).

LCMS: 349 [M+1]⁺

### Step-3

To a stirred solution of 5,6-dihydropyridin-2(1H)-one (0.324 g, 2.91 mmol, 0.9 eq.) in dry DCM at 0° C. was added TFA (0.27 mL, 6.0 eq.) and the mixture was allowed to stir at room temperature for 30 minutes. Progress of reaction was monitored by TLC and LCMS. Solvent was removed under reduced pressure to give oily residue. Residue was dissolved in ethanol (3 mL) and diluted with pentane (10 mL). Solid was filtered and lyophilized to give (E)-1-(3-(piperidin-4-yl)acryloyl)-6,7-dihydro-1H-azepin-2(5H)-one (150 mg, 75.75%).

LCMS: 249 [M+1]⁺

¹H NMR (400 MHz, DMSO-d₆) δ 8.58 (brs, 1H), 8.23 (brs, 1H), 6.85-6.55 (m, 3H), 6.00 (d, 1H), 3.85-3.76 (m, 2H), 3.40-3.22 (m, 2H), 3.00-2.81 (m, 2H), 2.40-2.25 (m, 2H), 1.95-1.79 (m, 5H), 1.60-1.40 (m, 2H).

### Example 38

### Preparation of (E)-1-(3-(pyridazin-4-yl)acryloyl)-5,6-dihydropyridin-2(1H)-one

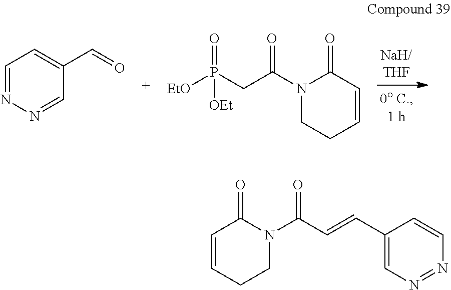 [SMILES: O=Cc1ccnnc1 ; CCOP(=O)(CC(=O)N1CCC=CC1=O)OCC ; O=C1C=CCCN1C(=O)/C=C/c1ccnnc1]

To a solution of diethyl 2-oxo-2-(2-oxo-5,6-dihydropyridin-1(2H)-yl)ethylphosphonate (350 mg, 1.27 mmol, 0.69 eq.) in THF (10 mL) at 0° C. was added NaH (80 mg, 2.00 mmol, 1.08 eq.) and the reaction mixture was allowed to stir at the same temperature for 10 minutes. To this mixture was added a solution of pyridazine-4-carbaldehyde (200 mg, 1.85 mmol, 1 eq.) in THF (5 mL) and the reaction mixture was allowed to stir at the same temperature for 1 h. Progress of reaction was monitored by TLC. After completion, reaction mixture was diluted with saturated aq. Ammonium chloride (30 mL) and extracted with ethyl acetate (3×30 mL). Combined organic layer was washed with brine (30 mL) and dried over anhydrous sodium sulfate. Removal of solvent under reduced pressure afforded crude which was purified by Combi-Flash on silica gel using ethyl acetate-hexane system as eluent to afford (E)-1-(3-(pyridazin-4-yl)acryloyl)-5,6-dihydropyridin-2(1H)-one (45 mg, 27%).

LCMS: 230 [M+1]⁺

¹H NMR (CDCl3, 400 MHz) δ 9.33 (s, 1H), 9.22 (s, 1H), 7.73 (d, 1H), 7.58 (d, 1H), 7.55 (d, 1H), 7.03-6.97 (m, 1H), 6.03 (d, 1H), 4.05-4.00 (m, 2H), 2.58-2.45 (m, 2H).

### Example 39

### Preparation 3-bromo-1-[(2E)-3-(pyrimidin-2-yl)prop-2-enoyl]-5,6-dihydropyridin-2(1H)-one

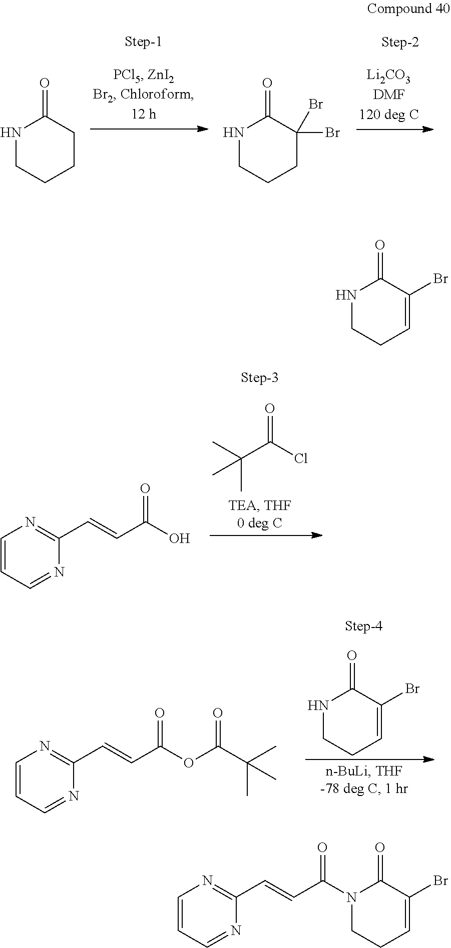

### Step-1

To a stirred solution of piperidin-2-one (10 g, 101.01 mmol, 1 eq.) in chloroform (300 mL) was added PCl₅ (42 g, 202.02 mmol, 2 eq.) at 0-5° C. for 10 minutes. To this suspension was added ZnI₂ (0.96 g, 3.03 mmol, 0.03 eq.) under nitrogen at 0-5° C. and then allowed to stir at room temperature for 1 h. To this reaction mixture was added a solution of Br₂ (32.11 g, 202.02 mmol, 2 eq.) in chloroform (5 mL) dropwise and the reaction mixture was stirred overnight. Progress of reaction was monitored by TLC and LCMS. After completion, reaction mixture was diluted with ice-cold water and extracted with dichloromethane (3×50 mL). Combined organic layer was washed with brine and dried over sodium sulphate. Removal of solvent under reduced pressure afforded 3,3-dibromopiperidin-2-one (12 g, 46.17%) as brown liquid which was used in the next step without purification.

LCMS: 258 [M+1]⁺

### Step-2

To a stirred solution of 3,3-dibromopiperidin-2-one (12 g, 46.87 mmol, 1 eq.) in DMF (100 mL) was added Li₂CO₃ (6.8 g, 93.75 mmol, 2 eq.) at room temperature and the resulting reaction mixture was stirred at 120° C. for 7 h. Progress of reaction was monitored by LCMS. After completion, reaction mixtures were diluted with ice-cold water (200 mL) and extracted with dichloromethane (3×100 mL). Combined organic layer was washed with brine (50 mL) and dried over anhydrous sodium sulfate. Removal of solvent under reduced pressure afford crude oil which was purified by Combi-Flash on silica gel using ethyl acetate-hexane system as eluent to afford 3-bromo-5,6-dihydropyridin-2(1H)-one (1 g, 12.18%) as brown oil.

LCMS: 177 [M+1]⁺

### Step-3

To a stirred solution of (2E)-3-(pyrimidin-2-yl)prop-2-enoic acid (200 mg, 1.33 mmol, 1 eq) in a THF (20 mL) at 0° C. was added triethylamine (0.2 mL, 1.47 mmol, 1.1 eq.) followed by 2,2-dimethylpropanoyl chloride (159 mg, 1.33 mmol, 1 eq.) and the resulting mixture was allowed to stir at the same temperature for mixture for 1 h. Progress of reaction was monitored by TLC. After completion, reaction mixture was filtered through cotton plug and used directly in the next reaction.

### Step-4

To a stirred solution of 3-bromo-5,6-dihydropyridin-2(1H)-one (234 mg, 1.329 mmol, 1 eq) in THF (20 mL) at −78° C. was added n-BuLi (0.53 mL, 1.329 mmol, 1 eq.) drop wise at and the resulting mixture was stirred at same temperature for 1 h. To this mixture was added a solution of 2,2-dimethylpropanoic (2E)-3-(pyrimidin-2-yl)prop-2-enoic anhydride (443 mg, 1.33 mmol, 1 eq.) in THF dropwise and the mixture was stirred at −78° C. for another 1 h. Progress of reaction was monitored by TLC and LCMS. After completion, reaction mixture was diluted with saturated ammonium chloride solution (50 mL) and extracted with dichloromethane (3×100 mL). Combined organic layer was dried over anhydrous sodium sulfate and concentrate under reduced pressure to yield crude product, which was purified by reversed phase HPLC to afford 3-bromo-1-[(2E)-3-(pyrimidin-2-yl)prop-2-enoyl]-5,6-dihydropyridin-2(1H)-one (20 mg, off white solid, 5% yield).

LCMS: 309[M+1]⁺

¹H NMR (400 MHz, DMSO-d₆) δ 8.88 (d, 2H), 7.90 (d, 1H), 7.60 (t, 1H), 7.49 (t, 1H), 7.38 (d, 1H), 3.99 (t, 2H) 2.52-2.60 (m, 2H).

### Example 40

### Preparation of (E)-1-(3-(1-methylpiperidin-4-yl)acryloyl)-5,6-dihydropyridin-2(1H)-one

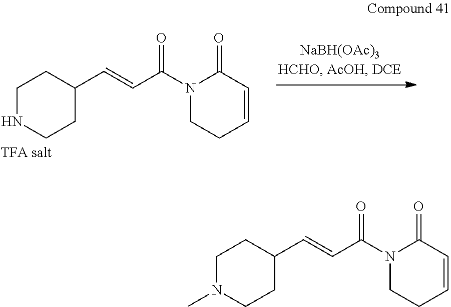 [SMILES: O=C1C=CCCN1C(=O)/C=C/C1CCNCC1 ; CN1CCC(/C=C/C(=O)N2CCC=CC2=O)CC1]

To a stirred solution of (E)-1-(3-(piperidin-4-yl)acryloyl)-5,6-dihydropyridin-2(1H)-one (0.100 g, 0.427 mmol, 1.0 eq.) in 1,2-dichloroethane (4 mL) was added HCHO in water (0.04 mL, 1.28 mol, 3.0 eq.), acetic acid (0.012 mL, 2.13 mol, 5.0 eq.). The reaction mixture was allowed to stir at room temperature for 1 h and followed by the addition of NaBH(OAc)₃ (0.271 g, 1.282 mmol, 3.0 eq.) and stirring was continued at room temperature for additional 1 h. Progress of the reaction was monitored by TLC and LCMS. After completion, the reaction mixture was diluted with water (30 mL) and extracted with ethyl acetate (2×30 mL). Organic layer was washed with brine solution (30 mL) and dried over anhydrous sodium sulphate. Removal of solvent under reduced pressure gave crude which was purified by reversed phase HPLC to afford (E)-1-(3-(1-methylpiperidin-4-yl)acryloyl)-5,6-dihydropyridin-2(1H)-one as a TFA salt (30 mg, 29%).

LCMS: 249 [M+1]⁺

¹H NMR (400 MHz, CD₃OD) δ 1.58-1.72 (m, 2H) 2.09 (d, 2H) 2.39-2.51 (m, 2H) 2.54 (d, 1H) 2.88 (s, 3H) 3.00-3.16 (m, 2H) 3.56 (d, 2H) 3.94 (t, 2H) 5.97 (d, 1H) 6.76-6.90 (m, 2H) 7.02-7.11 (m, 1H).

### Example 41

### Preparation of (E)-1-(2-methyl-3-(pyrimidin-2-yl)acryloyl)-5,6-dihydropyridin-2(1H)-one

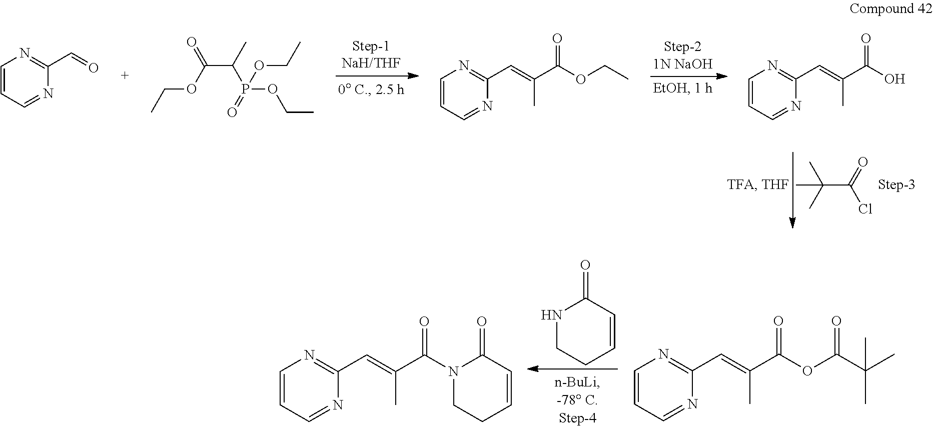

### Step-1

To a solution of ethyl 2-(diethoxyphosphoryl)propanoate (0.66 g, 2.77 mmol, 1.2 eq.) in THF (20 mL) was added sodium hydride (0.110 g, 2.77 mmol, 1.2 eq.) portionwise at 0° C. and the reaction mixture was allowed to stir at 0° C. for 20 minutes. To this solution was added pyrimidine-2-carbaldehyde (0.25 g, 2.31 mmol, 1 eq.). Reaction mixture stirred at 0° C. for 2 h. Progress of reaction was monitored by TLC. After completion, reaction mixture was diluted saturated ammonium chloride (30 mL) and extract with ethyl acetate (3×30 mL). Combined organic layer was washed with brine (50 mL) and dried over anhydrous sodium sulfate. Removal of solvent afforded crude which was purified by CombiFlash on silica gel using ethyl acetate-hexane system as eluent to afford ethyl (E)-2-methyl-3-(pyrimidin-2-yl)acrylate (0.2 g, 45%).

### Step-2

To a solution of ethyl (E)-2-methyl-3-(pyrimidin-2-yl)acrylate (0.2 g, 1.04 mmol, 1 eq.) in ethanol (10 mL) was added 1N sodium hydroxide (2.08 mL, 2.08 mmol, 2 eq.) at room temperature and the reaction mixture was allowed to stir at the same temperature for 1 h. Progress of reaction was monitored by TLC. After completion, reaction mixture was acidified with 1N HCl and extract with ethyl acetate (3×25 mL). Combined organic layer was washed with brine (50 mL) and dried over anhydrous sodium sulfate. Removal of solvent afforded (E)-2-methyl-3-(pyrimidin-2-yl)acrylic acid (0.180 g, crude) which was used in the next step without further purification.

### Step-3

To a solution of (E)-2-methyl-3-(pyrimidin-2-yl)acrylic acid (0.180 g, 1.09 mmol, 1.0 eq.) in dry THF was added triethylamine (0.18 mL, 1.30 mmol, 1.2 eq.) and pivaloyl chloride (0.12 mL, 0.980 mmol, 0.9 eq.) and the reaction mixture was stirred at 0° C. for 45 minutes. Progress of reaction was monitored by TLC. After completion, reaction mixture was filtered and the filtrate was used directly for the next step.

### Step-4

To a stirred solution of 5,6-dihydropyridin-2(1H)-one (0.105 g, 1.09 mmol, 1 eq.) in dry THF at −78° C. was added n-butyllithium (2.5 M in hexane, 0.43 ml, 1.09 mmol, 1 eq.) and the mixture was allowed to stir at the same temperature for 30 minutes. To this solution was added (E)-(E)-2-methyl-3-(pyrimidin-2-yl)acrylic pivalic anhydride (0.270 g, 1.09 mmol, 1 eq.). Reaction mixture was allowed to stir at −78° C. for 90 min. Progress of reaction was monitored by TLC. Reaction mixture was brought to 0° C., diluted with ammonium chloride solution (30 mL) and extracted with ethyl acetate (3×40 ml). Combined organic layer was washed with brine, dried over anhydrous Na₂SO₄ and evaporated to dryness under vacuum to afford crude which was purified by Combi-Flash to afford (E)-1-(2-methyl-3-(pyrimidin-2-yl)acryloyl)-5,6-dihydropyridin-2(1H)-one (80 mg, 30.30%).

LCMS: 244 [M+1]⁺

¹H NMR (CDCl₃, 400 MHz) δ 8.77 (d, 2H), 7.07 (t, 1H), 6.99-6.90 (m, 1H), 6.70 (s, 1H), 5.98 (d, 1H), 4.00-3.90 (m, 2H), 2.54 (brs, 5H).

### Example 42

### Preparation of (E)-5-methyl-1-(3-(pyrimidin-2-yl)acryloyl)-5,6-dihydropyridin-2(1H)-one

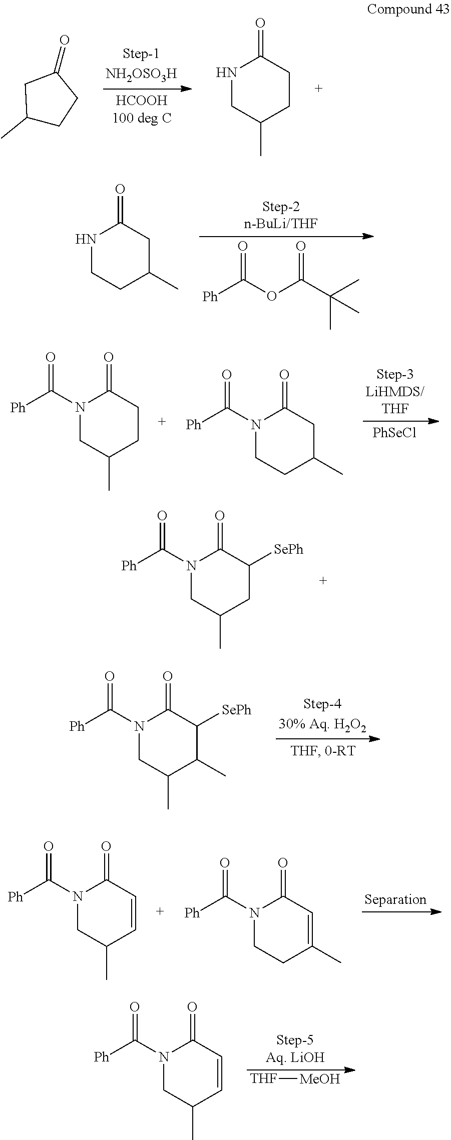

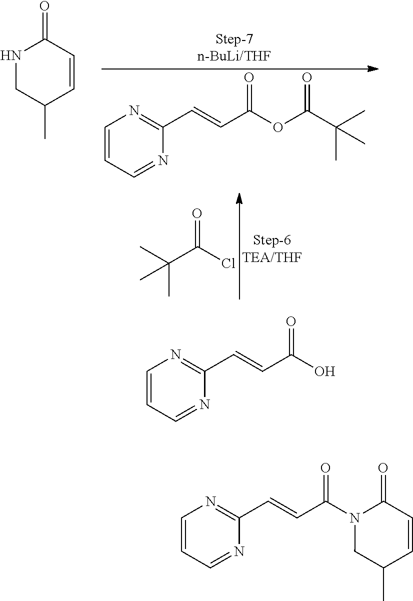

### Step-1

To a stirred solution of 3-methylcyclopentanone (5 g, 51.02 mmol, 1 eq.) in formic acid (100 mL) was added hydroxyl amine-O-sulfonic acid (8.65 g, 76.53 mmol, 1.5 eq.) and the reaction mixture was refluxed for 12 h. Progress of reaction was monitored by TLC. After completion, reaction mixture was cool to room temperature, diluted with water (50 mL), basified with 5M aqueous NaOH and extracted with dichloromethane (3×100 mL). Combined organic layer was washed with brine solution (50 mL) and dried over anhydrous sodium sulphate. Removal of solvent under reduced pressure afforded a mixture of 5-methylpiperidin-2-one and 4-methylpiperidin-2-one (5 g, 87%).

LCMS: 114 [M+1]⁺

### Step-2

To a stirred solution of a mixture of 5-methylpiperidin-2-one and 4-methylpiperidin-2-one (4.6 g, 40.98 mmol, 1 eq.) in anhydrous THF (50 mL) at −78° C. was added n-BuLi (1.8 M in THF, 3.8 mL, 6.86 mmol, 1.2 eq.) dropwise and the reaction mixture was allowed to stir at same temperature for 30 minutes. To this mixture was added a solution of freshly prepared benzoic pivalic anhydride (8.4 g, 40.98 mmol, 1 eq.) and allow the reaction mixture was stirred at same temperature for 1 h. Progress of reaction was monitored by TLC. After completion, reaction mixture was brought to 0° C., diluted with saturated aqueous solution of ammonium chloride (200 mL) and extracted with ethyl acetate (3×200 mL). Organic layer was washed with brine and dried over anhydrous sodium sulphate. Removal of solvent under reduced pressure afforded a mixture of 1-benzoyl-5-methylpiperidin-2-one and 1-benzoyl-4-methylpiperidin-2-one as crude (8 g, 90%).

LCMS: 218 [M+1]⁺

### Step-3

To a stirred solution of a mixture of 1-benzoyl-5-methylpiperidin-2-one and 1-benzoyl-4-methylpiperidin-2-one (1.1 g, 5.07 mmol, 1 eq.) in anhydrous THF (25 mL) at −78° C. was added LiHMDS (1M in THF, 10.13 mL, 10.13 mmol, 2 eq.) dropwise and the reaction mixture was allowed to stir at same temperature for 30 minutes. To this solution was added phenyl selenium chloride (1.94 g, 10.13 mmol, 2 eq.) in THF (5 mL) dropwise. Reaction mixture was allowed to stir at same temperature for 1 h. Progress of reaction was monitored by TLC. After completion reaction mixture was brought to 0° C. and diluted with saturated aqueous solution of ammonium chloride (50 mL) and extracted with ethyl acetate (3×100 mL). Combined organic layer was washed with brine (100 mL) dried over anhydrous sodium sulphate. Removal of solvent under reduced pressure afforded a mixture of 1-benzoyl-5-methyl-3-(phenylselanyl)piperidin-2-one and 1-benzoyl-4-methyl-3-(phenylselanyl)piperidin-2-one as crude (1.4 g, 77.77%).

LCMS 374 [M+1]⁺

### Step-4

To a stirred solution of a mixture of 1-benzoyl-5-methyl-3-(phenylselanyl)piperidin-2-one and 1-benzoyl-4-methyl-3-(phenylselanyl)piperidin-2-one (1.4 g, 3.76 mmol, 1 eq.) in THF (30 mL) at 0° C. was 30% aq. H₂O₂ (10 mL) dropwise and the resulting solution was allowed to stir at room temperature for 1 h. Progress of reaction was monitored by TLC. After completion, reaction mixture was diluted with water and extracted with ethyl acetate (3×100 mL). Combined organic layer was washed with brine (50 mL) and dried over anhydrous sodium sulphate. Removal of solvent under reduced pressure afforded 1-benzoyl-5-methyl-5,6-dihydropyridin-2(1H)-one and 1-benzoyl-4-methyl-5,6-dihydropyridin-2(1H)-one as crude which was purified by Combi-Flash on silica gel using ethyl acetate-hexane as eluent to afford 1-benzoyl-5-methyl-5,6-dihydropyridin-2(1H)-one (0.35 g, 43%).

LCMS 216 [M+1]⁺

### Step-5

To a stirred solution of 1-benzoyl-5-methyl-5,6-dihydropyridin-2(1H)-one (0.35 g, 1.63 mmol, 1 eq.) in a solution of 1:1 THF-MeOH (20 mL) was added LiOH (0.409 g, 9.77 mmol, 6 eq.) dissolved in minimum amount of water and the resulting mixture was allowed to stir at room temperature for 1 h. Progress of reaction was monitored by TLC. After completion, reaction mixture was diluted with water (50 mL) and extracted with ethyl acetate (3×50 mL). Combined organic layer was washed with brine (50 mL), dried over anhydrous sodium sulphate and concentrated under reduced pressure afforded crude which was purified by combi-Flash on silica gel using ethyl acetate-hexane as eluent to afford 5-methyl-5,6-dihydropyridin-2(1H)-one (0.130 g, 72%) LCMS 112[M+1]⁺

### Step-6

To a stirred solution of (2E)-3-(pyrimidin-2-yl)prop-2-enoic acid (0.120 g, 0.80 mmol, 1 eq.) in THF (30 mL) in a 100 mL round bottom flask at 0° C. was added TEA (0.13 mL, 0.98 mmol, 1.2 eq.) followed by pivaloyl chloride (0.08 ml, 0.729 mmol, 1 eq.) and the resulting mixture was stirred at the same temperature for 1 h. Progress of reaction was monitored by TLC. After completion, the mixture was transferred to the reaction mixture of the next step drop wise via a syringe equipped with a cotton plug at the bottom.

### Step-7

To a stirred solution 5-methyl-5,6-dihydropyridin-2(1H)-one (0.097 g, 0.88 mmol, 1 eq.) in anhydrous THF (10 mL) at −78° C. was added n-BuLi (2.5 M in THF, 0.3 mL, 0.88 mmol, 1 eq.) dropwise and the reaction mixture was allowed to stir at same temperature for 30 minute. To this solution was added freshly prepared anhydride of step-6 (0.21 g, 0.88 mmol, 1 eq.). The reaction mixture was allowed to stir at same temperature for 1 h. Progress of reaction was monitored by TLC. After completion reaction mixture was brought to 0° C. and diluted with saturated aqueous solution of ammonium chloride (50 mL) and extracted with ethyl acetate (3×50 mL). Organic layer was washed with brine and dried over anhydrous sodium sulphate. Removal of solvent under reduced pressure afforded crude which was purified by reversed phase HPLC to afford (E)-5-methyl-1-(3-(pyrimidin-2-yl)acryloyl)-5,6-dihydropyridin-2(1H)-one (20 mg, 21%).

LCMS: 244[M+1]⁺

¹H NMR (400 MHz, CD₃OD) δ 8.69 (d, 2H), 8.1 (d, 1H), 7.7 (d, 1H), 7.2 (t, 1H), 6.8 (m, 1H), 6.0 (m, 1H), 4.2 (m, 1H), 3.59 (m, 2H), 2.7 (bs, 1H), 1.19 (d, 3H).

### Example 43

### (E)-1-(3-(1-(cyclopropylmethyl)piperidin-4-yl)acryloyl)-5,6-dihydropyridin-2(1H)-one

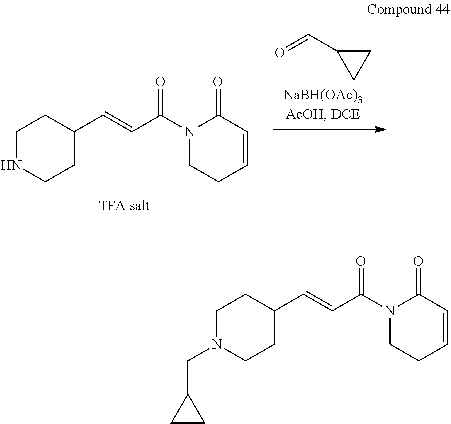 [SMILES: O=CC1CC1 ; O=C1C=CCCN1C(=O)/C=C/C1CCNCC1 ; O=C1C=CCCN1C(=O)/C=C/C1CCN(CC2CC2)CC1]

To a stirred solution of (E)-1-(3-(piperidin-4-yl)acryloyl)-5,6-dihydropyridin-2(1H)-one TFA salt (0.100 g, 0.427 mmol, 1.0 eq.) in DCE (4 mL), was added 2-Cyclopropanecarboxaldehyde (0.089 mL, 1.28 mmol, 3.0 eq.), acetic acid (0.061 ml, 2.14 mmol, 5.0 eq.). The reaction mixture was allowed to stir at room temperature for 1 h followed by the addition of NaBH(OAc)₃ (0.271 g, 1.282 mmol, 3.0 eq.) and stirring was continued at room temperature for additional 1 h. Progress of the reaction was monitored by TLC and LCMS. After completion, reaction mixture was diluted with water (30 mL) and extracted with ethyl acetate (2×30 ml). Organic layer was washed with brine solution (30 mL), dried over anhydrous sodium sulphate and concentrated under reduced pressure to obtain crude which was purified by reversed phase HPLC to afford (E)-1-(3-(1-(cyclopropylmethyl)piperidin-4-yl)acryloyl)-5,6-dihydropyridin-2(1H)-one as TFA salt (40 mg, 35%).

LCMS: 289 [M+1]⁺

¹H NMR (400 MHz, CD₃OD) δ 7.01-7.12 (m, 1H), 6.75-6.93 (m, 2H), 5.97 (d, 1H), 3.94 (t, 2H), 3.72 (d, 2H), 2.97-3.11 (m, 3H), 2.52-2.63 (m, 1H), 2.40-2.52 (m, 2H), 2.11 (d, 2H), 1.63-1.80 (m, 2H), 1.29 (brs., 1H), 1.12 (d, 1H), 0.71-0.84 (m, 2H), 0.37-0.53 (m, 2H)

### Example 44

### Preparation of (E)-1-(3-(1-(oxetan-3-yl)piperidin-4-yl)acryloyl)-5,6-dihydropyridin-2(1H)-one

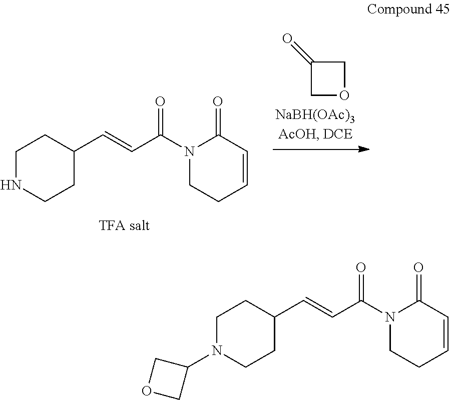 [SMILES: O=C1COC1 ; O=C1C=CCCN1C(=O)/C=C/C1CCNCC1 ; O=C1C=CCCN1C(=O)/C=C/C1CCN(C2COC2)CC1]

To a stirred solution of (E)-1-(3-(piperidin-4-yl)acryloyl)-5,6-dihydropyridin-2(1H)-one TFA salt (0.200 g, 0.85 mmol, 1.0 eq.) in DCE (4 mL), was added 3-Oxetanone (0.184 ml, 2.56 mmol, 3.0 eq.), acetic acid (0.24 mL, 4.27 mmol, 5.0 eq.). The reaction mixture was allowed to stir at room temperature for 1 h and followed by the addition of NaBH(OAc)₃ (0.543 g, 2.56 mol, 3.0 eq.) and stirring was continued at room temperature for additional 1 h. Progress of the reaction was monitored by TLC and LCMS. After completion, reaction mixture was diluted with water (30 mL) and extracted with ethyl acetate (2×30 mL). Organic layer was washed with brine solution (30 mL) and dried over anhydrous sodium sulphate. Removal of solvent under reduced pressure gave crude which was purified by reversed phase HPLC to afford (E)-1-(3-(1-(oxetan-3-yl)piperidin-4-yl)acryloyl)-5,6-dihydropyridin-2(1H)-one (40 mg, 17%) as TFA salt.

LCMS: 291 [M+1]⁺

¹H NMR (400 MHz, DMSO-d₆) δ 10.16 (brs., 1H), 7.04-7.14 (m, 1H), 6.76 (s, 2H), 5.94 (d, 1H), 4.74 (d, 3H), 4.34 (brs, 2H), 3.84 (t, 2H), 3.43 (d, 2H), 2.85 (brs., 2H), 2.44 (brs., 3H), 1.99 (d, 2H), 1.56 (d, 2H).

### Example 45

### Preparation of (E)-3-chloro-1-(2-methyl-3-(pyrimidin-2-yl)acryloyl)-5,6-dihydropyridin-2(1H)-one

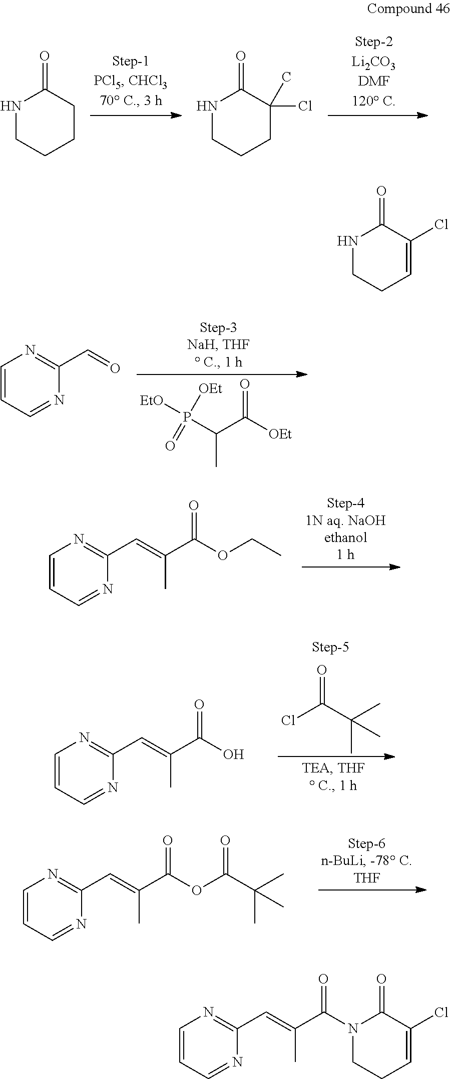

### Step-1

To a stirred solution of piperidin-2-one (5 g, 50.50 mmol, 1 eq) in chloroform (200 mL) was added with PCl₅ (31.5 g, 151.5 mmol, 3 eq) at 0-5° C. portion-wise and the resulting mixture was allowed to stir at 70° C. for 3 h. Progress of reaction was monitored by TLC and LCMS. After completion, reaction mixture was diluted with dichloromethane (50 mL) and mixture was poured on ice-cold water. Organic layer was separated and aqueous layer was again extracted with dichloromethane (3×100 mL). Combined organic layer was washed with brine (100 mL) and dried over anhydrous sodium sulfate. Removal of solvent under reduced pressure afforded 3,3-dichloropiperidin-2-one (10 g, crude)

LCMS: 167 (M+1)

### Step-2

To a stirred solution of 3,3-dichloropiperidin-2-one (5 g, 29.761 mmol, 1 eq) in DMF (30 mL) was added Li₂CO₃ (8.46 g, 119.04 mmol, 4 eq) at room temperature and the resulting reaction mixture was stirred at 120° C. for 7 h. Progress of reaction was monitored by TLC and LCMS. After completion, reaction mixture was diluted with ice-cold water and extracted with dichloromethane (3×100 mL). Combined organic layer was washed with brine (100 mL) and dried over anhydrous sodium sulphate and concentrate under reduced pressure to yield product 3-chloropiperidin-2-one (1 g, yellow liquid) which purified by Combi-Flash on silica gel using ethyl acetate-hexane system as eluent to afford 3-chloro-5,6-dihydropyridin-2(1H)-one (450 mg).

LCMS: 132 (M+H)

### Step-3

To a suspension of NaH (176 mg, 4.443 mmol, 1.2 eq) in THF (20 mL) at 0° C. was added ethyl 2-(diethoxyphosphoryl)propanoate (969 mg, 4.073 mmol, 1.1 eq) and the resulting mixture was stirred at the same temperature for 10 minutes. To this mixture was added pyrimidine-2-carbaldehyde (400 mg, 3.703 mmol, 1 eq) and the resulting reaction mixture for stirred at the same temperature for 1 h. Progress of reaction was monitored by TLC and LCMS. After completion, reaction mixture was diluted with ammonium chloride solution and extracted with ethyl acetate (3×50 mL). Combined organic layer was washed with brine (50 mL) and dried over anhydrous sodium sulphate. Removal of solvent under reduced afforded crude which was Combi-Flash on silica gel using ethyl acetate-hexane system as eluent to afford (E)-ethyl 2-methyl-3-(pyrimidin-2-yl)acrylate (350 mg).

LCMS: 193 (M+1)

### Step-4

To a stirred solution of (E)-ethyl 2-methyl-3-(pyrimidin-2-yl)acrylate (350 mg, 1.82 mmol, 1 eq) in ethanol (10 mL) was added 1N aq. NaOH solution (3.6 mL, 3.644 mmol, 2 eq) and the resulting reaction mixture was stirred at room temperature for 1 h. Progress of reaction was monitored by TLC and LCMS. After completion, reaction mixture was acidified with 1N aq.

HCl solution and extracted with ethyl acetate (3×50 mL). Combined organic layer was washed with brine solution (50 mL) and dried over anhydrous sodium sulphate. Removal of solvent under reduced pressure gave 2-methyl-3-(pyrimidin-2-yl)prop-2-enoic acid (300 mg).

LCMS: 165 (M+1)

### Step-5

To a solution of (2E)-2-methyl-3-(pyrimidin-2-yl)prop-2-enoic acid (100 mg, 0.61 mmol, 1 eq) in THF (10 mL) was added triethylamine (73 mg, 0.73 mmol, 1.2 eq) and stirred the reaction mixture at 0° C. for 10 minute. To this solution was added pivaloyl chloride (73 mg, 0.6097 mmol, 1 eq) dropwise and the mixture was allowed to stir at 0° C. for 1 h. Progress of reaction was monitored by TLC. After completion, reaction mixture was filtered and the filtrate was used in the next step.

### Step-6

To a stirred solution of 3-methyl-5,6-dihydropyridin-2(1H)-one (79 mg, 0.603 mmol, 1 eq) in THF (20 mL) was at −78° C. was added n-BuLi (0.24 mL, 0.603 mmol, 1 eq) dropwise and mixture was stirred 1 h. To this solution was added a solution of 2,2-dimethylpropanoic (2E)-2-methyl-3-(pyrimidin-2-yl)prop-2-enoic anhydride prepared above. Resulting mixture was stirred was stirred at the same temperature for 1 h. Progress of reaction was monitored by LCMS. After completion, reaction mixture was diluted with aq. ammonium chloride solution and extracted with ethyl acetate (3×50 mL). Combined organic layer was washed with brine (50 mL) and dried over sodium sulphate. Removal of solvent under reduced pressure gave crude product which was which was Combi-Flash on silica gel using ethyl acetate-hexane to afford (E)-3-chloro-1-(2-methyl-3-(pyrimidin-2-yl)acryloyl)-5,6-dihydropyridin-2(1H)-one (20 mg, 11.97%).

LCMS: 278.1 (M+1)

¹H NMR (400 MHz, CDCl₃) δ 8.74 (d, 2H), 7.03-7.13 (m, 2H), 6.72 (s, 1H), 3.99 (t, 2H), 2.57-2.72 (m, 2H), 2.49-2.57 (m, 3H).

### Example 46

### Preparation of 1-{(2E)-3-[1-(methylsulfonyl)piperidin-4-yl]prop-2-enoyl}-5,6-dihydropyridin-2(1H)-one

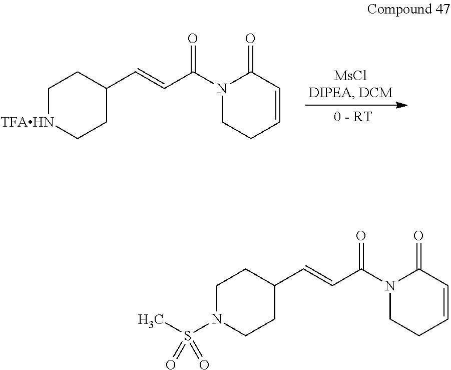

To a stirred solution of 1-[(2E)-3-(piperidin-4-yl)prop-2-enoyl]-5,6-dihydropyridin-2(1H)-one TFA salt (0.100 g, 0.28 mmol, 1.0 eq) in DCM (5.0 mL) was added diisopropylethylamine (0.074 mL, 0.43 mmol, 1.5 eq) dropwise at 0° C. and the mixture was allowed to stir at the same temperature for 30 minutes. To this mixture was added a solution of mesyl chloride (0.026 mL, 0.34 mmol, 1.2 eq) in DCM (2.0 mL) and the resulting mixture was allowed to stir at room temperature for 3 h. Progress of reaction was monitored by TLC. After completion, reaction mixture was evaporated to dryness under vacuum to afford crude which was purified by reversed phase HPLC to afford 1-{(2E)-3-[1-(methylsulfonyl)piperidin-4-yl]prop-2-enoyl}-5,6-dihydropyridin-2(1H)-one (0.028 g, 31.46%).

LCMS: 313 [M+1]⁺

¹H NMR (400 MHz, DMSO-d₆) δ 7.10-7.01 (m, 1H), 6.85-6.70 (m, 2H), 6.91 (d, 1H), 3.83 (t, 2H), 3.60-3.50 (m, 2H), 2.85 (s, 3H), 2.80-2.70 (m, 2H), 2.50-2.40 (m, 3H), 1.85-1.77 (m, 2H), 1.46-1.35 (m, 2H).

### Example 47

### Preparation of (E)-1-(3-(quinazolin-2-yl)acryloyl)-5,6-dihydropyridin-2(1H)-one

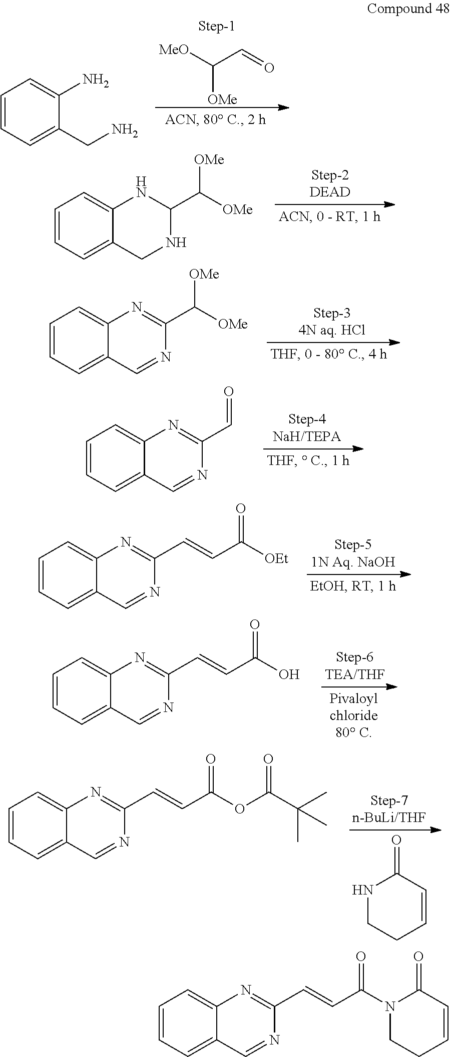

### Step-1

A solution of 2-(aminomethyl)aniline (10 g, 81.83 mmol, 1 eq.) and 2,2-dimethoxyacetaldehyde (8.52 g, 81.83 mmol, 1 eq.) in acetonitrile (150 mL) was allowed to stir at 80° C. for 2 h. Progress of reaction was monitored by TLC. After completion, acetonitrile was removed under reduced pressure, the residue was diluted with ice cold water (150 mL) and extracted with ethyl acetate (2×500 mL). Combined organic layer was washed with water (3×100 mL) followed by brine solution (100 mL), and dried over anhydrous sodium sulphate. Removal of solvent under reduced pressure reduced pressure afforded crude which was purified by Combi-Flash on silica gel (100-200 mesh) using ethyl acetate-hexane system as eluent to afford 2-(dimethoxymethyl)-1,2,3,4-tetrahydroquinazoline (10.6 g, 62%) as brown solid.

### Step-2

To a solution of 2-(dimethoxymethyl)-1,2,3,4-tetrahydroquinazoline (10.6 g, 50.89 mmol) in acetonitrile (50 mL) at 0° C. was added DEAD (19.5 g, 111.97 mmol, 2.2 eq.) portion-wise and then the resulting mixture was allowed to stir at room temperature for 1 h. Progress of reaction was monitored by TLC. After completion of reaction, reaction mixture was diluted with ice cold water (100 mL), acidified with 1N aq. HCl (100 mL) and extracted with ethyl acetate. Aqueous layer was basified with saturated sodium bicarbonate solution (100 mL) and extracted with ethyl acetate (3×200 mL). Combined organic layer was washed with water (100 mL) followed by brine (100 mL) and dried over anhydrous sodium sulphate. Removal of solvent under reduced pressure afforded 2-(dimethoxymethyl)quinazoline (6.2 g, 60%) as white solid.

### Step-3

To a solution of 2-(dimethoxymethyl)quinazoline (6.2 g, 30.35 mmol, 1 eq.) in THF (50 mL) at 0° C. was added 4N aq. HCl (5 mL) and the reaction mixture was allowed to stir at 80° C. for 4 h. Progress of reaction was monitored by TLC. After completion, reaction mixture was concentrated under reduced pressure up to dryness and purified by Combi-Flash on silica gel (100-200 mesh) using ethyl acetate-hexane system as eluent to afford quinazoline-2-carbaldehyde (2.5 g, 52%).

### Step-4

To a solution of triethyl phosphonoacetate (TEPA) (850 mg, 3.796 mmol, 1.2 eq) in THF (20 mL) was added NaH (163 mg, 4.113 mmol, 1.3 eq) at 0° C. and mixture was stirred for 5 minutes. To this mixture was then added a solution of quinazoline-2-carbaldehyde (500 mg, 3.164 mmol, 1 eq) in THF (2 mL) dropwise at 0° C. and the reaction mixture was stirred for 1 h. Progress of reaction was monitored by TLC. After completion, the reaction mixture was diluted with water (20 mL) and extracted with ethyl acetate (3×50 mL). Combined organic layer was washed with brine, dried over Na₂SO₄ and concentrated under reduced pressure to obtain crude which was purified by Combi-Flash on silica gel using ethyl acetate-hexane as eluent to afford ethyl (E)-ethyl 3-(quinazolin-2-yl)acrylate (300 mg, yellow liquid, 41.60%).

### Step-5

To the stirred solution of (E)-ethyl 3-(quinazolin-2-yl)acrylate (300 mg, 1.315 mmol, 1 eq) in EtOH (10 mL) at RT was added 1N aq. NaOH (2.63 mL, 2.63 mmol, 2 eq) and the resulting mixture was stirred for at RT for 2 h. Progress of the reaction was monitored by TLC. After completion, the reaction mixture was diluted with 1 N HCl (15 mL) and extracted with ethyl acetate (2×100 mL). Combined organic layer was washed with water, dried over anhydrous Na₂SO₄ and concentrated under reduced pressure to obtain (E)-3-(quinazolin-2-yl)acrylic acid (200 mg, 76%).

### Step-6

To a stirred solution of (E)-3-(quinazolin-2-yl)acrylic acid (90 mg, 0.45 mmol, 1 eq) in THF (5 mL) at 0° C. was added triethylamine (55 mg, 0.54 mmol, 1.2 eq) followed by pivaloyl chloride (56 mg, 0.45 mmol) and the reaction mixture was allowed to stir at 0° C. for 1 h. Progress of reaction was monitored by TLC. After completion, the reaction mixture was filtered and the filtrate was carried to next step without any further purification.

### Step-7

To the stirred solution of 5,6-dihydropyridin-2(1H)-one (44 mg, 0.45 mmol, 1 eq) in THF (5 mL) at −78° C. was added n-BuLi (2.5 M in hexane, 0.18 mL, 0.45 mmol, 1 eq) and the reaction mixture was stirred at the same temperature for 30 minutes. To this reaction mixture was added a solution of pivalic (E)-3-(quinazolin-2-yl)acrylic anhydride (128 mg, 0.45 mmol, 1 eq) in THF (10 mL) and the resulting mixture was allowed to stir at −78° C. for 2 h. Progress of the reaction was monitored by TLC and LCMS. After completion, the reaction mixture was diluted with water (20 mL) and extracted with ethyl acetate (3×50 mL). Combined organic layer was washed with brine, dried over Na₂SO₄ and concentrated under reduced pressure to obtain crude which was purified by Combi-Flash on silica gel using ethyl acetate-hexane system as eluent to afford (E)-1-(3-(quinazolin-2-yl)acryloyl)-5,6-dihydropyridin-2(1H)-one (21 mg, 17%).

LCMS: 280 [M+1]⁺

¹H NMR (CDCl₃, 400 MHz) δ 9.39 (s, 1H), 8.22 (d, 1H), 8.05 (d, 1H), 7.92-7.88 (m, 2H), 7.85 (d, 1H), 7.65 (t, 1H), 6.98 (dd, 1H), 6.08 (d, 1H), 4.09 (t, 2H), 2.55-2.45 (m, 2H).

### Example 48

### Preparation of 1-[(2E)-3-(4,5-dihydropyrimidin-2-yl)-2-methylprop-2-enoyl]-5,6-dihydropyridin-2(1H)-one

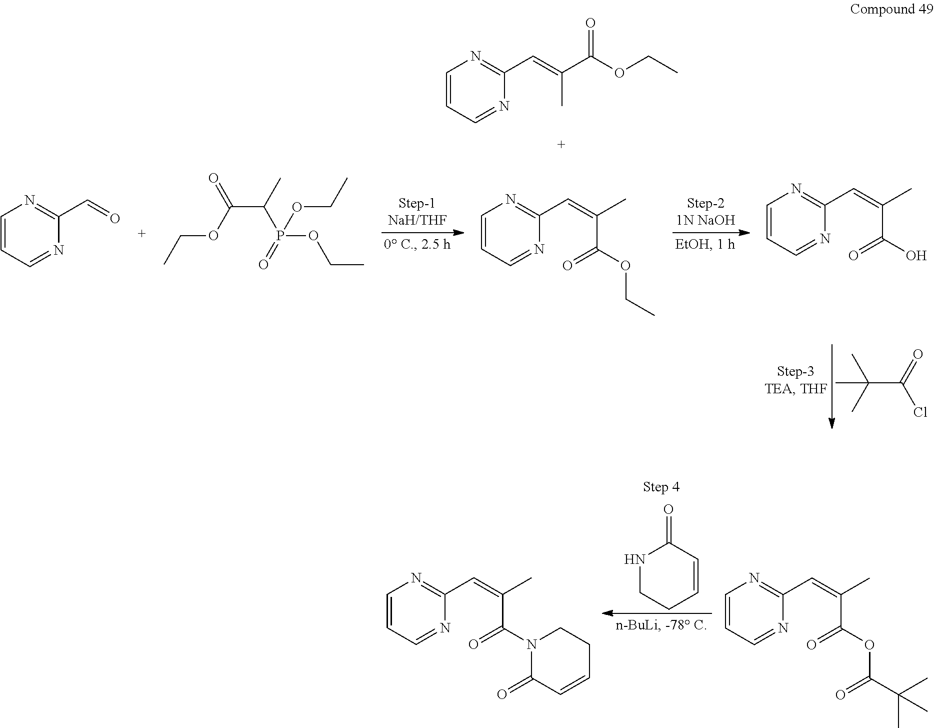

### Step-1

To a solution of ethyl 2-(diethoxyphosphoryl)propanoate (0.95 g, 4.07 mmol, 1.1 eq) in THF (20 mL) was added sodium hydride (0.176 g, 4.443 mmol, 1.2 eq) slowly at 0° C. and the reaction mixture was allowed to stir at 0° C. for 20 minutes. To this solution was added pyrimidine-2-carbaldehyde (0.4 g, 3.7 mmol, 1 eq) and the reaction mixture stirred at 0° C. for 1 h. Progress of reaction was monitored by TLC. After completion, reaction mixture was diluted saturated ammonium chloride solution (30 mL) and extracted with ethyl acetate (3×30 mL). Combined organic layer was washed with brine and dried over anhydrous sodium sulfate. Removal of solvent afforded crude which was purified by Combi-Flash on silica gel using ethyl acetate-hexane system as eluent to afford ethyl (E)-ethyl 2-methyl-3-(pyrimidin-2-yl)acrylate (0.35 g, 49%) and ethyl (Z)-ethyl 2-methyl-3-(pyrimidin-2-yl)acrylate (0.15 g, 21%).

LCMS: 193 [M+1]⁺

### Step-2

To a solution of ethyl (E)-2-methyl-3-(pyrimidin-2-yl)acrylate (0.26 g, 1.354 mmol, 1 eq) in ethanol (10 mL) was added 1N sodium hydroxide (2.7 mL, 2.71 mmol, 2 eq) at RT and the reaction mixture was allowed to stir at RT for 1 h. Progress of reaction was monitored by TLC. After completion, reaction mixture was acidified with 1N HCl (10 mL) and extracted with ethyl acetate (3×25 mL). Combined organic layer was washed with brine (50 mL) and dried over anhydrous sodium sulfate. Removal of solvent afforded (E)-2-methyl-3-(pyrimidin-2-yl)acrylic acid (0.07 g, 37%).

### Step-3

To a solution of (E)-2-methyl-3-(pyrimidin-2-yl)acrylic acid (0.07 g, 0.426 mmol, 1.0 eq.) in dry THF was added triethylamine (0.051 g, 0.511 mmol, 1.2 eq.) and pivaloyl chloride (0.051 g, 0.426 mmol, 1 eq.) and the reaction mixture was stirred at 0° C. for 45 minutes. Progress of reaction was monitored by TLC. After completion, reaction mixture was filtered and the filtrate was used directly for the next step.

### Step-4

To a stirred solution of 5,6-dihydropyridin-2(1H)-one (0.041 g, 0.426 mmol, 1 eq.) in dry THF at −78° C. was added n-butyllithium (2.5 M in hexane, 0.17 mL, 0.426 mmol, 1 eq.) and the mixture was allowed to stir at the same temperature for 1 h. To this solution was added (2E)-3-(4,5-dihydropyrimidin-2-yl)-2-methylprop-2-enoic 2,2-dimethylpropanoic anhydride (0.105 g, 0.426 mmol, 1 eq.) and the reaction mixture was allowed to stir at −78° C. for 1 h. Progress of reaction was monitored by TLC. After completion, reaction mixture was brought to 0° C., diluted with ammonium chloride solution (30 mL) and extracted with ethyl acetate (3×40 mL). Combined organic layer was washed with brine, dried over anhydrous Na₂SO₄ and evaporated to dryness under vacuum to afford crude which was purified by Combi-Flash to afford 1-[(2E)-3-(4,5-dihydropyrimidin-2-yl)-2-methylprop-2-enoyl]-5,6-dihydropyridin-2(1H)-one (10 mg, 10%).

LCMS: 266 [M+23]⁺

¹H NMR (400 MHz, CDCl₃) δ 8.55 (d, 2H), 6.98 (t, 1H), 6.83-6.93 (m, 1H), 6.36 (s, 1H), 5.92 (d, 1H), 4.15 (brs, 2H), 2.58-2.45 (m, 2H), 2.25 (s, 3H).

### Example 49

### Preparation of (E)-1-(3-(1-(4-fluorobenzoyl)piperidin-4-yl)acryloyl)-5,6-dihydropyridin-2(1H)-one

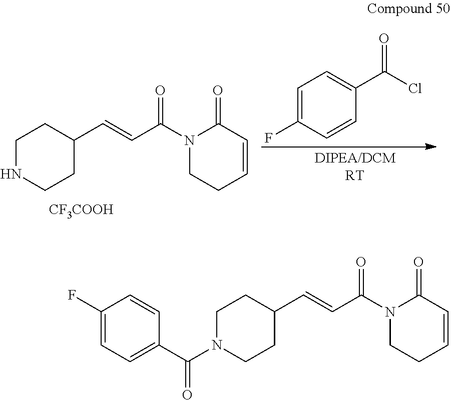

To a suspension of (E)-1-(3-(piperidin-4-yl)acryloyl)-5,6-dihydropyridin-2(1H)-one 2,2,2-trifluoroacetate (150 mg, 0.43 mmol, 1 eq) in dichloromethane (10 mL) was added 4-fluorobenzoyl chloride (68 mg, 0.43 mmol, 1 eq) followed by DIPEA (33 mg, 0.52 mmol, 1.2 eq) at 0° C. and the reaction mixture was allowed to stir at RT for 3 h. Progress of reaction was monitored by TLC and LCMS. After completion, reaction mixture was concentrated to get crude which was purified by Combi-Flash on silica gel (100-200 mesh) using ethyl acetate-hexane system as eluent to afford (E)-1-(3-(1-(4-fluorobenzoyl)piperidin-4-yl)acryloyl)-5,6-dihydropyridin-2(1H)-one (40 mg, 26%).

LCMS: 357 [M+1]⁺

¹H NMR (400 MHz, CDCl₃) δ 7.43-7.36 (m, 2H), 7.35-7.25 (m, 2H), 6.98-6.80 (m, 3H), 6.00 (d, 1H), 4.80-4.35 (m, 1H), 3.98 (t, 2H), 3.97-3.60 (m, 1H), 3.20-2.80 (m, 2H), 2.60-2.40 (m, 3H), 2.00-1.70 (m, 2H), 1.60-1.35 (m, 2H).

### Example 50

### Preparation of (E)-1-(3-(1-(4-fluorophenylsulfonyl)piperidin-4-yl)acryloyl)-5,6-dihydropyridin-2(1H)-one

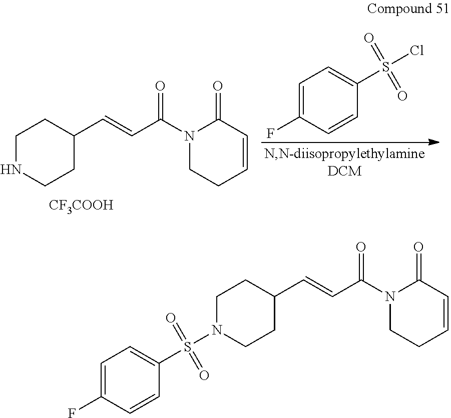

To a solution of (E)-1-(3-(piperidin-4-yl)acryloyl)-5,6-dihydropyridin-2(1H)-one 2,2,2-trifluoroacetate (0.150 g, 0.43 mmol, 1.0 eq.) and N,N-diisopropylethylamine (0.09 mL, 0.646 mmol, 1.5 eq.) in CH₂Cl₂ was added p-fluorobenzenesulfonyl chloride (0.100 mL, 0.58 mmol, 1.2 eq.) at 0° C. and the reaction mixture was stirred at room temperature for 4 h. Progress of reaction was monitored by TLC. After completion, reaction mixture was diluted with water (50 mL) and extracted with CH₂Cl₂ (3×30 ml). Combined organic layer was washed with brine (50 mL) and dried over Na₂SO₄. Removal of solvent under reduced pressure gave crude which was purified by Combi-Flash on silica gel using ethyl acetate-hexane system as eluent to afford (E)-1-(3-(1-(4-fluorophenylsulfonyl)piperidin-4-yl)acryloyl)-5,6-dihydropyridin-2(1H)-one (0.060 g, 36% yield).

LCMS: 393 [M+1]⁺

¹H NMR (400 MHz, CDCl₃) δ 7.71-7.84 (m, 2H) 7.22 (t, 2H) 6.87-6.96 (m, 1H) 6.74-6.85 (m, 2H) 5.99 (d, 1H) 3.96 (t, 2H) 3.78 (d, 2H) 2.33-2.49 (m, 4H) 2.16 (d, 1H) 1.85 (d, 2H) 1.57-1.66 (m, 2H) 1.21-1.35 (m, 2H) 0.88 (t, 2H).

### Example 51

### Preparation of 1-[(2E)-3-(1-methyl-1H-indol-3-yl)prop-2-enoyl]-5,6-dihydropyridin-2(1H)-one

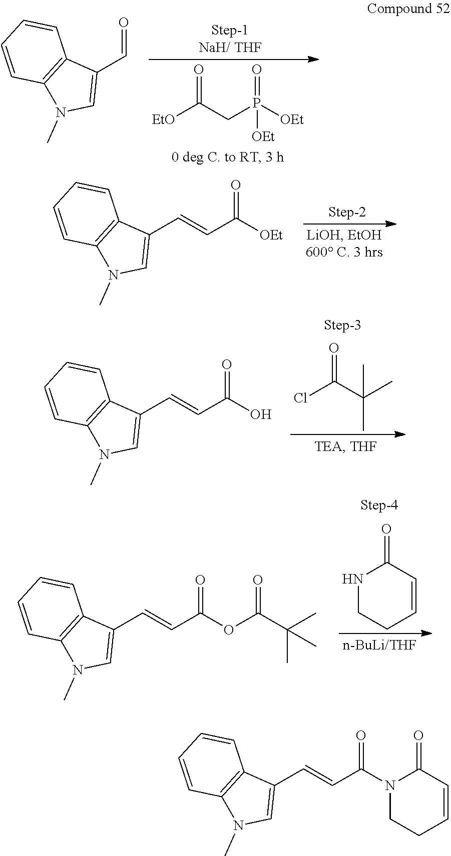

### Step-1

To a suspension of sodium hydride (60% in mineral oil, 0.380 g, 9.42 mmol, 1.5 eq) in THF (20 mL) was added ethyl 2-(diethoxyphosphoryl)acetate (1.86 g, 9.42 mmol, 1.5 eq) dropwise at 0° C. and the mixture was allowed to stir at the same temperature for 30 minutes. To this mixture was added a solution of 1-methyl-1H-indole-3-carbaldehyde (1.0 g, 6.28 mmol, 1.0 eq) in THF (5 mL) and the resulting mixture was allowed to stir at room temperature for 2 h. Progress of reaction was monitored by TLC. After completion, reaction mixture was diluted with saturated aq. NH₄Cl (100 mL) and extracted with ethyl acetate (3×50 mL). Combined organic layer was washed with brine, dried over anhydrous Na₂SO₄ and evaporated to dryness under vacuum to afford crude which was purified by Combi-Flash on silica gel (100-200 mesh) using ethyl acetate-hexane system as eluent to afford ethyl (2E)-3-(1-methyl-1H-indol-3-yl)prop-2-enoate (1.3 g, 90.27%).

LCMS: 230 [M+1]⁺

### Step-2

To a solution of ethyl (2E)-3-(1-methyl-1H-indol-3-yl)prop-2-enoate (1.3 g, 5.6 mmol, 1.0 eq) in ethanol (100 mL) was added lithium hydroxide (1.3 g) in water (6.0 mL) and the reaction mixture was stirred at 70° C. for 2 h. Progress of reaction was monitored by TLC. After completion, reaction mixture was concentrated under vacuum, diluted with water (20 mL), acidified with 1N HCL and extracted with ethyl acetate (3×50 mL). Combined organic layer was washed with brine solution (50 mL) and dried over anhydrous Na₂SO₄. Removal of solvent under reduced pressure afforded (2E)-3-(1-methyl-1H-indol-3-yl)prop-2-enoic acid (1.1 g, 96%) as crystalline solid.

LCMS: 202 [M+1]⁺

### Step-3

To a solution of (2E)-3-(1-methyl-1H-indol-3-yl)prop-2-enoic acid (0.30 g, 1.3 mmol, 1.0 eq.) in dry THF (20 mL) was added triethylamine (0.27 mL, 1.9 mmol, 1.5 eq.) followed by pivaloyl chloride (0.24 mL, 1.9 mmol, 1.5 eq.) and the reaction mixture was stirred at 0° C. for 45 minutes. Progress of reaction was monitored by TLC. After completion, reaction mixture was filtered, and the filtrate was used directly for the next step.

### Step-4

To a stirred solution of 5,6-dihydropyridin-2(1H)-one (0.13 g, 1.32 mmol, 0.9 eq.) in dry THF at −78° C. was added n-butyllithium (2.5 M in hexane, 0.54 mL, 1.1 eq.) and the mixture was allowed to stir at the same temperature for 30 minutes. To this solution was added a solution of 2,2-dimethylpropanoic (2E)-3-(1-methyl-1H-indol-3-yl)prop-2-enoic anhydride (0.42 g, 1.48 mmol, 1.1 eq.) in THF and the reaction mixture was allowed to stir at −78° C. for 1 h. Progress of reaction was monitored by TLC and LCMS. After completion, reaction mixture was brought to 0° C., diluted with water (30 mL) and extracted with ethyl acetate (3×50 mL). Combined organic layer was washed with brine, dried over anhydrous Na₂SO₄ and evaporated to dryness under vacuum to afford crude which was purified by Combi-Flash to afford 1-[(2E)-3-(1-methyl-1H-indol-3-yl)prop-2-enoyl]-5,6-dihydropyridin-2(1H)-one (0.019 g, 4.63%).

LCMS: 281 [M+1]⁺

¹H NMR (400 MHz, DMSO-d₆) δ 7.91-7.80 (m, 3H), 7.60-7.47 (m, 2H), 7.35-7.20 (m, 2H), 7.10-1.02 (m, 1H), 5.99 (d, 1H), 3.93 (t, 2H), 3.83 (s, 1H), 2.50-2.40 (m, 2H).

### Example 52

### Preparation of 1-[(2E)-3-(1-methyl-1H-pyrazol-3-yl)prop-2-enoyl]-5,6-dihydropyridin-2(1H)-one

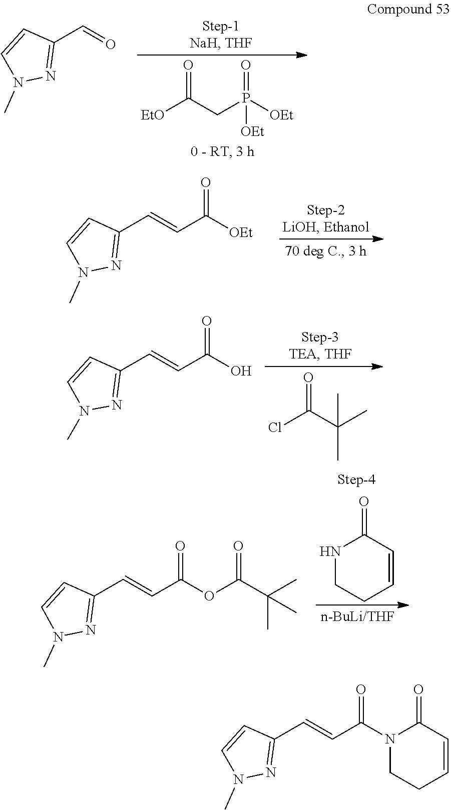

### Step-1

To a suspension of sodium hydride (60% in mineral oil, 0.44 g, 10.9 mmol, 1.2 eq) in THF (50 mL) was added ethyl 2-(diethoxyphosphoryl)acetate (2.44 g, 10.9 mmol, 1.2 eq) dropwise at 0° C. and the mixture was allowed to stir at the same temperature for 30 minutes. To this mixture was added a solution of 1-methyl-1H-pyrazole-3-carbaldehyde (1.0 g, 9.09 mmol, 1.0 eq) in THF (5 mL) and the resulting mixture was allowed to stir at room temperature for 2 h. Progress of reaction was monitored by TLC. After completion, reaction mixture was diluted with saturated aq. NH₄Cl (100 mL) and extracted with ethyl acetate (3×50 mL). Combined organic layer was washed with brine, dried over anhydrous Na₂SO₄ and evaporated to dryness under vacuum to afford crude which was purified by Combi-Flash on silica gel using ethyl acetate-hexane system as eluent to afford ethyl (2E)-3-(1-methyl-1H-indol-3-yl)prop-2-enoate (0.70 g, 61.11%).

LCMS: 181 [M+1]⁺

### Step-2

To a solution of ethyl (2E)-3-(1-methyl-1H-pyrazol-3-yl)prop-2-enoate (0.70 g, 3.88 mmol, 1.0 eq) in ethanol (100 mL) was added a solution of lithium hydroxide (0.28 g, 11.65 mmol, 3.0 eq) in water (6.0 mL) and the reaction mixture was stirred at 70° C. for 2 h. Reaction mixture was concentrated under vacuum and then acidified with 1N HCl (10 mL). The mixture was diluted with water (30 mL) and extracted with ethyl acetate (3×50 mL). Combined organic layer was washed with brine solution (50 mL) and dried over anhydrous Na₂SO₄. Removal of solvent under reduced pressure gave (2E)-3-(1-methyl-H-pyrazol-3-yl)prop-2-enoic acid (0.32 g, 54%) as crystalline solid.

LCMS: 153 [M+1]⁺

### Step-3

To a solution of (2E)-3-(1-methyl-1H-pyrazol-3-yl)prop-2-enoic acid (0.30 g, 1.97 mmol, 1.0 eq.) in dry THF (20 mL) was added triethylamine (0.33 ml, 2.3 mmol, 1.2 eq.) followed by pivaloyl chloride (0.29 ml, 2.3 mmol, 1.2 eq.) and the reaction mixture was stirred at 0° C. for 45 minutes. Progress of reaction was monitored by TLC. After completion, reaction mixture was filtered and the filtrate was used directly for the next step.

### Step-4

To a stirred solution of 5,6-dihydropyridin-2(1H)-one (0.17 g, 1.9 mmol, 1.0 eq.) in dry THF at −78° C. was added nBuLi (0.77 mL, 1.92 mmol, 1.1 eq.) and the mixture was allowed to stir at the same temperature for 30 minutes. To this solution was added a solution of 2,2-dimethylpropanoic (2E)-3-(1-methyl-1H-pyrazol-3-yl)prop-2-enoic anhydride (0.46 g, 1.7 mmol, 1.0 eq.) in THF and the reaction mixture was allowed to stir at room temperature for 1 h. Progress of reaction was monitored by TLC and LCMS. After completion, reaction mixture was evaporated to dryness under vacuum to afford crude which was purified by reversed phase HPLC to afford 1-[(2E)-3-(1-methyl-1H-pyrazol-3-yl)prop-2-enoyl]-5,6-dihydropyridin-2(1H)-one (0.043 g, 9.34%).

LCMS: 232 [M+1]⁺

¹H NMR (400 MHz, DMSO-d₆) δ 7.75 (s, 1H), 7.42 (d, 1H), 7.26 (d, 1H), 7.10-7.00 (m, 1H), 6.60 (s, 1H), 5.98 (d, 1H), 3.90-3.80 (m, 5H), 2.55-2.40 (m, 1H).

### Example 53

### Preparation of 1-[(2E)-3-(1-benzoylpiperidin-4-yl)prop-2-enoyl]-5,6-dihydropyridin-2(1H)-one

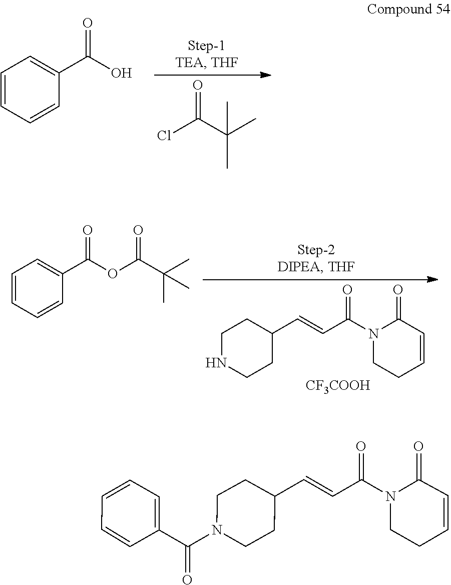

### Step-1

To a solution of benzoic acid (0.20 g, 1.63 mmol, 1.0 eq.) in dry THF (20 mL) was added triethylamine (0.34 mL, 2.45 mmol, 1.5 eq.) followed by pivaloyl chloride (0.30 mL, 2.45 mmol, 1.5 eq.) and the reaction mixture was stirred at 0° C. for 45 minutes. Progress of reaction was monitored by TLC. After completion, reaction mixture was filtered, and the filtrate was used directly for the next step.

### Step-2

To a stirred solution of 1-[(2E)-3-(piperidin-4-yl)prop-2-enoyl]-5,6-dihydropyridin-2(1H)-one (0.10 g, 0.28 mmol, 1.0 eq.) in dry THF at 0° C. was added DIPEA (0.77 mL, 0.34 mmol, 1.2 eq.) (20 mL) and the mixture was allowed to stir at the same temperature for 30 minutes. To this solution was added a solution of benzoic 2,2-dimethylpropanoic anhydride (0.33 g, 0.28 mmol, 1.0 eq.) in THF and the reaction mixture was allowed to stir at 0° C. for 1 h. Progress of reaction was monitored by TLC and LCMS. After completion, reaction mixture was evaporated to dryness under vacuum to afford crude which was purified by Combi-Flash to afford 1-[(2E)-3-(1-benzoylpiperidin-4-yl)prop-2-enoyl]-5,6-dihydropyridin-2(1H)-one (0.065 g, 11.6%).

LCMS: 339 [M+1]⁺

¹H NMR (400 MHz, DMSO-d₆) δ 7.45-7.35 (m, 5H), 7.10-7.01 (m, 1H), 6.85-6.70 (m, 4H), 5.90 (d, 1H), 4.45 (brs, 1H), 3.88-3.80 (m, 2H), 3.60 (brs, 1H), 3.20-2.70 (m, 2H), 2.60-2.40 (m, 1H), 1.80-1.60 (m, 2H), 1.40-1.20 (m, 2H).

### Example 54

### Preparation of 1-[(2E)-3-(8-methoxy-2,3-dihydro-1,4-benzodioxin-6-yl)-2-methylprop-2-enoyl]-5,6-dihydropyridin-2(1H)-one

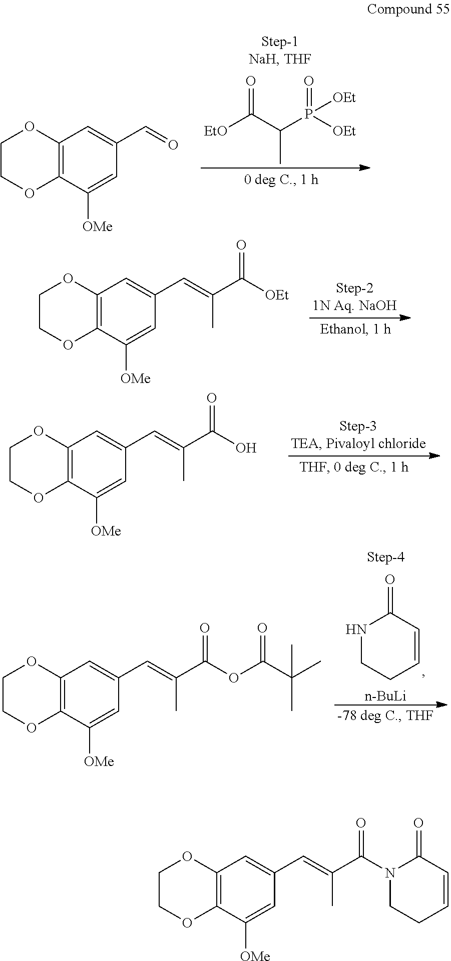

### Step-1

To a stirred ice cold solution of NaH (0.543 g, 13.60 mmol, 1.2 eq) in THF (20 mL) was added ethyl 2-(diethoxyphosphoryl)propanoate (2.96 g, 12.47 mmol, 1.1 eq) and resulting mixture was stirred at 0° C. for 10 minutes. To this mixture was added a solution of 8-methoxy-2,3-dihydro-1,4-benzodioxine-6-carbaldehyde (2.2 g, 11.34 mmol, 1 eq) and reaction mixture was allowed to stir at the same temperature for 1 h. Progress of reaction was monitored by TLC and LCMS. After completion, reaction mixture was diluted with saturated aq. ammonium chloride solution (100 mL) and extracted with ethyl acetate (3×100 mL). Combined organic layer was dried over anhydrous. Removal of solvent under reduced pressure afforded crude which was purified by Combi-Flash on silica gel using ethyl acetate-hexane system as eluent to afford ethyl (2E)-3-(8-methoxy-2,3-dihydro-1,4-benzodioxin-6-yl)-2-methylprop-2-enoate (2.3 g, 73%).

### Step-2

To a stirred solution of ethyl (2E)-3-(8-methoxy-2,3-dihydro-1,4-benzodioxin-6-yl)-2-methylprop-2-enoate (2.3 g, 8.27 mmol, 1 eq) in ethanol (20 mL) was added 1N aq. NaOH (16.54 mL) and the resulting mixture was stirred at room temperature for 1 h. Progress of reaction was monitored by TLC and LCMS. After completion, reaction mixture was diluted with water (50 mL) and acidified with 1N aq. HCl and extracted with ethyl acetate (3×100 mL). Combined organic layer was washed with brine (50 mL) and dried over anhydrous sodium sulfate. Removal of solvent under reduced pressure afforded (2E)-3-(8-methoxy-2,3-dihydro-1,4-benzodioxin-6-yl)-2-methylprop-2-enoic acid (2 g, 95%) which was used in the next step without further purification.

### Step-3

To a stirred solution of (2E)-3-(8-methoxy-2,3-dihydro-1,4-benzodioxin-6-yl)-2-methylprop-2-enoic acid (300 mg, 1.2 mmol, 1 eq) in THF (20 mL) at 0° C. was added triethylamine (145 mg, 1.44 mmol, 1.2 eq) followed by 2,2-dimethylpropanoyl chloride (144 mg, 1.2 mmol, 1 eq) and the resulting mixture was allowed to stir at the same temperature for mixture for 1 h. Progress of reaction was monitored by TLC. After completion, reaction mixture was filtered through cotton plug and used directly in the next reaction.

### Step-4

To a stirred solution of 5,6-dihydropyridin-2(1H)-one (116 mg, 1.2 mmol, 1 eq) in THF (20 mL) at −78° C. was added n-BuLi (0.48 mL, 1.2 mmol, 1 eq) dropwise at and the resulting mixture was stirred at same temperature for 1 h. To this mixture was added a solution of 2,2-dimethylpropanoic-3-(8-methoxy-2,3-dihydro-1,4-benzodioxin-6-yl)methylprop-2-enoic anhydride (400 mg, 1.2 mmol, 1 eq) in THF (20 mL) dropwise and the mixture was stirred at −78° C. for another 1 h. Progress of reaction was monitored by TLC and LCMS. After completion, reaction mixture was diluted with saturated ammonium chloride solution (50 mL) and extracted with dichloromethane (3×100 mL). Combined organic layer was dried over anhydrous sodium sulfate and concentrated under reduced pressure to yield crude product, which was purified by Combi-Flash on silica gel using ethyl acetate-hexane system as eluent to afford 1-[(2E)-3-(8-methoxy-2,3-dihydro-1,4-benzodioxin-6-yl)-2-methylprop-2-enoyl]-5,6-dihydropyridin-2(1H)-one (120 mg, 30.39%)

LCMS: 330.2 (M+H)

¹H NMR (400 MHz, CDCl₃) δ 6.93 (dd, 1H), 6.81 (s, 1H), 6.65 (s, 1H), 6.54 (s, 1H), 5.99 (d, 1H), 4.29-4.39 (m, 2H), 4.27 (d, 2H), 3.78-3.96 (m, 5H), 2.53 (d, 2H), 2.13 (s, 3H).

### Example 55

### Preparation of (E)-1-(3-(1-methyl-1H-pyrazol-4-yl)acryloyl)-5,6-dihydropyridin-2(1H)-one

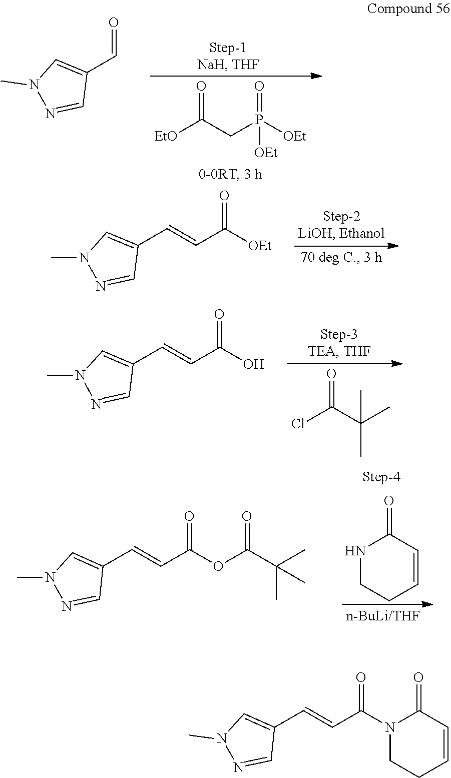

### Step-1

To a solution of ethyl 2-(diethoxyphosphoryl)acetate (5.4 g, 22.7 mmol, 1 eq) in THF (100 mL) at 0° C. was added sodium hydride (60% in mineral oil, 1.2 g, 29.5 mmol, 1.3 eq) portion-wise and the mixture was allowed to stir at the same temperature for 30 minutes. To this mixture was added a solution of 1-methyl-1H-pyrazole-4-carbaldehyde (2.5 g, 22.7 mmol, 1.0 eq) in THF (10 mL) and the resulting mixture was allowed to stir at 0° C. for 1 h. Progress of reaction was monitored by TLC. After completion, reaction mixture was diluted with saturated aq. NH₄Cl (100 mL) and extracted with ethyl acetate (3×100 mL). Combined organic layer was washed with brine, dried over anhydrous Na₂SO₄ and evaporated to dryness under vacuum to afford ethyl (E)-3-(1-methyl-1H-pyrazol-4-yl)acrylate (3.9 g, 95%) which was used in the next step without further purification.

LCMS: 181 [M+1]⁺

### Step-2

To a solution of ethyl (E)-3-(1-methyl-1H-pyrazol-4-yl)acrylate (3.90 g, 21.67 mmol, 1.0 eq) in ethanol (100 mL) was added a solution of lithium hydroxide (1.6 g, 65.01 mmol, 3.0 eq) in water (25 mL) and the reaction mixture was stirred at 70° C. for 2 h. Reaction mixture was concentrated under vacuum and then acidified with 1N aq. HCl. The mixture was diluted with water (100 mL) and extracted with ethyl acetate (5×100 mL). Combined organic layer was washed with brine solution (50 mL) and dried over anhydrous Na₂SO₄. Removal of solvent under reduced pressure gave (E)-3-(1-methyl-1H-pyrazol-4-yl)acrylic acid (2.5 g, 76%) as crystalline solid.

LCMS: 153 [M+1]⁺

### Step-3

To a solution of (E)-3-(1-methyl-1H-pyrazol-4-yl)acrylic acid (1.5 g, 9.9 mmol, 1.0 eq.) in dry THF (50 mL) was added triethylamine (1.68 mL, 12 mmol, 1.2 eq.) followed by pivaloyl chloride (1.23 mL, 9.9 mmol, 1 eq.) and the reaction mixture was stirred at 0° C. for 45 minutes. Progress of reaction was monitored by TLC. After completion, reaction mixture was filtered and the filtrate was used directly for the next step.

### Step-4

To a stirred solution of 5,6-dihydropyridin-2(1H)-one (0.96 g, 9.9 mmol, 1.0 eq.) in dry THF (60 mL) at −78° C. was added n-BuLi (3.9 mL, 9.9 mmol, 1 eq.) and the mixture was allowed to stir at the same temperature for 30 minutes. To this solution was added a solution of (E)-(E)-3-(1-methyl-1H-pyrazol-4-yl)acrylic pivalic anhydride (9.9 mmol, 1.0 eq.) in THF and the reaction mixture was allowed to stir at room temperature for 1 h. Progress of reaction was monitored by TLC and LCMS. After completion, reaction mixture was evaporated to dryness under vacuum to afford crude which was purified by Combi-Flash on silica gel using ethyl acetate-hexane system as eluent to afford 1.4 g (E)-1-(3-(1-methyl-1H-pyrazol-4-yl)acryloyl)-5,6-dihydropyridin-2(1H)-one with 90% LCMS purity. The residue was recrystallized with ethyl acetate and pentane to afford 99% pure (E)-1-(3-(1-methyl-1H-pyrazol-4-yl)acryloyl)-5,6-dihydropyridin-2(1H)-one (1.09 g, 48%).

LCMS: 232 [M+1]⁺

¹H NMR (400 MHz, DMSO-d₆) δ 7.75 (s, 1H), 7.42 (d, 1H), 7.26 (d, 1H), 7.10-7.00 (m, 1H), 6.60 (s, 1H), 5.98 (d, 1H), 3.90-3.80 (m, 5H), 2.55-2.40 (m, 1H).

### Example 56

### Preparation of (E)-1-(3-(1-(phenylsulfonyl)piperidin-4-yl)acryloyl)-5,6-dihydropyridin-2(H)-one

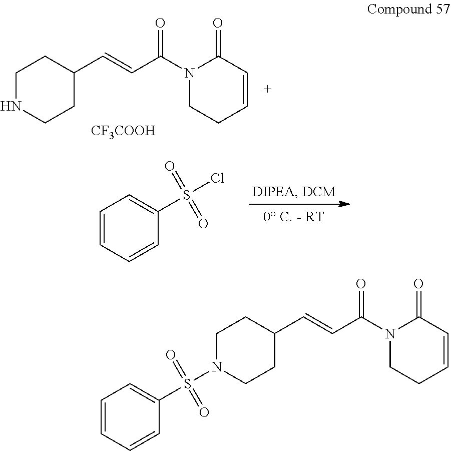

To a solution of (E)-1-(3-(piperidin-4-yl)acryloyl)-5,6-dihydropyridin-2(1H)-one 2,2,2-trifluoroacetate (0.1 g, 0.29 mmol, 1.0 eq.) in dichloromethane (20 mL) at 0° C. was added DIPEA (0.057 mL, 0.43 mmol, 1.5 eq.) followed by benzene sulfonyl chloride (51 mg, 0.29 mmol, 1 eq.) and the reaction mixture was stirred at room temperature for 2 h. Progress of reaction was monitored by TLC. After completion, reaction mixture was diluted with water (50 mL) and extracted with dichloromethane (3×20 mL). Combined organic layer was washed with brine (20 mL) and dried over anhydrous sodium sulfate. Removal of solvent gave crude which was purified by Combi-Flash on silica gel using ethyl acetate-hexane system as eluent to afford (E)-1-(3-(1-(phenylsulfonyl)piperidin-4-yl)acryloyl)-5,6-dihydropyridin-2(1H)-one (50 mg, 46%).

LCMS: 375 [M+1]⁺

¹H NMR (400 MHz, CDCl₃) δ 7.79 (d, 2H), 7.65-7.50 (m, 3H), 6.95-6.78 (m, 4H), 3.96 (t, 2H), 3.80 (d, 2H), 2.50-2.30 (m, 4H), 2.20-2.08 (m, 1H), 1.90-1.79 (m, 2H), 1.67-1.50 (m, 2H).

### Example 57

### Preparation of N-(4-fluorophenyl)-4-[(1E)-3-oxo-3-(6-oxo-3,6-dihydropyridin-1(2H)-yl)prop-1-en-1-yl]piperidine-1-carboxamide

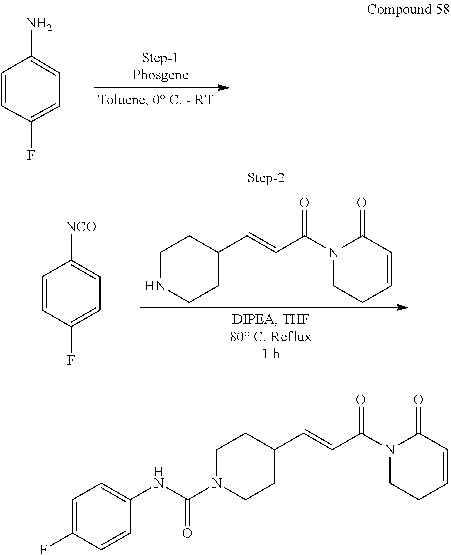

### Step-1

To a stirred solution of 4-fluoroaniline (1.2 g, 10.81 mmol, 1 eq) in dry toluene (10 mL) was added phosgene (20% in toluene, 5.29 ml 10.81 mmol 1 eq) dropwise at 0° C. and the resulting mixture was stirred for 20 minutes. To this mixture was then added a solution of triethylamine (1.69 mL 12.97 mmol 1.2 eq) in toluene (5 mL) and the resulting mixture was stirred at room temperature for 1 h. Progress of reaction was monitored by TLC. After completion, reaction mixture was concentrated under reduced pressure to yield 1-fluoro-4-isocyanatobenzene which was used in the next step without purification.

### Step-2

To a stirred solution of 1-[(2E)-3-(piperidin-4-yl)prop-2-enoyl]-5,6-dihydropyridin-2(1H)-one (0.1 g, 0.287 mmol, 1 eq) in THF (5 mL) was added DIPEA (0.075 mL, 0.430 mmol, 1.5 eq) and the resulting mixture was stirred for 10 minutes. To this mixture was then added a solution of 1-fluoro-4-isocyanatobenzene (0.039 g, 0.287 mmol, 1 eq) in THF (5 mL) and mixture was stirred under reflux 1 h. Progress of reaction was monitored by TLC. After completion, reaction mixture was diluted with ethyl acetate (80 mL), washed with brine (2×50 mL) and dried over sodium sulphate. Removal of solvent under reduced pressure afforded crude which was purified by Combi-Flash on silica gel using ethyl acetate-hexane system as eluent to afford N-(4-fluorophenyl)-4-[(1E)-3-oxo-3-(6-oxo-3,6-dihydropyridin-1(2H)-yl)prop-1-en-1-yl]piperidine-1-carboxamide (50 mg, 47.16% yield).

LCMS: 371 [M+1]⁺

¹H NMR (400 MHz, DMSO-d₆) δ 8.51 (s, 1H), 7.45 (dd, 2H), 6.99-7.13 (m, 2H), 6.68-6.84 (m, 2H), 5.93 (d, 1H), 4.11 (d, 2H), 3.84 (t, 2H), 2.85 (t, 2H), 2.42 (d, 3H), 1.73 (d, 2H), 1.17-1.38 (m, 3H).

### Example 58

### Preparation of 1-{(2E)-3-[1-(2-hydroxy-2-methylpropyl)piperidin-4-yl]prop-2-enoyl}-5,6-dihydropyridin-2(1H)-one

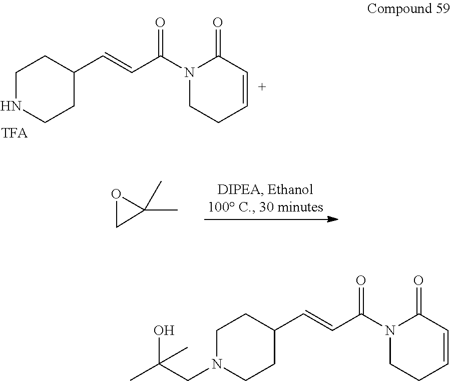

To a stirred solution of a (E)-1-(3-(piperidin-4-yl)acryloyl)-5,6-dihydropyridin-2(1H)-one 2,2,2-trifluoroacetate (200 mg, 0.57 mmol, 1 eq) in ethanol (10 mL) was added DIPEA (583 mg, 5.7 mmol, 10 eq) and the resulting mixture was stirred for 5 minute. To this mixture was added 2,2-dimethyloxirane (252 mg, 3.5 mmol, 6.1 eq) and reaction mixture was stirred at 100° C. for 30 minutes. Progress of reaction was monitored by TLC and LCMS. After completion, reaction mixture was concentrated under vacuum to afford crude oil which was purified by reversed phase chromatography to afford 1-{(2E)-3-[1-(2-hydroxy-2-methylpropyl)piperidin-4-yl]prop-2-enoyl}-5,6-dihydropyridin-2(1H)-one (20 mg, 11%).

LCMS: 307.3 [M+H]⁺

¹H NMR (400 MHz, CDCl₃) δ 7.00-6.86 (m, 2H), 6.81 (d, 1H), 6.00 (d, 1H), 4.01-3.95 (m, 2H), 3.00-2.92 (m, 2H), 2.46-2.20 (m, 6H), 1.82-1.70 (m, 2H), 1.65-1.50 (m, 2H), 1.19 (s, 6H).

### Example 59

### Preparation of 1-{3-[1-(4-fluorobenzyl)piperidin-4-yl]prop-2-enoyl}-5,6-dihydropyridin-2(1H)-one

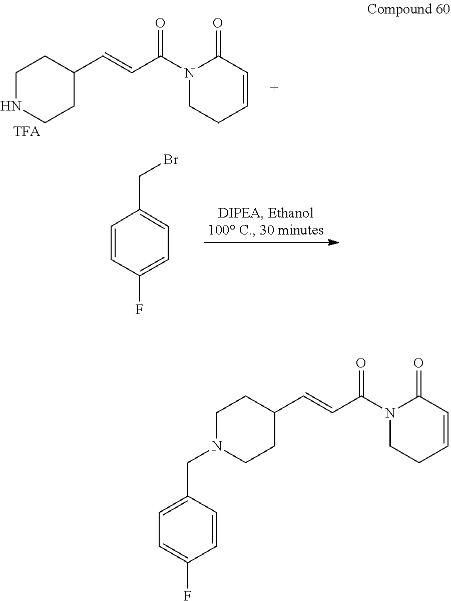 [SMILES: O=C1C=CCCN1C(=O)/C=C/C1CCNCC1 ; Fc1ccc(CBr)cc1 ; O=C1C=CCCN1C(=O)/C=C/C1CCN(Cc2ccc(F)cc2)CC1]

To a stirred solution of a (E)-1-(3-(piperidin-4-yl)acryloyl)-5,6-dihydropyridin-2(1H)-one 2,2,2-trifluoroacetate (200 mg, 0.57 mmol, 1 eq) in ethanol (10 mL) was added DIPEA (583 mg, 5.7 mmol, 10 eq) and the resulting mixture was stirred for 5 minute. To this mixture was added 1-(bromomethyl)-4-fluorobenzene (176 mg, 0.93 mmol, 1.6 eq) and heated the resulting reaction mixture at 100° C. for 60 minutes. Progress of reaction was monitored by TLC and LCMS. After completion, reaction mixture was concentrated under vacuum to afford crude oil which was purified by reversed phase chromatography to afford 1-{3-[1-(4-fluorobenzyl)piperidin-4-yl]prop-2-enoyl}-5,6-dihydropyridin-2(1H)-one (20 mg, 10%).

LCMS: 344.3 [M+H]

¹H NMR (400 MHz, CD₃OD) 7.53 (dd, 2H) 7.23 (t, 2H) 6.99-7.11 (m, 1H) 6.70-6.91 (m, 2H) 5.96 (d, 1H) 4.25 (brs, 2H) 3.93 (t, 2H) 3.43 (brs, 2H) 3.01 (brs, 2H) 2.54 (brs, 1H) 2.47 (d, 2H) 2.05 (d, 2H) 1.68 (brs, 2H).

### Example 60

### Preparation of 1-{3-[1-(4-fluorophenyl)piperidin-4-yl]prop-2-enoyl}-5,6-dihydropyridin-2(1H)-one

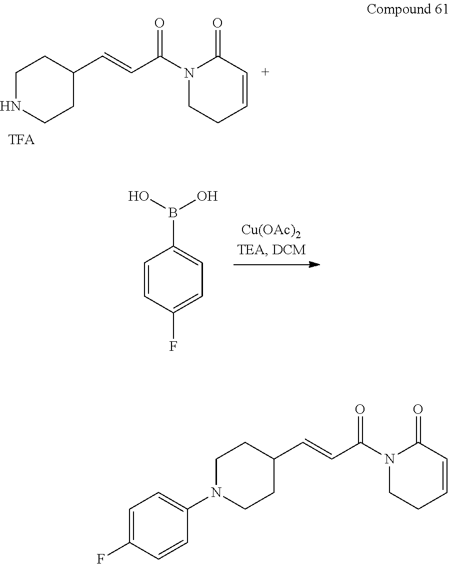 [SMILES: O=C1C=CCCN1C(=O)/C=C/C1CCNCC1 ; OB(O)c1ccc(F)cc1 ; O=C1C=CCCN1C(=O)/C=C/C1CCN(c2ccc(F)cc2)CC1]

To a stirred solution of a 1-[3-(piperidin-4-yl)prop-2-enoyl]-5,6-dihydropyridin-2(1H)-one TFA salt (100 mg, 0.29 mmol, 1 eq) in DCM (20 mL) was added TEA (0.12 mL, 0.58 mmol, 2 eq) the resulting mixture was stirred for 5 minutes. To this mixture was added 4-fluorophenyl boronic acid (80 mg, 0.58 mmol, 2 eq) and reaction mixture was stirred at room temperature overnight. Progress of reaction was monitored by TLC and LCMS. After completion, reaction mixture was diluted with water (50 mL) and extracted with ethyl acetate (3×50 mL). Combined organic layer was dried over anhydrous sodium sulfate. Removal of solvent under reduced pressure gave crude which was purified by Combi-Flash on silica gel to afford 1-{3-[1-(4-fluorophenyl)piperidin-4-yl]prop-2-enoyl}-5,6-dihydropyridin-2(1H)-one (15 mg, 16%).

LCMS: 329.2 [M+H]

¹H NMR (400 MHz, CDCl₃) δ 7.05-6.85 (m, 7H), 6.02 9 (d, 1H), 4.02-3.97 (m, 2H), 3.60-3.55 (m, 2H), 2.78-2.65 (m, 2H), 2.46-2.25 (m, 3H), 1.95-1.187 (m, 2H), 1.78-1.56 (m, 2H).

### Example 61

### Preparation of (E)-1-(3-(1-neopentylpiperidin-4-yl)acryloyl)-5,6-dihydropyridin-2(1H)-one

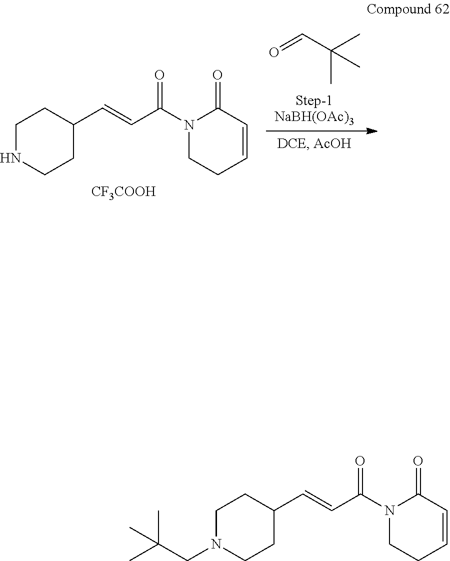

To a stirred solution of (E)-1-(3-(piperidin-4-yl)acryloyl)-5,6-dihydropyridin-2(1H)-one TFA Salt (0.150 g, 0.641 mmol, 1.0 eq) in DCE (20 mL) were added pivalaldehyde (0.165 ml, 1.923 mmol, 3.0 eq) and acetic acid (0.096 mL, 3.205 mmol, 5.0 eq). The reaction mixture was allowed to stir at RT for 1 h and followed by the addition of sodium triacetoxyborohydride (0.408 g, 1.933 mmol, 3.0 eq) and stirring was continued further at room temperature for 1 h. Progress of the reaction was monitored by TLC and LCMS. After completion of the reaction, the reaction mixture was diluted with water (30 mL) and extracted with ethyl acetate (2×30 mL). Organic layer was washed with brine solution (30 mL). Organic layer was dried over anhydrous sodium sulphate and concentrated under reduced pressure to obtain crude, which was purified by reversed phase HPLC to afford (E)-1-(3-(1-neopentylpiperidin-4-yl)acryloyl)-5,6-dihydropyridin-2(1H)-one (0.010 g, 5.1%) was obtained as off white solid.

LCMS: 305 [M+1]⁺

¹H NMR (400 MHz, DMSO-d₆) δ 7.04-7.10 (m, 1H) 6.76-6.83 (m, 1H) 6.66-6.73 (m, 1H) 5.92 (d, 1H) 3.83 (t, 2H) 2.76 (d, 2H) 2.42 (d, 2H) 2.18-2.25 (m, 2H) 2.09 (br. s., 1H) 1.62 (d, 2H) 1.32-1.41 (m, 2H) 0.83 (s, 9H).

### Example 62

### Preparation of (E)-1-(3-(5-methoxypyrimidin-2-yl)acryloyl)-5,6-dihydropyridin-2(1H)-one

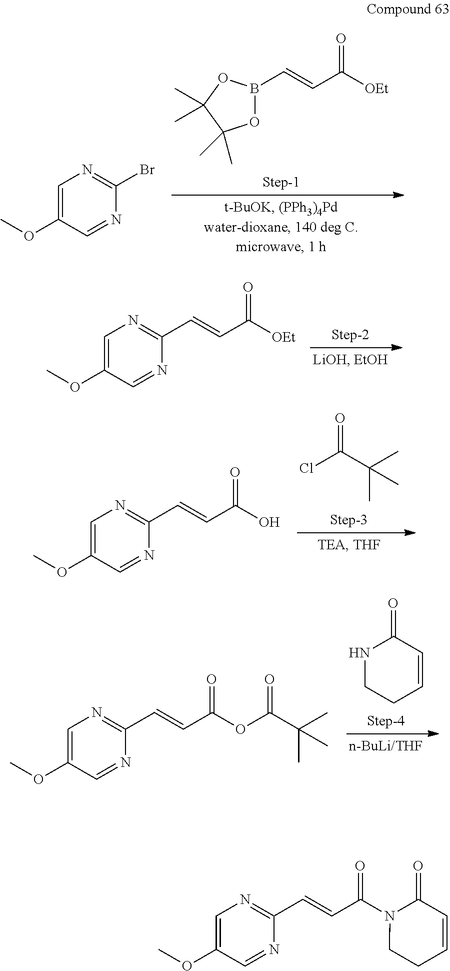

### Step-1

To a solution of 2-bromo-5-methoxypyrimidine (0.5 g, 2.66 mmol, 1.0 eq) in 1,4-dioxane (15 mL) were added ethyl (E)-3-(4,4,5,5-tetramethyl-1,3,2-dioxaborolan-2-yl)acrylate (0.721 g, 3.19 mmol, 1.2 eq) and potassium tert-butoxide (0.595 g, 5.32 mmol, 2.0 eq) in water (2 mL) and the reaction mixture was deoxygenated using nitrogen gas for 10 minutes. Tetrakis(triphenylphosphine)palladium(0) (0.153 g, 0.132 mmol, 0.05 eq) was added and the reaction mixture was again deoxygenated for 10 minutes. Reaction mixture was allowed to stir at 140° C. for 1 h in microwave and progress of reaction was monitored by TLC, after completion the reaction mixture was cooled to RT. The mixture was diluted with water (30 mL) and extracted with ethyl acetate (3×30 mL). Combined organic layer was washed with brine solution (50 mL) and dried over anhydrous Na₂SO₄. Solvent was concentrated under reduced pressure to afford crude which was purified by Combi-Flash on silica gel using ethyl acetate-hexane as eluent to afford ethyl (E)-3-(5-methoxypyrimidin-2-yl)acrylate (0.4 g, 72.72%).

LCMS: 209 [M+1]⁺

### Step-2

To a solution of ethyl (E)-3-(5-methoxypyrimidin-2-yl)acrylate (0.4 g, 1.92 mmol, 1.0 eq) in ethanol (40 mL) was added a solution of lithium hydroxide (0.138 g, 5.769 mmol, 3.0 eq) in water (10.0 mL) and the reaction mixture was stirred at 70° C. for 2 h. Progress of reaction was monitored by TLC. After completion the reaction mixture was cooled to RT, The reaction mixture was concentrated under vacuum and then acidified with 1N HCl (10 mL). The mixture was diluted with water (30 mL) and extracted with ethyl acetate (3×30 ml). Combined organic layer was washed with brine solution (50 mL) and dried over anhydrous Na₂SO₄. Concentrated the solvent under reduced pressure gave (E)-3-(5-methoxypyrimidin-2-yl)acrylic acid (0.320 g, 86.70%) as crystalline solid.

LCMS: 181 [M+1]⁺

### Step-3

To a solution of (E)-3-(5-methoxypyrimidin-2-yl)acrylic acid (0.3 g, 1.665 mmol, 1.0 eq.) in dry THF (10 mL) was added triethylamine (0.257 mL, 1.833 mmol, 1.1 eq.) followed by pivaloyl chloride (0.219 mL, 1.833 mmol, 1.1 eq.) and the reaction mixture was stirred at 0° C. for 45 minutes. Progress of reaction was monitored by TLC. After completion, reaction mixture was filtered and the filtrate was used directly for the next step.

### Step-4

To a stirred solution of 5,6-dihydropyridin-2(1H)-one (0.100 g, 1.03 mmol, 1.0 eq.) in dry THF (10 mL) at −78° C. was added n-butyllithium (2.5 M in hexane, 0.45 mL, 1.13 mmol, 1.1 eq.) and the mixture was allowed to stir at the same temperature for 30 minutes. To this solution was added a solution of (E)-(E)-3-(5-methoxypyrimidin-2-yl)acrylic pivalic anhydride (0.299 g, 1.134 mmol, 1.1 eq.) in THF (10 mL) and the reaction mixture was allowed to stir at −78° C. for 1 h. Progress of reaction was monitored by TLC. After completion, reaction mixture was brought to 0° C., diluted with water (30 mL) and extracted with ethyl acetate (3×30 mL). Combined organic layer was washed with brine, dried over anhydrous Na₂SO₄ and evaporated to dryness under vacuum to afford crude which was purified by Combi-Flash to afford (E)-1-(3-(5-methoxypyrimidin-2-yl)acryloyl)-5,6-dihydropyridin-2(1H)-one (0.140 g, 52.23%) was obtained as off white solid.

LCMS: 260 [M+1]⁺

¹H NMR (400 MHz, CDCl₃) □ δ 8.42 (s, 2H), 7.95 (d, 1H), 7.67 (d, 1H), 6.91-7.00 (m, 1H), 6.05 (d, 1H), 4.05 (t, 2H), 3.95 (s, 3H), 2.55-2.45 (m, 2H).

### Example 63

### Preparation of tert-butyl (E)-4-methyl-4-(3-oxo-3-(6-oxo-3,6-dihydropyridin-1(2H)-yl)prop-1-en-1-yl)piperidine-1-carboxylate

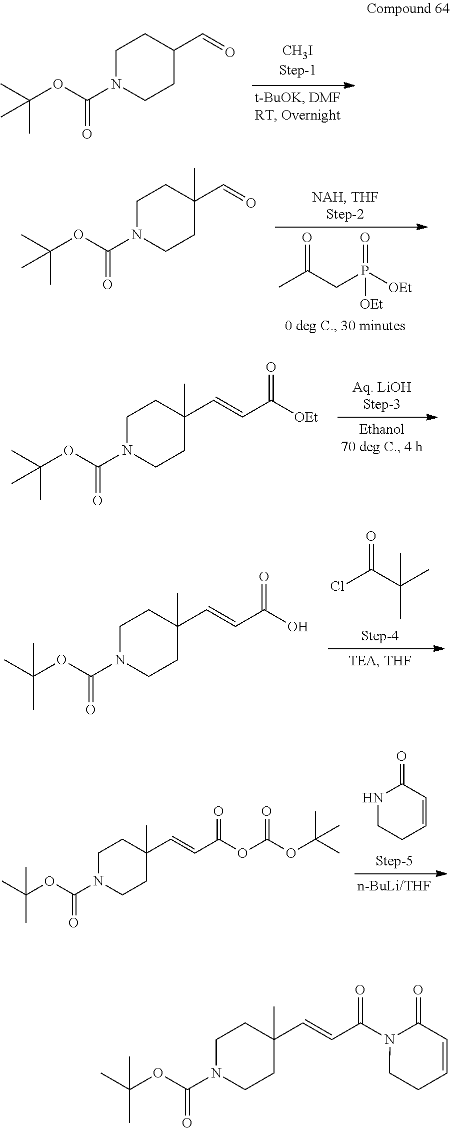

### Step-1

To a solution of tert-butyl 4-formylpiperidine-1-carboxylate (3 g, 14.08 mmol) in DMF (30 mL) were added t-BuOK (1.56 g, 28 mmol, 2 eq) and methyl iodide (3.98 g, 28 mmol, 2 eq) at 0° C. The reaction mixture was stirred at RT overnight. Progress of reaction was monitored by TLC. After completion, reaction mixture was diluted with brine (50 mL) and extracted EtOAc (2×50 mL). Combine organic layer was washed with brine and dried over anhydrous sodium sulfate. Removal of solvent under reduced pressure gave tert-butyl 4-formyl-4-methylpiperidine-1-carboxylate (3 g, 94%) which was used in the next step without purification.

LCMS: 228 [M+1]⁺

### Step-2

To a suspension of sodium hydride (60% in mineral oil, 793 mg 19.83 mmol, 1.5 eq) in THF (50 mL) ethyl (diethoxyphosphoryl)acetate (3.77 g, 16.83 mmol, 1.3 eq) dropwise at 0° C. and the mixture was allowed to stir at the same temperature for 30 minutes. To this mixture was added a solution of tert-butyl 4-formyl-4-methylpiperidine-1-carboxylate (3 g, 13.22 mmol, 1.0 eq) in THF (5 mL) and the resulting mixture was allowed to stir at room temperature for 1 h. Progress of reaction was monitored by TLC. After completion, reaction mixture was diluted with saturated aq. NH₄Cl (100 mL) and extracted with ethyl acetate (3×50 mL). Combined organic layer was washed with brine, dried over anhydrous Na₂SO₄ and evaporated to dryness under vacuum to afford crude which was purified by Combi-Flash using ethyl acetate-hexane system as eluent to afford tert-butyl 4-[(1E)-3-ethoxy-3-oxoprop-1-en-1-yl]-4-methylpiperidine-1-carboxylate (1.4 g, 36%).

LCMS: 298 [M+1]⁺

### Step-3

To a solution of tert-butyl 4-[(1E)-3-ethoxy-3-oxoprop-1-en-1-yl]-4-methylpiperidine-1-carboxylate (1.1 g, 3.7 mmol, 1.0 eq) in ethanol (200 mL) was added a solution of Lithium hydroxide (444 mg, 18.5 mmol, 5 eq) in water (20 mL) and the reaction mixture was stirred at 70° C. for 4 h. Progress of reaction was monitored by TLC. After completion, reaction mixture was concentrated under vacuum and then acidified with 2N HCl to make pH up to 3. The mixture was diluted saturated aq. NH₄Cl (30 mL) and extracted with dichloromethane (3×30 mL). Combined organic layer was washed with brine solution (50 mL) and dried over anhydrous Na₂SO₄. Removal of solvent under reduced pressure afforded (2E)-3-{4-methyl-1-[(propan-2-yloxy)carbonyl]piperidin-4-yl}prop-2-enoic acid as crystalline solid (0.65 g, 65%).

LCMS: 270 [M+1]⁺

### Step-4

To a solution of (E)-3-(1-(tert-butoxycarbonyl)piperidin-4-yl)-2-methylacrylic acid (200 mg, 0.743 mmol, 1.0 eq) in dry THF was added triethylamine (0.1 mL, 0.817 mmol, 1.1 eq) followed by pivaloyl chloride (98 μL, 0.817 mmol, 1.1 eq) and the reaction mixture was stirred at 0° C. for 45 minutes. Progress of reaction was monitored by TLC. After completion, reaction mixture was filtered and the filtrate was used directly for the next step.

### Step-5

To a stirred solution of 5,6-dihydropyridin-2(1H)-one (80 mg, 0.82 mmol, 1.1 eq.) in dry THF at −78° C. was added n-butyllithium (2.5 M in hexane, 0.33 ml, 0.82 mmol, 1.1 eq.) and the mixture was allowed to stir at the same temperature for 30 minutes. To this solution was added a solution of anhydride (0.743 mmol, 1 eq) in THF and the reaction mixture was allowed to stir at −78° C. for 1 h. Progress of reaction was monitored by TLC and LCMS. After completion, reaction mixture was brought to 0° C., diluted with water (30 mL) and extracted with ethyl acetate (3×30 mL). Combined organic layer was washed with brine, dried over anhydrous Na₂SO₄ and evaporated to dryness under vacuum to afford crude which was purified by reversed phase HPLC to afford tert-butyl (E)-4-methyl-4-(3-oxo-3-(6-oxo-3,6-dihydropyridin-1(2H)-yl)prop-1-en-1-yl)piperidine-1-carboxylate (50 mg, 19%).

LCMS: 349 [M+1]⁺

¹H NMR (400 MHz, CDCl₃) δ 6.86-7.01 (m, 2H) 6.73-6.83 (m, 1H) 6.00 (dd, 1H) 3.98 (br. s, 2H), 3.54 (brs, 2H), 3.29 (t, 2H), 2.45 (dt, 2H), 1.64-1.80 (m, 2H), 1.57 (brs, 2H) 1.45 (s, 9H), 1.11 (s, 3H).

### Example 64

### Preparation of (E)-1-(3-(2,3-dihydro-[1,4]dioxino[2,3-b]pyrazin-6-yl)acryloyl)-5,6-dihydropyridin-2(1H)-one

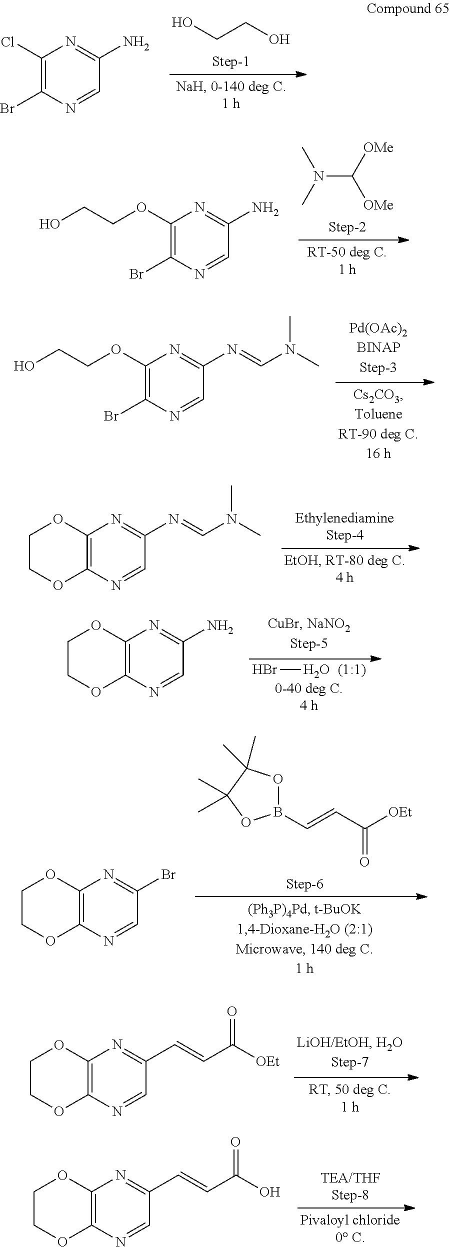

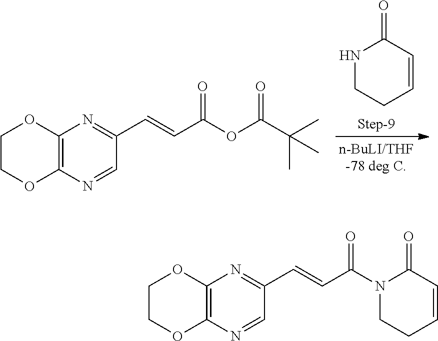 [SMILES: CC(C)(C)C(=O)OC(=O)/C=C/c1cnc2c(n1)OCCO2 ; O=C1C=CCCN1 ; O=C1C=CCCN1C(=O)/C=C/c1cnc2c(n1)OCCO2]

### Step-1

NaH (60% in mineral oil) (1.84 g, 76.73 mmol, 2.0 eq) was added slowly to ethylene glycol (50 mL) at 0° C. over a period of 15 minutes under inert atmosphere and reaction mixture was allowed to stir at the same temperature for 30 minutes. To this mixture was added. Then 5-bromo-6-chloropyrazin-2-amine (8 g, 38.36 mmol, 1 eq) at 0° C. and the resulting mixture was allowed to stir 140° C. for 1 h. Progress of reaction was monitored by TLC. After completion, reaction mixture was diluted with ice cold water (500 mL) and extract with ethyl acetate (2×500 mL). Combined organic layer was washed with brine and dried over anhydrous sodium sulfate. Removal of solvent under reduce pressure afforded 2-(6-amino-3-bromopyrazin-2-yloxy)ethanol (8.97 g, 99.66%) as white solid which was used in the next step without purification.

### Step-2

A mixture of 2-(6-amino-3-bromopyrazin-2-yloxy)ethanol (8.97 g, 38.33 mmol, 1.0 eq.) and DMF-DMA (15 mL) was allowed to stir at 50° C. for 1 h. Progress of reaction was monitored by TLC. After completion, reaction mixture was diluted with ice-cold water (500 mL) and extract with ethyl acetate (2×500 mL). Combined organic layer was washed with brine and dried over anhydrous sodium sulfate. Removal of solvent under reduced pressure afforded gave (E)-N′-(5-bromo-6-(2-hydroxyethoxy)pyrazin-2-yl)-N,N-dimethylformimidamide (11 g, 99.66%) as white solid.

### Step-3

To a solution of (E)-N′-(5-bromo-6-(2-hydroxyethoxy)pyrazin-2-yl)-N,N-dimethylformimidamide (11 g, 38 mmol, 1 eq) in toluene (80 ml) was added Cs₂CO₃ (37.33 g, 114 mmol, 3.0 eq) at room temperature. The reaction mixture was deoxygenated by purging nitrogen for 5 minutes. To this mixture were added Pd(OAc)₂ (850 mg, 3 mmol, 0.1 eq) and BINAP (4.75 g, 7 mmol, 0.2 eq) and reaction mixture was again deoxygenated allowed by purging nitrogen for 10 minutes. Then the reaction mixture was stirred at 90° C. for 16 h. Progress of reaction was monitored by TLC. After completion, reaction mixture was diluted with water (200 mL) and extract with ethyl acetate (3×250 mL). Combined organic layer was washed with brine and dried over anhydrous sodium sulfate. Removal of solvent under reduced pressure afforded crude which was purified by Combi-Flash on silica gel using ethyl acetate-hexane system as eluent to afford (E)-N′-(2,3-dihydro-[1,4]dioxino[2,3-b]pyrazin-6-yl)-N,N-dimethylformimidamide (2.5 g, 31.60%).

### Step-4

To a solution of (E)-N′-(2,3-dihydro-[1,4]dioxino[2,3-b]pyrazin-6-yl)-N,N-dimethylformimidamide (4 g, 19 mmol, 1 eq) in EtOH (30 mL) was added ethylenediamine (2 mL, 38 mmol, 2 eq) at RT and the resulting reaction mixture was allowed to stir at 80° C. for 4 h. Progress of reaction was monitored by TLC. After completion, solvent was evaporated under reduced pressure, diluted with water (100 mL) and extracted with ethyl acetate (3×150 mL). Combined organic layer was washed with brine (100 mL) and dried over anhydrous sodium sulfate. Removal of solvent under reduced pressure afforded 2,3-dihydro-[1,4]dioxino[2,3-b]pyrazin-6-amine (2.85 g, 96.93%) as off white solid.

### Step-5

To a solution of 2,3-dihydro-[1,4]dioxino[2,3-b]pyrazin-6-amine (2 g, 13.05 mmol, 1 eq) in a 1:1 mixture of 48% aq. HBr in water (10 mL) and water at 0° C. was added a solution NaNO₂ (1.35 g, 19.58 mmol, 1.5 eq) portion-wise. The reaction mixture was allowed to stir at the same temperature for 5 minutes. To this mixture was then added CuBr (3.73 g, 26.11 mmol, 2 eq) under nitrogen atmosphere at 0° C. and the reaction mixture was allowed to stir at 0° C. for 1 h. Progress of reaction was monitored by TLC. After completion, reaction mixture was diluted with water (30 mL) and extracted with ethyl acetate (3×250 mL). Combined organic layer was washed with brine and dried over anhydrous sodium sulfate. Removal of solvent under reduced pressure afforded 6-bromo-2,3-dihydro-[1,4]dioxino[2,3-b]pyrazine (300 mg, 10.93%) as brown solid.

### Step-6

To a solution of 6-bromo-2,3-dihydro-[1,4]dioxino[2,3-b]pyrazine (250 mg, 1.15 mmol, 1 eq) in a mixture of 1,4-dioxane (5 mL) and H₂O (2 mL) were added (E)-ethyl 3-(4,4,5,5-tetramethyl-1,3,2-dioxaborolan-2-yl)acrylate (312.6 mg, 1.38 mmol, 1.2 eq) and t-BuOK (193.91 mg, 1.72 mmol, 1.5 eq) at room temperature. The reaction mixture was deoxygenated by purging nitrogen for 5 minutes. To this mixture was added (Ph₃P)₄Pd (66.6 mg, 0.05 mmol, 0.05 eq) and mixture was again deoxygenated by purging nitrogen for 10 minutes. The reaction mixture was stirred at 140° C. under microwave for 1 h. Progress of reaction was monitored by TLC. After completion, reaction mixture was diluted with water (50 mL) and extract with ethyl acetate (3×50 mL). Combined organic layer was washed with brine and dried over anhydrous sodium sulfate. Removal of solvent under reduced pressure afforded crude which was purified by Combi-Flash on silica gel using ethyl acetate-hexane system as eluent to afford (E)-ethyl 3-(2,3-dihydro-[1,4]dioxino[2,3-b]pyrazin-6-yl)acrylate (170 mg, 62.73%)

### Step-7

To a solution of (E)-ethyl 3-(2,3-dihydro-[1,4]dioxino[2,3-b]pyrazin-6-yl)acrylate (250 mg, 0.97 mmol, 1 eq) in EtOH (10 mL) at RT was added a solution of LiOH (116.54 mg, 4.86 mmol, 5 eq) in water (1 mL) and the resulting mixture was allowed to stir at 50° C. for 1 h. Progress of reaction was monitored by TLC. After completion, reaction mixture was concentrated to remove ethanol, washed with diethyl ether (2×10 mL), acidified with 1N HCl up to pH 6 and extracted with mixture of ethanol and ethyl acetate (1:4) (3×50 mL). Combined organic layer was washed with brine and dried over anhydrous sodium sulfate. Removal of solvent afforded crude (E)-3-(2,3-dihydro-[1,4]dioxino[2,3-b]pyrazin-6-yl)acrylic acid (170 mg, 77.27%) as white solid which was used in the next step without purification.

### Step-8

To a solution of (E)-3-(2,3-dihydro-[1,4]dioxino[2,3-b]pyrazin-6-yl)acrylic acid (0.170 g, 0.81 mmol, 1.0 eq.) in dry THF were added triethylamine (0.098 g, 0.97 mmol, 1.2 eq) and pivaloyl chloride (0.098 g, 0.81 mmol, 1 eq). The reaction mixture was stirred at 0° C. for 45 minutes. Progress of reaction was monitored by TLC. After completion, reaction mixture was filtered and the filtrate was used directly for the next step.

### Step-9

To a stirred solution of 5,6-dihydropyridin-2(1H)-one (0.079 g, 0.81 mmol, 1 eq.) in dry THF at −78° C. was added n-butyl lithium (2.5 M in hexane, 0.32 mL, 0.81 mmol, 1 eq) and the mixture was allowed to stir at the same temperature for 30 minutes. To this solution was added anhydride (0.238 g, 0.81 mmol, 1 eq). Reaction mixture was allowed to stir at −78° C. for 90 minutes. Progress of reaction was monitored by TLC. After completion, reaction mixture was brought to 0° C., diluted with ammonium chloride solution (30 mL) and extracted with ethyl acetate (3×50 mL). Combined organic layer was washed with brine, dried over anhydrous Na₂SO₄ and evaporated to dryness under vacuum to afford crude which was purified by Combi-Flash on silica gel using ethyl acetate-hexane as eluent to afford (E)-1-(3-(2,3-dihydro-[1,4]dioxino[2,3-b]pyrazin-6-yl)acryloyl)-5,6-dihydropyridin-2(1H)-one (60 mg, 26%).

LCMS: 288 [M+1]⁺

¹H NMR (400 MHz, CDCl₃) δ 7.95 (s, 1H), 7.70 (d, 1H), 7.59 (d, 1H), 6.95-6.88 (m, 1H), 6.03 (d, 1H), 4.57-4.44 (m, 4H), 4.02 (t, 2H), 2.50-2.40 (m, 2H).

### Example 65

### Preparation of 1-{[(E)-2-(pyrimidin-2-yl)ethenyl]sulfonyl}-5,6-dihydropyridin-2(1H)-one

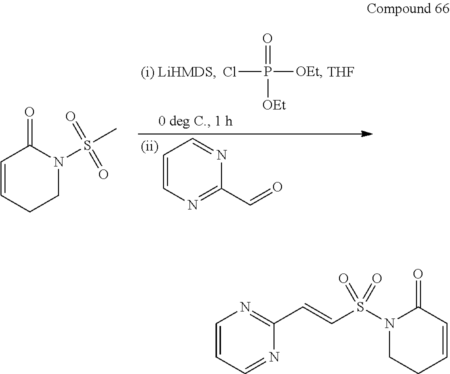 [SMILES: CCOP(=O)(Cl)OCC ; CS(=O)(=O)N1CCC=CC1=O ; O=Cc1ncccn1 ; O=C1C=CCCN1S(=O)(=O)/C=C/c1ncccn1 ; "THF"]

To a solution of 1-(methylsulfonyl)-5,6-dihydropyridin-2(1H)-one (150 mg, 0.85 mmol, 1.0 eq) and diethyl phosphorochloridate (147.8 mg, 0.85 mmol, 1 eq) in THF (10 mL) at 0° C. was added LiHMDS (1M in THF) (1.7 mL, 1.70 mmol, 2.0 eq) and the reaction mixture was stirred at 0° C. for 1 h. To this mixture was added a solution of pyrimidine-2-carbaldehyde (91.8 mg, 0.85 mmol, 1 eq) in THF (3 mL) and the reaction mixture was stirred at 0° C. for 1 h. Progress of reaction was monitored by TLC. Reaction mixture was quenched with saturated ammonium chloride solution (20 mL) and extracted with ethyl acetate (3×50 mL). Combined organic layer was washed with brine solution (50 mL) and dried over anhydrous Na₂SO₄. Removal of solvent under reduced pressure gave crude which was purified by Combi-Flash on silica gel using ethyl acetate-hexane as eluent to afford 1-{[(E)-2-(pyrimidin-2-yl)ethenyl]sulfonyl}-5,6-dihydropyridin-2(1H)-one (12 mg, 5.3%)

LCMS: 266 [M+1]⁺

¹H NMR (400 MHz, CD₃OD) δ 8.85 (d, 2H), 7.90 (d, 1H), 7.59 (d, 1H), 7.45 (t, 1H), 6.90-7.18 (m, 1H), 5.93 (d, 1H), 4.00 (t, 2H), 2.47-2.77 (m, 2H).

### Example 66

### Preparation of 1-{(2E)-3-[1-(pyrimidin-2-yl)piperidin-4-yl]prop-2-enoyl}-5,6-dihydropyridin-2(1H)-one

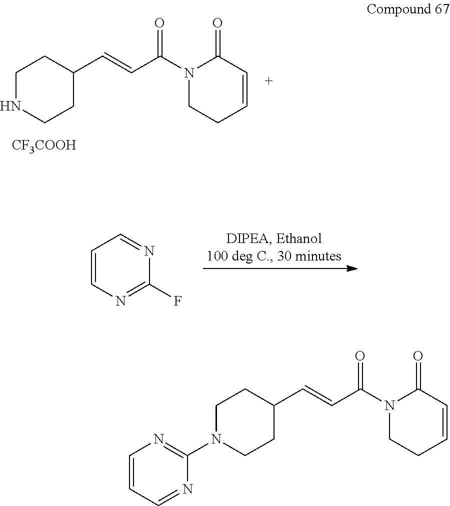 [SMILES: "CF3COOH" ; O=C1C=CCCN1C(=O)/C=C/C1CCNCC1 ; Fc1ncccn1 ; O=C1C=CCCN1C(=O)/C=C/C1CCN(c2ncccn2)CC1]

To a stirred solution of 1-[(2E)-3-(piperidin-4-yl)prop-2-enoyl]-5,6-dihydropyridin-2(1H)-one TFA salt (100 mg, 0.29 mmol, 1 eq) in ethanol (5 mL) was added DIPEA (0.22 mL, 1.281 mmol, 4.4 eq) and 2-fluoropyrimidine (45 mg, 0.469 mmol, 1.6 eq) and the reaction mixture was allowed to stir at 100° C. in microwave for 30 minutes. Progress of reaction was monitored by LCMS and TLC. After completion, reaction mixture was concentrated under reduced pressure to yield crude product which further purified by Combi-Flash on silica gel using ethyl acetate-hexane as eluent to yield 1-{(2E)-3-[1-(pyrimidin-2-yl)piperidin-4-yl]prop-2-enoyl}-5,6-dihydropyridin-2(1H)-one (30 mg, 23%).

LCMS: 313 [M+H]⁺

¹H NMR (400 MHz, CHCl₃) δ 8.29 (d, 2H), 6.92 (m, 3H), 7.00-6.80 (m, 3H), 6.42 (t, 1H), 6.00 (d, 1H), 4.80-4.70 (m, 2H), 3.98 (t, 2H), 3.02-2.88 (m, 2H), 2.60-2.39 (m, 3H), 1.90-1.80 (m, 2H), 1.63-1.40 (m, 2H).

### Example 67

### Preparation of (E)-4-(3-oxo-3-(6-oxo-3,6-dihydropyridin-1(2H)-yl)prop-1-en-1-yl)piperidine-1-carboxamide

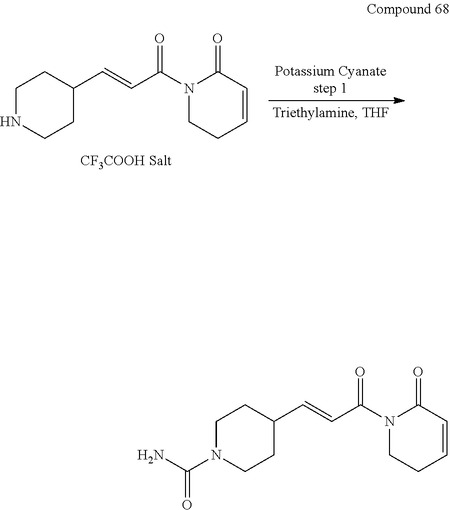

To a solution of the product of (E)-1-(3-(piperidin-4-yl)acryloyl)-5,6-dihydropyridin-2(1H)-one 2,2,2-trifluoroacetate (0.3 g, 0.862 mmol, 1.0 eq.) in tetrahydrofuran (20 mL) was added triethylamine (0.150 g, 2.586 mmol, 3.0 eq.) and potassium cyanate (0.209 g, 2.586 mmol, 3.0 eq.) and the reaction mixture was stirred at room temperature for 4 h. Progress of reaction was monitored by TLC. Reaction mixture was diluted with water (50 mL) and extracted with ethyl acetate (3×50 mL). Combined organic layer was washed with saturated aqueous sodium bicarbonate solution followed by brine (50 mL) and dried over anhydrous sodium sulfate. Removal of solvent under reduced pressure afforded crude which was purified by Combi-Flash on silica gel using ethyl acetate-hexane system as eluent to afford the (E)-4-(3-oxo-3-(6-oxo-3,6-dihydropyridin-1(2H)-yl)prop-1-en-1-yl)piperidine-1-carboxamide (0.030 g, 12.60% yield).

LCMS: 278 [M+1]⁺

¹H NMR (400 MHz, DMSO-d6) δ 7.03-7.10 (m, 1H), 6.74-6.82 (m, 1H), 6.70 (d, 1H), 5.88-5.95 (m, 2H), 3.92 (d, 2H), 3.83 (t, 2H), 3.16 (s, 1H), 2.65-2.76 (m, 2H), 2.42 (dt, 2H), 1.61-1.70 (m, 3H), 1.12-1.24 (m, 2H).

### Example 68

### Preparation of 1-[(2E)-2-methyl-3-(1-methyl-1H-pyrazol-4-yl)prop-2-enoyl]-5,6-dihydropyridin-2(1H)-one

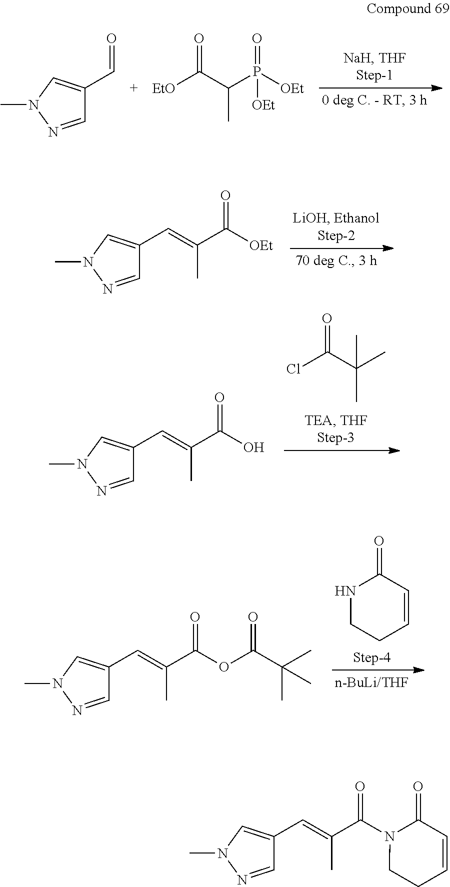

### Step-1

To a suspension of sodium hydride (60% in mineral oil), (472 mg, 11.8 mmol, 1.2 eq) in THF (50 mL) was added ethyl 2-(diethoxyphosphoryl)propanoate (2.16 g, 9.08 mmol, 1.2 eq) dropwise at 0° C. and the mixture was allowed to stir at the same temperature for 30 minutes. To this mixture was added a solution of 1-methyl-1H-pyrazole-4-carbaldehyde (1.0 g, 9.08 mmol, 1.0 eq) in THF (5 mL) and the resulting mixture was allowed to stir at room temperature for 2 h. Progress of reaction was monitored by TLC. After completion, reaction mixture was diluted with saturated aq. NH₄Cl (100 mL) and extracted with ethyl acetate (3×50 mL). Combined organic layer was washed with brine (50 mL), dried over anhydrous Na₂SO₄ and evaporated to dryness under vacuum to afford crude which was purified by Combi-Flash on silica gel using ethyl acetate-hexane system as eluent to afford ethyl (2E)-2-methyl-3-(1-methyl-1H-pyrazol-4-yl)prop-2-enoate (1.6 g, 90.91%).

### Step-2

To a solution of ethyl (2E)-2-methyl-3-(1-methyl-1H-pyrazol-4-yl)prop-2-enoate (1.6 g, 8.22 mmol, 1.0 eq) in ethanol (50 mL) was added a solution of lithium hydroxide (0.28 g, 11.65 mmol, 3.0 eq) in water (6.0 mL) and the reaction mixture was stirred at 70° C. for 2 h. Reaction mixture was concentrated under vacuum and then acidified with 1N HCl (10 mL). The mixture was diluted with water (30 mL) and extracted with ethyl acetate (3×50 mL). Combined organic layer was washed with brine solution (50 mL) and dried over anhydrous Na₂SO₄. Removal of solvent under reduced pressure gave (2E)-2-methyl-3-(1-methyl-H-pyrazol-4-yl)prop-2-enoic acid (1.0 g 73%) as crystalline solid.

### Step-3

To a solution of (2E)-2-methyl-3-(1-methyl-1H-pyrazol-4-yl)prop-2-enoic acid (200 mg, 1.20 mmol, 1.0 eq.) in dry THF (10 mL) was added triethylamine (0.35 mL, 2.76 mmol, 1.2 eq.) followed by pivaloyl chloride (0.20 mL, 1.44 mmol, 1.2 eq.) and the reaction mixture was stirred at 0° C. for 45 minutes. Progress of reaction was monitored by TLC. After completion, reaction mixture was filtered and the filtrate was used directly for the next step.

### Step-4

To a stirred solution of 5,6-dihydropyridin-2(1H)-one (100 mg, 1.03 mmol, 1.0 eq.) in dry THF (10 mL) at −78° C. was added nBuLi (0.50 mL, 1.13 mmol, 1.2 eq.) and the mixture was allowed to stir at the same temperature for 30 minutes. To this solution was added a solution of 2,2-dimethylpropanoic (2E)-2-methyl-3-(1-methyl-1H-pyrazol-4-yl)prop-2-enoic anhydride (205 mg, 1.23 mmol, 12 eq.) in THF and the reaction mixture was allowed to stir at room temperature for 1 h. Progress of reaction was monitored by TLC and LCMS. After completion, reaction mixture was evaporated to dryness under vacuum to afford crude which was purified combi-flash chromatography (0-50% EtOAc-Hexane) to afford 1-[(2E)-2-methyl-3-(1-methyl-1H-pyrazol-4-yl)prop-2-enoyl]-5,6-dihydropyridin-2(1H)-one (50 mg, 25%).

LCMS: 246[M+1]⁺

¹H NMR (400 MHz, CHCl₃) δ 7.63 (s, 1H), 7.52 (s, 1H), 6.94-7.07 (m, 1H), 6.91 (dd, 1H), 5.98 (d, 1H), 4.06-4.14 (m, 1H), 3.98 (s, 3H), 3.90 (t, 2H), 2.52 (d, 2H), 2.08 (s, 3H).

### Example 69

### Preparation of (E)-4-(3-(3,3-dimethyl-6-oxo-3,6-dihydropyridin-1(2H)-yl)-3-oxoprop-1-en-1-yl)-N-(4-fluorophenyl)piperidine-1-carboxamide

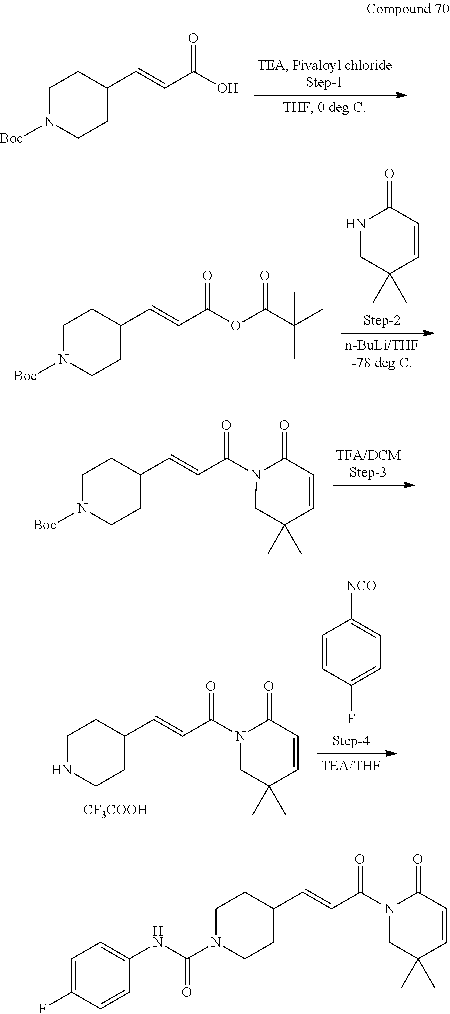

### Step-1

To a solution of (E)-3-(1-(tert-butoxycarbonyl)piperidin-4-yl)acrylic acid (0.193 g, 0.717 mmol, 1.0 eq.) in dry THF (10 mL) was added triethylamine (0.257 mL, 0.717 mmol, 1.0 eq.) followed by pivaloyl chloride (0.086 mL, 0.717 mmol, 1.0 eq.) and the reaction mixture was stirred at 0° C. for 45 minutes. Progress of reaction was monitored by TLC. After completion, reaction mixture was filtered and the filtrate was used directly for the next step.

### Step-2

To a stirred solution of 5,5-dimethyl-5,6-dihydropyridin-2(1H)-one (0.90 g, 0.72 mmol, 1.0 eq.) in dry THF (10 mL) at −78° C. was added n-butyllithium (2.5 M in hexane, 0.28 mL, 0.72 mmol, 1.0 eq.) and the mixture was allowed to stir at the same temperature for 30 minutes. To this solution was added a solution of (E)-(E)-3-(1-(tert-butoxycarbonyl)piperidin-4-yl)acrylic pivalic anhydride (0.254 g, 0.72 mmol, 1.0 eq.) in THF (10 mL) and the reaction mixture was allowed to stir at −78° C. for 1 h. Progress of reaction was monitored by TLC and LCMS. After completion, reaction mixture was brought to 0° C., diluted with water (30 mL) and extracted with ethyl acetate (3×30 mL). Combined organic layer was washed with brine, dried over anhydrous Na₂SO₄ and evaporated to dryness under vacuum to afford crude which was purified by reversed phase HPLC to afford tert-butyl (E)-4-(3-(3,3-dimethyl-6-oxo-3,6-dihydropyridin-1(2H)-yl)-3-oxoprop-1-en-1-yl)piperidine-1-carboxylate (0.2 g, 69.20%) was obtained as off white solid.

LCMS: 363 [M+1]⁺

Step-3 To a tert-butyl (E)-4-(3-(3,3-dimethyl-6-oxo-3,6-dihydropyridin-1(2H)-yl)-3-oxoprop-1-en-1-yl)piperidine-1-carboxylate (0.16 g, 0.48 mmol, 1.0 eq.) was dissolved in dichloromethane (10 mL) was added trifluoroacetic acid (3.0 mL) at 0° C. and resulting reaction mixture was stirred at room temperature for 2 h. Progress of reaction was monitored by TLC. After completion the reaction mixture was evaporated under reduce pressure to dryness, triturated with Et₂O to afford (E)-1-(3-(piperidin-4-yl)acryloyl)-5,6-dihydropyridin-2(1H)-one (0.015 g, 9.86%).

LCMS: 263 [M+1]⁺

### Step-4

To a suspension of (E)-1-(3-(piperidin-4-yl)acryloyl)-5,6-dihydropyridin-2(1H)-one (0.1 g, 0.381 mmol, 1.0 eq) in tetrahydrofuran (10 mL) was added triethylamine (1.39 mL, 0.762 mmol, 2.0 eq). The mixture was cooled to 0° C., and 1-fluoro-4-isocyanatobenzene (1.10 mL, 0.762 mmol, 2.0 eq) was added slowly. The reaction was allowed to warm to room temperature and stirred for 1 h. Progress of reaction was monitored by TLC. After completion, reaction mixture was diluted with water (30 mL) and extracted with ethyl acetate (3×30 mL). Combined organic layer was washed with brine, dried over anhydrous Na₂SO₄ and evaporated to dryness under vacuum to afford crude which was purified by Combi-Flash to using ethyl acetate-hexane system as eluent to afford (E)-4-(3-(3,3-dimethyl-6-oxo-3,6-dihydropyridin-1(2H)-yl)-3-oxoprop-1-en-1-yl)-N-(4-fluorophenyl)piperidine-1-carboxamide (0.015 g, 9.86%) was obtained as off white solid.

LCMS: 400 [M+1]⁺

¹H NMR (400 MHz, CDCl₃) δ ppm 7.27-7.32 (m, 2H), 6.98 (t, 2H), 6.92-6.94 (m, 1H), 6.64 (d, 1H), 6.29 (br. s., 1H), 5.84 (d, 1H), 4.07 (d, 2H), 3.74 (s, 2H), 2.92-3.04 (m, 2H), 2.45 (br. s., 1H), 1.88 (d, 2H), 1.43-1.53 (m, 2H), 1.21-1.32 (m, 3H), 1.15 (s, 5H), 0.89 (d, 1H).

### Example 70

### Preparation of (Z)-tert-butyl 4-(2-methyl-3-oxo-3-(2-oxo-5,6-dihydropyridin-1(2H)-yl)prop-1-enyl)piperidine-1-carboxylate

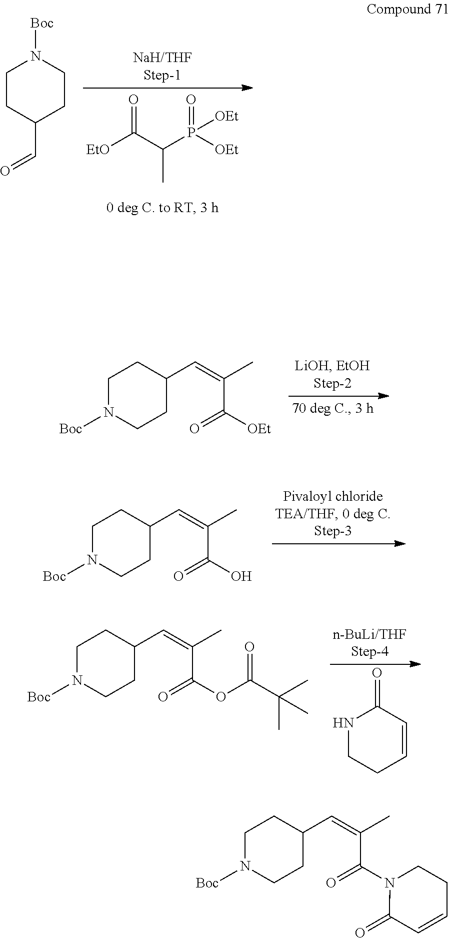

### Step-1

To a suspension of sodium hydride (60% in mineral oil, 0.487 g, 12.81 mmol, 1.3 eq) in THF (50 mL) was added ethyl 2-(diethoxyphosphoryl)propanoate (2.23 g, 9.37 mmol, 1.0 eq) dropwise at 0° C. and the mixture was allowed to stir at the same temperature for 30 minutes. To this mixture was added a solution of tert-butyl 4-formylpiperidine-1-carboxylate (2.0 g, 9.37 mmol, 1.0 eq) in THF (5 mL) and the resulting mixture was allowed to stir at RT for 2 h. Progress of reaction was monitored by TLC. After completion, reaction mixture was diluted with saturated aq. NH₄Cl (100 mL) and extracted with ethyl acetate (3×50 mL). Combined organic layer was washed with brine, dried over anhydrous Na₂SO₄ and evaporated to dryness under vacuum to afford crude which was purified by Combi-Flash on silica gel using ethyl acetate-hexane system as eluent to afford (Z)-tert-butyl 4-(3-ethoxy-2-methyl-3-oxoprop-1-enyl)piperidine-1-carboxylate (1.34 g, 48.2%).

LCMS: 298 [M+1]⁺

### Step-2

To a solution of (Z)-tert-butyl 4-(3-ethoxy-2-methyl-3-oxoprop-1-enyl)piperidine-1-carboxylate (0.75 g, 2.52 mmol, 1.0 eq) in ethanol (8.0 mL) was added lithium hydroxide (0.643 g, 8.0 mmol, 3.0 eq) in water (1.0 ml) and the reaction mixture was stirred at 70° C. for 2 h.

Reaction mixture was concentrated under vacuum and then acidified with 1N HCl. The mixture was diluted with water and extracted with ethyl acetate (3×50 mL). The combined organic layer was washed with brine solution (50 mL) and dried over anhydrous Na₂SO₄. Removal of solvent under reduced pressure to obtain (Z)-tert-butyl 4-(3-ethoxy-2-methyl-3-oxoprop-1-enyl)piperidine-1-carboxylate (0.570 g, 83.82%) as crystalline solid.

LCMS: 270 [M+1]⁺

### Step-3

To a solution of (Z)-tert-butyl 4-(3-ethoxy-2-methyl-3-oxoprop-1-enyl)piperidine-1-carboxylate (0.20 g, 0.74 mmol, 1.0 eq.) in dry THF (10 mL) was added triethylamine (0.124 mL, 0.089 mmol, 1.2 eq.) followed by pivaloyl chloride (0.091 mL, 0.74 mmol, 1.0 eq.) and the reaction mixture was stirred at 0° C. for 45 minutes. Progress of reaction was monitored by TLC. After completion, reaction mixture was filtered through syringe filter and the filtrate was used directly for the next step.

### Step-4

To a stirred solution of 5,6-dihydropyridin-2(1H)-one (0.071 g, 0.74 mmol, 1.0 eq.) in dry THF (20 mL) at −78° C. was added n-BuLi (0.30 mL, 0.74 mmol, 1.0 eq.) and the mixture was allowed to stir at the same temperature for 30 minutes. To this solution was added a solution of respective anhydride (0.262 g, 0.74 mmol, 1.0 eq.) in THF and the reaction mixture was allowed to stir at RT for 1 h. Progress of reaction was monitored by TLC and LCMS. After completion, reaction mixture was evaporated to dryness under vacuum to afford crude which was purified by reversed phase HPLC to afford (Z)-tert-butyl 4-(2-methyl-3-oxo-3-(2-oxo-5,6-dihydropyridin-1(2H)-yl)prop-1-enyl)piperidine-1-carboxylate (0.13 g, 49.6%).

LCMS: 349 [M+1]⁺

¹H NMR (400 MHz, DMSO-d₆) δ 6.90-7.00 (m, 1H), 6.00 (d, 1H), 5.18 (d, 1H), 4.10-3.92 (m, 4H), 2.70-2.55 (m, 2H), 2.50-2.40 (m, 2H), 2.20-2.05 (m, 1H), 1.91 (s, 3H), 1.60-1.50 (m, 4H), 1.30-1.18 (m, 7H), 0.90-0.78 (m, 2H).

### Example 71

### Preparation of tert-butyl (E)-4-(2-methyl-3-oxo-3-(6-oxo-3,6-dihydropyridin-1(2H)-yl)prop-1-en-1-yl)piperidine-1-carboxylate

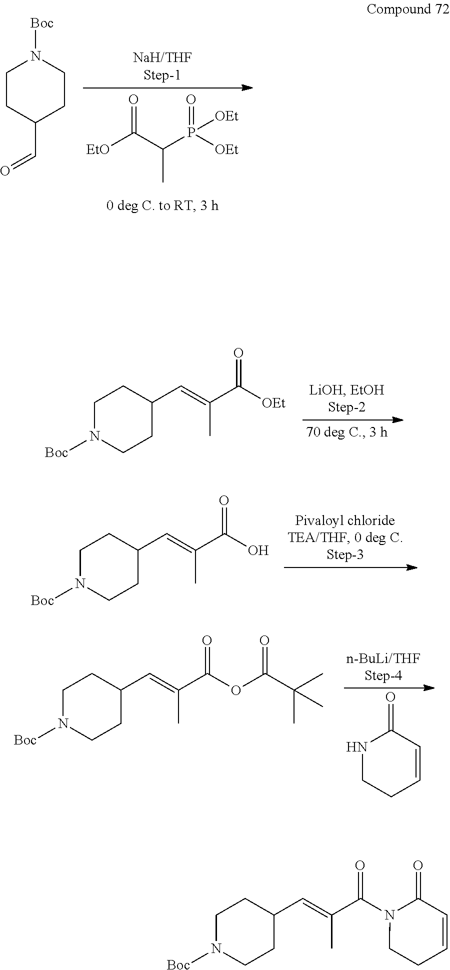

### Step-1

To a suspension of sodium hydride (60% in mineral oil, 0.487 g, 12.81 mmol, 1.3 eq) in THF (50 mL) was added ethyl 2-(diethoxyphosphoryl)propanoate (2.23 g, 9.37 mmol, 1.0 eq) dropwise at 0° C. and the mixture was allowed to stir at the same temperature for 30 minutes. To this mixture was added a solution of tert-butyl 4-formylpiperidine-1-carboxylate (2.0 g, 9.37 mmol, 1.0 eq) in THF (5 mL) and the resulting mixture was allowed to stir at RT for 2 h. Progress of reaction was monitored by TLC. After completion, reaction mixture was diluted with saturated aq. NH₄Cl (100 mL) and extracted with ethyl acetate (3×50 mL). Combined organic layer was washed with brine, dried over anhydrous Na₂SO₄ and evaporated to dryness under vacuum to afford crude which was purified by Combi-Flash on silica gel using ethyl acetate-hexane system as eluent to afford (Z)-tert-butyl 4-(3-ethoxy-2-methyl-3-oxoprop-1-enyl)piperidine-1-carboxylate (1.34 g, 48.2%).

LCMS: 298 [M+1]⁺

### Step-2

To a solution of tert-butyl 4-[(1E)-3-ethoxy-2-methyl-3-oxoprop-1-en-1-yl]piperidine-1-carboxylate (0.55 g, 1.85 mmol, 1.0 eq) in ethanol (5 mL) was added lithium hydroxide (0.543 g, 5.5 mmol, 3.0 eq) in water (1 mL) and the reaction mixture was stirred at 70° C. for 2 h. Reaction mixture was concentrated under vacuum and then acidified with 1N HCl. The mixture was diluted with water and extracted with ethyl acetate (3×50 mL). Combined organic layer was washed with brine solution (50 mL) and dried over anhydrous Na₂SO₄. Removal of solvent under reduced pressure gave (2E)-3-[1-(tert-butoxycarbonyl)piperidin-4-yl]-2-methylprop-2-enoic acid (0.24 g, 48.2%) as crystalline solid which was used in the next step without purification.

LCMS: 270 [M+1]⁺

### Step-3

To a solution of (2E)-3-[1-(tert-butoxycarbonyl)piperidin-4-yl]-2-methylprop-2-enoic acid (0.23 g, 0.85 mmol, 1 eq) in dry THF (20 mL) was added triethylamine (0.142 mL, 1.02 mmol, 1.2 eq.) followed by pivaloyl chloride (0.105 mL, 2.3 mmol, 1.0 eq.) and the reaction mixture was stirred at 0° C. for 45 minutes. Progress of reaction was monitored by TLC. After completion, reaction mixture was filtered and the filtrate was used directly for the next step.

### Step-4

To a stirred solution of 5,6-dihydropyridin-2(1H)-one (0.82 g, 0.84 mmol, 1.0 eq.) in dry THF (20 mL) at −78° C. was added n-BuLi (0.34 mL, 0.84 mmol, 1 eq) and the mixture was allowed to stir at the same temperature for 30 minutes. To this solution was added a solution of anhydride (0.30 g, 0.84 mmol, 1.0 eq.) in THF and the reaction mixture was allowed to stir at RT for 1 h. Progress of reaction was monitored by TLC and LCMS. After completion, reaction mixture was evaporated to dryness under vacuum to afford crude which was purified by reversed phase HPLC to afford tert-butyl (E)-4-(2-methyl-3-oxo-3-(6-oxo-3,6-dihydropyridin-1(2H)-yl)prop-1-en-1-yl)piperidine-1-carboxylate (0.2 g, 67.8%).

LCMS: 349 [M+1]⁺

¹H NMR (400 MHz, CDCl₃) δ 6.85-6.95 (m, 1H), 5.96 (d, 1H), 5.70 (d, 1H), 4.07 (brs, 2H), 3.83 (t, 2H), 2.85-2.70 (m, 2H), 2.50-2.37 (m, 2H), 1.87 (s, 3H), 1.70-1.58 (m, 2H), 1.42 (s, 9H), 1.39-1.20 (m, 3H).

### Example 72

### Preparation of (E)-N-(4-fluorophenyl)-4-(2-methyl-3-oxo-3-(6-oxo-3,6-dihydropyridin-1(2H)-yl)prop-1-en-1-yl)piperidine-1-carboxamide

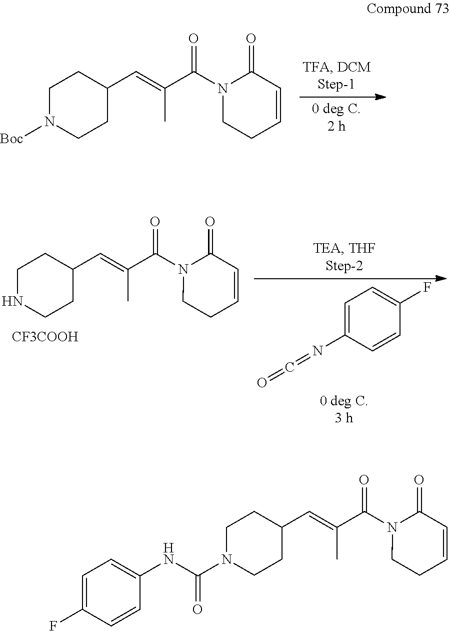 [SMILES: C/C(=C\C1CCN(C(=O)OC(C)(C)C)CC1)C(=O)N1CCC=CC1=O ; "CF3COOH" ; C/C(=C\C1CCNCC1)C(=O)N1CCC=CC1=O ; O=C=Nc1ccc(F)cc1 ; C/C(=C\C1CCN(C(=O)Nc2ccc(F)cc2)CC1)C(=O)N1CCC=CC1=O]

### Step-1

To a solution of tert-butyl 4-[(1E)-3-ethoxy-3-oxoprop-1-en-1-yl]piperidine-1-carboxylate (0.20 g, 0.57 mmol, 1.0 eq) in DCM (10 mL) was added TFA (0.196 mL, 0.17 mmol, 3.0 eq) at 0° C. and RM was allowed to stir at the same temperature for 2 h. Progress of reaction was monitored by TLC. After completion, solvent was removed under reduced pressure to obtain (E)-1-(2-methyl-3-(piperidin-4-yl)acryloyl)-5,6-dihydropyridin-2(1H)-one (0.11 g, 55.0%) as solid which was used in the next step without purification.

LCMS: 249 [M+1]⁺

### Step-2

To a solution of (E)-1-(2-methyl-3-(piperidin-4-yl)acryloyl)-5,6-dihydropyridin-2(1H)-one (110 mg, 0.30 mmol, 1.0 eq.) in dry THF (15 mL) was added DIPEA (0.08 mL, 0.45 mmol, 1.5 eq) followed by 1-fluoro-4-isocyanatobenzene (62 mg, 0.45 mmol, 1.5 eq) and the reaction mixture was stirred at RT for 3 h. Progress of reaction was monitored by TLC. After completion, reaction mixture was diluted with water (20 mL) and extracted with ethyl acetate (3×50 mL). Combined organic layer was washed with brine, dried over anhydrous Na₂SO₄ and evaporated to dryness under vacuum to afford crude which was purified by Combi-Flash on silica gel using ethyl acetate-hexane system as eluent to afford (E)-N-(4-fluorophenyl)-4-(2-methyl-3-oxo-3-(6-oxo-3,6-dihydropyridin-1(2H)-yl)prop-1-en-1-yl)piperidine-1-carboxamide.

LCMS: 386 [M+1]⁺

¹H NMR (400 MHz, DMSO-d₆) δ 8.50 (brs, 1H), 7.48-7.40 (m, 2H), 7.10-7.00 (m, 3H), 5.88 (d, 1H), 5.67 (d, 1H), 4.10-4.00 (m, 2H), 3.68 (t, 2H), 2.90-2.80 (m, 2H), 2.40-2.50 (m, 2H), 1.80 (t, 3H), 1.60-1.52 (m, 2H), 1.30-1.15 (m, 3H).

### Example 73

### Preparation of (E)-1-(3-(1-(pyridin-2-yl)piperidin-4-yl)acryloyl)-5,6-dihydropyridin-2(1H)-one

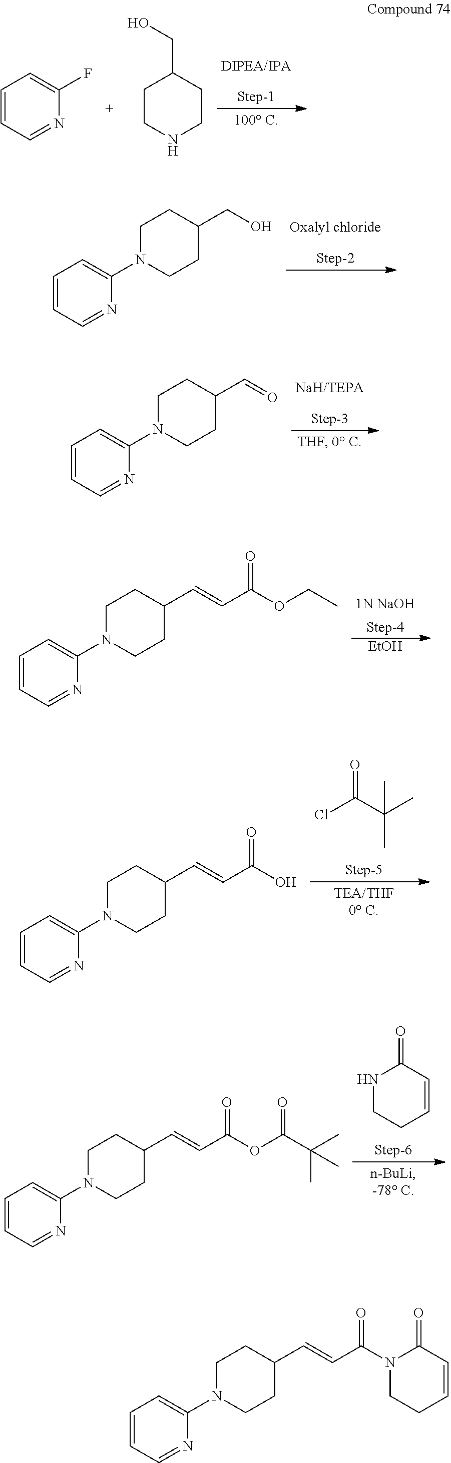

### Step-1

To a solution of 2-fluoropyridine (2 g, 20.61 mmol, 1 eq) in 2-propanol (20 mL) were added DIPEA (10.77 mL, 61.83 mmol, 3 eq) and piperidin-4-ylmethanol (2.38 g, 20.61 mmol, 1 eq) and the reaction mixture was allowed to stir at 100° C. for 3 h. Progress of reaction was monitored by NMR. After completion reaction mixture was diluted with water extracted with ethyl acetate (3×20 mL). Combined organic layer was washed with brine (50 mL) and dried over anhydrous sodium sulfate. Removal of solvent afforded crude (1-(pyridin-2-yl)piperidin-4-yl)methanol (1.5 g, 37.97%) which was used in the next step without purification.

### Step-2

To a solution of oxalyl chloride (1.34 mL, 15.62 mmol, 2 eq) in DCM (50 mL) at −78° C. was added DMSO (2.18 mL, 30.86 mmol, and 3.95 eq.) dropwise and the mixture was allowed to stir at the same temperature for 30 minutes. To this solution was added (1-(pyridin-2-yl)piperidin-4-yl)methanol (1.5 g, 7.81 mmol, 1 eq.). Reaction mixture was allowed to stir at −78° C. for 45 min. After that add TEA (4.30 mL, 30.86 mmol, 3.95 eq.) at same temperature. Reaction mixture was allowed to stir at RT for 30 minutes. Progress of reaction was monitored by TLC. Reaction mixture was diluted with water (30 mL) and extracted with DCM (3×40 mL). Combined organic layer was washed with brine and dried over anhydrous sodium sulfate. Removal of solvent to afford crude reaction mixture was purified by Combi-Flash to afford 1-(pyridin-2-yl)piperidine-4-carbaldehyde (1 g, 67.56%). Step-3

To a solution of triethylphosphonoacetate (1.25 mL, 6.31 mmol, 1.2 eq) in THF (40 mL) at 0° C. was added NaH (0.315 g, 7.89 mmol, and 1.5 eq.) portion-wise and the mixture was allowed to stir at the same temperature for 20 minutes. To this solution was added 1-(pyridin-2-yl)piperidine-4-carbaldehyde (1 g, 5.26 mmol, 1 eq.). Reaction mixture was allowed to stir at 0° C. for 15 min followed by stirring at RT for 1 h. Progress of reaction was monitored by TLC. Reaction mixture was diluted with water (30 mL) and extracted with ethyl acetate (3×50 mL). Combined organic layer was washed with brine and dried over anhydrous sodium sulfate. Removal of solvent to afford crude reaction mixture was purified by Combi-Flash to afford ethyl (E)-3-(1-(pyridin-2-yl)piperidin-4-yl)acrylate (0.650 g, 47%).

### Step-4

To a solution of ethyl (E)-3-(1-(pyridin-2-yl)piperidin-4-yl)acrylate (0.650 g, 2.5 mmol, 1 eq) in ethanol (10 mL) was added 1N NaOH (5 mL, 5.0 mmol, and 2 eq.) dropwise and the mixture was allowed to stir at the RT for 4 h. Progress of reaction was monitored by TLC. Reaction mixture was acidifying using 1N HCl up to pH 6 and extracted using mixture of ethanol and ethyl acetate (20:80) (3×50 mL). Combined organic layer was washed with brine and dried over anhydrous sodium sulfate. Removal of solvent afforded crude (E)-3-(1-(pyridin-2-yl)piperidin-4-yl)acrylic acid (0.3 g, 51.72%).

### Step-5

To a solution of (E)-3-(1-(pyridin-2-yl)piperidin-4-yl)acrylic acid (0.1 g, 0.430 mmol, 1.0 eq.) in dry THF was added triethylamine (0.071 mL, 0.516 mmol, 1.2 eq.) and pivaloyl chloride (0.047 mL, 0.387 mmol, 0.9 eq.) the reaction mixture was stirred at 0° C. for 45 minutes. Progress of reaction was monitored by TLC. After completion, reaction mixture was filtered and the filtrate was used directly for the next step.

### Step-6

To a stirred solution of 5,6-dihydropyridin-2(1H)-one (0.041 g, 0.430 mmol, 1 eq.) in dry THF at −78° C. was added n-butyl lithium (2.5 M in hexane, 0.17 mL, 0.430 mmol, 1 eq.) and the mixture was allowed to stir at the same temperature for 30 minutes. To this solution was added (E)-pivalic (E)-3-(1-(pyridin-2-yl)piperidin-4-yl)acrylic anhydride (0.136 g, 0.430 mmol, 1 eq.). Reaction mixture was allowed to stir at −78° C. for 90 min. Progress of reaction was monitored by TLC. Reaction mixture was brought to 0° C., diluted with ammonium chloride solution (30 mL) and extracted with ethyl acetate (3×40 mL). combined organic layer was washed with brine, dried over anhydrous Na₂SO₄ and evaporated to dryness under vacuum to afford crude which was purified by Combi-Flash to afford (E)-1-(3-(1-(pyridin-2-yl)piperidin-4-yl)acryloyl)-5,6-dihydropyridin-2(1H)-one (30 mg, 22.56%).

LCMS: 312 [M+1]⁺

¹H NMR (400 MHZ, CDCl3) δ 8.19 (d, 1H), 7.43 (d, 1H), 7.00-6.80 (m, 3H), 6.65 (d, 1H), 6.60-6.55 (m, 1H), 6.00 (d, 1H), 4.36-4.24 (m, 2H), 3.98 (t, 2H), 2.93-2.82 (m, 2H), 2.50-2.39 (m, 3H), 1.90-1.80 (m, 2H), 1.80-1.40 (m, 2H).

### Example 74

### Preparation of 1-{[(E)-2-(1-methyl-1H-pyrazol-4-yl)ethenyl]sulfonyl}-5,6-dihydropyridin-2(1H)-one

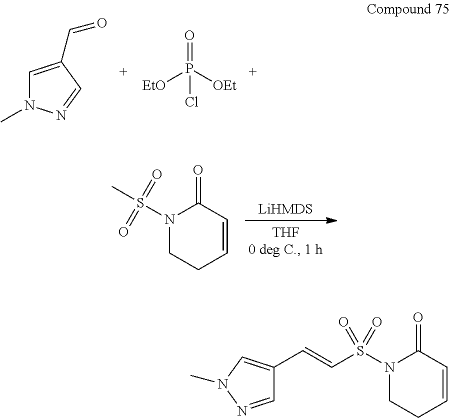

To a solution of 1-(methylsulfonyl)-5,6-dihydropyridin-2(1H)-one (200 mg, 1.14 mmol, 1.0 eq), diethyl phosphorochloridate (197 mg, 1.14 mmol, 1 eq) and 1-methyl-1H-pyrazole-4-carbaldehyde (100 mg, 0.91 mmol, 0.8 eq) in THF (10 mL) at 0° C. was added LiHMDS 1M in THF (2.5 mL, 2.50 mmol, 2.0 eq). The reaction mixture was stirred at 0° C. for 1 h. Progress of reaction was monitored by TLC. Reaction mixture was quenched by saturated ammonium chloride solution 20 mL and extracted with ethyl acetate (3×50 mL). Combined organic layer was washed with brine solution (50 mL) and dried over anhydrous Na₂SO₄. Removal of solvent under reduced pressure gave the crude which was purified by flash column chromatograpy (0-70% EtOAc-hexane) as eluent to 1-{[(E)-2-(1-methyl-H-pyrazol-4-yl)ethenyl]sulfonyl}-5,6-dihydropyridin-2(1H)-one (90 mg, 29.50%).

LCMS: 268[M+1]⁺

¹H NMR (400 MHz, CDCl₃) δ 7.69 (s, 1H), 7.59 (s, 1H), 7.55 (d, 1H), 6.91 (d, 1H), 6.81-6.86 (m, 1H), 5.97 (d, 1H), 3.96 (t, 2H), 3.92 (s, 3H), 2.51-2.57 (m, 2H),

### Example 75

### Preparation of 1-{[(E)-2-(8-methoxy-2,3-dihydro-1,4-benzodioxin-6-yl)ethenyl]sulfonyl}-5,6-dihydropyridin-2(1H)-one

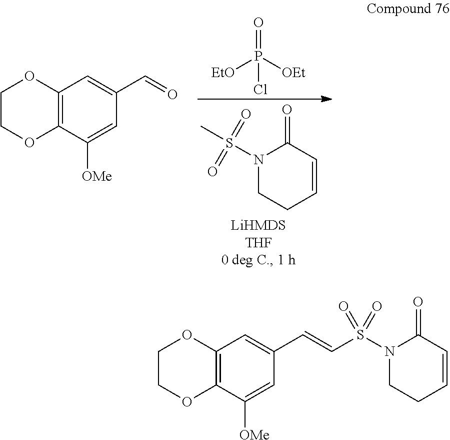

To a solution of 1-(methylsulfonyl)-5,6-dihydropyridin-2(1H)-one (250 mg, 1.43 mmol, 1.0 eq), diethyl phosphorochloridate (247 mg, 1.43 mmol, 1 eq) and 8-methoxy-2,3-dihydro-1,4-benzodioxine-6-carbaldehyde (222 mg, 1.14 mmol, 0.8 eq) in THF (10 mL) at 0° C. was added LiHMDS 1M in THF (3.15 mL, 3.15 mmol, 2.2 eq). The reaction mixture was stirred at 0° C. for 1 h. Progress of reaction was monitored by TLC. Reaction mixture was quenched by saturated ammonium chloride solution 20 mL and extracted with ethyl acetate (3×50 mL). Combined organic layer was washed with brine solution (50 mL) and dried over anhydrous Na₂SO₄. Removal of solvent under reduced pressure gave the crude which was purified by Combi-Flash on silica gel using ethyl acetate-hexane system as eluent to afford 1-{[(E)-2-(8-methoxy-2,3-dihydro-1,4-benzodioxin-6-yl)ethenyl]sulfonyl}-5,6-dihydropyridin-2(1H)-one (20 mg, 5%).

LCMS: 352[M+1]⁺

¹H NMR (400 MHz, CDCl3) δ 7.51 (d, 1H), 7.23 (d, 1H), 6.89-5.99 (m, 1H), 6.74 (s, 1H), 6.65 (s, 1H), 6.00 (d, 1H), 4.40-4.23 (m, 4H), 3.97 (t, 2H), 3.89 (s, 3H), 2.60-2.50 (m, 2H).

### Example 76

### Preparation of (E)-1-(3-(1-(cyclopropylmethyl)piperidin-4-yl)-2-methylacryloyl)-5,6-dihydropyridin-2(1H)-one

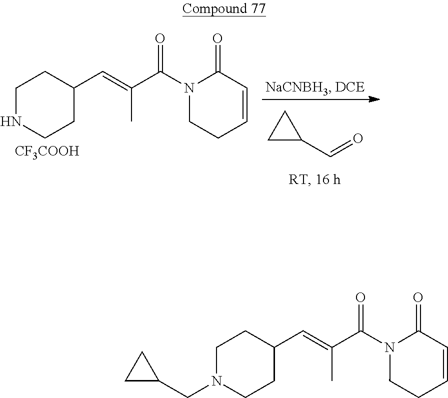

To a solution of (E)-1-(2-methyl-3-(piperidin-4-yl)acryloyl)-5,6-dihydropyridin-2(1H)-one 2,2,2-trifluoroacetate (80 mg, 0.22 mmol, 1.0 eq) in DCE (15 mL) was added (0.08 mL, 1.1 mmol, 5 eq) followed by AcOH (39 mg, 0.66 mmol, 3 eq) and the reaction mixture was stirred at RT for 1 h. To this mixture was added NaCNBH₃ (41 mg, 0.66 mmol, 3 eq) and the resulting mixture was allowed to stir for 16 h. Progress of reaction was monitored by TLC. After completion, reaction mixture was diluted with aq. sodium bicarbonate (20 mL) and extracted with ethyl acetate (3×50 mL). Combined organic layer was washed with brine, dried over anhydrous Na₂SO₄ and evaporated to dryness under vacuum to afford crude which was purified by reversed phase HPLC to afford (E)-1-(3-(1-(cyclopropylmethyl)piperidin-4-yl)-2-methylacryloyl)-5,6-dihydropyridin-2(1H)-one (10 mg, 15%).

LCMS: 303 [M+1]⁺

¹H NMR (400 MHz, CD₃OD) δ 7.10-7.00 (m, 1H), 5.91 (d, 1H), 5.70 (d, 1H), 3.80 (t, 2H), 3.42-3.35 (m, 2H), 2.80-2.42 (m, 6H), 1.98-1.80 (m, 6H), 1.70-1.50 (m, 2H), 1.10-0.95 (m, 1H), 0.70-0.65 (m, 2H), 0.38-0.30 (m, 2H).

### Example 77

### Preparation of (Z)-1-(3-(1-(4-fluorophenyl)piperidin-4-yl)-2-methylacryloyl)-5,6-dihydropyridin-2(1H)-one

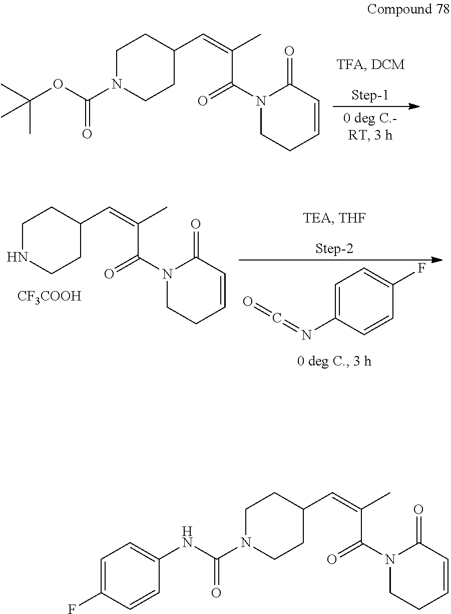

### Step-1

To a solution of tert-butyl 4-[(1E)-3-ethoxy-3-oxoprop-1-en-1-yl]piperidine-1-carboxylate (0.40 g, 1.14 mmol, 1.0 eq) in DCM (20 mL) was added CF₃COOH (2.0 mL, 3.44 mmol, 3.0 eq) at 0° C. and reaction mixture was allowed to stir at the same temperature for 10 minutes. Ice batch was removed and then reaction mixture was allowed to stir at RT for 3 h. Progress of reaction was monitored by TLC. After completion, removal of CF₃COOH under reduced pressure gave ethyl (2E)-3-(piperidin-4-yl)prop-2-enoate (0.2 g, 57.7%) as solid which was used in the next step without purification.

LCMS: 249 [M+1]⁺

### Step-2

To a solution of (Z)-1-(2-methyl-3-(piperidin-4-yl)acryloyl)-5,6-dihydropyridin-2(1H)-one 2,2,2-trifluoroacetate (0.1 g, 0.28 mmol, 1.0 eq.) in dry THF (15 mL) was added TEA (0.057 mL, 0.41 mmol, 1.5 eq.) followed by 1-fluoro-4-isocyanatobenzene (0.56 g, 0.41 mmol, 1.5 eq) and the reaction mixture was stirred at RT for 3 h. Progress of reaction was monitored by TLC. After completion, reaction mixture was diluted with water (20 mL) and extracted with ethyl acetate (3×50 mL). Combined organic layer was washed with brine, dried over anhydrous Na₂SO₄ and evaporated to dryness under vacuum to afford crude which was purified by Combi-Flash on silica gel using ethyl acetate-hexane system as eluent to afford (Z)-1-(3-(1-(4-fluorophenyl)piperidin-4-yl)-2-methylacryloyl)-5,6-dihydropyridin-2(1H)-one (65 mg, 61%).

LCMS: 386 [M+1]⁺

¹H NMR (400 MHz, DMSO-d₆) δ 8.43 (s, 1H), 7.45-7.39 (m, 2H), 7.16-6.90 (m, 3H), 5.95 (d, 1H), 5.11 (d, 1H), 4.08-3.97 (m, 2H), 3.90-3.80 (m, 2H), 2.78-2.40 (m, 2H), 2.22-2.10 (m, 1H), 1.60 (s, 3H), 1.45-1.10 (m, 4H).

### Example 78

### Preparation of (Z)-1-(3-(1-(cyclopropylmethyl)piperidin-4-yl)-2-methylacryloyl)-5,6-dihydropyridin-2(1H)-one

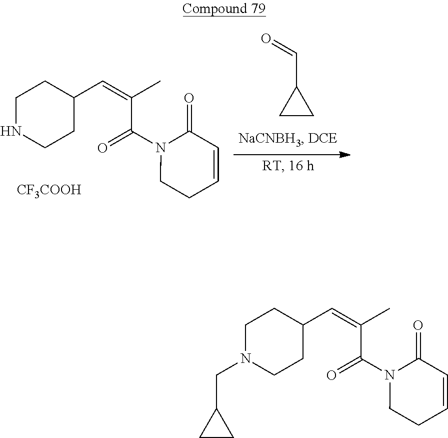 [SMILES: "CF3COOH" ; C/C(=C/C1CCNCC1)C(=O)N1CCC=CC1=O ; O=CC1CC1 ; C/C(=C/C1CCN(CC2CC2)CC1)C(=O)N1CCC=CC1=O]

### Step-1

To a solution of (Z)-1-(2-methyl-3-(piperidin-4-yl)acryloyl)-5,6-dihydropyridin-2(1H)-one 2,2,2-trifluoroacetate (0.05 g, 0.14 mmol, 1.0 eq.) in DCE (15 mL) was added (0.048 mL, 0.70 mmol, 5.0 eq.) followed by AcOH (24 mg, 0.40 mmol, 3.0 eq) and the reaction mixture was stirred at RT for 1 h. To this mixture was added NaCNBH₃ (29 mg, 0.46 mmol, 3.4 eq) and reaction mixture was allowed to stir for 16 h. Progress of reaction was monitored by TLC. After completion, reaction mixture was diluted with aq. sodium bicarbonate (20 mL) and extracted with ethyl acetate (3×50 mL). Combined organic layer was washed with brine, dried over anhydrous Na₂SO₄ and evaporated to dryness under vacuum to afford crude which was purified by reversed phase HPLC to afford (Z)-1-(3-(1-(cyclopropylmethyl)piperidin-4-yl)-2-methylacryloyl)-5,6-dihydropyridin-2(1H)-one (20 mg, 50%).

LCMS: 303 [M+1]⁺

¹H NMR (400 MHz, CD₃OD) δ 8.58 (brs, 1H), 7.18-7.02 (m, 1H), 5.98 (d, 1H), 5.18 (d, 1H), 3.98 (t, 2H), 3.30-3.00 (m, 2H), 2.60-2.40 (m, 4H), 2.39-2.33 (m, 2H), 2.21-2.10 (m, 1H), 2.87 (s, 3H), 1.75-1.40 (m, 4H), 1.02-0.90 (m, 1H), 0.62-0.55 (m, 2H), 0.22-0.17 (m, 2H).

### Example 79

### Preparation of (E)-1-(3-(6,7-dihydro-[1,4]dioxino[2,3-d]pyrimidin-2-yl)acryloyl)-5,6-dihydropyridin-2(1H)-one

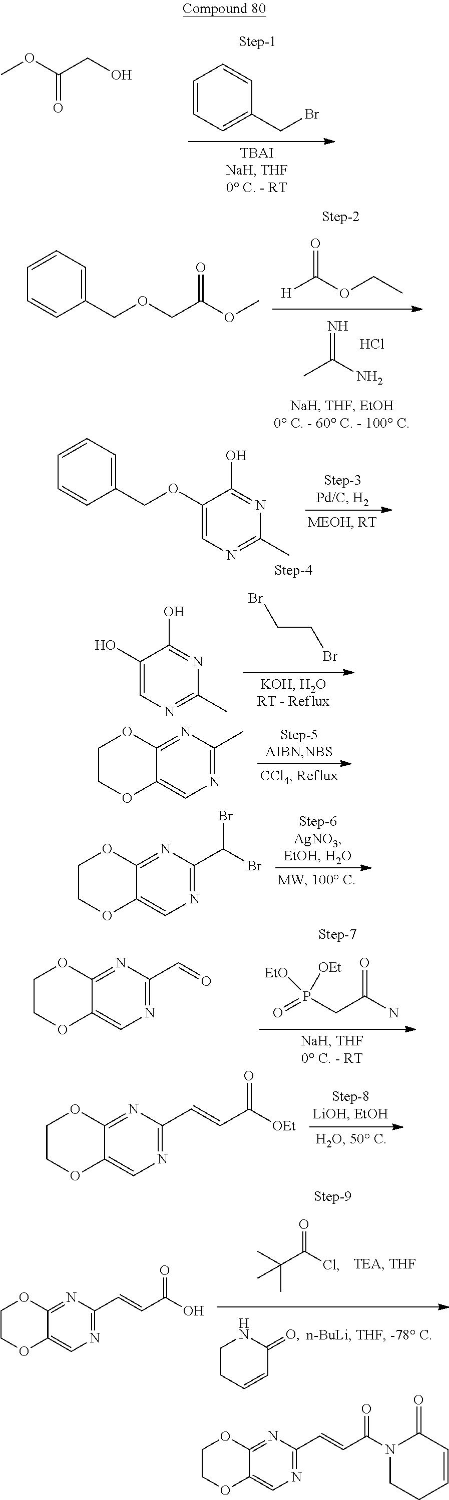

### Step-1

To a solution of methyl 2-hydroxyacetate (20 g, 0.22 mol, 1.0 eq.) in dry THF (250 mL) was added NaH (60% in mineral oil) (6.4 g, 0.26 mol, 1.2 eq.) at 0° C. over a period of 15 minutes. The reaction mixture was allowed to stir at that temperature for 30 minutes. To that stirred solution were added TBAI (8.20 g, 0.022 mol, 0.1 eq.) followed by (bromomethyl)benzene (38 g, 0.22 mol, 1.0 eq.) at that temperature and allowed to stir at RT for 16 h. Progress of reaction was monitored by TLC (10% ethyl acetate-hexane). After completion, reaction mixture was diluted with ice-cold water (100 mL) and extracted with ethyl acetate (3×250 mL). Combined organic layer was washed with brine and dried over anhydrous sodium sulfate. Removal of solvent under reduced pressure afforded crude which was purified by Combi-Flash to afford methyl 2-(benzyloxy)acetate (28.4 g, 71%).

### Step-2

To a solution of methyl 2-(benzyloxy)acetate (28.4 g, 0.157 mol, 1.0 eq.) and ethyl formate (11.67 g, 0.157 mol, 1.0 eq.) in dry THF (250 mL) was added NaH (60% in mineral oil) (4.92 g, 0.207 mol, 1.3 eq.) at 0° C. over a period of 15 minutes. The reaction mixture was allowed to stir at 50° C. for 30 minutes. Excess of solvent was evaporated under reduced pressure to get an oily residue. Sodium ethoxide was prepared by using Na (3.62 g, 0.157 mol, 1.0 eq.) and ethanol (150 mL) at 0° C. To it, acetimidamide.HCl (14.91 g, 0.157 mol, 1.0 eq.) was added at that temperature and allowed to stir at RT for 30 minutes. Acetimidamide solution in ethanol was filtered to remove solid and then it was added to the oily residue. The resulting reaction mixture was allowed to stir at 100° C. for 16 h. Progress of reaction was monitored by TLC (100% ethyl acetate). After completion, reaction mixture was diluted with ice-cold water (500 mL) and extract using ethyl acetate (2×150 mL). Aqueous layer was acidified with glacial acetic acid (pH=4-5). Solid precipitate was filtered, washed with water, dried under vacuum and azeotroped with toluene to afford 5-(benzyloxy)-2-methylpyrimidin-4-ol (15 g, 44%) as a white solid.

### Step-3

To a solution of 5-(benzyloxy)-2-methylpyrimidin-4-ol (15 g, 69.34 mmol, 1.0 eq.) in methanol (250 mL) was added Pd/C (3 g, 28.19 mmol, 0.4 eq.) at RT. The reaction mixture was allowed to stir under hydrogen atmosphere at room temperature for 2 h. Progress of reaction was monitored by TLC (100% ethyl acetate). After completion, reaction mixture was filtered through celite-bed and filtrate was evaporated under reduce pressure to afford 2-methylpyrimidine-4,5-diol (7 g, 80%) as a brown solid.

### Step-4

To a solution of 2-methylpyrimidine-4,5-diol (7 g, 55.55 mmol, 1.0 eq.) in water (100 mL) was added KOH (10.26 g, 183.33 mmol, 3.3 eq.) followed by 1,2-dibromoethane (23.5 g, 125 mmol, 2.25 eq.) at RT. The reaction mixture was allowed to stir at 100° C. temperature for 16 h. Progress of reaction was monitored by TLC (100% ethyl acetate). After completion, reaction mixture was diluted with water (20 mL) and extracted with ethyl acetate (3×200 mL). Combined organic layer was washed with brine and dried over anhydrous sodium sulfate. Removal of solvent under reduced pressure afforded crude which was purified by Combi-Flash to afford 2-methyl-6,7-dihydro-[1,4]dioxino[2,3-d]pyrimidine (4 g, 47.4%).

### Step-5

To a solution of 2-methyl-6,7-dihydro-[1,4]dioxino[2,3-d]pyrimidine (4 g, 26.28 mmol, 1.0 eq.) in CCl₄ (40 mL) was added AIBN (1.29 g, 7.88 mmol, 0.3 eq.) followed by NBS (14.03 g, 78.84 mmol, 3.0 eq.) at RT. The reaction mixture was allowed to stir at 80° C. temperature for 2.5 h. Progress of reaction was monitored by TLC (50% ethyl acetate-hexane). After completion, solvent was evaporated under reduced pressure, poured on ice-water (50 mL) and extracted with ethyl acetate (3×150 mL). Combined organic layer was washed with brine and dried over anhydrous sodium sulfate. Removal of solvent under reduced pressure afforded crude which was purified by Combi-Flash to afford 2-(dibromomethyl)-6,7-dihydro-[1,4]dioxino[2,3-d]pyrimidine (3 g, 36.85%).

### Step-6

To a solution of 2-(dibromomethyl)-6,7-dihydro-[1,4]dioxino[2,3-d]pyrimidine (3 g, 9.67 mmol, 1.0 eq.) in EtOH (30 mL) at RT was added a solution of AgNO₃ (3.29 g, 19.35 mmol, 2.0 eq.) in water (20 mL). The reaction mixture was allowed to stir at 100° C. in microwave for 30 minutes. Progress of reaction was monitored by TLC (100% ethyl acetate). After completion the reaction mixture was filtered through syringe filter and the filtrate was evaporated under reduced pressure, dried and azetroped to afford 6,7-dihydro-[1,4]dioxino[2,3-d]pyrimidine-2-carbaldehyde (0.8 g, 50%) which was used in the next step without purification.

### Step-7

To a solution of ethyl 2-(diethoxyphosphoryl)acetate (1.3 g, 5.77 mmol, 1.2 eq.) in dry THF (30 mL) was added NaH (60% in mineral oil) (0.138 g, 5.77 mmol, 1.2 eq.) at 0° C. over a period of 10 minutes. The reaction mixture was allowed to stir at that temperature for 30 minutes. To that stirred solution was added 6,7-dihydro-[1,4]dioxino[2,3-d]pyrimidine-2-carbaldehyde (0.8 g, 4.81 mmol, 1.0 eq.) at that temperature and allowed to stir at RT for another 30 minutes. Progress of reaction was monitored by TLC (50% ethyl acetate-hexane). After completion, reaction mixture was diluted with ice-cold water (50 mL) and extracted using ethyl acetate (3×100 mL). Combined organic layer was washed with brine and dried over anhydrous sodium sulfate. Removal of solvent is done under reduce pressure to afford crude which was purified by Combi-Flash to afford ethyl (E)-3-(6,7-dihydro-[1,4]dioxino[2,3-d]pyrimidin-2-yl)acrylate (0.7 g, 61.94%).

### Step-8

To a solution of ethyl (E)-3-(6,7-dihydro-[1,4]dioxino[2,3-d]pyrimidin-2-yl)acrylate (0.4 g, 1.69 mmol, 1.0 eq.) in EtOH (10 mL) at RT was added a solution of LiOH (0.203 g, 8.46 mmol, 5.0 eq.) in water (1 mL) and the resulting mixture was allowed to stir at 50° C. for 1 h. Progress of reaction was monitored by TLC (100% ethyl acetate). After completion, reaction mixture was concentrated to remove ethanol, washed with diethyl ether (2×10 mL), acidified with 1N HCl up to pH 6 and extracted with mixture of ethanol and ethyl acetate (1:4) (3×50 mL). Combined organic layer was washed with brine and dried over anhydrous sodium sulfate. Removal of solvent afforded crude (E)-3-(6,7-dihydro-[1,4]dioxino[2,3-d]pyrimidin-2-yl)acrylic acid (0.3 g, 85%) as white solid which was used in the next step without purification.

### Step-9

To a solution of (E)-3-(6,7-dihydro-[1,4]dioxino[2,3-d]pyrimidin-2-yl)acrylic acid (0.350 g, 1.68 mmol, 1.0 eq.) in dry THF were added triethylamine (0.204 g, 2.01 mmol, 1.2 eq.) and pivaloyl chloride (0.203 g, 1.68 mmol, 1.0 eq.). The reaction mixture was stirred at 0° C. for 45 minutes. Progress of reaction was monitored by TLC. After completion, reaction mixture was filtered and the filtrate was used directly for the next step.

To a stirred solution of 5,6-dihydropyridin-2(1H)-one (0.164 g, 1.68 mmol, 1.0 eq.) in dry THF at −78° C. was added n-butyl lithium (2.5 M in hexane, 0.68 mL, 1.68 mmol, 1.0 eq.) and the mixture was allowed to stir at the same temperature for 30 minutes. To this solution was added anhydride (0.490 g, 1.68 mmol, 1.0 eq.). Reaction mixture was allowed to stir at −78° C. for 90 minutes. Progress of reaction was monitored by TLC. After completion, reaction mixture was brought to 0° C., diluted with ammonium chloride solution (30 mL) and extracted with ethyl acetate (3×50 mL). Combined organic layer was washed with brine, dried over anhydrous Na₂SO₄ and evaporated to dryness under vacuum to afford crude which was purified by Combi-Flash on silica gel using ethyl acetate-hexane as eluent to afford (E)-1-(3-(6,7-dihydro-[1,4]dioxino[2,3-d]pyrimidin-2-yl)acryloyl)-5,6-dihydropyridin-2(1H)-one (0.092 g, 20%).

LCMS: 288 [M+1]⁺

¹H NMR (400 MHz, CDCl₃) δ 8.22 (s, 1H), 7.87 (d, 1H), 7.55 (d, 1H), 6.96-6.88 (m, 1H), 6.01 (d, 1H), 4.58-4.50 (m, 2H), 4.38-4.30 (m, 2H), 2.50-2.40 (m, 2H).

### Example 80

### Preparation of (E)-1-(3-(3,5-dimethyl-1H-pyrazol-4-yl)acryloyl)-5,6-dihydropyridin-2(1H)-one

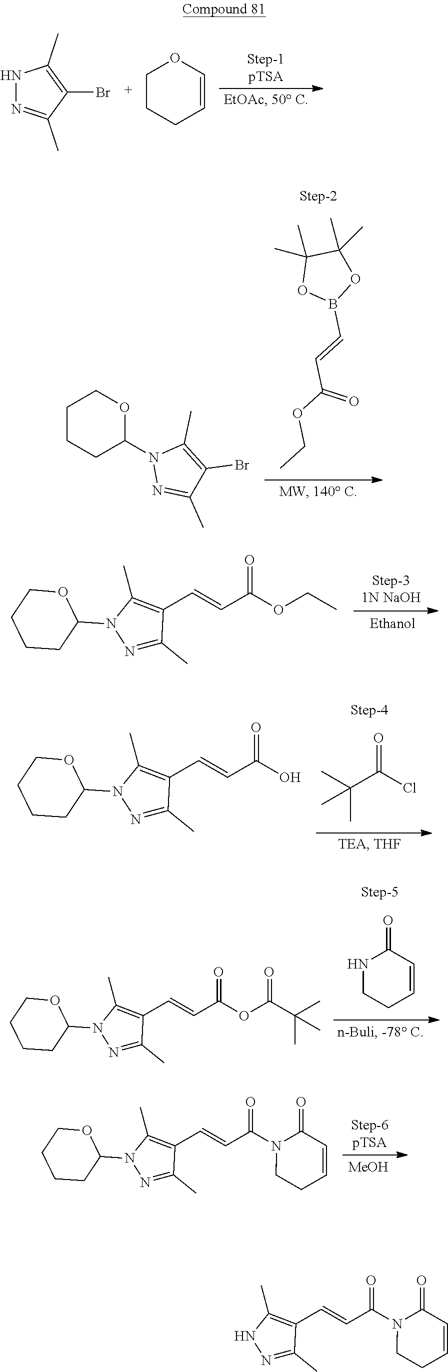

### Step-1

To a solution of 4-bromo-3,5-dimethyl-1H-pyrazole (3 g, 17.14 mmol, 1 eq) in ethyl acetate (40 mL) were added p-TSA (0.3 g, 1.71 mmol, 0.1 eq) and 3,4-dihydro-2H-pyran (4.7 mL, 51.42 mmol, 3 eq and the reaction mixture was allowed to stir at 50° C. for 3 h. Progress of reaction was monitored by TLC. After completion, reaction mixture was diluted with water and extracted with ethyl acetate (3×70 mL). Combined organic layer was washed with brine and dried over anhydrous sodium sulfate. Removal of solvent under reduce afforded crude 4-bromo-3,5-dimethyl-1-(tetrahydro-2H-pyran-2-yl)-1H-pyrazole which was purified by crystallization in ethyl acetate-pentane afford pure 4-bromo-3,5-dimethyl-1-(tetrahydro-2H-pyran-2-yl)-1H-pyrazole (2.5 g, 56.43%).

### Step-2

To a solution of 4-bromo-3,5-dimethyl-1-(tetrahydro-2H-pyran-2-yl)-1H-pyrazole (0.5 g, 2.86 mmol) in dioxane (6 mL) was added ethyl (E)-3-(4,4,5,5-tetramethyl-1,3,2-dioxaborolan-2-yl)acrylate (0.77 g, 3.42 mmol, 1.2 eq) and tert-butoxide (0.481 g, 1.5 eq) in water (3 mL) and the reaction mixture was deoxygenated using nitrogen gas for 10 minutes. Tetrakis(triphenylphosphine)palladium(0)(0.165 g, 0.14 mmol, 0.05 eq) was added and the reaction mixture was again deoxygenated for 10 minutes. Reaction mixture was allowed to stir at 140° C. for 1 h in microwave. Reaction mixture was cooled to RT, diluted with water (50 mL) and extract using ethyl acetate (3×20 mL). Combined organic layer was washed with brine and dried over anhydrous sodium sulfate. Removal of solvent under reduced pressure afforded crude which was purified by Combi-Flash to afford ethyl (E)-3-(3,5-dimethyl-1-(tetrahydro-2H-pyran-2-yl)-1H-pyrazol-4-yl)acrylate (1.2 g).

### Step-3

To a solution of ethyl (E)-3-(3,5-dimethyl-1-(tetrahydro-2H-pyran-2-yl)-1H-pyrazol-4-yl)acrylate (1.2 g, 5.47 mmol, 1 eq) in ethanol (20 mL) was added 1N NaOH (6 mL) dropwise and the mixture was allowed to stir at the RT for overnight. Progress of reaction was monitored by TLC. Reaction mixture was acidifying using 1N HCl up to pH 6 and extracted using mixture of ethanol and ethyl acetate (20:80) (3×50 mL). Combined organic layer was washed with brine and dried over anhydrous sodium sulfate. Removal of solvent afforded crude (E)-3-(3,5-dimethyl-1-(tetrahydro-2H-pyran-2-yl)-1H-pyrazol-4-yl)acrylic acid (0.520 g, 38.23%).

### Step-4

To a solution of (E)-3-(3,5-dimethyl-1-(tetrahydro-2H-pyran-2-yl)-1H-pyrazol-4-yl)acrylic acid (0.5 g, 1.99 mmol, 1.0 eq.) in dry THF was added triethylamine (0.33 mL, 2.38 mmol, 1.2 eq.) and pivaloyl chloride (0.22 mL, 1.79 mmol, 0.9 eq.) the reaction mixture was stirred at 0° C. for 45 minutes. Progress of reaction was monitored by TLC. After completion, reaction mixture was filtered and the filtrate was used directly for the next step.

### Step-5

To a stirred solution of 5,6-dihydropyridin-2(1H)-one (0.193 g, 1.99 mmol, 1 eq.) in dry THF at −78° C. was added n-butyl lithium (2.5 M in hexane, 0.79 mL, 1.99 mmol, 1 eq.) and the mixture was allowed to stir at the same temperature for 30 minutes. To this solution was added (E)-(E)-3-(3,5-dimethyl-1-(tetrahydro-2H-pyran-2-yl)-1H-pyrazol-4-yl)acrylic pivalic anhydride (0.665 g, 1.99 mmol, 1 eq.). Reaction mixture was allowed to stir at −78° C. for 90 min. Progress of reaction was monitored by TLC. Reaction mixture was brought to 0° C., diluted with ammonium chloride solution (30 mL) and extracted with ethyl acetate (3×50 mL). combined organic layer was washed with brine, dried over anhydrous Na₂SO₄ and evaporated to dryness under vacuum to afford crude which was purified by Combi-Flash to afford (E)-1-(3-(3,5-dimethyl-1-(tetrahydro-2H-pyran-2-yl)-1H-pyrazol-4-yl)acryloyl)-5,6-dihydropyridin-2(1H)-one (0.320 g, 48.92%).

### Step-6

To a solution of (E)-1-(3-(3,5-dimethyl-1-(tetrahydro-2H-pyran-2-yl)-1H-pyrazol-4-yl)acryloyl)-5,6-dihydropyridin-2(1H)-one (0.3 g, 0.91 mmol, 1 eq) in methanol (15 mL) was added p-TSA (0.188 g, 1.09 mmol, 1.2 eq). Allow the reaction mixture to stir at Room temperature for 2 h. Progress of reaction was monitored by TLC. After completion pour water and extract using ethyl acetate (3×30 mL). Combined organic layer was washed with brine and dried over anhydrous sodium sulfate. Removal of solvent is done under reduce pressure. To afford crude (E)-1-(3-(3,5-dimethyl-1H-pyrazol-4-yl)acryloyl)-5,6-dihydropyridin-2(1H)-one which was purified by crystallization in ethyl acetate-pentane to afford pure (E)-1-(3-(3,5-dimethyl-1H-pyrazol-4-yl)acryloyl)-5,6-dihydropyridin-2(1H)-one (0.1 g, 44.84%).

LCMS: 246 [M+1]⁺

¹H NMR (400 MHz, CDCl₃) δ 7.75 (d, 1H), 7.25 (d, 1H), 6.97-6.87 (m, 1H), 6.02 (d, 1H), 4.05 (t, 2H), 2.46-2.41 (m, 2H), 2.39 (s, 6H).

### Example 81

### Preparation (E)-N-(4-fluorophenyl)-4-(2-((6-oxo-3,6-dihydropyridin-1(2H)-yl)sulfonyl)vinyl)piperidine-1-carboxamide

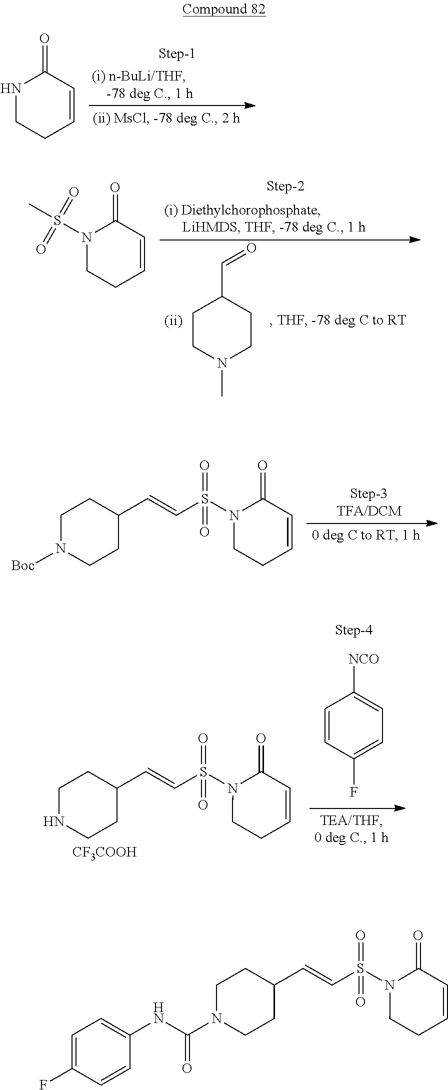

### Step-1

To a stirred solution of 5,6-dihydropyridin-2(1H)-one (1 g, 10.3 mmol, 1.0 eq.) in dry THF at −78° C. was added n-butyllithium (2.5M in hexane, 4.2 ml, 10.30 mmol, 1 eq.) and the mixture was allowed to stir at the same temperature for 1 h. To this solution was added a solution of methane sulfonyl chloride (0.92 ml, 1.67 mmol, 1.1 eq.) in THF and the reaction mixture was allowed to stir at −78° C. for 1 h. Progress of reaction was monitored by TLC and LCMS. After completion, reaction mixture was quenched aq. NH₄Cl solution (50 mL) and extracted with ethyl acetate (3×30 mL). Combined organic layer was washed with brine (50 mL), dried over anhydrous Na₂SO₄ and evaporated to dryness under vacuum to afford crude which was purified silica gel chromatography using ethyl acetate-hexane system as eluent to afford 1-(methylsulfonyl)-5,6-dihydropyridin-2(1H)-one (700 mg, 38.88%).

LCMS: 176 [M+1]⁺

### Step-2

To a suspension of 1-(methylsulfonyl)-5,6-dihydropyridin-2(1H)-one (0.5 g, 2.857 mmol 1 eq) in THF (50 mL) and diethyl chloro phosphate (0.49 g, 2.857 mol, 1 eq) was added LiHMDS at −78° C. and the mixture was allowed to stir at the same temperature for 1 h. To this mixture was added a solution of tert-butyl 4-formylpiperidine-1-carboxylate (0.6 g, 2.857 mol, 1.0 eq) in THF (5 mL) and the resulting mixture was allowed to stir at room temperature for 1 h. Progress of reaction was monitored by TLC. After completion, reaction mixture was diluted with saturated aq. NH₄Cl (100 mL) and extracted with ethyl acetate (3×50 mL). Combined organic layer was washed with brine, dried over anhydrous Na₂SO₄ and evaporated to dryness under vacuum to afford crude which was purified by Combi-Flash using ethyl acetate-hexane system as eluent to afford product tert-butyl (E)-4-(2-((6-oxo-3,6-dihydropyridin-1(2H)-yl)sulfonyl) vinyl) piperidine-1-carboxylate (80 mg, 7.5%).

LCMS: 371 [M+1]⁺

### Step-3

To a stirred solution (90 mg, 0.245 mmol, 1 eq) in DCM (5 mL) at 0° C. was added TFA (0.5 mL) dropwise and the resulting reaction mixture was stirred at RT for 1 h. Progress of reaction was monitored by TLC. After completion, reaction mixture was concentrate under reduced pressure to afford (E)-1-((2-(piperidin-4-yl)vinyl)sulfonyl)-5, dihydropyridin-2(1H)-one which was used in the next step without purification (70 mg, 84%).

LCMS: 271[M+1]⁺

### Step-4

To a stirred solution of (E)-1-((2-(piperidin-4-yl)vinyl)sulfonyl)-5, dihydropyridin-2(1H)-one (80 mg, 0.295 mmol, 1 eq) in THF (5 mL) was cooled up to 0° C. and added TEA (76 μL, 0.590 mmol, 2 eq) and stirred for 10 minute then added 1-fluoro-4-isocyanatobenzene (8 mg, 0.762 mmol, 2 eq) already dissolved in THF (5 mL) and stirred for 20 minute. Reaction was monitored by TLC and LCMS, after completion of reaction, reaction mixture was diluted with ethyl acetate (40 mL) and washed with brine (3×20 mL). Organic layer was dried over sodium sulphate and concentrated under reduced pressure to yield crude which is purified by Combi-Flash on silica gel using ethyl acetate-hexane system as eluent to afford (E)-N-(4-fluorophenyl)-4-(2-((6-oxo-3,6-dihydropyridin-1(2H)-yl)sulfonyl)vinyl)piperidine-1-carboxamide (22 mg, 25%).

LCMS: 408 [M+1]⁺

¹H NMR (400 MHz, DMSO-d₆) δ 8.50 (s, 1H), 7.46-7.40 (m, 2H), 7.10-6.95 (m, 3H), 6.90-6.80 (m, 1H), 6.76 (d, 1H), 5.86 (d, 1H), 4.17-4.03 (m, 2H), 3.80 (t, 2H), 2.89-2.79 (m, 2H), 2.60-2.40 (m, 2H), 1.60-1.50 (m, 2H), 1.40-1.20 (m, 3H).

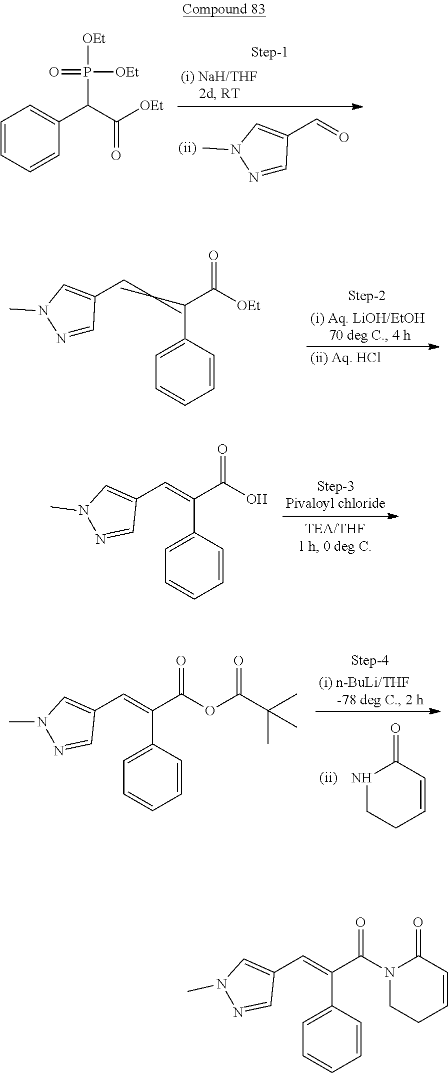

### Step-1

To a suspension of sodium hydride (60% in mineral oil, 545 mg 13.63 mmol, 1.5 eq) in THF (50 mL) was added ethyl(diethoxyphosphoryl)(phenyl)acetate (3.37 g, 10.90 mmol, 1.2 eq) dropwise at 0° C. and the mixture was allowed to stir at the same temperature for 30 minutes. To this mixture was added a solution of 1-methyl-1H-pyrazole-4-carbaldehyde (1 g, 9.09 mmol, 1.0 eq) in THF (5 mL) and the resulting mixture was allowed to stir at room temperature for 2 days. Progress of reaction was monitored by TLC. After completion, reaction mixture was diluted with saturated aq. NH₄Cl (100 mL) and extracted with ethyl acetate (3×50 ml). Combined organic layer was washed with brine, dried over anhydrous Na₂SO₄ and evaporated to dryness under vacuum to afford crude which was purified by Combi-Flash ethyl acetate-hexane system as eluent to afford ethyl (2E)-3-(1-methyl-1H-pyrazol-4-yl)-2-phenylprop-2-enoate (0.5 g, 21.55%).

LCMS: 257 [M+1]⁺

### Step-2

To a solution of ethyl (2E)-3-(1-methyl-1H-pyrazol-4-yl)-2-phenylprop-2-enoate (90 mg, 0.351 mmol, 1.0 eq) in ethanol (20 mL) was added a solution of lithium hydroxide (42 mg, 1.76 mmol, 5 eq) in water (2 mL) and the reaction mixture was stirred at 70° C. for 4 h. Progress of reaction was monitored by TLC. After completion, reaction mixture was concentrated under vacuum and then acidified with 2N HCl make pH up to 3. The mixture was diluted saturated aq. NH₄Cl (30 mL) and extracted with dichloromethane (3×30 mL). Combined organic layer was washed with brine solution (50 mL) and dried over anhydrous Na₂SO₄. Removal of solvent under reduced pressure to obtain (2E)-3-(1-methyl-1H-pyrazol-4-yl)-2-phenylprop-2-enoic acid (70 mg, 87.50%).

LCMS: 229 [M+1]⁺

### Step-3

To a solution of (2E)-3-(1-methyl-H-pyrazol-4-yl)-2-phenylprop-2-enoic acid (70 mg, 0.307 mmol, 1.0 eq.) in dry THF was added triethylamine (46 μl, 0.337 mmol, 1.1 eq.) followed by pivaloyl chloride (40 mg, 0.337 mmol, 1.1 eq.) and the reaction mixture was stirred at 0° C. for 45 minutes. Progress of reaction was monitored by TLC. After completion, reaction mixture was filtered and the filtrate was used directly for the next step.

### Step-4

To a stirred solution of 5,6-dihydropyridin-2(1H)-one 32 mg, 0.337 mmol, 1.1 eq.) in dry THF at −78° C. was added n-butyl lithium (2.5 M in hexane, 0.14 mL, 0.337 mmol, 1.1 eq.) and the mixture was allowed to stir at the same temperature for 30 minutes. To this solution was added a solution of 2,2-dimethylpropanoic (2E)-3-(1-methyl-1H-pyrazol-4-yl)-2-phenylprop-2-enoic anhydride (0.307 mmol, 1 eq.) in THF and the reaction mixture was allowed to stir at −78° C. for 1 h. Progress of reaction was monitored by TLC and LCMS. After completion, reaction mixture was brought to 0° C., diluted with water (30 mL) and extracted with ethyl acetate (3×30 mL). Combined organic layer was washed with brine, dried over anhydrous Na₂SO₄ and evaporated to dryness under vacuum to afford crude which was purified by reversed phase HPLC to afford 1-[(2E)-3-(1-methyl-1H-pyrazol-4-yl)-2-phenylprop-2-enoyl]-5,6-dihydropyridin-2(1H)-one (12 mg 12.76%).

LCMS: 308 [M+1]⁺

¹H NMR (400 MHz, CDCl₃) δ 7.31-7.42 (m, 4H), 7.13 (s, 1H), 7.04 (s, 1H), 6.84 (s, 1H), 6.76-6.83 (m, 1H), 5.80 (d, 1H), 3.88 (t, 2H), 3.73 (s, 3H), 2.55-2.55 (d, 2H).

### Example 83

### Preparation of (Z)-1-(3-(1-methyl-1H-pyrazol-4-yl)-2-phenylacryloyl)-5,6-dihydropyridin-2(1H)-one

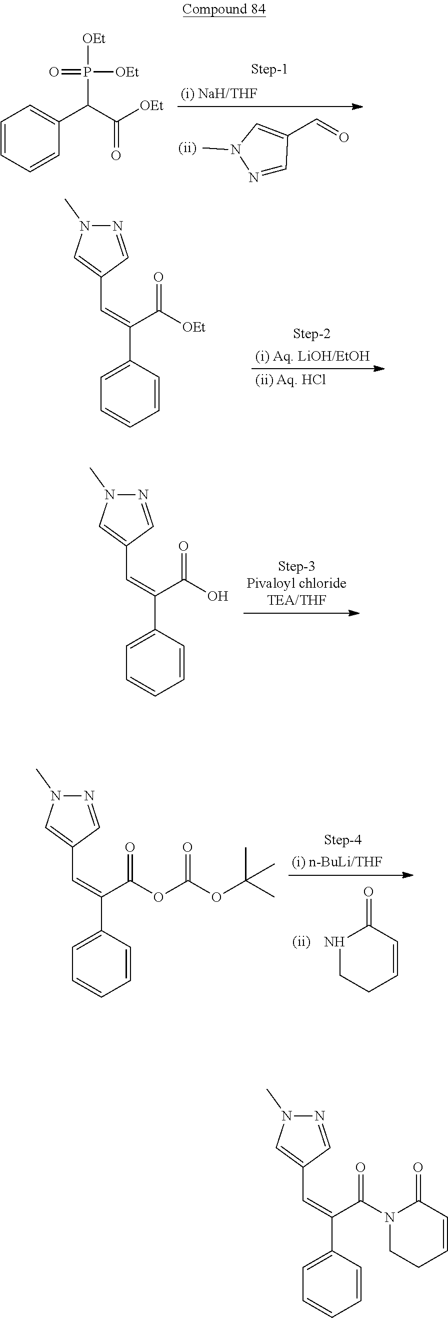

### Step-1

To a suspension of sodium hydride (60% in mineral oil, 545 mg 13.63 mmol, 1.5 eq) in THF (50 mL) was added ethyl(diethoxyphosphoryl)(phenyl)acetate (3.37 g, 10.90 mmol, 1.2 eq) dropwise at 0° C. and the mixture was allowed to stir at the same temperature for 30 minutes. To this mixture was added a solution of 1-methyl-1H-pyrazole-4-carbaldehyde (1 g, 9.09 mmol, 1.0 eq) in THF (5 mL) and the resulting mixture was allowed to stir at room temperature for 2 days. Progress of reaction was monitored by TLC. After completion, reaction mixture was diluted with saturated aq. NH₄Cl (100 mL) and extracted with ethyl acetate (3×50 mL). Combined organic layer was washed with brine, dried over anhydrous Na₂SO₄ and evaporated to dryness under vacuum to afford crude which was purified by Combi-Flash using ethyl acetate-hexane system as eluent to afford (Z)-ethyl 3-(1-methyl-1H-pyrazol-4-yl)-2-phenylacrylate (0.5 g, 21.55%).

LCMS: 257 [M+1]⁺

### Step-2

To a solution of ethyl (2E)-3-(1-methyl-H-pyrazol-4-yl)-2-phenylprop-2-enoate (150 mg, 0.585 mmol, 1.0 eq) in ethanol (30 mL) was added a solution of lithium hydroxide (70 mg, 2.929 mmol, 5 eq) in water (3 mL) and the reaction mixture was stirred at 70° C. for 4 h. Progress of reaction was monitored by TLC. After completion, reaction mixture was concentrated under vacuum and then acidified with 2N HCl make pH up to 3. The mixture was diluted with saturated aq. NH₄Cl (30 mL) and extracted with dichloromethane (3×30 mL). The combined organic layer was washed with brine solution (50 mL) and dried over anhydrous Na₂SO₄. Removal of solvent under reduced pressure to obtain (Z)-3-(1-methyl-1H-pyrazol-4-yl)-2-phenylacrylic acid (120 mg, 90.22%).

LCMS: 229 [M+1]⁺

### Step-3

To a solution of (2E)-3-(1-methyl-H-pyrazol-4-yl)-2-phenylprop-2-enoic acid (120 mg, 0.526 mmol, 1.0 eq.) in dry THF was added triethylamine (81 μl 10.578 mmol, 1.1 eq.) followed by pivaloyl chloride (69 mg 0.578 mmol, 1.1 eq.) and the reaction mixture was stirred at 0° C. for 45 minutes. Progress of reaction was monitored by TLC. After completion, reaction mixture was filtered and the filtrate was used directly for the next step.

### Step-4

To a stirred solution of 5,6-dihydropyridin-2(1H)-one (56 mg, 0.578 mmol, 1.1 eq.) in dry THF at −78° C. was added n-butyl lithium (2.5M in hexane, 0.23 mL, 0.578 mmol, 1.1 eq.) and the mixture was allowed to stir at the same temperature for 30 minutes. To this solution was added a solution of 2,2-dimethylpropanoic (2E)-3-(1-methyl-1H-pyrazol-4-yl)-2-phenylprop-2-enoic anhydride (0.526 mmol, 1 eq.) in THF and the reaction mixture was allowed to stir at −78° C. for 1 h. Progress of reaction was monitored by TLC and LCMS. After completion, reaction mixture was brought to 0° C., diluted with water (30 mL) and extracted with ethyl acetate (3×30 mL). Combined organic layer was washed with brine, dried over anhydrous Na₂SO₄ and evaporated to dryness under vacuum to afford crude which was purified by reversed phase HPLC to afford (Z)-1-(3-(1-methyl-1H-pyrazol-4-yl)-2-phenylacryloyl)-5,6-dihydropyridin-2(1H)-one (50 mg, 31.05%).

LCMS: 308 [M+1]⁺

¹H NMR (400 MHz CDCl₃) δ 7.47 (s, 1H), 7.37-7.44 (m, 3H), 7.33 (t, 2H), 7.24-7.29 (m, 1H), 6.76-6.87 (m, 1H), 6.66 (s, 1H), 5.80 (d, 1H), 4.16 (t, 2H) 3.86 (s, 3H) 2.50-2.35 (d, 2H).

### Example 84

### Preparation of (E)-5,5-dimethyl-1-(3-(1-methyl-1H-pyrazol-4-yl)acryloyl)-5,6-dihydropyridin-2(1H)-one

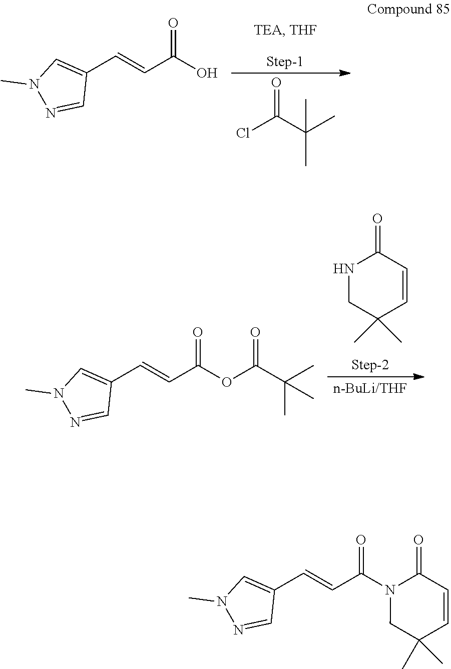

### Step-1

To a solution of (E)-3-(1-methyl-1H-pyrazol-4-yl)acrylic acid (85 mg, 0.56 mmol, 1.0 eq.) in dry THF (10 mL) was added triethylamine (0.093 mL, 0.67 mmol, 1.2 eq.) followed by pivaloyl chloride (0.61 mL, 0.67 mmol, 1.2 eq.) and the reaction mixture was stirred at 0° C. for 45 minutes. Progress of reaction was monitored by TLC. After completion, reaction mixture was filtered and the filtrate was used directly for the next step.

### Step-2

To a stirred solution of 5,5-dimethyl-5,6-dihydropyridin-2(1H)-one (70 mg, 0.56 mmol, 1.0 eq.) in dry THF at −78° C. was added n-BuLi (0.23 mL, 0.56 mmol, 1 eq.) and the mixture was allowed to stir at the same temperature for 30 minutes. To this solution was added a solution of (E)-(E)-3-(1-methyl-1H-pyrazol-4-yl)acrylic pivalic anhydride (0.56 mmol, 1.0 eq.) in THF and the reaction mixture was allowed to stir at room temperature for 1 h. Progress of reaction was monitored by TLC and LCMS. After completion, reaction mixture was diluted with saturated aq. ammonium chloride solution (50 mL) and extracted with ethyl acetate (3×50 mL). Combined organic layer was washed with brine (50 mL) and dried over anhydrous sodium sulfate. Removal of solvent gave crude which was purified by Combi-Flash on silica gel using ethyl acetate-hexane system as eluent to afford (E)-5,5-dimethyl-1-(3-(1-methyl-1H-pyrazol-4-yl)acryloyl)-5,6-dihydropyridin-2(1H)-one (9 mg, 6.3%).

LCMS: 260 [M+1]⁺

¹H NMR (400 MHz, DMSO-d₆) δ 7.75 (s, 1H), 7.68 (d, 1H), 7.60 (d, 1H), 7.30 (d, 1H), 6.61 (d, 1H), 5.81 (d, 1H), 3.90 (s, 3H), 3.79 (s, 2H), 1.18 (s, 6H).

### Example 85

### Preparation of (E)-1-(3-(1-cyclopropyl-1H-pyrazol-4-yl)acryloyl)-5,6-dihydropyridin-2(1H)-one

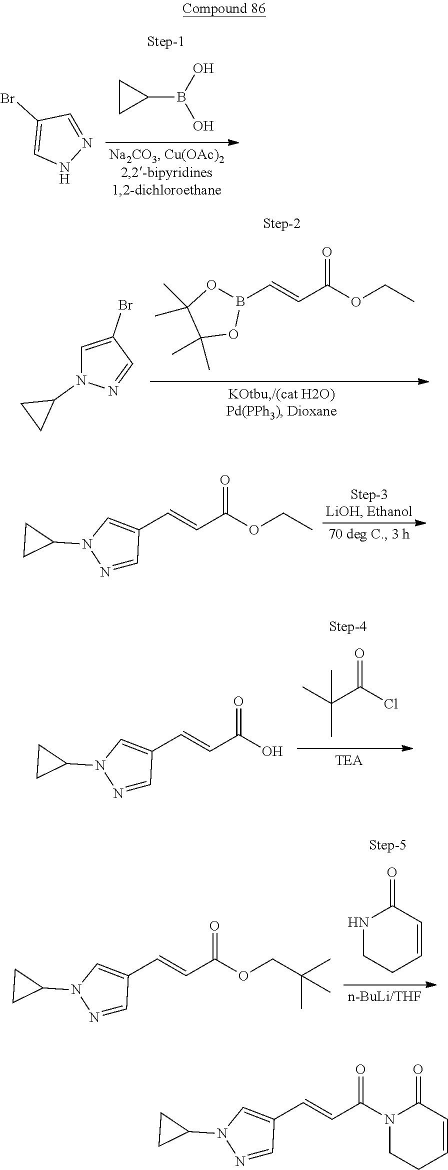

### Step-1

A mixture of 4-bromo-1H-pyrazole (0.50 g), cyclopropylboronic acid (1.0 g, 6.80 mmol, 1.0 eq), copper(II) acetate (0.5 g, 7.48 mmol, 1.1 eq.), 2,2′-bipyridine (1.0 g, 7.48 mmol, 1.1 eq.), and sodium carbonate (0.5 g, 14.96 mmol, 2.2 eq) in 1,2-dichloroethane (15 mL) was stirred under reflux temperature for 4 h. To this mixture was again added cyclopropylboronic acid (0.5 g, 14.96 mmol, 2.2 eq.) and the mixture was further stirred under reflux overnight. Progress of reaction was monitored by TLC. Reaction mixture was cooled to RT, diluted with aqueous NH₄Cl solution (30 mL) and extracted with dichloromethane (3×30 mL). Combined organic layer was dried over Na₂SO₄ and concentrated under vacuum to afford crude which was purified by Combi-Flash on silica gel using ethyl acetate-hexane as eluent to afford 4-bromo-1-cyclopropyl-1H-pyrazole (0.7 g, 55.55%) as colorless oil.

### Step-2

To a solution of 4-bromo-1-cyclopropyl-1H-pyrazole (0.5 g, 2.68 mmol) in dioxane (10 mL) was added ethyl (E)-3-(4,4,5,5-tetramethyl-1,3,2-dioxaborolan-2-yl)acrylate (0.73 g, 3.22 mmol, 1.2 eq) and potassium tert-butoxide (0.602 g, 5.37 mmol, 1.5 eq) in water (3 mL) and the reaction mixture was deoxygenated using nitrogen gas for 10 minutes. Tetrakis(triphenylphosphine)palladium(0) (0.154 g, 0.135 mmol, 0.05 eq) was added and the reaction mixture was again deoxygenated for 10 minutes. Reaction mixture was allowed to stir at 140° C. for 1 h in microwave. Reaction mixture was cooled to RT, diluted with water (50 mL) and extract using ethyl acetate (3×30 mL). Combined organic layer was washed with brine and dried over anhydrous sodium sulfate. Removal of solvent under reduced pressure afforded crude which was purified by Combi-Flash to afford ethyl (E)-3-(1-cyclopropyl-1H-pyrazol-4-yl) acrylate (0.370 g, 66.90%).

LCMS: 207 [M+1]⁺

### Step-3

To a solution of ethyl (E)-3-(1-cyclopropyl-1H-pyrazol-4-yl) acrylate (0.35 g, 1.699 mmol, 1.0 eq) in ethanol (100 mL) was added a solution of lithium hydroxide (0.122 g, 5.097 mmol, 3.0 eq) in water (10.0 mL) and the reaction mixture was stirred at 70° C. for 2 h. Reaction mixture was concentrated under vacuum and then acidified with 1N HCl (10 mL). The mixture was diluted with water (30 mL) and extracted with ethyl acetate (3×30 mL). Combined organic layer was washed with brine solution (50 mL) and dried over anhydrous Na₂SO₄. Removal of solvent under reduced pressure gave (E)-3-(1-cyclopropyl-1H-pyrazol-4-yl)acrylic acid (0.260 g, 86%) as crystalline solid.

LCMS: 179 [M+1]⁺

### Step-4

To a solution of (E)-3-(1-cyclopropyl-1H-pyrazol-4-yl)acrylic acid (0.25 g, 1.404 mmol, 1.0 eq.) in dry THF (20 mL) was added triethylamine (0.164 mL, 1.17 mmol, 1.2 eq.) followed by pivaloyl chloride (0.185 mL, 1.54 mmol, 1.1 eq.) and the reaction mixture was stirred at 0° C. for 45 minutes. Progress of reaction was monitored by TLC. After completion, reaction mixture was filtered and the filtrate was used directly for the next step.

### Step-5

To a stirred solution of 5,6-dihydropyridin-2(1H)-one (0.100 g, 1.030 mmol, 1.0 eq.) in dry THF (15.0 mL) at −78° C. was added nBuLi (0.45 mL, 1.134 mmol, 1.1 eq.) and the mixture was allowed to stir at the same temperature for 30 minutes. To this solution was added a solution of (E)-(E)-3-(1-cyclopropyl-1H-pyrazol-4-yl)acrylic pivalic anhydride (0.297 g, 1.134 mmol, 1.1 eq.) in THF and the reaction mixture was allowed to stir at room temperature for 1 h. Progress of reaction was monitored by TLC and LCMS. After completion, reaction mixture was evaporated to dryness under vacuum to afford crude which was purified by reversed phase HPLC to afford (E)-1-(3-(1-cyclopropyl-1H-pyrazol-4-yl)acryloyl)-5,6-dihydropyridin-2(1H)-one (0.033 g, 12.45c %) was obtained as off white solid.

LCMS: 258 [M+1]⁺

¹H NMR (400 MHz, CD₃OD) δ 8.00 (s, 1H) 7.75 (s, 1H) 7.58 (d, 1H) 7.22 (d, 1H) 7.01-7.12 (m, 1H) 5.99 (d, 1H) 3.96 (t, 2H) 3.61-3.72 (m, 1H) 2.48 (d, 2H) 1.00-1.14 (m, 4H).

### Example 86

### Preparation of (Z)-1-(2-methyl-3-(piperidin-4-yl)acryloyl)-5,6-dihydropyridin-2(1H)-one 2,2,2-trifluoroacetate

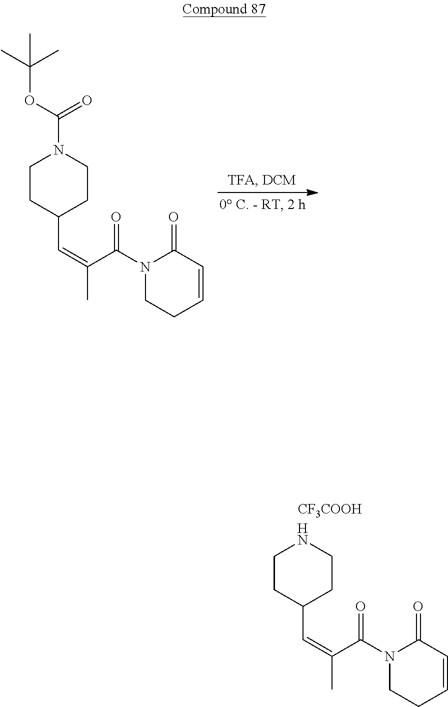 [SMILES: C/C(=C/C1CCN(C(=O)OC(C)(C)C)CC1)C(=O)N1CCC=CC1=O ; "CF3COOH" ; C/C(=C/C1CCNCC1)C(=O)N1CCC=CC1=O]

To a solution of (Z)-tert-butyl 4-(2-methyl-3-oxo-3-(2-oxo-5,6-dihydropyridin-1(2H)-yl)prop-1-enyl)piperidine-1-carboxylate (0.38 g, 1.09 mmol, 1.0 eq) in DCM (10 mL) was added TFA (0.37 mL, 3.2 mmol, 3.0 eq) at 0° C. and then the reaction mixture was allowed to stir at RT for 2 h. Progress of reaction was monitored by TLC. After completion, removal of solvent under reduced pressure afforded (Z)-1-(2-methyl-3-(piperidin-4-yl)acryloyl)-5,6-dihydropyridin-2(1H)-one 2,2,2-trifluoroacetate (0.21 g, 53.16%) as a solid.

LCMS: 249 [M+1]⁺

¹H NMR (400 MHz, DMSO-d₆) δ 8.55 (brs, 1H), 8.20 (brs, 1H), 7.10-7.00 (m, 1H), 5.86 (d, 1H), 5.60 (d, 1H), 3.82 (t, 2H), 3.30-3.20 (m, 2H), 3.00-2.82 (m, 2H), 2.60-2.40 (m, 3H), 1.80 (s, 3H), 1.70-1.62 (m, 2H), 1.50-1.35 (m, 2H).

### Example 87

### Preparation of (E)-1-(2-methyl-3-(piperidin-4-yl)acryloyl)-5,6-dihydropyridin-2(1H)-one 2,2,2-trifluoroacetate

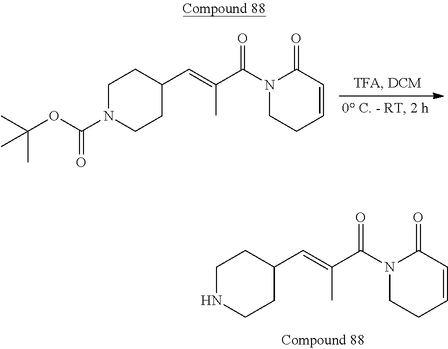 [SMILES: C/C(=C\C1CCN(C(=O)OC(C)(C)C)CC1)C(=O)N1CCC=CC1=O ; C/C(=C\C1CCNCC1)C(=O)N1CCC=CC1=O]

To a solution of (E)-tert-butyl 4-(2-methyl-3-oxo-3-(2-oxo-5,6-dihydropyridin-1(2H)-yl)prop-1-enyl)piperidine-1-carboxylate (0.19 g, 0.54 mmol, 1.0 eq) in DCM (10 mL) was added TFA (0.186 mL, 1.63 mmol, 3.0 eq) at 0° C. and reaction mixture was allowed to stir at RT for 3 h. Progress of reaction was monitored by TLC. After completion, removal of solvent under reduced pressure gave (E)-1-(2-methyl-3-(piperidin-4-yl)acryloyl)-5,6-dihydropyridin-2(1H)-one 2,2,2-trifluoroacetate (0.14 g, 71.06%) as solid.

LCMS: 249 [M+1]⁺

¹H NMR (400 MHz, DMSO-d₆) δ 8.55 (brs, 1H), 8.20 (brs, 1H), 7.10-7.00 (m, 1H), 5.86 (d, 1H), 5.60 (d, 1H), 3.75 (t, 2H), 3.30-3.20 (m, 2H), 3.00-2.82 (m, 2H), 2.60-2.40 (m, 3H), 1.80 (s, 3H), 1.70-1.62 (m, 2H), 1.50-1.35 (m, 2H).

### Example 88

### Preparation of (E)-1-(3-(1H-pyrazol-4-yl)acryloyl)-5,6-dihydropyridin-2(1H)-one

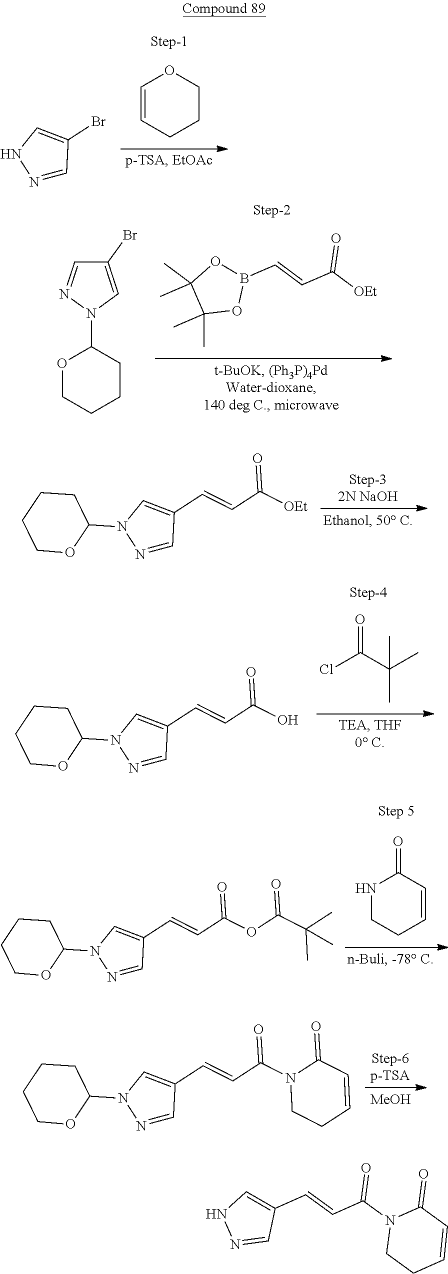

### Step-1

To a solution of 4-bromo-1H-pyrazole (5 g, 34.01 mmol, 1 eq) in ethyl acetate (50 mL) were added p-TSA (0.585 g, 3.40 mmol, 0.1 eq) and 3,4-dihydro-2H-pyran (9.34 mL, 102.05 mmol, 3 eq) and the reaction mixture was allowed to stir at 50° C. for 4 h. Progress of reaction was monitored by TLC. After completion, reaction mixture was diluted with water (200 mL) and extract using ethyl acetate (3×70 mL). Combined organic layer was washed with brine (100 mL) and dried over anhydrous sodium sulfate. Removal of solvent under reduce pressure gave crude which was purified by Combi-Flash on silica gel using ethyl acetate-hexane system as eluent to afford 4-bromo-1-(tetrahydro-2H-pyran-2-yl)-1H-pyrazole (5 g, 63.21%).

### Step-2

To a solution of 4-bromo-1-(tetrahydro-2H-pyran-2-yl)-1H-pyrazole (0.5 g, 2.16 mmol) in 1,4-dioxane (6 mL) was added ethyl (E)-3-(4,4,5,5-tetramethyl-1,3,2-dioxaborolan-2-yl)acrylate (0.587 g, 2.59 mmol, 1.2 eq) and tert-butoxide (0.363 g, 1.5 eq) in water (3 mL) and the reaction mixture was deoxygenated using nitrogen gas for 10 minutes. Tetrakis(triphenylphosphine)palladium(0) (0.124 g, 0.10 mmol, 0.05 eq) was added and the reaction mixture was again deoxygenated for 10 minutes. Reaction mixture was allowed to stir at 140° C. for 1 h in microwave. Reaction mixture was cooled to RT, diluted with water (100 mL) and extract with ethyl acetate (3×20 mL). Combined organic layer was washed with brine (100 mL) and dried over anhydrous sodium sulfate. Removal of solvent under reduced pressure afforded crude which was purified by Combi-Flash to afford ethyl (E)-3-(1-(tetrahydro-2H-pyran-2-yl)-1H-pyrazol-4-yl) acrylate (0.3 g, 55.45%)

### Step-3

To a solution of ethyl (E)-3-(1-(tetrahydro-2H-pyran-2-yl)-1H-pyrazol-4-yl) acrylate (0.3 g, 1.19 mmol, 1 eq) in ethanol (10 mL) was added 2N NaOH (3 mL) dropwise and the mixture was allowed to stir at the RT for overnight. Progress of reaction was monitored by TLC. Reaction mixture was acidified using 1N HCl up to pH 6 and extracted using mixture of ethanol-ethyl acetate (20%) (3×50 mL). Combined organic layer was washed with brine and dried over anhydrous sodium sulfate. Removal of solvent afforded crude (E)-3-(1-(tetrahydro-2H-pyran-2-yl)-1H-pyrazol-4-yl)acrylic acid (0.160 g, 60.15%) which was used in the next step without further purification.

### Step-4

To a solution of (E)-3-(1-(tetrahydro-2H-pyran-2-yl)-1H-pyrazol-4-yl)acrylic acid (0.160 g, 0.72 mmol, 1.0 eq.) in dry THF was added triethylamine (0.120 mL, 0.86 mmol, 1.2 eq.) and pivaloyl chloride (0.079 mL, 0.65 mmol, 0.9 eq.) the reaction mixture was stirred at 0° C. for 45 minutes. Progress of reaction was monitored by TLC. After completion, reaction mixture was filtered and the filtrate was used directly for the next step.

### Step-5

To a stirred solution of 5,6-dihydropyridin-2(1H)-one (0.070 g, 0.72 mmol, 1 eq.) in dry THF at −78° C. was added n-butyl lithium (2.5M in hexane, 0.29 mL, 0.72 mmol, 1 eq.) and the mixture was allowed to stir at the same temperature for 30 minutes. To this solution was added (E)-(E)-3-(1-(tetrahydro-2H-pyran-2-yl)-1H-pyrazol-4-yl)acrylic pivalic anhydride (0.220 g, 0.72 mmol, 1 eq.). Reaction mixture was allowed to stir at −78° C. for 90 min. Progress of reaction was monitored by TLC. Reaction mixture was brought to 0° C., diluted with ammonium chloride solution (30 mL) and extracted with ethyl acetate (3×40 mL). Combined organic layer was washed with brine, dried over anhydrous Na₂SO₄ and evaporated to dryness under vacuum to afford crude which was purified by Combi-Flash to afford (E)-1-(3-(1-(tetrahydro-2H-pyran-2-yl)-1H-pyrazol-4-yl)acryloyl)-5,6-dihydropyridin-2(1H)-one (85 mg, 39.35%).

### Step-6

To a solution of (E)-1-(3-(1-(tetrahydro-2H-pyran-2-yl)-1H-pyrazol-4-yl)acryloyl)-5,6-dihydropyridin-2(1H)-one (85 mg, 0.28 mmol, 1 eq.) in methanol (6 mL) was added p-TSA (58 mg, 0.33 mmol, 1.2 eq). The reaction mixture was allowed to stir at room temperature for 2 h. Progress of reaction was monitored by TLC. After completion, the mixture was poured into water and extracted using ethyl acetate (3×20 mL). Combined organic layer was washed with brine and dried over anhydrous sodium sulfate. Removal of solvent was done under reduce pressure to afford crude material, which was purified by Combi-Flash to afford (E)-1-(3-(1H-pyrazol-4-yl)acryloyl)-5,6-dihydropyridin-2(1H)-one (10 mg, 16.39%).

LCMS: 218 [M+1]⁺

¹H NMR (400 MHz, CDCl₃) δ 7.85 (brs, 1H), 7.75 (d, 1H), 7.32 (d, 1H), 6.98-6.90 (m, 1H), 6.05 (d, 1H), 4.03 (t, 2H), 2.50-2.43 (m, 2H).

### Example 89

### Preparation of 1 11-{(2E)-3-[1-(2,2,2-trifluoroethyl)-1H-pyrazol-4-yl]prop-2-enoyl}-5,6-dihydropyridin-2(1H)-one

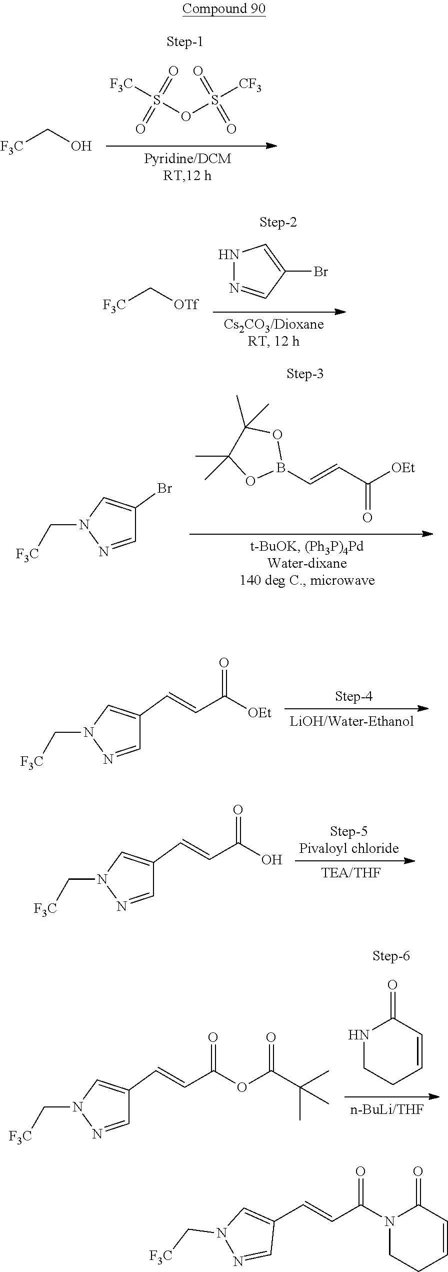

### Step-1

To a solution of 2,2,2-trifluoroethanol (2 g, 20.00 mmol, 1.0 eq.) in dry DCM (50 mL) was added pyridine (1.6 ml, 20.00 mmol, 1.0 eq.) at 0° C. followed by addition of triflic anhydride (6.4 g, 23.00 mmol, 1.15 eq.) and the reaction mixture was stirred at 0° C. for 5 minutes then at RT for 12 h. Progress of reaction was monitored by TLC. After completion, reaction mixture was diluted with water (100 mL) and extracted with DCM (3×50 mL). Combined organic layer was washed with brine solution (50 mL) and dried over anhydrous Na₂SO₄. Removal of solvent under reduced pressure gave 2,2,2-trifluoroethyl trifluoromethanesulfonate (2 g, 43.10%) which was used in the next step without further purification.

### Step-2

To a solution of 4-bromo-1H-pyrazole (500 mg, 3.40 mmol, 1.0 eq.) in dioxane (50 mL) was added cesium carbonate (2.2 g, 6.80 mmol, 2.0 eq.) and the reaction mixture was stirred at RT for 15 minutes. To this mixture was added 2,2,2-trifluoroethyl trifluoromethanesulfonate (986 mg, 4.25 mmol, 1.25 eq.) and the reaction mixture was stirred at RT for 12 h. Progress of reaction was monitored by TLC. After completion, reaction mixture was filtered and the filtrate was evaporated under reduced pressure to afford 4-bromo-1-(2,2,2-trifluoroethyl)-1H-pyrazole (600 mg, 77.14%). Which was used in the next step without purification.

### Step-3

To a solution of 4-bromo-1-(2,2,2-trifluoroethyl)-1H-pyrazole (600 mg, 2.62 mmol) and ethyl (2E)-3-(4,4,5,5-tetramethyl-1,3,2-dioxaborolan-2-yl)acrylate (710 mg, 3.14 mmol, 1.2 eq) in 1,4-dioxane (10 mL) was added potassium tertbutoxide (587 mg, 5.2 mmol, 2.0 eq) and the reaction mixture was deoxygenated by purging nitrogen for 10 minutes. To this mixture was added (PPh₃)₄Pd (151 mg, 0.13 mmol, 0.15 eq) and mixture was again deoxygenated by purging nitrogen for 10 minutes. The reaction mixture was then allowed to stir under microwave at 140° C. for 1.5 h. Reaction mixture was cooled to RT, diluted with EtOAc (50 mL) and washed with brine (3×50 mL). Removal of solvent under reduced pressure gave crude which was purified by Combi-flash on silica gel using EtOAc-hexane as eluent to afford ethyl (2E)-3-[4-bromo-1-(2,2,2-trifluoroethyl)-1H-pyrazol-3-yl]prop-2-enoate (400 mg, 61.34%).

### Step-4

To a solution of ethyl (2E)-3-[4-bromo-1-(2,2,2-trifluoroethyl)-1H-pyrazol-3-yl]prop-2-enoate (300 mg, 1.20 mmol, 1.0 eq) in ethanol (10 mL) was added a solution of lithium hydroxide (87 mg, 3.62 mmol, 3.0 eq) in water (2.0 mL) and the reaction mixture was stirred at 70° C. for 2 h. Reaction mixture was concentrated under vacuum and then acidified with 1N HCl (10 mL). The mixture was diluted with water (30 mL) and extracted with ethyl acetate (3×50 mL). Combined organic layer was washed with brine solution (50 mL) and dried over anhydrous Na₂SO₄. Removal of solvent under reduced pressure gave (2E)-3-[1-(2,2,2-trifluoroethyl)-1H-pyrazol-4-yl]prop-2-enoic acid (260 mg, 97.74%) as crystalline solid.

### Step-5

To a solution of 2E)-3-[1-(2,2,2-trifluoroethyl)-1H-pyrazol-4-yl]prop-2-enoic acid (260 mg, 1.18 mmol, 1.0 eq.) in dry THF (10 mL) was added triethylamine (0.22 mL, 1.41 mmol, 1.2 eq.) followed by pivaloyl chloride (0.14 mL, 1.18 mmol, 1.0 eq.) and the reaction mixture was stirred at 0° C. for 45 minutes. Progress of reaction was monitored by TLC. After completion, reaction mixture was filtered and the filtrate was used directly for the next step.

### Step-6

To a stirred solution of 5,6-dihydropyridin-2(1H)-one (114 mg, 1.17 mmol, 1.0 eq.) in dry THF (10 mL) at −78° C. was added nBuLi (0.50 mL, 1.17 mmol, 1.0 eq.) and the mixture was allowed to stir at the same temperature for 30 minutes. To this solution was added a solution of 22,2-dimethylpropanoic (2E)-3-[1-(2,2,2-trifluoroethyl)-1H-pyrazol-4-yl]prop-2-enoic anhydride (357 mg, 1.17 mmol, 1.0 eq) in THF and the reaction mixture was allowed to stir at room temperature for 1 h. Progress of reaction was monitored by TLC and LCMS. After completion, reaction mixture was evaporated to dryness under vacuum to afford crude which was purified Combi-Flash chromatography (0-50% EtOAc-Hexane) to afford 111-{(2E)-3-[1-(2,2,2-trifluoroethyl)-1H-pyrazol-4-yl]prop-2-enoyl}-5,6-dihydropyridin-2(1H)-one (120 mg, 34.18%).

LCMS: 300 [M+1]⁺

¹H NMR (400 MHz, CHLOROFORM-d) d=7.82 (s, 1H), 7.72 (s, 1H), 7.63 (d, 1H), 7.33-7.25 (m, 1H), 6.96-6.91 (m, 1H), 6.03 (d, 1H), 4.70 (q, 2H), 4.02 (t, 2H), 2.47 (td, 2H).

### Example 90

### Preparation of (E)-tert-butyl 4-(3-oxo-3-(2-oxo-5,6-dihydropyridin-1(2H)-yl)-2-phenylprop-1-enyl)piperidine-1-carboxylate

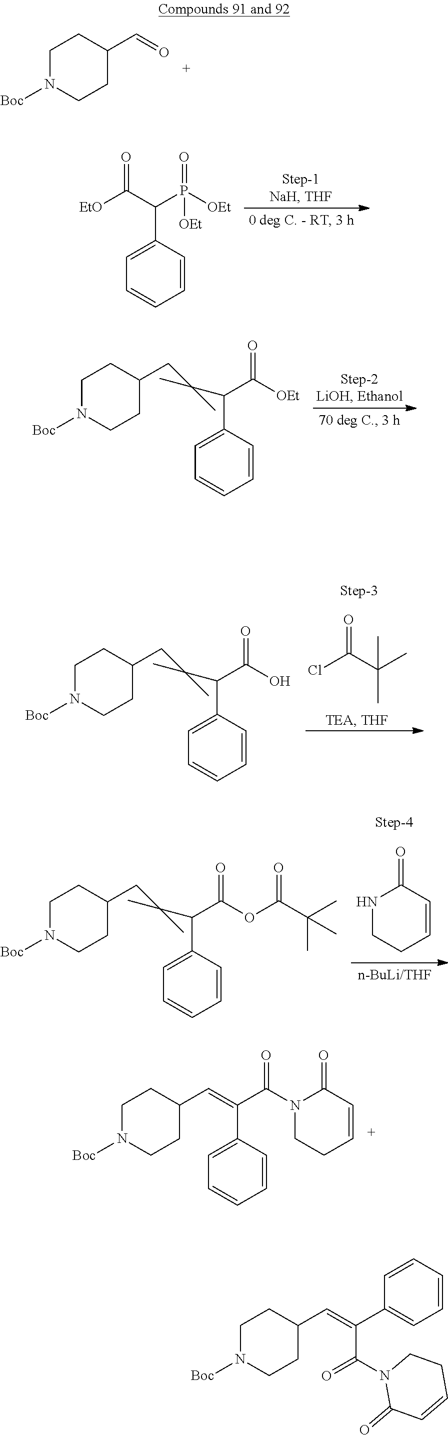

### Step-1

To a suspension of sodium hydride (60% in mineral oil) (450 mg, 11.26 mmol, 1.2 eq) in THF (50 mL) was added ethyl (diethoxyphosphoryl)(phenyl)acetate (3.57 g, 11.26 mmol, 1.2 eq) dropwise at 0° C. and the mixture was allowed to stir at the same temperature for 30 minutes. To this mixture was added a solution of tert-butyl 4-formylpiperidine-1-carboxylate (2.0 g, 9.38 mmol, 1.0 eq) in THF (5 mL) and the resulting mixture was allowed to stir at room temperature for 2 h. Progress of reaction was monitored by TLC. After completion, reaction mixture was diluted with saturated aq. NH₄Cl (100 mL) and extracted with ethyl acetate (3×50 mL). Combined organic layer was washed with brine, dried over anhydrous Na₂SO₄ and evaporated to dryness under vacuum to afford crude which was purified by Combi-Flash on silica gel using ethyl acetate-hexane system as eluent to afford a mixture of (E) and (Z)-tert-butyl 4-(3-ethoxy-3-oxo-2-phenylprop-1-enyl)piperidine-1-carboxylate (900 mg, 30.00%).

### Step-2

To a solution of ((E) and (Z)-tert-butyl 4-(3-ethoxy-3-oxo-2-phenylprop-1-enyl)piperidine-1-carboxylate (900 mg, 2.50 mmol, 1.0 eq) in ethanol (50 mL) was added 2M NaOH solution (10 mL) and the reaction mixture was stirred at 70° C. for 0.5 h. Reaction mixture was concentrated under vacuum and then acidified with 1N HCl (10 mL). The mixture was diluted with water (30 mL) and extracted with ethyl acetate (3×50 mL). Combined organic layer was washed with brine solution (50 mL) and dried over anhydrous Na₂SO₄. Removal of solvent under reduced pressure gave (E) and (Z)-3-(1-(tert-butoxycarbonyl)piperidin-4-yl)-2-phenylacrylic acid (680 mg 82.02%) as crystalline solid

### Step-3

To a solution of (E) and (Z)-3-(1-(tert-butoxycarbonyl)piperidin-4-yl)-2-phenylacrylic acid (680 mg, 2.05 mmol, 1.0 eq.) in dry THF (10 mL) was added triethylamine (0.32 mL, 2.25 mmol, 1.1 eq.) followed by pivaloyl chloride (1.0 mL, 2.05 mmol, 1.0 eq.) and the reaction mixture was stirred at 0° C. for 45 minutes. Progress of reaction was monitored by TLC. After completion, reaction mixture was filtered and the filtrate was used directly for the next step.

### Step-4

To a stirred solution of 5,6-dihydropyridin-2(1H)-one (175 mg, 1.80 mmol, 1.0 eq.) in dry THF (10 mL) at −78° C. was added nBuLi (0.70 mL, 1.80 mmol, 1.0 eq.) and the mixture was allowed to stir at the same temperature for 30 minutes. To this solution was added a solution of (E)-3-(1-(tert-butoxycarbonyl)piperidin-4-yl)-2-phenylacrylic pivalic anhydride (748 mg, 1.80 mmol, 1.0 eq.) in THF and the reaction mixture was allowed to stir at room temperature for 1 h. Progress of reaction was monitored by TLC and LCMS. After completion, reaction mixture was evaporated to dryness under vacuum to afford crude which was purified HPLC column chromatography to afford (E)-tert-butyl 4-(3-oxo-3-(2-oxo-5,6-dihydropyridin-1(2H)-yl)-2-phenylprop-1-enyl)piperidine-1-carboxylate (50 mg, 20%) and (Z)-tert-butyl 4-(3-oxo-3-(2-oxo-5,6-dihydropyridin-1(2H)-yl)-2-phenylprop-1-enyl)piperidine-1-carboxylate (50 mg, 20%).

### Analytical Data

### Compound 91

LCMS: 411[M+1]⁺

¹H NMR (400 MHz, CDCl₃) δ 7.40-7.20 (m, 5H), 6.85-6.75 (m, 1H), 5.90 (d, 1H), 5.80 (d, 1H), 4.10-3.95 (m, 2H), 3.90 (t, 2H), 2.70-2.52 (m, 2H), 2.45-2.36 (m, 2H), 1.55 (s, 9H), 1.70-1.20 (m, 5H).

### Compound 92

LCMS: 411[M+1]⁺

¹H NMR (400 MHz, CDCl₃) δ 7.32-7.20 (m, 5H), 6.87-6.75 (m, 1H), 5.80 (d, 1H), 5.67 (d, 1H), 4.19-3.95 (m, 4H), 2.70-2.52 (m, 2H), 2.45-2.36 (m, 2H), 1.55 (s, 9H), 1.70-1.20 (m, 5H).

### Example 91

### Preparation of (E)-3-methyl-1-(3-(1-methyl-1H-pyrazol-4-yl)acryloyl)-5,6-dihydropyridin-2(1H)-one

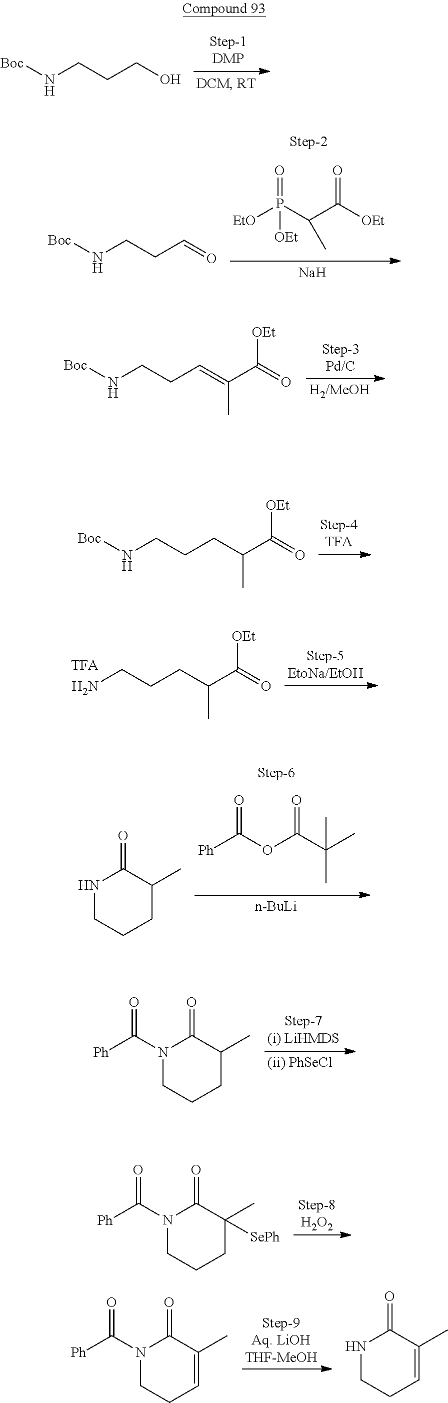

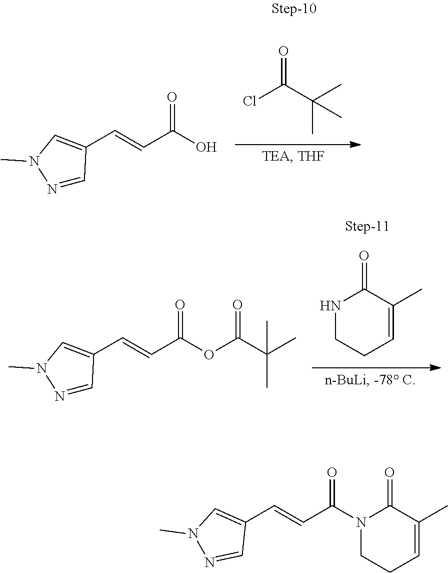

### Step-1

To a solution of tert-butyl (3-hydroxypropyl)carbamate (5 g, 28.57 mmol, 1 eq) in DCM (50 mL) was added Dess-Martin periodinane (16 g, 37.14 mmol, 1.3 eq) and the reaction mixture was allowed to stir at room temperature for 2 h. Progress of reaction was monitored by proton NMR. After completion, reaction mixture was diluted with hexane (500 mL) and stirred for 15 minutes. Reaction mixture was filtered, organic layer was washed with sat. NaHCO₃(2×100 mL). Organic layer was washed with brine (100 mL) and dried over anhydrous sodium sulfate. Removal of solvent afforded crude tert-butyl (3-oxopropyl)carbamate (4.1 g, 82.99%) which was used in the next step without further purification.

### Step-2

To a solution of ethyl 2-(diethoxyphosphoryl)propanoate (2.9 g, 16.8 mmol, 1 eq) in THF (30 mL) was added NaH (0.8 g, 20 mmol, 1.3 eq) at 0° C. and the reaction mixture was allowed to stir at the same temperature for 10 minutes. To this solution was added tert-butyl (3-oxopropyl)carbamate (4.1 g, 16.8 mmol, 1 eq) and the reaction mixture was allowed to stir at room temperature for 30 minutes. Progress of reaction was monitored by proton NMR. After completion, reaction mixture was diluted with a Aq. ammonium chloride solution (50 mL) and extracted with ethyl acetate (3×100 mL). Combined organic layer was washed with brine (100 mL) and dried over anhydrous sodium sulfate. Removal of solvent afforded crude ethyl (E)-5-((tert-butoxycarbonyl)amino)-2-methylpent-2-enoate (3.9 g, 66.10%) which was used in the next step without further purification.

### Step-3

To a solution of ethyl (E)-5-((tert-butoxycarbonyl)amino)-2-methylpent-2-enoate (5.15 g, 20 mmol, 1 eq) in MeOH (100 mL) was added Pd/C (0.6 g) and the reaction mixture was allowed to stir at room temperature under H₂ atmosphere for 16 h. Progress of reaction was monitored by proton NMR. After completion, reaction mixture was passed through celite bed. Removal of solvent afforded crude ethyl 5-((tert-butoxycarbonyl)amino)-2-methylpentanoate (4.64 g, 90.09%) which was used in the next step without further purification.

### Step-4

To a solution of ethyl 5-((tert-butoxycarbonyl)amino)-2-methylpentanoate (4.64 g, 17.89 mmol, 1 eq) in DCM (10 mL) was added TFA (10 mL, 130 mmol, 7.3 eq) dropwise and the reaction mixture was allowed to stir at room temperature for 1 h. Progress of reaction was monitored by proton NMR. After completion, removal of solvent and azeotrope with toluene afforded crude ethyl 5-amino-2-methylpentanoate (6.3 g, crude) which was used in the next step without further purification.

### Step-5

To a solution of ethyl 5-amino-2-methylpentanoate (5.8 g, 36.42 mmol, 1 eq) in ethanol (120 mL) was added NaH (1.74 g, 72.85 mmol, 2 eq) portion-wise at 0° C. and the reaction mixture was allowed to stir 0° C. for 10 minutes followed by stirring at 70° C. for 2 h. Progress of reaction was monitored by proton NMR. After completion, removal of solvent under reduced pressure afforded crude which was purified by CombiFlash on silica gel using ethyl acetate-hexane system as eluent to afford 3-methylpiperidin-2-one (1.46 g, 35.44%).

### Step-6

To a stirred solution of 3-methylpiperidin-2-one (0.5 g, 4.41 mmol, 1 eq.) in dry THF at −78° C. was added n-butyl lithium (2.5 M in hexane, 1.94 mL, 4.85 mmol, 1.1 eq.) and the mixture was allowed to stir at the same temperature for 30 minutes. To this solution was added benzoic pivalic anhydride (0.592 g, 4.85 mmol, 1.1 eq.). Reaction mixture was allowed to stir at −78° C. for 90 min. Progress of reaction was monitored by TLC. Reaction mixture was brought to 0° C., diluted with ammonium chloride solution (30 mL) and extracted with ethyl acetate (3×30 mL). Combined organic layer was washed with brine, dried over anhydrous Na₂SO₄ and evaporated to dryness under vacuum to afford crude which was purified by CombiFlash to afford 1-benzoyl-3-methylpiperidin-2-one (0.480 g, 50%).

### Step-7

To a stirred solution of 1-benzoyl-3-methylpiperidin-2-one (0.230 g, 1.06 mmol, 1 eq.) in dry THF at −78° C. was added LiHMDS (1 M in THF, 1.27 mL, 1.27 mmol, 1.2 eq.) and the mixture was allowed to stir at the same temperature for 30 minutes. To this solution was added phenyl hypochloroselenoite (0.241 g, 1.27 mmol, 1.2 eq.). Reaction mixture was allowed to stir at −78° C. for 90 min. Progress of reaction was monitored by TLC and LCMS. Reaction mixture was brought to 0° C., diluted with water (25 mL) and extracted with ethyl acetate (3×40 mL). Combined organic layer was washed with brine, dried over anhydrous Na₂SO₄ and evaporated to dryness under vacuum to afford crude which was purified by Combi-Flash to afford 1-benzoyl-3-methyl-3-(phenylselanyl)piperidin-2-one (0.330 g, 83.45%).

### Step-8

To a stirred solution of 1-benzoyl-3-methyl-3-(phenylselanyl)piperidin-2-one (5 g, 13.42 mmol, 1 eq.) in dry THF (40 mL) at 0° C. was added H₂O₂(30%) (15 mL) and the mixture was allowed to stir at the same temperature for 10 minutes followed by stirring at RT for 30 minutes. Progress of reaction was monitored by TLC and LCMS. After completion, reaction mixture was diluted with water (100 mL) and extracted with ethyl acetate (3×50 mL). Combined organic layer was washed with brine (50 mL), dried over anhydrous Na₂SO₄ and evaporated to dryness under vacuum to afford crude 1-benzoyl-3-methyl-5,6-dihydropyridin-2(1H)-one (1.4 g, crude) which was used in next step without purification.

### Step-9

To a stirred solution of 1-benzoyl-3-methyl-5,6-dihydropyridin-2(1H)-one (1.4 g, 6.50 mmol, 1 eq.) in mixture of MeOH (8 mL) and THF (8 mL) and the mixture was allowed to stir at room temperature for 10 minutes followed by addition of LiOH (0.8 g, 39.02 mmol, 8 eq.). The resulting reaction mixture was stirred at room temperature for 3 h. Progress of reaction was monitored by TLC. Reaction mixture was diluted with water and extracted with ethyl acetate (6×40 mL). Combined organic layer was washed with brine, dried over anhydrous Na₂SO₄ and evaporated to dryness under vacuum. Crude was purified by CombiFlash to afford 3-methyl-5,6-dihydropyridin-2(1H)-one (0.5 g, 69.25%).

### Step-10

To a solution of (E)-3-(1-methyl-1H-pyrazol-4-yl)acrylic acid (0.273 g, 1.79 mmol, 1.0 eq.) in dry THF (12 mL) was added triethylamine (0.3 mL, 2.15 mmol, 1.2 eq.) and pivaloyl chloride (0.24 mL, 1.97 mmol, 1.1 eq.) the reaction mixture was stirred at 0° C. for 45 minutes. Progress of reaction was monitored by TLC. After completion, reaction mixture was filtered and the filtrate was used directly for the next step.

### Step-11

To a stirred solution of 3-methyl-5,6-dihydropyridin-2(1H)-one (0.2 g, 1.79 mmol, 1 eq.) in dry THF (15 mL) at −78° C. was added n-butyllithium (2.5 M in hexane, 0.71 mL, 1.79 mmol, 1 eq.) and the mixture was allowed to stir at the same temperature for 30 minutes. To this solution was added (E)-(E)-3-(1-methyl-1H-pyrazol-4-yl)acrylic pivalic anhydride (0.402 g, 1.79 mmol, 1 eq.). Reaction mixture was allowed to stir at −78° C. for 2 h. Progress of reaction was monitored by TLC and LCMS. Reaction mixture was brought to 0° C., diluted with sat. ammonium chloride solution and extracted with ethyl acetate (3×30 mL). Combine organic layer was washed with brine, dried over anhydrous Na₂SO₄ and evaporated to dryness under vacuum. To afford crude which was purified by Combi-Flash afford (E)-3-methyl-1-(3-(1-methyl-H-pyrazol-4-yl)acryloyl)-5,6-dihydropyridin-2(1H)-one (3 mg, 0.72%).

LCMS: 246 [M+1]⁺

¹H NMR (400 MHz, CDCl3) δ 7.75 (s, 1H), 7.61 (s, dH), 7.59 (s, 1H), 7.23 (d, 1H), 6.65 (s, 1H), 3.99 (t, 2H), 3.90 (s, 3H), 2.42-2.34 (m, 2H), 1.95 (s, 3H).

### Example 92

### Preparation of (E)-6,6-dimethyl-1-(3-(3,4,5-trimethoxyphenyl)acryloyl)-5,6-dihydropyridin-2(1H)-one

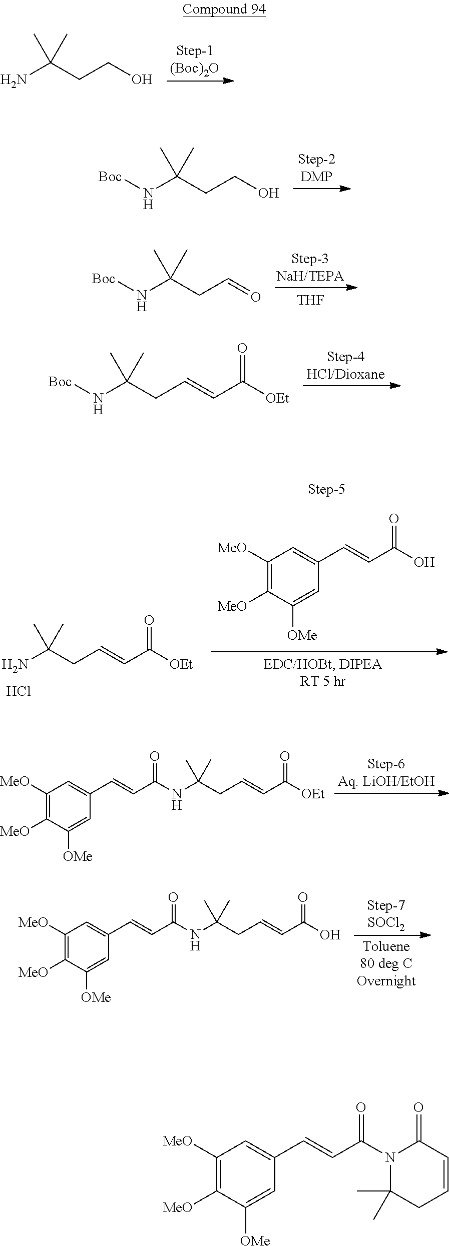

### Step-1

To a solution of 3-amino-3-methyl-butan-1-ol (5.0 g, 48.54 mmol) in CH₂Cl₂ (150 mL) was added di-tert-butyl dicarbonate (13.22 g, 60.27 mmol) at RT and resulting mixture was stirred for 18 h. Progress of reaction was monitored by TLC. After completion, reaction mixture was concentrated under vacuum to afford tert-butyl (4-hydroxy-2-methylbutan-2-yl)carbamate as an amber oil (9.8 g) which was used in the next step without purification.

### Step-2

To a solution of the tert-butyl (4-hydroxy-2-methylbutan-2-yl)carbamate (2 g, 9.8 mmol) in DCM (18 mL) at 0° C. was added Dess-Martin periodinane (7.2 g, 17.14 mmol). The reaction mixture was stirred at RT for 3 h. Progress of reaction was monitored TLC. After completion, reaction mixture was quenched with aq. NaHCO₃, and extracted with EtOAc (2×50 mL). Combined organic layer was washed with brine (2×20 mL), dried over sodium sulfate. Removal of solvent gave crude which was purified by silica gel column chromatography using ethyl acetate-hexane as eluent to afford tert-butyl (2-methyl-4-oxobutan-2-yl)carbamate as a yellow solid (1.8 g).

### Step-3

To a suspension of sodium hydride (60% in mineral oil, 0.56 g 13.87 mmol, 1.2 eq) in THF (50 mL) was added triethylphosphonoacetate (2.58 g, 11.52 mmol, 1.2 eq) dropwise at 0° C. and the mixture was allowed to stir at the same temperature for 30 minutes. To this mixture was added a solution of tert-butyl (2-methyl-4-oxobutan-2-yl)carbamate (2 g, 11.52 mmol, 1.0 eq) in THF (5 mL) and the resulting mixture was allowed to stir at room temperature for 1 h. Progress of reaction was monitored by TLC. After completion, reaction mixture was diluted with saturated aq. NH₄Cl (100 mL) and extracted with ethyl acetate (3×50 ml). Combined organic layer was washed with brine, dried over anhydrous Na₂SO₄ and evaporated to dryness under vacuum to afford crude which was purified by Combi-Flash on silica gel using ethyl acetate-hexane system as eluent to afford ethyl (E)-5-((tert-butoxycarbonyl)amino)-5-methylhex-2-enoate (2 g).

### Step-4

To a solution of ethyl (E)-5-((tert-butoxycarbonyl)amino)-5-methylhex-2-enoate (2 g) 1,4-dioxane (8 mL) at RT was added 20% HCl in dioxane (8 mL) and reaction mixture was allowed to stir at RT for 2 h. Progress of reaction was monitored by TLC. After completion, reaction mixture was removed under vacuum and the resulting yellow solid was triturated with EtOAc to afford ethyl (E)-5-amino-5-methylhex-2-enoate hydrochloride as a yellow solid (1.5 g).

### Step-5

To a solution of (E)-ethyl 5-amino-5-methylhex-2-enoate hydrochloride (1 g, 4.83 mmol) in DCM (30 mL) was added DIPEA (1.38 ml, 8.40 mmol), EDC.HCL (960 mg, 5.04 mmol), HOBt (770 mg, 5.04 mmol) and ethyl (E)-5-amino-5-methylhex-2-enoate hydrochloride (870 mg, 4.20 mmol) at 0° C. and reaction mixture was stirred at RT for 5 h. Progress of reaction was monitored by TLC. After completion, reaction mixture was diluted with water (50 mL) and extracted with EtOAc (3×50 mL). Combined organic layer was over sodium sulfate and concentrated under vacuum to afford crude which was purified by Combi-Flash on silica gel using ethyl acetate-hexane system as eluent to afford ethyl (E)-5-methyl-5-((E)-3-(3,4,5-trimethoxyphenyl)acrylamido)hex-2-enoate (0.8 g).

### Step-6

To a solution of ethyl (E)-5-methyl-5-((E)-3-(3,4,5-trimethoxyphenyl)acrylamido)hex-2-enoate (800 mg, 2.04 mmol, 1.0 eq) in ethanol (30 mL) was added a solution of lithium hydroxide (244 mg 10.20 mmol, 5 eq) in water (3 mL) and the reaction mixture was stirred at room temperature for 4 h. Reaction mixture was concentrated under vacuum and then acidified with 2N HCl make pH up to 3. The mixture was diluted saturated aq. NH₄Cl (30 mL) and extracted with dichloromethane (3×30 mL). The combined organic layer was washed with brine solution (50 mL) and dried over anhydrous Na₂SO₄. Removal of solvent under reduced vacuum gave product (E)-5-methyl-5-(3-(3,4,5-trimethoxyphenyl)acrylamido)hexanoic acid as crystalline solid (0.7 g).

### Step-7

A mixture of (E)-5-methyl-5-((E)-3-(3,4,5-trimethoxyphenyl)acrylamido)hex-2-enoic acid (150 mg, 0.413 mmol 1 eq) and thionyl chloride (0.14 mL, 2.07 mmol, 5 eq) in toluene (10 mL) was stirred at 80° C. for 16 h. Reaction mixture was cooled to RT, diluted with EtOAc and washed with NaHCO₃(3×50 ml). The combined organic layer was washed with brine solution (50 mL) and dried over anhydrous Na₂SO₄. Removal of solvent under reduced pressure gave crude which was purified by Combi-Flash on silica gel using ethyl acetate-hexane system as eluent to afford (E)-6,6-dimethyl-1-(3-(3,4,5-trimethoxyphenyl)acryloyl)-5,6-dihydropyridin-2(1H)-one (20 mg).

LCMS: 346 [M+1]⁺

¹H NMR (400 MHz, CD₃OD) δ ppm 7.55 (d, 1H), 6.97 (s, 2H), 6.96-6.82 (m, 1H), 6.80 (d, 1H), 6.00 (d, 1H), 3.85 (s, 6H), 3.60 (s, 3H), 2.40-2.35 (m, 2H).

### Example 93

### In Vitro Cell Viability Assay

The present disclosure provides a method for inhibiting cell viability, as measured by ATP levels. The method for inhibiting cell viability is drawn to contacting various cancer cells with the compounds described herein at various concentrations. The cancer cell lines were maintained in culture conditions at 37° C. in an atmosphere of 5% CO₂ in air. The cells growing in an exponential growth phase were harvested and counted for plating. Compounds were incubated in two cancer cell types representing multiple myeloma (MOLP-8) and triple negative breast cancer (DU4475). Cells were plated at an optimized cell numbers per well in 96-well plates and allowed to attach to the plates overnight at approximately 80% confluency.

After addition of compounds, plates were incubated for 7-day. At the end of incubation period on day 8, media was removed and replaced with a solution of CellTiter-Glo™ reagent (Promega®) according to protocol. Luminescence was read using an EnVision® multi-label plate reader (Perkin-Elmer®), and signal intensity was calculated relative to positive and negative control wells. Cisplatin was included as the positive control. A dose-response calculation was performed in a non-linear regression fit and by using the Hill equation to calculate a concentration-response curve and 50% inhibitory concentration (IC₅₀) values for each compound.

The average IC₅₀ values of piperlongumine in multiple myeloma (MOLP-8) and triple negative breast cancer (DU4475) cells were approximately 1.01 uM and 1.24 uM, respectively. The average IC₅₀ values of compounds 1-15 in multiple myeloma (MOLP-8) and triple negative breast cancer (DU4475) cells are shown in Table 1.

### Example 94

### Determination of Potency of Compounds in Cell Viability Assay Using Colorectal Cancer Cells

The blue dye resazurin-based assay was performed to determine cell viability and used according to manufacture protocol (Sigma®). HCT116 (colorectal cancer) cells were seeded in the DMEM medium (10% FBS serum). Approximately 1,500 cells were plated per well in 96 well plates. Cells were allowed to grow at 37° C. for 24 hr in 5% CO₂ environment in a humidified incubator. Serially diluted compounds (100 μL) were added to the culture plate after 24 hr. After addition of compounds, plates were incubated for 72 hr. Experiment was terminated at the designated incubation time by replacing the medium with 100 μL of 1 mM of resazurin (Sigma®) prepared in culture medium (DMEM) and were further incubated in culture conditions for 4-6 hours. Fluorescence was recorded using a multimodal plate reader (Biotek Synergy Neo®) at an excitation wavelength of 535 nm and emission wavelength of 590 nm to obtain relative fluorescence units. Data analysis was done by subtracting the background fluorescence (medium blank) value from each reading and then normalizing with the vehicle control (DMSO treated cells) to obtain percent survival/proliferation. IC₅₀ was calculated from percent survival values using GraphPad Prism® (see Table 1).

### Example 95

### In Vivo Pharmacokinetic Analysis

Age 8 to 9 weeks Balb/C female mice were used for this study. The mice were maintained in a special pathogen-free environment and in disposable cages. Animal room was set to maintain temperature and relative humidity at 23° C.±3° C. and 55%±15%, respectively. Housing room was on a 12:12 light/dark cycle. Water was supplied ad libitum to each cage via water bottles. All mice were fed rodent diet with 18% protein. All animals were weighed before commencement of the study.

Compound 1 was stored at 4° C. protected from light. Dosing solutions were used within 2 hours of preparation. Compound 1 was formulated within 2 hours prior to dosing by adding the pre-calculated volume of PEG 400 (40% of total solution volume). Formulated compound 1 was vortexed and sonicated until a clear solution was formed. To this clear formulation, saline (60% of total solution volume) was added and vortexed until a clear solution was formed. The Compound 1 solution was filtered using a sterile syringe filter (0.2 μm coming filter). All Compound 1 solutions were mixed thoroughly prior to pulling up into syringes and prior to dosing. 12 mice were dosed intravenously at 5 mg/kg. 12 mice were dosed intraperitoneally at 10 mg/kg. 12 mice were dosed orally at 10 mg/kg. Animals were sacrificed for blood and organs sampling. Under isoflurane anesthesia, blood was collected via retro-orbital route. Blood samples were then centrifuged for 5 minutes at a minimum of 2,000×g. Plasma supernatant (a minimum of 50 μL or as much as feasible to collect) was collected. Plasma was stored at −80° C.

Bioanalytical analysis of plasma levels of Compound 1 was performed by mass spectrometry. Calibration standards were prepared by spiking the test compound into blank plasma. Calibrator concentrations were 5,000, 2,000, 1,000, 200, 100, 20, 10, 2 and 1 ng/mL. Calibration standard samples were processed along with the test samples.

Twenty μL aliquots of plasma test samples were treated with 100 μL of methanol: acetonitrile (5:95 v:v) containing internal standard (12.5 ng/mL verapamil). The mixtures were vortexed on a shaker for 15 minutes and subsequently centrifuged at 4,000 rpm for 15 minutes. An aliquot of 50 μL of the supernatant was mixed with 100 μL of water with 0.1% formic acid for injection to the LC/MS/MS (liquid chromatography/tandem-mass spectrometry).

The concentration of Compound 1 was determined by a UPLC-TIS-MS-MS method (ultra performance liquid chromatography—turbo ion spray—tandem mass spectrometry). The UPLC comprised a Shimadzu LC-30AD HPLC (high performance liquid chromatography). Chromatographic separation was achieved using a Waters UPLC column (Acquity UPLC BEH C18, 50×2.1 mm, 1.7 μM). Mobile phase A was water with 0.1% formic acid; mobile phase B was acetonitrile with 0.1% formic acid. The gradient program was as follows: 20% B (0-0.2 min), 20-95% B (0.2-1.2 min), 95% B (1.2-1.8 min), 95-20% B (1.81-2.2 min). The flow rate was 0.6 mL/minute. The column was maintained at 50° C.

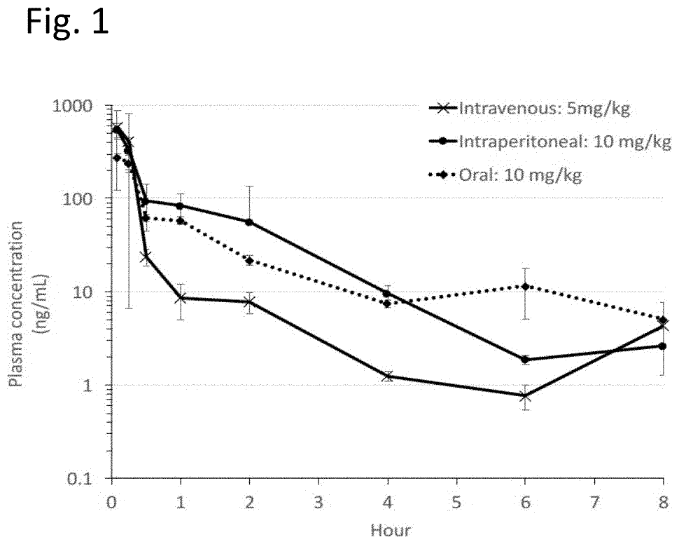

Mass spectrometric analysis was performed using an AB Sciex API5000 with Turbo Ion Spray interface operating in a positive ionization mode. Quantification was performed using multiple reaction monitoring (MRM) method with the transitions of (m/z) 316.104→(m/z) 219.014 for Compound 1 and (m/z) 455.346→(m/z) 165 for verapamil (internal standard). Pharmacokinetics of Compound 1 ((E)-1-(3-(8-methoxy-2,3-dihydrobenzo[b][1,4]dioxin-6-yl)acryloyl)-5,6-dihydropyridin-2(1H)-one) in the plasma of the mice dosed intravenously (IV; 5 mg/kg), intraperitoneally (IP; 10 mg/kg) and orally (PO; 10 mg/kg) (n=3 per time point) is illustrated in FIG. 1. Plasma half-life (t_{1/2}) of Compound 1 and piperlongumine is shown in Table 2 below.

TABLE 2

| Route | Dose (mg/kg) | Compound 1 t_{1/2} (hr) | Piperlongumine t_{1/2} (hr) |
|---|---|---|---|
| IV | 5 | 1.66 | 1.26 |
| IP | 10 | 1.14 | 1.07 |
| PO | 10 | 2.16 | 1.31 |

As illustrated in FIG. 1 and Table 2, Compound 1 exhibited increased in vivo plasma half-life (t_{1/2}) as compared to piperlongumine. Consistent with these results, in vitro microsome stability analyses revealed that Compounds 1 and compounds in Table 1 having R¹ functional group according to Formula (I) demonstrated potency and/or metabolic stability. Particularly, a subset of compounds containing heterocyclic R¹ exhibited a combined potency and microsomal stability that is at least 2-fold greater than piperlongumine. Enhanced in vivo cytotoxicity, bioavailability, and stability of the compounds disclosed herein allow for lower and less frequent dosing, thus promoting a better clinical outcome.

### Example 96

### Compound Effect in Collagen-Induced Arthritis in Female Lewis Rats

Methods

Animals were anesthetized with isoflurane and given intradermal injections of 400 μl (2×200 μl) of Porcine Type II collagen in Freund's incomplete adjuvant spread equally over two sites on the back on study days 0. On day 7, a boost injection was given; intradermal injections of 100 μl of Porcine Type II collagen in Freund's incomplete adjuvant. Collagen was prepared by making a 2 mg/ml solution in 0.01 N Acetic acid. Equal volumes of collagen and Freund's incomplete adjuvant were emulsified by hand mixing with the syringes partially submerged in ice water for 10 minutes, at which point a bead of this material will hold its form when placed in water.

On study day 6, the animals were randomized by body weight into treatment groups and dosing was initiated. The rats were then dosed on study days 6 through 17 either daily (QD) by the oral (PO) route with vehicle (40% PEG400/saline) or baricitinib (3 mg/kg), twice daily (BID) PO with Compound 56 (20 or 40 mg/kg), or QD by the intraperitoneal (IP) route with Compound 56 (20 mg/kg).

Efficacy evaluation was based on animal body weights, ankle caliper measurements, and final hind paw weights. Terminal serum was analyzed for anti-type II collagen antibody (IgG) concentrations.

The rats were weighed on day 6 for randomization, on day 9, and daily on days 11 through 17.

Caliper measurements of right and left ankle diameters were taken on study days 9 (baseline) and 11 through 17. Ankle caliper measurements were made with a digital micrometer (Digitrix II; Fowler & NSK). Baseline measurements were taken using one ankle with values rounded to one thousandth of an inch. Measurements were confirmed as clinically normal (0.260-0.264 in) by comparison with historical values for rats based on a range of body weights. Baseline measurements were then applied to both ankles, and these values remained with the animal so long as the ankle was clinically normal with good definition of all the ankle bones and no evidence of inflammation.

Statistical analysis was performed using a one-way analysis of variance with Dunnett's multiple comparisons test.

Results

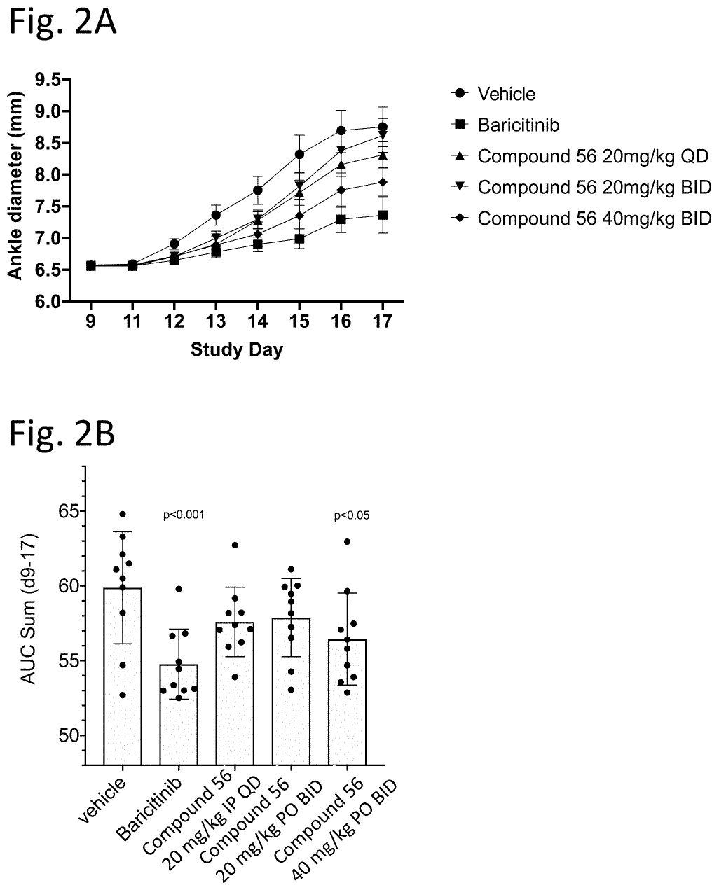

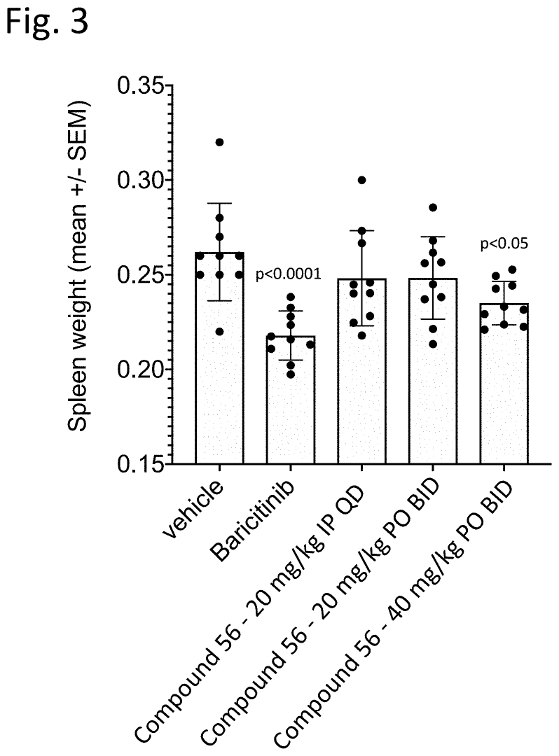

The ankle diameters measured over time for each treatment condition are shown in FIG. 2A and resulting area under the curve (AUC) calculations for days 9-17 are shown in FIG. 2B. Measurements of spleen weight relative to body weight, an indicator of inflammation, is shown in FIG. 3.

Twice daily oral treatment with 40 mg/kg Compound 56 resulted in statistically significant reductions in ankle diameter measurements on study days 13 through 16 as compared to vehicle control rats with a correspondingly significant reduction (48%) in ankle diameter area under the curve (AUC) calculations.

Treatment at 40 mg/kg also resulted in a statistical reduction in spleen weights relative to body weight. Animal body weight loss, final paw weights, relative thymus weights, and serum anti-type II collagen antibody (IgG) concentrations in rats treated with Compound 56 at 40 mg/kg did not differ statistically from the vehicle control group.

Results of twice daily oral treatment at 20 mg/kg were not significant. Daily treatment with 20 mg/kg Compound 56 dosed intraperitoneally resulted in a statistical reduction in ankle caliper measurements on day 13 with no other statistically significant findings, although both conditions showed a trend towards reduced ankle diameter and relative spleen weight.

Daily oral treatment with baricitinib at 3 mg/kg resulted in significant inhibition (42%) of body weight loss and significant reduction of ankle diameter measurements (72% reduction of AUC) and final hind-paw weights (71%), along with statistically significant reductions in relative spleen weights as compared to vehicle control rats (FIG. 3). All animals survived to termination.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled. Such modifications are intended to fall within the scope of the appended claims.

All references, patent and non-patent, cited herein are incorporated herein by reference in their entireties and for all purposes to the same extent as if each individual publication or patent or patent application was specifically and individually indicated to be incorporated by reference in its entirety for all purposes.

1. A composition comprising a compound of Formula I or a pharmaceutically acceptable salt thereof: 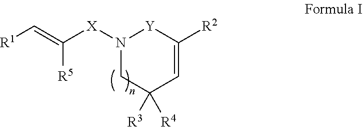
wherein n is an integer of 1,
X and Y are independently C(O) or S(O)₂;
R¹ is selected from the group consisting of:
i) a 3-9 member saturated or partially unsaturated cycloalkyl group having 0-5 heteroatom(s);
ii) a 5 member monocyclic heteroaryl group having 2-3 heteroatoms;
iii) a 6 member monocyclic heteroaryl group having 1-3 heteroatom(s), wherein said 6 member monocyclic heteroaryl group has:
a) 1-3 heteroatom(s) when X or Y is S(O)₂ or when at least one of R², R³ and R⁴ is not hydrogen;
b) two heteroatoms when said 6 member monocyclic heteroaryl group is pyridazinyl or pyrimidyl; or
c) three heteroatoms when X or Y is independently C(O);
iv) a 7-9 member monocyclic unsaturated cycloheteroalkyl group having 1-3 heteroatom(s);
v) a 7-13 member polycyclic heteroaryl group having 1-5 heteroatom(s), wherein said 7-13 member heteroaryl group has:
a) 1-5 heteroatom(s) when at least one of X or Y is S(O)₂ and at least one of R², R³ and R⁴ is not hydrogen; or
b) 2-5 heteroatoms when X and Y are C(O);
vi) a C₁-C₄ alkyl group; and
vii) a phenyl group when at least one of R², R³ and R⁴ is —CN, wherein said heteroatom(s) contained in each cyclic group is independently selected from the group consisting of N, O, and S; and wherein each cyclic group is optionally substituted with halo, alkyl, alkenyl, alkynyl, alkoxy, acyl, acyloxy, carboxylic acid, ester, amine, amide, carbonate, carbamate, carbonyl, sulfonyl, cyano, nitro, thioether, thioester, cycloalkyl, cycloheteroalkyl, heterocyclyl, aryl, or heteroaryl, any of which is unsubstituted or substituted with halo, hydroxyl, sulfhydryl, nitro, nitroso, cyano, azido, alkyl, alkenyl, cycloalkyl, cycloheteroalkyl, heterocyclyl, aryl or heteroaryl;
R² is selected from the group consisting of hydrogen, halo, hydroxyl, alkyl, alkenyl, alkynyl, alkoxy, acyl, acyloxy, carboxylic acid, ester, amine, amide, carbonate, carbamate, cyano, nitro, thioether, thioester, cycloalkyl, cycloheteroalkyl, heterocyclyl, aryl, and heteroaryl, each of which is optionally substituted with halo, hydroxyl, sulfhydryl, nitro, nitroso, cyano, azido, alkyl, alkenyl, cycloalkyl, cycloheteroalkyl, heterocyclyl, aryl or heteroaryl;
each of R³ and R⁴ is independently selected from the group consisting of hydrogen, halo, hydroxyl, alkyl, alkenyl, and alkynyl, any of which is optionally substituted with halo, hydroxyl, alkyl, or cycloalkyl; or R³ taken together with one or more atoms of R⁴ forms a cycloalkyl or cycloheteroalkyl, each of which is optionally substituted with halo, hydroxyl, alkyl, alkenyl, alkynyl, or alkoxy; and
R⁵ is selected from the group consisting of hydrogen, halo, hydroxyl, alkyl, alkenyl, alkynyl, alkoxy, acyl, acyloxy, carboxylic acid, ester, amine, amide, carbonate, carbamate, cyano, nitro, thioether, thioester, cycloalkyl, cycloheteroalkyl, heterocyclyl, aryl, and heteroaryl, each of which is optionally substituted with halo, hydroxyl, sulfhydryl, nitro, nitroso, cyano, azido, alkyl, alkenyl, cycloalkyl, cycloheteroalkyl, heterocyclyl, aryl or heteroaryl.

2. The composition of claim 1, wherein R¹ is a 3-9 member saturated or partially unsaturated cycloalkyl group having 0-5 heteroatom(s).

3-11. (canceled)

12. The composition of claim 1, wherein R¹ is a 5 member monocyclic heteroaryl group having 2-3 heteroatoms independently selected from N, O, or S.

13-19. (canceled)

20. The composition of claim 1, wherein R¹ is a 6 member monocyclic heteroaryl group having 1-3 heteroatom(s) when at least one of X or Y is S(O)₂.

21. The composition of claim 1, wherein R¹ is a 6 member monocyclic heteroaryl group having 1-3 heteroatom(s) when at least one of R², R³ and R⁴ is not hydrogen.

22-24. (canceled)

25. The composition of claim 1, wherein R¹ is pyridazinyl.

26. The composition of claim 1, wherein R¹ is pyrimidyl.

27. The composition of claim 1, wherein R¹ is a 6 member monocyclic heteroaryl group having three heteroatoms independently selected from N, O, or S when at least one of X or Y is independently C(O).

28. (canceled)

29. (canceled)

30. The composition of claim 1, wherein R¹ is a 7-9 member monocyclic unsaturated cycloheteroalkyl group having 1-3 heteroatom(s) selected from the group consisting of azepinyl, diazepinyl, oxepinyl, thiepinyl, thiazepinyl, azocinyl, oxocinyl, thiocinyl, azoninyl, oxoninyl, and thioninyl.

31. The composition of claim 1, wherein R¹ is a 7-13 member polycyclic heterocyclyl group having 1-5 heteroatom(s) independently selected from N, O, or S, wherein at least one of X or Y is S(O)₂ and at least one of R², R³ and R⁴ is not hydrogen.

32. The composition of claim 1, wherein R¹ is a 7-13 member polycyclic heteroaryl group having 2-5 heteroatoms independently selected from N, O, or S, wherein both X and Y are C(O).

33-48. (canceled)

49. A composition comprising a compound of Formula I or a pharmaceutically acceptable salt thereof: 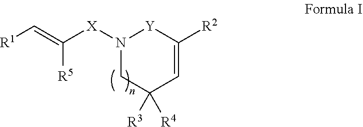
wherein n is an integer of 0 or 2;
X or Y is independently C(O) or S(O)₂;
R¹ is selected from the group consisting of:
a) a branched C₁-C₄ alkyl group;
b) a saturated or partially unsaturated C₃-C₉ cycloalkyl group having no heteroatom;
c) a saturated, partially unsaturated or unsaturated 3-13 membered heterocyclyl group having 1-5 heteroatom(s) independently selected from the group consisting of N, O, and S; and
d) a phenyl group when at least one of R², R³ and R⁴ is —CN,
wherein each group is optionally substituted with halo, alkyl, alkenyl, alkynyl, alkoxy, acyl, acyloxy, carboxylic acid, ester, amine, amide, carbonate, carbamate, carbonyl, sulfonyl, cyano, nitro, thioether, thioester, cycloalkyl, cycloheteroalkyl, aryl, heteroaryl, or heterocyclyl, any of which is unsubstituted or substituted with halo, hydroxyl, sulfhydryl, nitro, nitroso, cyano, azido, alkyl, alkenyl, cycloalkyl, cycloheteroalkyl, aryl, heteroaryl, or heterocyclyl;
R² is selected from the group consisting of hydrogen, halo, hydroxyl, alkyl, alkenyl, alkynyl, alkoxy, acyl, acyloxy, carboxylic acid, ester, amine, amide, carbonate, carbamate, cyano, nitro, thioether, thioester, cycloalkyl, cycloheteroalkyl, heterocyclyl, aryl, and heteroaryl, each of which is optionally substituted with halo, hydroxyl, sulfhydryl, nitro, nitroso, cyano, azido, alkyl, alkenyl, cycloalkyl, cycloheteroalkyl, heterocyclyl, aryl or heteroaryl;
each of R³ and R⁴ is independently selected from the group consisting of hydrogen, halo, hydroxyl, alkyl, alkenyl, alkynyl, alkoxy, cycloalkyl, and cycloheteroalkyl, any of which is optionally substituted with halo, hydroxyl, alkyl, or cycloalkyl; or R³ taken together with one or more atoms of R⁴ forms a cycloalkyl or cycloheteroalkyl, each of which is optionally substituted with halo, hydroxyl, alkyl, alkenyl, alkynyl, or alkoxy; and
R⁵ is selected from the group consisting of hydrogen, halo, hydroxyl, alkyl, alkenyl, alkynyl, alkoxy, acyl, acyloxy, carboxylic acid, ester, amine, amide, carbonate, carbamate, cyano, nitro, thioether, thioester, cycloalkyl, cycloheteroalkyl, heterocyclyl, aryl, and heteroaryl, each of which is optionally substituted with halo, hydroxyl, sulfhydryl, nitro, nitroso, cyano, azido, alkyl, alkenyl, cycloalkyl, cycloheteroalkyl, heterocyclyl, aryl or heteroaryl.

50-78. (canceled)

79. A composition comprising a compound of Formula I or a pharmaceutically acceptable salt thereof, 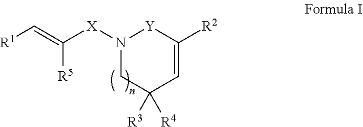
wherein n is an integer of 1, 2, or 3;
X or Y is independently C(O) or S(O)₂, wherein at least one of X or Y is S(O)₂;
R¹ is a 3-13 member heterocyclyl group having 1-5 heteroatom(s) independently selected from the group consisting of N, O, and S, wherein the 3-13 member heterocyclyl group is optionally substituted halo, alkyl, alkenyl, alkynyl, alkoxy, acyl, acyloxy, carboxylic acid, ester, amine, amide, carbonate, carbamate, carbonyl, sulfonyl, cyano, nitro, thioether, thioester, cycloalkyl, cycloheteroalkyl, aryl, heteroaryl, or heterocyclyl, any of which is unsubstituted or substituted with halo, hydroxyl, sulfhydryl, nitro, nitroso, cyano, azido, alkyl, alkenyl, cycloalkyl, cycloheteroalkyl, aryl, heteroaryl, or heterocyclyl;
R² is selected from the group consisting of hydrogen, halo, hydroxyl, alkyl, alkenyl, alkynyl, alkoxy, acyl, acyloxy, carboxylic acid, ester, amine, amide, carbonate, carbamate, cyano, nitro, thioether, thioester, cycloalkyl, cycloheteroalkyl, aryl, heteroaryl, or heterocyclyl, each of which is optionally substituted with halo, hydroxyl, sulfhydryl, nitro, nitroso, cyano, azido, alkyl, alkenyl, cycloalkyl, cycloheteroalkyl, aryl, heteroaryl, or heterocyclyl;
each of R³ and R⁴ is independently selected from the group consisting of hydrogen, halo, hydroxyl, alkyl, alkenyl, alkynyl, alkoxy, cycloalkyl, and cycloheteroalkyl, any of which is optionally substituted with halo, hydroxyl, alkyl, or cycloalkyl; or R³ taken together with one or more atoms of R⁴ forms a cycloalkyl or cycloheteroalkyl, each of which is optionally substituted with halo, hydroxyl, alkyl, alkenyl, alkynyl, or alkoxy; and
R⁵ is selected from the group consisting of hydrogen, halo, hydroxyl, alkyl, alkenyl, alkynyl, alkoxy, acyl, acyloxy, carboxylic acid, ester, amine, amide, carbonate, carbamate, cyano, nitro, thioether, thioester, cycloalkyl, cycloheteroalkyl, heterocyclyl, aryl, and heteroaryl, each of which is optionally substituted with halo, hydroxyl, sulfhydryl, nitro, nitroso, cyano, azido, alkyl, alkenyl, cycloalkyl, cycloheteroalkyl, heterocyclyl, aryl or heteroaryl,
and wherein at least one of R², R³ and R⁴ is not hydrogen.

80-99. (canceled)

100. The compound selected from the group consisting of:

| No. | Structure | Name |
|---|---|---|
| 1 | 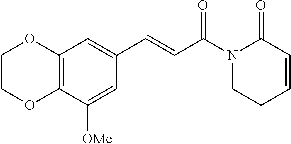 [SMILES: COc1cc(/C=C/C(=O)N2CCC=CC2=O)cc2c1OCCO2] | (E)-1-(3-(8-methoxy-2,3- dihydrobenzo[b][1,4]dioxin-6-yl)acryloyl)-5,6- dihydropyridin-2(1H)-one |
| 2 | 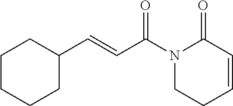 [SMILES: O=C1C=CCCN1C(=O)/C=C/C1CCCCC1] | (E)-1-(3-cyclohexylacryloyl)-5,6-dihydropyridin- 2(1H)-one |
| 3 | 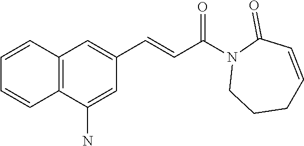 [SMILES: Nc1cc(/C=C/C(=O)N2CCCC=CC2=O)cc2ccccc12] | (E)-1-(3-(quinolin-3-yl)acryloyl)-1,5,6,7-tetrahydro- 2H-azepin-2-one |
| 4 | 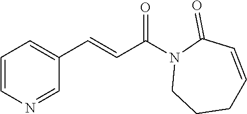 [SMILES: O=C1C=CCCCN1C(=O)/C=C/c1cccnc1] | (E)-1-(3-(pyridin-3-yl)acryloyl)-1,5,6,7-tetrahydro- 2H-azepin-2-one |
| 5 | 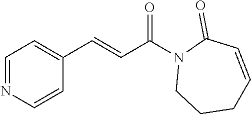 [SMILES: O=C1C=CCCCN1C(=O)/C=C/c1ccncc1] | (E)-1-(3-(pyridin-4-yl)acryloyl)-1,5,6,7-tetrahydro- 2H-azepin-2-one |
| 6 | 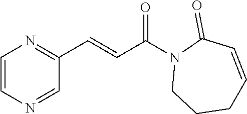 [SMILES: O=C1C=CCCCN1C(=O)/C=C/c1cnccn1] | (E)-1-(3-(pyrazin-2-yl)acryloyl)-1,5,6,7-tetrahydro- 2H-azepin-2-one |
| 7 | 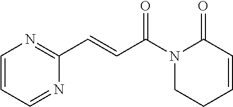 [SMILES: O=C1C=CCCN1C(=O)/C=C/c1ncccn1] | (E)-1-(3-(pyrimidin-2-yl)acryloyl)-5,6- dihydropyridin-2(1H)-one |
| 8 | 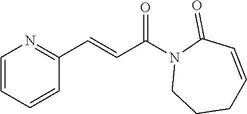 [SMILES: O=C1C=CCCCN1C(=O)/C=C/c1ccccn1] | (E)-1-(3-(pyridin-2-yl)acryloyl)-1,5,6,7-tetrahydro- 2H-azepin-2-one |
| 9 | 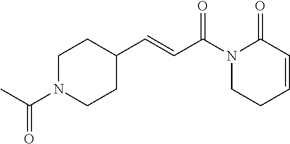 [SMILES: CC(=O)N1CCC(/C=C/C(=O)N2CCC=CC2=O)CC1] | (E)-1-(3-(1-acetylpiperidin-4-yl)acryloyl)-5,6- dihydropyridin-2(1H)-one |
| 10 | 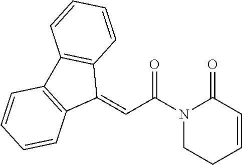 [SMILES: O=C1C=CCCN1C(=O)C=C1c2ccccc2-c2ccccc21] | 1-(2-(9H-fluoren-9-ylidene)acetyl)-5,6- dihydropyridin-2(1H)-one |
| 11 | 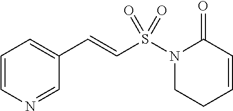 [SMILES: O=C1C=CCCN1S(=O)(=O)/C=C/c1cccnc1] | (E)-1-((2-(pyridin-3-yl)vinyl)sulfonyl)-5,6- dihydropyridin-2(1H)-one |
| 12 | 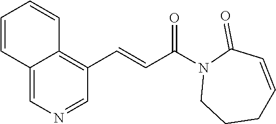 [SMILES: O=C1C=CCCCN1C(=O)/C=C/c1cncc2ccccc12] | (E)-1-(3-(isoquinolin-4-yl)acryloyl)-1,5,6,7- tetrahydro-2H-azepin-2-one |
| 13 | 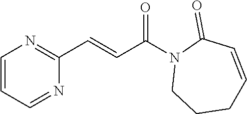 [SMILES: O=C1C=CCCCN1C(=O)/C=C/c1ncccn1] | (E)-1-(3-(pyrimidin-2-yl)acryloyl)-1,5,6,7- tetrahydro-2H-azepin-2-one |
| 14 | 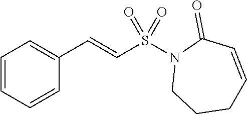 [SMILES: O=C1C=CCCCN1S(=O)(=O)/C=C/c1ccccc1] | (E)-1-(styrylsulfonyl)-1,5,6,7-tetrahydro-2H-azepin- 2-one |
| 15 | 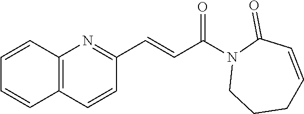 [SMILES: O=C1C=CCCCN1C(=O)/C=C/c1ccc2ccccc2n1] | (E)-1-(3-(quinolin-2-yl)acryloyl)-1,5,6,7-tetrahydro- 2H-azepin-2-one |
| 16 | 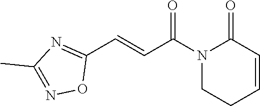 [SMILES: Cc1noc(/C=C/C(=O)N2CCC=CC2=O)n1] | (E)-1-(3-(3-methyl-1,2,4-oxadiazol-5-yl)acryloyl)- 5,6-dihydropyridin-2(1H)-one |
| 17 | 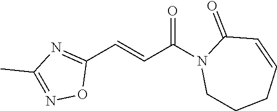 [SMILES: Cc1noc(/C=C/C(=O)N2CCCC=CC2=O)n1] | (E)-1-(3-(3-methyl-1,2,4-oxadiazol-5-yl)acryloyl)- 1,5,6,7-tetrahydro-2H-azepin-2-one |
| 18 | 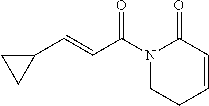 [SMILES: O=C1C=CCCN1C(=O)/C=C/C1CC1] | (E)-1-(3-cyclopropylacryloyl)-5,6-dihydropyridin- 2(1H)-one |
| 19 | 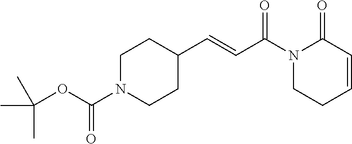 [SMILES: CC(C)(C)OC(=O)N1CCC(/C=C/C(=O)N2CCC=CC2=O)CC1] | tert-butyl (E)-4-(3-oxo-3-(6-oxo-3,6-dihydropyridin- 1(2H)-yl)prop-1-en-1-yl)piperidine-1-carboxylate |
| 20 | 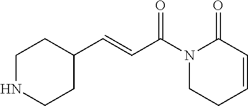 [SMILES: O=C1C=CCCN1C(=O)/C=C/C1CCNCC1] | (E)-1-(3-(piperidin-4-yl)acryloyl)-5,6- dihydropyridin-2(1H)-one |
| 21 | 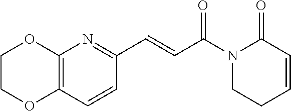 [SMILES: O=C1C=CCCN1C(=O)/C=C/c1ccc2c(n1)OCCO2] | (E)-1-(3-(2,3-dihydro-[1,4]dioxino[2,3-b]pyridin-6- yl)acryloyl)-5,6-dihydropyridin-2(1H)-one |
| 22 | 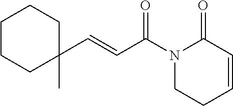 [SMILES: CC1(/C=C/C(=O)N2CCC=CC2=O)CCCCC1] | (E)-1-(3-(1-methylcyclohexyl)acryloyl)-5,6- dihydropyridin-2(1H)-one |
| 23 | 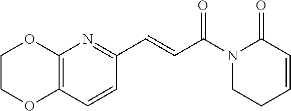 [SMILES: O=C1C=CCCN1C(=O)/C=C/c1ccc2c(n1)OCCO2] | (E)-1-(3-(2,3-dihydro-[1,4]dioxino[2,3-b]pyridin-6- yl)acryloyl)-5,6-dihydropyridin-2(1H)-one |
| 24 | 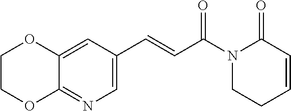 [SMILES: O=C1C=CCCN1C(=O)/C=C/c1cnc2c(c1)OCCO2] | (E)-1-(3-(2,3-dihydro-[1,4]dioxino[2,3-b]pyridin-7- yl)acryloyl)-5,6-dihydropyridin-2(1H)-one |
| 25 | 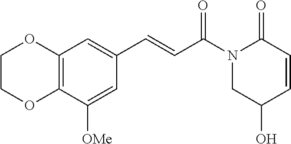 [SMILES: COc1cc(/C=C/C(=O)N2CC(O)C=CC2=O)cc2c1OCCO2] | 5-hydroxy-1-[(2E)-3-(8-methoxy-2,3-dihydro-1,4- benzodioxin-6-yl)prop-2-enoyl]-5,6-dihydropyridin- 2(1H)-one |
| 26 | 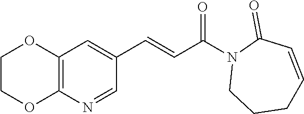 [SMILES: O=C1C=CCCCN1C(=O)/C=C/c1cnc2c(c1)OCCO2] | (E)-1-(3-(2,3-dihydro-[1,4]dioxino[2,3-b]pyridin-7- yl)acryloyl)-6,7-dihydro-1H-azepin-2(5H)-one |
| 27 | 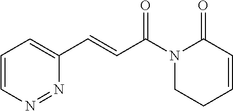 [SMILES: O=C1C=CCCN1C(=O)/C=C/c1cccnn1] | (E)-1-(3-(pyridazin-3-yl)acryloyl)-5,6- dihydropyridin-2(1H)-one |
| 28 | 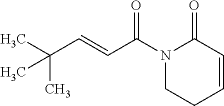 | (E)-1-(4,4-dimethylpent-2-enoyl)-5,6- dihydropyridin-2(1H)-one |
| 29 | 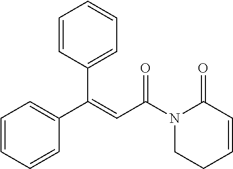 [SMILES: O=C1C=CCCN1C(=O)C=C(c1ccccc1)c1ccccc1] | 1-(3,3-diphenylacryloyl)-5,6-dihydropyridin-2(1H)- one |
| 30 | 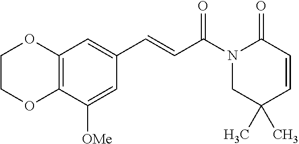 [SMILES: COc1cc(/C=C/C(=O)N2CC(C)(C)C=CC2=O)cc2c1OCCO2] | (E)-1-(3-(8-methoxy-2,3- dihydrobenzo[b][1,4]dioxin-6-yl)acryloyl)-5,5- dimethyl-5,6-dihydropyridin-2(1H)-one |
| 31 | 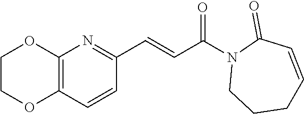 [SMILES: O=C1C=CCCCN1C(=O)/C=C/c1ccc2c(n1)OCCO2] | (E)-1-(3-(2,3-dihydro-[1,4]dioxino[2,3-b]pyridin-6- yl)acryloyl)-1,5,6,7-tetrahydroazepin-2-one |
| 32 | 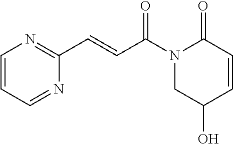 [SMILES: O=C1C=CC(O)CN1C(=O)/C=C/c1ncccn1] | 5-hydroxy-1-[(2E)-3-(pyrimidin-2-yl)prop-2-enoyl]- 5,6-dihydropyridin-2(1H)-one |
| 33 | 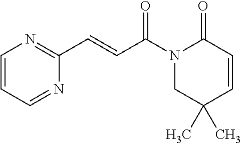 [SMILES: CC1(C)C=CC(=O)N(C(=O)/C=C/c2ncccn2)C1] | (E)-5,5-dimethyl-1-(3-(pyrimidin-2-yl)acryloyl)-5,6- dihydropyridin-2(1H)-one |
| 34 | 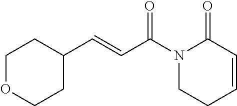 [SMILES: O=C1C=CCCN1C(=O)/C=C/C1CCOCC1] | 1-[(2E)-3-(tetrahydro-2H-pyran-4-yl)prop-2-enoyl]- 5,6-dihydropyridin-2(1H)-one |
| 35 | 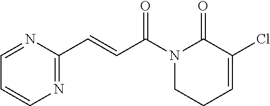 [SMILES: O=C(/C=C/c1ncccn1)N1CCC=C(Cl)C1=O] | (E)-3-chloro-1-(3-(pyrimidin-2-yl)acryloyl)-5,6- dihydropyridin-2(1H)-one |
| 36 | 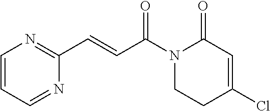 [SMILES: O=C1C=C(Cl)CCN1C(=O)/C=C/c1ncccn1] | (E)-4-chloro-1-(3-(pyrimidin-2-yl)acryloyl)-5,6- dihydropyridin-2(1H)-one |
| 37 | 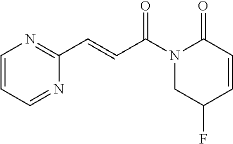 [SMILES: O=C1C=CC(F)CN1C(=O)/C=C/c1ncccn1] | (E)-5-fluoro-1-(3-(pyrimidin-2-yl)acryloyl)-5,6- dihydropyridin-2(1H)-one |
| 38 | 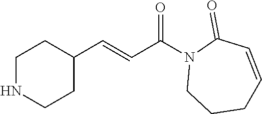 [SMILES: O=C1C=CCCCN1C(=O)/C=C/C1CCNCC1] | (E)-1-(3-(piperidin-4-yl)acryloyl)-6,7-dihydro-1H- azepin-2(5H)-one |
| 39 | 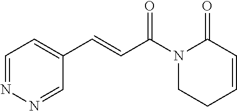 [SMILES: O=C1C=CCCN1C(=O)/C=C/c1ccnnc1] | (E)-1-(3-(pyridazin-4-yl)acryloyl)-5,6- dihydropyridin-2(1H)-one |
| 40 | 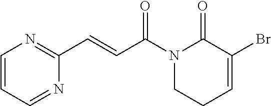 [SMILES: O=C(/C=C/c1ncccn1)N1CCC=C(Br)C1=O] | 3-bromo-1-[(2E)-3-(pyrimidin-2-yl)prop-2-enoyl]- 5,6-dihydropyridin-2(1H)-one |
| 41 | 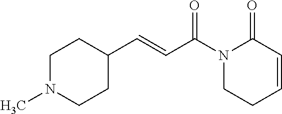 [SMILES: CN1CCC(/C=C/C(=O)N2CCC=CC2=O)CC1] | (E)-1-(3-(1-methylpiperidin-4-yl)acryloyl)-5,6- dihydropyridin-2(1H)-one |
| 42 | 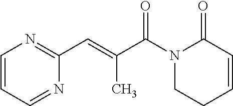 [SMILES: C/C(=C\c1ncccn1)C(=O)N1CCC=CC1=O] | (E)-1-(2-methyl-3-(pyrimidin-2-yl)acryloyl)-5,6- dihydropyridin-2(1H)-one |
| 43 | 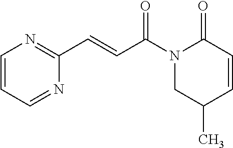 [SMILES: CC1C=CC(=O)N(C(=O)/C=C/c2ncccn2)C1] | (E)-5-methyl-1-(3-(pyrimidin-2-yl)acryloyl)-5,6- dihydropyridin-2(1H)-one |
| 44 | 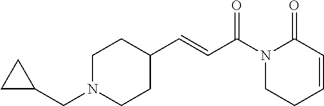 [SMILES: O=C1C=CCCN1C(=O)/C=C/C1CCN(CC2CC2)CC1] | (E)-1-(3-(1-(cyclopropylmethyl)piperidin-4- yl)acryloyl)-5,6-dihydropyridin-2(1H)-one |
| 45 | 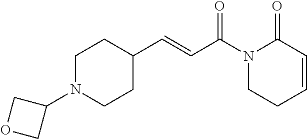 [SMILES: O=C1C=CCCN1C(=O)/C=C/C1CCN(C2COC2)CC1] | (E)-1-(3-(1-(oxetan-3-yl)piperidin-4-yl)acryloyl)-5,6- dihydropyridin-2(1H)-one |
| 46 | 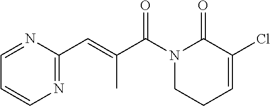 [SMILES: C/C(=C\c1ncccn1)C(=O)N1CCC=C(Cl)C1=O] | (E)-3-chloro-1-(2-methyl-3-(pyrimidin-2- yl)acryloyl)-5,6-dihydropyridin-2(1H)-one |
| 47 | 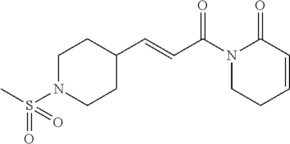 [SMILES: CS(=O)(=O)N1CCC(/C=C/C(=O)N2CCC=CC2=O)CC1] | 1-{(2E)-3-[1-(methylsulfonyl)piperidin-4-yl]prop-2- enoyl}-5,6-dihydropyridin-2(1H)-one |
| 48 | 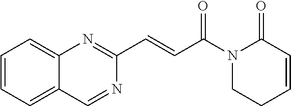 [SMILES: O=C1C=CCCN1C(=O)/C=C/c1ncc2ccccc2n1] | (E)-1-(3-(quinazolin-2-yl)acryloyl)-5,6- dihydropyridin-2(1H)-one |
| 49 | 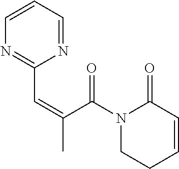 [SMILES: C/C(=C/c1ncccn1)C(=O)N1CCC=CC1=O] | 1-[(2E)-3-(4,5-dihydropyrimidin-2-yl)-2-methylprop- 2-enoyl]-5,6-dihydropyridin-2(1H)-one |
| 50 | 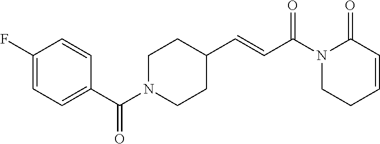 [SMILES: O=C(c1ccc(F)cc1)N1CCC(/C=C/C(=O)N2CCC=CC2=O)CC1] | (E)-1-(3-(1-(4-fluorobenzoyl)piperidin-4- yl)acryloyl)-5,6-dihydropyridin-2(1H)-one |
| 51 | 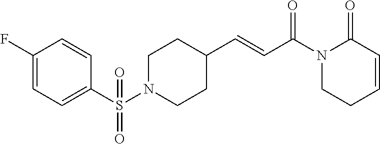 [SMILES: O=C1C=CCCN1C(=O)/C=C/C1CCN(S(=O)(=O)c2ccc(F)cc2)CC1] | (E)-1-(3-(1-(4-fluorophenylsulfonyl)piperidin-4- yl)acryloyl)-5,6-dihydropyridin-2(1H)-one |
| 52 | 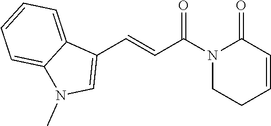 [SMILES: Cn1cc(/C=C/C(=O)N2CCC=CC2=O)c2ccccc21] | 1-[(2E)-3-(1-methyl-1H-indol-3-yl)prop-2-enoyl]- 5,6-dihydropyridin-2(1H)-one |
| 53 | 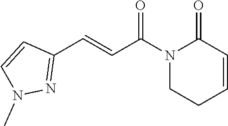 [SMILES: Cn1ccc(/C=C/C(=O)N2CCC=CC2=O)n1] | 1-[(2E)-3-(1-methyl-1H-pyrazol-3-yl)prop-2-enoyl]- 5,6-dihydropyridin-2(1H)-one |
| 54 | 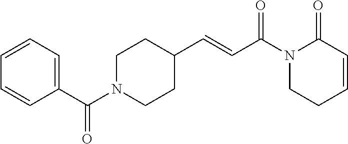 [SMILES: O=C(c1ccccc1)N1CCC(/C=C/C(=O)N2CCC=CC2=O)CC1] | 1-[(2E)-3-(1-benzoylpiperidin-4-yl)prop-2-enoyl]- 5,6-dihydropyridin-2(1H)-one |
| 55 | 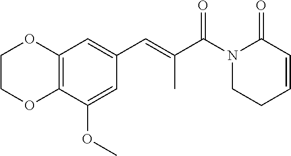 [SMILES: COc1cc(/C=C(\C)C(=O)N2CCC=CC2=O)cc2c1OCCO2] | 1-[(2E)-3-(8-methoxy-2,3-dihydro-1,4-benzodioxin- 6-yl)-2-methylprop-2-enoyl]-5,6-dihydropyridin- 2(1H)-one |
| 56 | 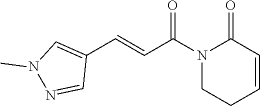 [SMILES: Cn1cc(/C=C/C(=O)N2CCC=CC2=O)cn1] | (E)-1-(3-(1-methyl-1H-pyrazol-4-yl)acryloyl)-5,6- dihydropyridin-2(1H)-one |
| 57 | 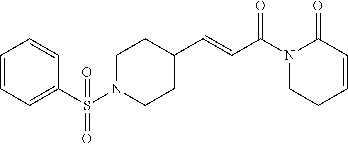 [SMILES: O=C1C=CCCN1C(=O)/C=C/C1CCN(S(=O)(=O)c2ccccc2)CC1] | (E)-1-(3-(1-(phenylsulfonyl)piperidin-4-yl)acryloyl)- 5,6-dihydropyridin-2(1H)-one |
| 58 | 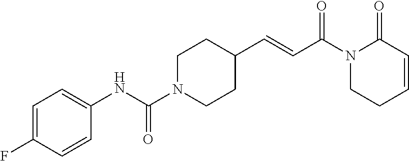 [SMILES: O=C(Nc1ccc(F)cc1)N1CCC(/C=C/C(=O)N2CCC=CC2=O)CC1] | N-(4-fluorophenyl)-4-[(1E)-3-oxo-3-(6-oxo-3,6- dihydropyridin-1(2H)-yl)prop-1-en-1-yl]piperidine- 1-carboxamide |
| 59 | 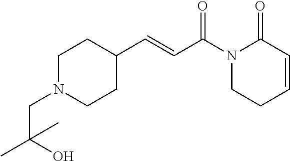 [SMILES: CC(C)(O)CN1CCC(/C=C/C(=O)N2CCC=CC2=O)CC1] | 1-{(2E)-3-[1-(2-hydroxy-2-methylpropyl)piperidin- 4-yl]prop-2-enoyl}-5,6-dihydropyridin-2(1H)-one |
| 60 | 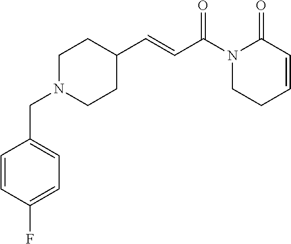 [SMILES: O=C1C=CCCN1C(=O)/C=C/C1CCN(Cc2ccc(F)cc2)CC1] | 1-{3-[1-(4-fluorobenzyl)piperidin-4-yl]prop-2- enoyl}-5,6-dihydropyridin-2(1H)-one |
| 61 | 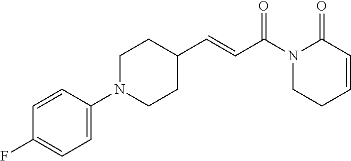 [SMILES: O=C1C=CCCN1C(=O)/C=C/C1CCN(c2ccc(F)cc2)CC1] | 1-{3-[1-(4-fluorophenyl)piperidin-4-yl]prop-2- enoyl}-5,6-dihydropyridin-2(1H)-one |
| 62 | 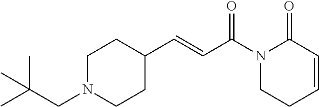 [SMILES: CC(C)(C)CN1CCC(/C=C/C(=O)N2CCC=CC2=O)CC1] | (E)-1-(3-(1-neopentylpiperidin-4-yl)acryloyl)-5,6- dihydropyridin-2(1H)-one |
| 63 | 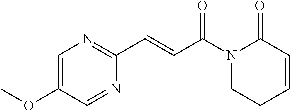 [SMILES: COc1cnc(/C=C/C(=O)N2CCC=CC2=O)nc1] | (E)-1-(3-(5-methoxypyrimidin-2-yl)acryloyl)-5,6- dihydropyridin-2(1H)-one |
| 64 | 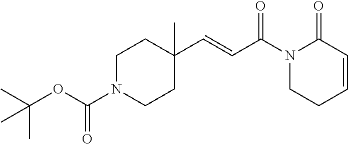 [SMILES: CC1(/C=C/C(=O)N2CCC=CC2=O)CCN(C(=O)OC(C)(C)C)CC1] | tert-butyl (E)-4-methyl-4-(3-oxo-3-(6-oxo-3,6- dihydropyridin-1(2H)-yl)prop-1-en-1-yl)piperidine- 1-carboxylate |
| 65 | 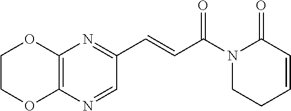 [SMILES: O=C1C=CCCN1C(=O)/C=C/c1cnc2c(n1)OCCO2] | (E)-1-(3-(2,3-dihydro-[1,4]dioxino[2,3-b]pyrazin-6- yl)acryloyl)-5,6-dihydropyridin-2(1H)-one |
| 66 | 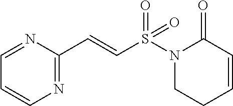 [SMILES: O=C1C=CCCN1S(=O)(=O)/C=C/c1ncccn1] | 1-{[(E)-2-(pyrimidin-2-yl)ethenyl]sulfonyl}-5,6- dihydropyridin-2(1H)-one |
| 67 | 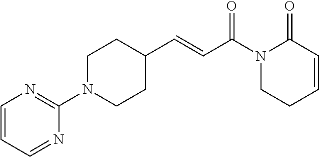 [SMILES: O=C1C=CCCN1C(=O)/C=C/C1CCN(c2ncccn2)CC1] | 1-{(2E)-3-[1-(pyrimidin-2-yl)piperidin-4- yl]prop-2-enoyl}-5,6-dihydropyridin-2(1H)- one |
| 68 | 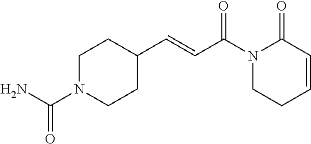 [SMILES: NC(=O)N1CCC(/C=C/C(=O)N2CCC=CC2=O)CC1] | (E)-4-(3-oxo-3-(6-oxo-3,6-dihydropyridin-1(2H)- yl)prop-1-en-1-yl)piperidine-1-carboxamide |
| 69 | 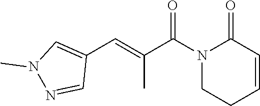 [SMILES: C/C(=C\c1cnn(C)c1)C(=O)N1CCC=CC1=O] | 1-[(2E)-2-methyl-3-(1-methyl-1H-pyrazol-4-yl)prop- 2-enoyl]-5,6-dihydropyridin-2(1H)-one |
| 70 | 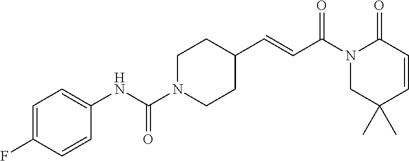 [SMILES: CC1(C)C=CC(=O)N(C(=O)/C=C/C2CCN(C(=O)Nc3ccc(F)cc3)CC2)C1] | (E)-4-(3-(3,3-dimethyl-6-oxo-3,6-dihydropyridin- 1(2H)-yl)-3-oxoprop-1-en-1-yl)-N-(4- fluorophenyl)piperidine-1-carboxamide |
| 71 | 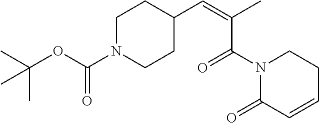 [SMILES: C/C(=C/C1CCN(C(=O)OC(C)(C)C)CC1)C(=O)N1CCC=CC1=O] | (Z)-tert-butyl 4-(2-methyl-3-oxo-3-(2-oxo-5,6- dihydropyridin-1(2H)-yl)prop-1-enyl)piperidine-1- carboxylate |
| 72 | 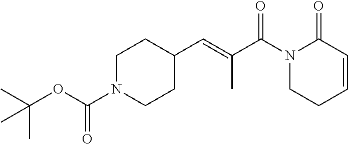 [SMILES: C/C(=C\C1CCN(C(=O)OC(C)(C)C)CC1)C(=O)N1CCC=CC1=O] | tert-butyl (E)-4-(2-methyl-3-oxo-3-(6-oxo-3,6- dihydropyridin-1(2H)-yl)prop-1-en-1-yl)piperidine- 1-carboxylate |
| 73 | 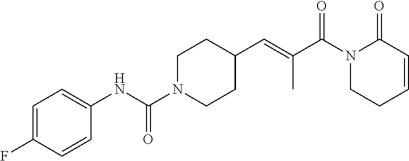 [SMILES: C/C(=C\C1CCN(C(=O)Nc2ccc(F)cc2)CC1)C(=O)N1CCC=CC1=O] | (E)-N-(4-fluorophenyl)-4-(2-methyl-3-oxo-3-(6-oxo- 3,6-dihydropyridin-1(2H)-yl)prop-1-en-1- yl)piperidine-1-carboxamide |
| 74 | 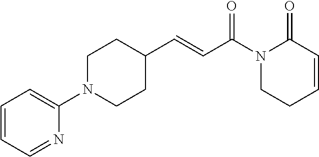 [SMILES: O=C1C=CCCN1C(=O)/C=C/C1CCN(c2ccccn2)CC1] | (E)-1-(3-(1-(pyridin-2-yl)piperidin-4-yl)acryloyl)- 5,6-dihydropyridin-2(1H)-one |
| 75 | 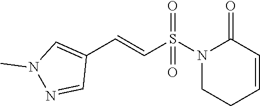 [SMILES: Cn1cc(/C=C/S(=O)(=O)N2CCC=CC2=O)cn1] | 1-{[(E)-2-(1-methyl-1H-pyrazol-4- yl)ethenyl]sulfonyl}-5,6-dihydropyridin-2(1H)-one |
| 76 | 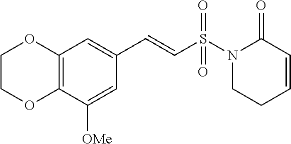 [SMILES: COc1cc(/C=C/S(=O)(=O)N2CCC=CC2=O)cc2c1OCCO2] | 1-{[(E)-2-(8-methoxy-2,3-dihydro-1,4-benzodioxin- 6-yl)ethenyl]sulfonyl}-5,6-dihydropyridin-2(1H)-one |
| 77 | 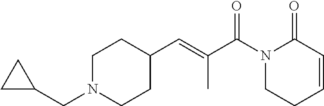 [SMILES: C/C(=C\C1CCN(CC2CC2)CC1)C(=O)N1CCC=CC1=O] | (E)-1-(3-(1-(cyclopropylmethyl)piperidin-4-yl)-2- methylacryloyl)-5,6-dihydropyridin-2(1H)-one |
| 78 | 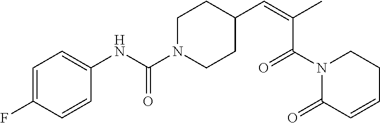 [SMILES: C/C(=C/C1CCN(C(=O)Nc2ccc(F)cc2)CC1)C(=O)N1CCC=CC1=O] | (Z)-1-(3-(1-(4-fluorophenyl)piperidin-4-yl)-2- methylacryloyl)-5,6-dihydropyridin-2(1H)-one |
| 79 | 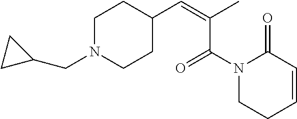 [SMILES: C/C(=C/C1CCN(CC2CC2)CC1)C(=O)N1CCC=CC1=O] | (Z)-1-(3-(1-(cyclopropylmethyl)piperidin-4-yl)-2- methylacryloyl)-5,6-dihydropyridin-2(1H)-one |
| 80 | 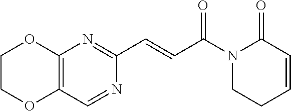 [SMILES: O=C1C=CCCN1C(=O)/C=C/c1ncc2c(n1)OCCO2] | (E)-1-(3-(6,7-dihydro-[1,4]dioxino[2,3-d]pyrimidin- 2-yl)acryloyl)-5,6-dihydropyridin-2(1H)-one |
| 81 | 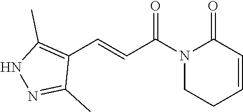 [SMILES: Cc1n[nH]c(C)c1/C=C/C(=O)N1CCC=CC1=O] | (E)-1-(3-(3,5-dimethyl-1H-pyrazol-4-yl)acryloyl)- 5,6-dihydropyridin-2(1H)-one |
| 82 | 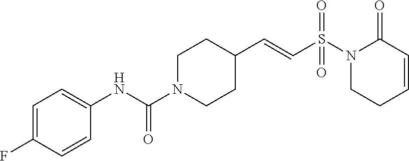 [SMILES: O=C(Nc1ccc(F)cc1)N1CCC(/C=C/S(=O)(=O)N2CCC=CC2=O)CC1] | (E)-N-(4-fluorophenyl)-4-(2-((6-oxo-3,6- dihydropyridin-1(2H)-yl)sulfonyl)vinyl)piperidine-1- carboxamide |
| 83 | 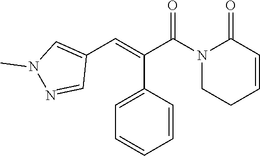 [SMILES: Cn1cc(/C=C(/C(=O)N2CCC=CC2=O)c2ccccc2)cn1] | 1-[(2E)-3-(1-methyl-1H-pyrazol-4-yl)-2-phenylprop- 2-enoyl]-5,6-dihydropyridin-2(1H)-one |
| 84 | 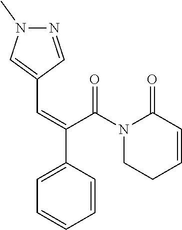 [SMILES: Cn1cc(/C=C(\C(=O)N2CCC=CC2=O)c2ccccc2)cn1] | (Z)-1-(3-(1-methyl-1H-pyrazol-4-yl)-2- phenylacryloyl)-5,6-dihydropyridin-2(1H)-one |
| 85 | 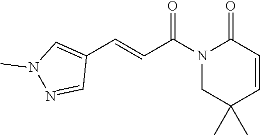 [SMILES: Cn1cc(/C=C/C(=O)N2CC(C)(C)C=CC2=O)cn1] | (E)-5,5-dimethyl-1-(3-(1-methyl-1H-pyrazol-4- yl)acryloyl)-5,6-dihydropyridin-2(1H)-one |
| 86 | 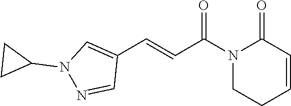 [SMILES: O=C1C=CCCN1C(=O)/C=C/c1cnn(C2CC2)c1] | (E)-1-(3-(1-cyclopropyl-1H-pyrazol-4-yl)acryloyl)- 5,6-dihydropyridin-2(1H)-one |
| 87 | 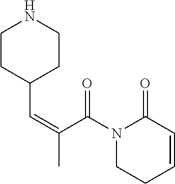 [SMILES: C/C(=C/C1CCNCC1)C(=O)N1CCC=CC1=O] | (Z)-1-(2-methyl-3-(piperidin-4-yl)acryloyl)-5,6- dihydropyridin-2(1H)-one 2,2,2-trifluoroacetate |
| 88 | 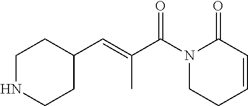 [SMILES: C/C(=C\C1CCNCC1)C(=O)N1CCC=CC1=O] | (E)-1-(2-methyl-3-(piperidin-4-yl)acryloyl)-5,6- dihydropyridin-2(1H)-one 2,2,2-trifluoroacetate |
| 89 | 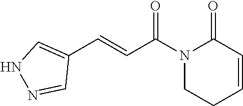 [SMILES: O=C1C=CCCN1C(=O)/C=C/c1cn[nH]c1] | (E)-1-(3-(1H-pyrazol-4-yl)acryloyl)-5,6- dihydropyridin-2(1H)-one |
| 90 | 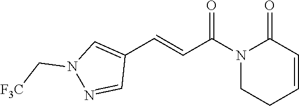 [SMILES: O=C1C=CCCN1C(=O)/C=C/c1cnn(CC(F)(F)F)c1] | 1 11-{(2E)-3-[1-(2,2,2-trifluoroethyl)-1H-pyrazol-4- yl]prop-2-enoyl}-5,6-dihydropyridin-2(1H)-one |
| 91 | 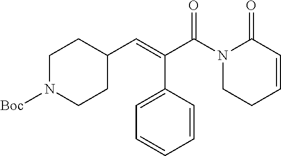 [SMILES: CC(C)(C)OC(=O)N1CCC(/C=C(/C(=O)N2CCC=CC2=O)c2ccccc2)CC1] | (E)-tert-butyl 4-(3-oxo-3-(2-oxo-5,6-dihydropyridin- 1(2H)-yl)-2-phenylprop-1-enyl)piperidine-1- carboxylate |
| 92 | 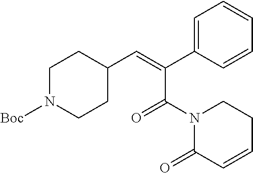 [SMILES: CC(C)(C)OC(=O)N1CCC(/C=C(\C(=O)N2CCC=CC2=O)c2ccccc2)CC1] | (Z)-tert-butyl 4-(3-oxo-3-(2-oxo-5,6-dihydropyridin- 1(2H)-yl)-2-phenylprop-1-enyl)piperidine-1- carboxylate |
| 93 | 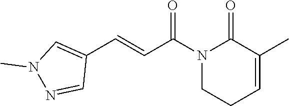 | (E)-3-methyl-1-(3-(1-methyl-1H-pyrazol-4- yl)acryloyl)-5,6-dihydropyridin-2(1H)-one |
| 94 | 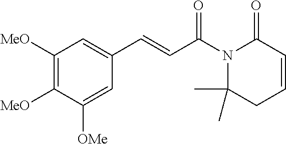 [SMILES: COc1cc(/C=C/C(=O)N2C(=O)C=CCC2(C)C)cc(OC)c1OC] | (E)-6,6-dimethyl-1-(3-(3,4,5- trimethoxyphenyl)acryloyl)-5,6-dihydropyridin- 2(1H)-one |
| 95 | 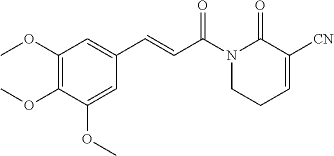 [SMILES: COc1cc(/C=C/C(=O)N2CCC=C(C#N)C2=O)cc(OC)c1OC] | (E)-2-oxo-1-(3-(3,4,5-trimethoxyphenyl)acryloyl)- 1,2,5,6-tetrahydropyridine-3-carbonitrile |
| 96 | 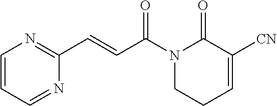 [SMILES: N#CC1=CCCN(C(=O)/C=C/c2ncccn2)C1=O] | (E)-2-oxo-1-(3-(pyrimidin-2-yl)acryloyl)-1,2,5,6- tetrahydropyridine-3-carbonitrile |
| 97 | 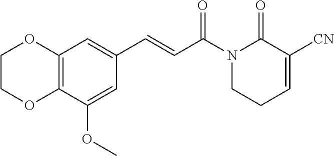 [SMILES: COc1cc(/C=C/C(=O)N2CCC=C(C#N)C2=O)cc2c1OCCO2] | (E)-1-(3-(8-methoxy-2,3- dihydrobenzo[b][1,4]dioxin-6-yl)acryloyl)-2-oxo- 1,2,5,6-tetrahydropyridine-3-carbonitrile |
| 98 | 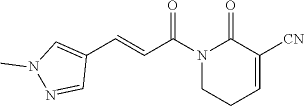 [SMILES: Cn1cc(/C=C/C(=O)N2CCC=C(C#N)C2=O)cn1] | (E)-1-(3-(1-methyl-1H-pyrazol-4-yl)acryloyl)-2-oxo- 1,2,5,6-tetrahydropyridine-3-carbonitrile |

or a pharmaceutically acceptable salt thereof.

101. A pharmaceutical composition comprising a composition of claim 1, in combination with a pharmaceutically acceptable carrier, diluent or excipient.

102. A method of treating an autoimmune or inflammatory disease in a subject in need thereof, the method comprising administering to the subject a therapeutically effective amount of a composition of claim 1.

103. A method of reducing inflammation in a subject in need thereof, the method comprising administering to the subject a therapeutically effective amount of a composition of claim 1.

104-119. (canceled)

120. A method of treating a subject having cancer, the method comprising administering to said subject a therapeutically effective amount of a compound according to claim 1, or a pharmaceutically acceptable salt thereof, wherein said cancer is selected from the group of consisting of lymphoma, osteosarcoma, melanoma, breast cancer, renal cancer, prostate cancer, colorectal cancer, thyroid cancer, ovarian cancer, pancreatic cancer, neuronal cancer, lung cancer, uterine cancer, and gastrointestinal cancer.
